(19) United States
(12) Patent Application Publication
(10) Pub. No.: US 2011/0190267 A1
(43) Pub. Date: Aug. 4, 2011

(54) PRODRUGS OF OPIOIDS AND USES THEREOF

(75) Inventors: Richard Franklin, Fleet (GB); Karl Swift, Gloucestershire (GB); Bernard T. Golding, Newcastle Upon Tyne (GB); Robert G. Tyson, Durham (GB)

(73) Assignee: Shire Pharmaceuticals, Inc., Florence, KY (US)

(21) Appl. No.: 12/978,210

(22) Filed: Dec. 23, 2010

Related U.S. Application Data
(60) Provisional application No. 61/292,362, filed on Jan. 5, 2010.

(51) Int. Cl. A61K 31/55; C07D 223/04; C07D 401/12; C07D 489/02; A61K 31/439; C07C 271/58; A61K 31/27; C07D 221/28; C07D 211/34; A61K 31/451; A61P 25/04

(52) U.S. Cl. 514/212.01; 540/611; 540/597; 514/217.04; 546/44; 514/282; 560/29; 514/488; 546/74; 514/289; 546/234; 514/331

(57) ABSTRACT

The present invention concerns prodrugs of opioid analgesics and pharmaceutical compositions containing such prodrugs. Methods for providing more consistent pain relief by increasing the bioavailability of the opioid analgesic with the aforementioned prodrugs are provided. The invention also provides for decreasing the adverse GI side effects of opioid analgesics.

This application claims priority to U.S. provisional application No. 61/292,362, filed Jan. 5, 2010, the contents of which are hereby incorporated by reference in their entirety.

### FIELD OF THE INVENTION

The present invention relates to opioid prodrugs, their synthesis and use, and other subject matter. The invention provides amongst other things opioid prodrugs which aim to improve the opioid's systemic availability and/or minimize the adverse gastrointestinal (GI) side-effects associated with the administration of the parent compound.

### BACKGROUND OF THE INVENTION

Appropriate treatment of pain continues to represent a major challenge for both patients and healthcare professionals. Optimal pharmacologic management of pain requires selection of the appropriate analgesic drug that achieves rapid efficacy with minimal side effects. Opioid analgesics offer perhaps the most important option in the treatment of nociceptive pain and remain the gold standard of treatment.

A major shortcoming of many of the opioids is that they suffer from poor oral bioavailability due to first pass glucuronidation of the commonly present phenolic function. This has been shown, for example, with oxymorphone (Sloan et al. (2005). Supp Care Cancer 13, 57-65), meptazinol (Norbury et al. (1983). Eur J Clin Pharmacol 25, 77-80) and buprenorphine (Kintz and Marquet (2002). pp 1-11 in Buprenorphine Therapy in Opiate Addiction, Humana press). Such poor oral bioavailability results in variable blood levels of the respective opioid, and therefore, variable patient response—a highly undesirable feature in the treatment of pain where rapid and reliable relief is demanded.

Various types of prodrugs have historically been proposed to minimize first pass metabolism and so improve the oral bioavailability of opioids. These have included simple ester conjugates which are frequently hydrolyzed by plasma esterases extremely quickly. Such rapid hydrolysis by plasma esterases limits the utility of ester linked prodrugs and denies the necessary transient protection of the opioid against first past metabolism.

The rapidity of hydrolysis of ester conjugates is illustrated by work on the morphine ester prodrug morphine-3-propionate. Morphine has a poor oral bioavailability due to extensive first pass glucuronidation at the 3 and the 6 positions, resulting in much inter and intra subject variability in analgesic response after an oral dose of the drug (Hoskin (1989). Br. J. Clin Pharmacol 27, 499-505). The plasma and tissue stability of the 3-propionate prodrug was investigated, and it was found to be hydrolyzed in human plasma with a half-life of less than 5 minutes (Goth et al. (1997). International Journal of Pharmaceutics 154, 149-155).

Meptazinol is another opioid with poor oral bioavailability (<10%). The low oral bioavailability has been attributed to high first pass glucuronidation (Norbury et al. (1983) Eur. J. Clin. Pharmacol. 25, 77-80). Attempts have been made to overcome this problem by the use of ester linked meptazinol prodrugs (Lu et al. (2005). Biorg. and Med. Chem. Letters 15, 2607-2609 and Xie et al. (2005). Biorg. and Med. Chem. Letters 15, 493-4956). However, only one of these prodrugs—((Z)-3-[2-(propionyloxy)phenyl]-2-propenoic ester) showed a significant increase in bioavailability over meptazinol itself, when tested in a rat model. However, to the Applicants knowledge, no further data has been published on this prodrug. These workers did subsequently publish on the utility of a phenyl carbamate derivative of (−) meptazinol but only with the aim of increasing the inherent in vitro potency of the compounds as an inhibitor of acetyl choline esterase and not as a prodrug (Chou Z et al (2007) Chinese Patent Application Number 200710038209.7).

An alternative strategy for creating a prodrug from the hydroxylic/phenolic function present in the opioids is the formation of O-alkyl (alkyl ether) or aryl ether conjugates. However, such derivatives appear to be very resistant to hydrolysis and metabolic activation. This is best illustrated by the 3-methyl ether prodrug of morphine—codeine. While codeine was not originally developed as a prodrug of morphine, it was subsequently found to give rise to small quantities of morphine. It has been estimated that less than 5% of an oral dose of codeine is converted to morphine—reflecting the slowness with which O-dealkylation takes place (Vree et al. (1992). Biopharma Drug Dispos. 13, 445-460 and Quiding et al. (1993). Eur. J. Clin. Pharmacol. 44, 319-323). The same phenomenon was observed for the corresponding dihydromorphine prodrug—dihydrocodeine, with less than 2% of an oral dose of dihydrocodeine being converted to dihydromorphine (Balikova et al. (2001). J. Chromatog. Biomed. Sci. Appl. 752, 179-186).

A further disadvantage of the O-alkyl ether prodrugging strategy is that the dealkylation of these opioids is effected by cytochrome P450 2D6 (Cyp2D6), a polymorphically expressed enzyme (Schmidt et al. (2003). Int. J. Clin. Pharmacol. Ther. 41, 95-106). This polymorphic enzyme expression inevitably results in substantial variation in patient exposure to the respective active metabolite (e.g., morphine and dihydromorphine). For example, low/negligible exposure to morphine derived from codeine has been reported amongst a large group of patients deficient in Cyp2D6 activity, potentially impacting the analgesic efficacy of codeine (Poulsen et al. (1998). Eur. Clin. Pharmacol. 54, 451-454).

An ideal prodrug moiety and linkage for a particular opioid would afford theoptimal balance of protection against first pass metabolism and susbquent efficient release of the active drug. There therefore remains a real need in the treatment of severe pain with opioids for products which retain all the inherent pharmacological advantages of the opioids, but which avoid or reduce their principal limitations of (1) low and erratic systemic availability after oral dosing and (2) induction of adverse GI side effects, including emesis and chronic constipation.

### SUMMARY OF THE INVENTION

According to one aspect, the present invention provides a method of treating a disorder in a subject in need thereof with an opioid. The method comprises orally administering a therapeutically effective amount (e.g., an analgesically effective amount) of an opioid prodrug or a pharmaceutically acceptable salt thereof to the subject, wherein the opioid prodrug comprises an opioid analgesic covalently bonded via a carbamate or thiocarbamate linkage, preferably via a carbamate linkage, to an amino benzoic acid (ABA) or an analogue thereof. The disorder may be one treatable with an opioid. For example, the disorder may be pain, e.g. neuropathic pain or nociceptive pain.

According to another aspect, the present invention provides a method for increasing the oral bioavailability of an opioid analgesic which has a significantly lower bioavailability when administered in its underivatized form. The method comprises administering, to a subject in need thereof, an opioid prodrug or a pharmaceutically acceptable salt thereof to a subject in need thereof, wherein the opioid prodrug is comprised of an opioid analgesic covalently bonded via a carbamate or thiocarbamate linkage, preferably a carbamate linkage, to amino benzoic acid (ABA) or an analogue thereof. In one embodiment, upon oral administration, the oral bioavailability of the opioid derived from the prodrug is at least 200% greater than that of the opioid, when administered in its underivatized form. The amount of the opioid administered is preferably a therapeutically effective amount (e.g., an analgesic effective amount).

According to another aspect, the present invention provides a method for reducing the inter- or intra-subject variability of an opioid's plasma levels. The method comprises administering to a subject, or group of subjects, in need thereof, a therapeutically effective amount (e.g., an analgesic effective amount) of an opioid prodrug or a pharmaceutically acceptable salt thereof, wherein the prodrug comprises an opioid analgesic covalently bonded via a carbamate or thiocarbamate, preferably a carbamate linkage, linkage to PABA or an analogue thereof.

According to another aspect, the present invention provides a method for minimizing the gastrointestinal side effects normally associated with administration of an opioid analgesic. The method comprises orally administering an opioid prodrug or a pharmaceutically acceptable salt thereof to a subject in need thereof, wherein the opioid prodrug is comprised of an opioid analgesic covalently bonded via a carbamate or thiocarbamate, preferably a carbamate linkage, linkage to an amino benzoic acid (ABA) or an analogue thereof. Upon oral administration, the prodrug or pharmaceutically acceptable salt minimizes, if not completely then partially avoids, the gastrointestinal side effects usually seen after oral administration of the unbound opioid analgesic. The amount of the opioid prodrug administered is preferably a therapeutically effective amount (e.g., an analgesic effective amount). In one embodiment, the opioid is meptazinol.

According to another aspect, the present invention provides an opioid prodrug of Formula I:

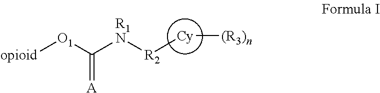

or pharmaceutically acceptable salt thereof, wherein,

opioid is an opioid with a hydroxylic or oxo oxygen (an enolisable carbonyl group), or an active metabolite thereof;

O₁ is a hydroxylic oxygen atom or an oxo oxygen atom (the oxo oxygen atom of an enolisable carbonyl group) present in the unbound opioid molecule;

A is ═O or ═S;

R₁ is selected from hydrogen, alkyl and substituted alkyl;

R₂ is absent or a C₁-C₃ n-alkyl, which is optionally substituted one or more groups selected from

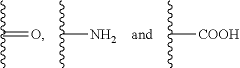

(or salts or C₁-C₆ alkyl esters thereof);

Cy is a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring;

n is 1, 2, or 3;

one occurrence of R₃ is independently selected from

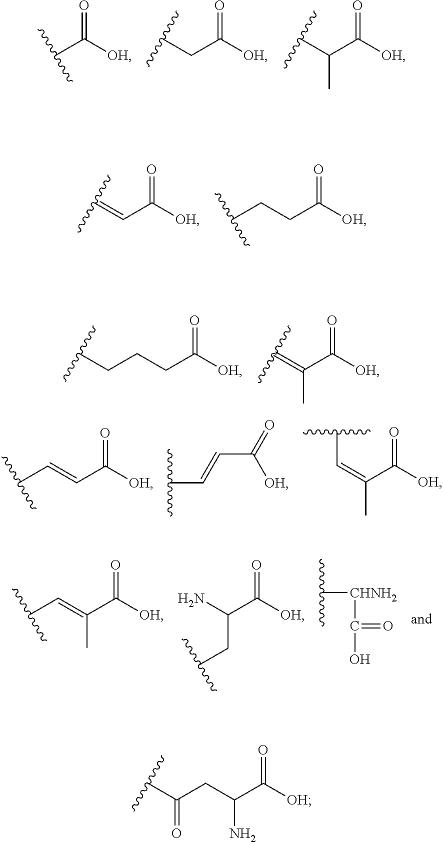

and further occurrences of R₃ are further selected from halogen, C₁-C₆ alkyl, substituted C₁-C₆ alkyl, C₁-C₆ alkyl esters, substituted C₁-C₆ alkyl esters,

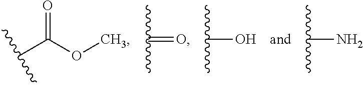

(i.e., further occurrences of R₃ are selected from the group provided for the first occurrence and this additional group).

According to another aspect, the present invention provides to an opioid prodrug of Formula I(A):

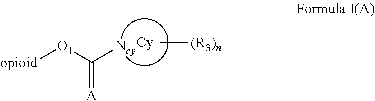

or pharmaceutically acceptable salt thereof, wherein,

opioid, O₁, A, R₃, Cy and n are defined as provided for Formula I; and

N_{cy} is a nitrogen atom present in the Cy group.

According to another aspect, the present invention provides opioid prodrugs of Formula I(B):

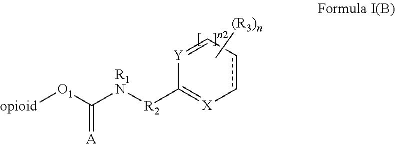

or pharmaceutically acceptable salt thereof, wherein,

opioid, n, A, O₁, R₁, R₂ and R₃ are as defined in Formula I;

the dashed bond “ - - - ” refers to an optional bond;

n2 is 0 or 1; and

X and Y are independently selected from N, S, O, and C, wherein any valency of said N, S, O or C atom which is not bonded to a neighbouring ring atom is bonded to H or an R₃.

The opioid drug is covalently bonded to the rest of the prodrug at a hydroxyl group via a carbamate linkage. In an embodiment, the opioid drug is covalently bonded to the rest of the prodrug at a phenolic hydroxyl group via a carbonate linkage.

According to another aspect, the present invention provides an opioid prodrug having a structure according to Formula (II):

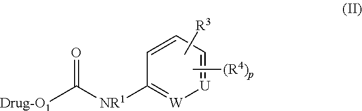

or a pharmaceutically acceptable salt thereof, wherein:

the term “Drug-O₁” is an opioid drug having a phenolic hydroxyl residue and O₁ is said phenolic hydroxyl residue of the opioid;

R³ is selected from the group consisting of: —(CR′R″)ᵣCOOH and

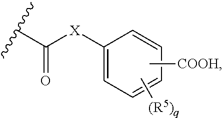

wherein X is —O— or —NR⁶— and wherein R′ and R″ are each independently selected from the group consisting of: H, hydroxy, carboxy, carboxamido, imino, alkanoyl, cyano, cyanomethyl, nitro, amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

R¹ and R⁶ are each independently selected from the group consisting of: H, C₁₋₄ alkyl (e.g. methyl, ethyl or propyl), C₁₋₄ haloalkyl (e.g. trifluoromethyl), C₁₋₄ alkoxy (e.g. methoxy, ethoxy or propoxy), C₁₋₄ haloalkoxy (e.g. trifluoromethoxy);

R⁴ and R⁵ are each independently selected from the group consisting of: hydroxy, carboxy, carboxamido, imino, alkanoyl, cyano, cyanomethyl, nitro, amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

W and U are each independently selected from the group consisting of: —CR′═ and —N═;

p is 0, 1 or 2;

q is 0, 1 or 2; and

r is 0, 1 or 2;

wherein each moiety R′ is independently selected from the others.

According to another aspect, the present invention relates to a method of making a prodrug of the invention comprising:

(i) preparing an isocyanate derivative of an ABA or ABA analogue;

(ii) reacting the isocyanate derivative of ABA or ABA derivative with a phenolic opioid.

The opioid drug is covalently bonded to the rest of the prodrug at a hydroxyl group via a carbamate linkage.

One embodiment of both Formulae I and I(B) includes a prodrug where n is 1, R1 is hydrogen and R2 is methylene or absent.

In one embodiment, the opioid prodrug moiety is selected from one of the prodrug moieties provided in Table 1.

TABLE 1
Various prodrugs of the present invention

| | Prodrug Moiety | Structure When Bound to Opioid |
|---|---|---|
| 1 | 2-amino benzoic acid | 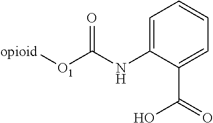 |
| 2 | 3-amino benzoic acid | 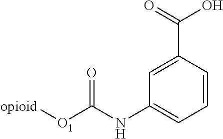 |
| 3 | 4-amino benzoic acid (PABA) | 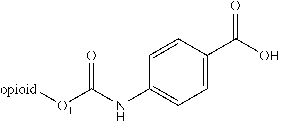 |
| 4 | 4-aminomethyl benzoic acid | 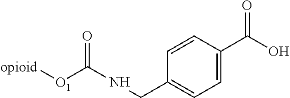 |
| 5 | 4-amino salicylic acid | 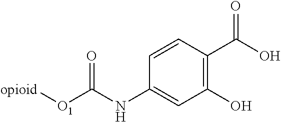 |
| 6 | 4-amino cyclohexanoic acid | 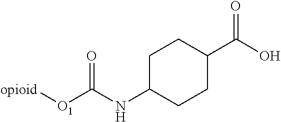 |
| 7 | 4-amino- phenyl acetic acid | 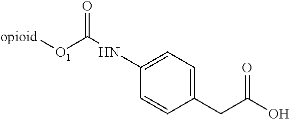 |
| 8 | 4-amino- hippuric acid | 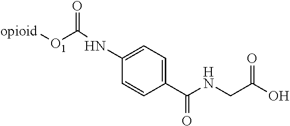 |
| 9 | 4-Amino-2- Chlorobenzoic Acid | 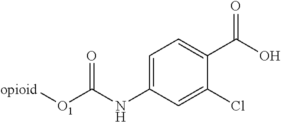 |
| 10 | 6-Aminonicotinic Acid | 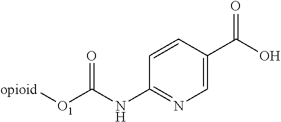 |
| 11 | 4-amino cyclohexanoic acrylic acid and 4-aminomethyl cyclohexanoic acrylic acid | 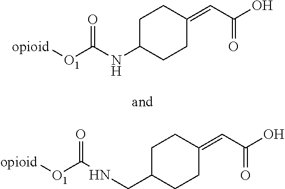 |
| 12 | 4-Amino methyl salicylate | 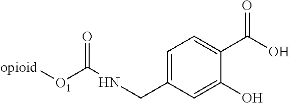 |
| 13 | 2-(4-aminophenyl) propanoic acid | 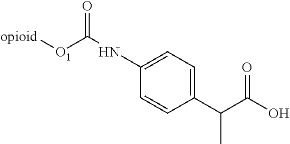 |
| 14 | 2-amino thiazole-4- acetic acid | 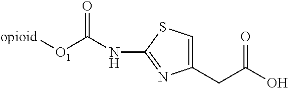 |
| 15 | 2-amino-4- (2-aminophenyl)- 4-oxobutanoic acid | 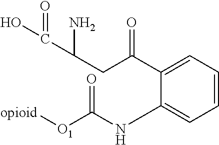 |
| 16 | 4-amino 2-fluorobenzoic acid | 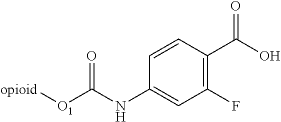 |
| 17 | 4-amino N-methyl benzoic acid carbamate | 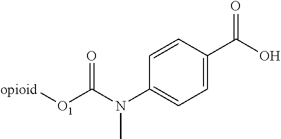 |
| 18 | 4-amino 2-methylbenzoic acid | 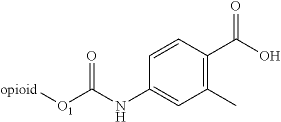 |
| 19 | (5-aminoimidazole carboxylic acid) carbamate | 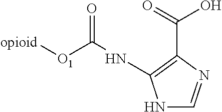 |
| 20 | side-chain-(S)- tryptophan carbamate | 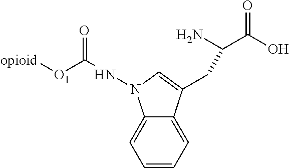 |
| 21 | (4-hydroxyproline) carbamate | 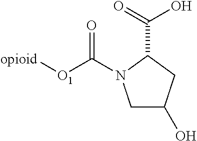 |
| 22 | urocanic acid carbamate | 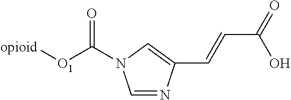 |
| 23 | (indole-3- acetic acid) carbamate | 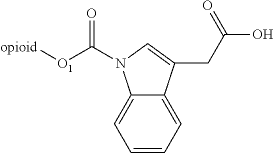 |
| 24 | orotic acid carbamate | 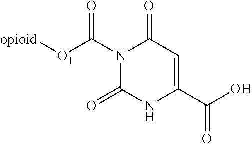 |
| 25 | PABA thiocarbamate | 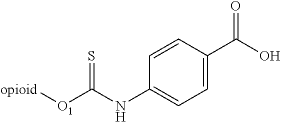 |
| 26 | (5-aminothiophene- 2-carboxylic acid) carbamate | 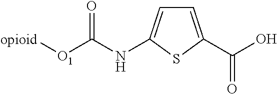 |
| 27 | pipecolic acid carbamate | 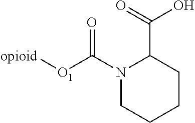 |

In various embodiments, the opioid is selected from oxycodone, hydrocodone, hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine and a phenolically hydroxylated, e.g. a 2-, 3- or 4-phenolically hydroxylated, phenazepine analgesic, e.g., a phenolically hydroxylated, e.g. a 2-, 3- or 4-phenolically hydroxylated ethoheptazine, proheptazine, metethoheptazine or metheptazine, or any other analgesic. Alternatively the opioid may be a narcotic antagonist for example, alvimopan, de-glycinated alvimopan, naloxone, N-methyl naloxone, nalorphine, naltrexone or N-methyl naltrexone.

In various embodiments, the opioid is selected from oxycodone, hydrocodone, hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol and a phenolically hydroxylated, e.g. a 2-, 3- or 4-phenolically hydroxylated, phenazepine analgesic, e.g., a phenolically hydroxylated, e.g. a 2-, 3- or 4-phenolically hydroxylated ethoheptazine, proheptazine, metethoheptazine or metheptazine, or any other analgesic. Alternatively the opioid may be a narcotic antagonist for example, alvimopan, de-glycinated alvimopan, naloxone, nalorphine or naltrexone.

In various embodiments, the opioid is selected from, hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine and a phenolically hydroxylated, e.g. a 2-, 3- or 4-phenolically hydroxylated phenazepine analgesic, e.g., a phenolically hydroxylated, e.g. a 2-, 3- or 4-phenolically hydroxylated of ethoheptazine, proheptazine, metethoheptazine or metheptazine, or any other analgesic. Alternatively the opioid may be a narcotic antagonist for example alvimopan, de-glycinated alvimopan, naloxone, N-methyl naloxone, nalorphine, naltrexone or N-methyl naltrexone.

In various embodiments, the opioid is selected from, hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol and a phenolically hydroxylated, e.g. a 2-, 3- or 4-phenolically hydroxylated phenazepine analgesic, e.g., a phenolically hydroxylated, e.g. a 2-, 3- or 4-phenolically hydroxylated of ethoheptazine, proheptazine, metethoheptazine or metheptazine, or any other analgesic. Alternatively the opioid may be a narcotic antagonist for example alvimopan, de-glycinated alvimopan, naloxone, nalorphine or naltrexone.

The term 2-, 3- or 4-phenolically hydroxylated phenazepine analgesic means a compound having the general structure:

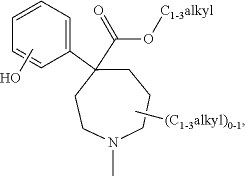

wherein each C₁₋₃ alkyl group is independently selected from the group consisting of: methyl, ethyl and n-propyl, optionally methyl and ethyl.

In an embodiment, the opioid is selected from the group consisting of: meptazinol, tapentadol, nalbuphine, butorphanol and naloxone.

In another embodiment, the opioid is an opioid antagonist. In a further embodiment, the opioid antagonist is selected from naloxone, nalorphine and naltrexone.

In an embodiment, the opioid drug is selected from the group consisting of: oxycodone, hydrocodone, hydromorphone, butorphanol, dezocine, dextrorphan, hydroxyopethidine, ketobemidone, levorphanol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine and a phenolically hydroxylated, e.g. a 2-, 3- or 4-phenolically hydroxylated, phenazepine analgesic, e.g., a phenolically hydroxylated, e.g. a 2-, 3- or 4-phenolically hydroxylated ethoheptazine, proheptazine, metethoheptazine, metheptazine, a narcotic antagonist for example, alvimopan, de-glycinated alvimopan, naloxone, N-methyl naloxone, nalorphine, naltrexone and N-methyl naltrexone, or a pharmaceutically acceptable salt of the aforegoing, and the term “salts” includes acid addition salts or addition salts of free bases; suitable pharmaceutically acceptable salts in this embodiment include, but are not limited to, metal salts for example sodium, potassium and cesium salts; alkaline earth metal salts for example calcium and magnesium salts; organic amine salts for example triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) of the prodrugs of the invention for prodrugs including an opioid listed immediately above include, but are not limited to inorganic acid salts for example the hydrochloride, hydrobromide, sulfate, phosphate; organic acid salts for example trifluoroacetate and maleate salts; sulfonates for example methanesulfonate, ethanesulfonate, benzenesulfonate, p-toluenesulfonate, camphor sulfonate and naphthalenesulfonate; and amino acid salts for example arginate, gluconate, galacturonate, alaninate, asparginate and glutamate salts (see, for example, Berge, et al. “Pharmaceutical Salts,” J. Pharma. Sci. 1977; 66:1).

Thus, the present invention relates in some embodiments to the use of a ring-containing moiety, for example an amino benzoic acid (ABA), e.g. para-amino benzoic acid, linked via a carbamate linkage to an opioid, to treat pain; and to minimize or reduce adverse GI effects by avoidance of direct contact between the opioid and opioid receptors (or other receptors) in the gut. Additionally, the prodrugs provided herein may be used to improve oral bioavailability, and/or to sustain delivery a pharmacologically effective amount of the drug into the blood stream for the reduction or elimination of pain. The sustained delivery may be achieved by the presence of quantities of unhydrolyzed prodrug in plasma providing a reservoir for continued generation of the active drug. This may ensure maintenance of plasma drug levels and thus reduced frequency of drug dosage, which would be expected to improve patient compliance. Additionally the prodrugs of the invention may minimize adverse GI effects by avoidance of direct contact between the active opioid and opioid receptors or other receptors within the gut lumen.

These and other embodiments are disclosed or are apparent from and encompassed by, the following Detailed Description.

### BRIEF DESCRIPTION OF THE DRAWINGS

FIG. 1 is a graph showing the pH lability profile of meptazinol PABA carbamate.

FIG. 2 is a graph showing the meptazinol plasma concentration vs. time profile in dogs (n=6) after oral administration of either meptazinol itself (1 mg free base meptazinol/kg) or meptazinol PABA carbamate (1 mg free base meptazinol equivs./kg).

FIG. 3 is a graph showing the meptazinol plasma concentration vs. time profile in dogs (n=5) after oral administration of either meptazinol itself (group of dogs from example 26) at 1 mg free base meptazinol/kg or meptazinol PABA carbamate (1 mg free base meptazinol equivs./kg, see example 27).

FIG. 4 is a graph showing the meptazinol plasma concentration vs. time profile in monkeys (n=6) after oral administration of either meptazinol itself (2 mg free base meptazinol/kg) or meptazinol PABA carbamate (2 mg free base meptazinol equivs./kg).

FIG. 5 is a graph showing the meptazinol plasma concentration vs. time profile in rats (n=5) after oral administration of either meptazinol itself (1 mg free base/kg) or meptazinol PABA carbamate (1 mg free base meptazinol equivs./kg).

FIG. 6 is graph showing the in vitro formation and subsequent metabolic clearance of meptazinol in hepatocytes from rat, dog, monkey and man.

FIG. 7 is graph showing the buprenorphine plasma concentration vs time profile in monkeys (n=5) after oral administration of either buprenorphine itself at 0.2 mg free base buprenorphine/kg or buprenorphine PABA carbamate (0.2 mg free base buprenorphine equivs./kg.

FIG. 8 is graph showing the buprenorphine plasma concentration vs time profile in dogs (n=5) after oral administration of either buprenorphine itself at 0.1 mg free base buprenorphine/kg or buprenorphine PABA carbamate (0.1 mg free base buprenorphine equivs./kg.

FIG. 9 is graph showing the buprenorphine plasma concentration vs time profile in rats (n=5) after oral administration of either buprenorphine itself at 5.0 mg free base buprenorphine/kg or buprenorphine PABA carbamate (5.0 mg free base buprenorphine equivs./kg.

FIG. 10 is graph showing the in vitro formation and subsequent metabolic clearance of buprenorphine in hepatocytes from rat, dog, monkey and man

FIG. 11 is graph showing the log dose vs response (analgesia) after oral administration of buprenorphine or buprenorphine PABA carbamate in the rat tail flick test.

### DETAILED DESCRIPTION OF THE INVENTION

### Definitions

As used herein:

The term “amino acid” refers both to proteinogenic and non-proteinogenic amino acids. The side chains can be in either the (R) or the (S) configuration (i.e., either D or L amino acids, or both, are contemplated for use in the present invention).

A “proteinogenic amino acid” can be incorporated into proteins during translation. A proteinogenic amino acid generally has the formula

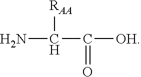

R_{AA} is referred to as the amino acid side chain, or in the case of a proteinogenic amino acid, as the proteinogenic amino acid side chain. The proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine, histidine, selenocysteine and pyrrolysine.

A “non-proteinogenic amino acid” is an organic compound that is not among those encoded by the standard genetic code, or incorporated into proteins during translation, for example, amino benzoic acid (ABA). Non-proteinogenic amino acids, thus, include amino acids or analogues of amino acids other than the 22 proteinogenic amino acids used for protein biosynthesis and include, but are not limited to, the D-isostereomers of amino acids. Examples of non-proteinogenic amino acids include, but are not limited to: para amino benzoic acid, 2-amino benzoic acid, anthranilic acid, 3-amino benzoic acid, 4-aminomethyl benzoic acid, 4-amino salicylic acid (PAS), 4-amino cyclohexanoic acid 4-amino-phenyl acetic acid, 4-amino-hippuric acid, 4-amino-2-chlorobenzoic acid, 6-aminonicotinic acid, methyl-6-aminonicotinate, 4-amino methyl salicylate, 2-amino thiazole-4-acetic acid, 2-amino-4-(2-aminophenyl)-4-oxobutanoic acid (L-kynurenine), citrulline, homocitrulline, hydroxyproline, homoarginine, homoproline, ornithine, 4-amino-phenylalanine, norleucine, cyclohexylalanine, α-aminoisobutyric acid, acetic acid, O-methyl serine (i.e., an amino acid side chain having the formula

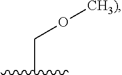

N-methyl-alanine, N-methyl-glycine, N-methyl-glutamic acid, tert-butylglycine, α-aminobutyric acid, tert-butylalanine, α-aminoisobutyric acid, 2-aminoisobutyric acid 2-aminoindane-2-carboxylic acid, selenomethionine, acetylamino alanine (i.e., an amino acid sidechain having the formula

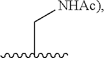

β-alanine, β-(acetylamino)alanine, β-aminoalanine, β-chloroalanine, phenylglycine, lanthionine, dehydroalanine, γ-amino butyric acid, and derivatives thereof wherein the amine nitrogen has been mono- or di-alkylated.

The term “opioid” refers to a natural (e.g. morphine), semi-synthetic (e.g. buprenorphine) or synthetic (e.g. meptazinol) drug that acts by binding to one or more of the opioid receptors in the brain, thus displacing an endogenous analgesic ligand, namely an enkephalin or endorphin, and having a therapeutically useful pain-relieving effect.

The term “narcotic antagonist” refers to a non-natural compound which will displace an opioid from its binding site and so reverse the effects of an opioid analgesic.

The term “amino” refers to a

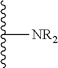

group, wherein each R is independently selected from the group consisting of: H and C₁-C₁₀ alkyl. For example, the term “amino” may refer to a

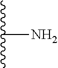

group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl, e.g. a C₁, C₂, C₃, C₄, C₅, C₆, C₇, C₈, C₉ or C₁₀ alkyl. For example, C₁₋₁₀ alkyl means a straight or branched saturated hydrocarbon chain containing, for example, at least 1, and at most 10, carbon atoms. Examples of “alkyl” groups, as used herein include, but are not limited to, methyl, ethyl, n-propyl, n-butyl, n-pentyl, isobutyl, isopropyl, t-butyl, hexyl, heptyl, octyl, nonyl and decyl.

The term “alkyl ester,” includes, for example, groups of the formulae

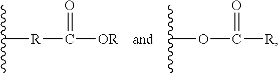

wherein each occurrence of R is independently a straight or branched C₁-C₁₀ alkyl group as defined immediately above.

The term “substituted alkyl” as used herein denotes alkyl radicals wherein at least one hydrogen is replaced by one more substituents such as, but not limited to, hydroxy, alkoxy (for example, C₁-C₁₀ alkoxy, e.g. methoxy or ethoxy), aryl (for example, phenyl), heterocycle, halogen (for example, F, Cl or Br), haloalkyl (for example, C₁-C₁₀ fluoroalkyl, e.g. trifluoromethyl or pentafluoroethyl), cyano, cyanomethyl, nitro, amino (e.g. a

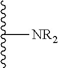

group, wherein each R is independently selected from the group consisting of: H and C₁-C₁₀ alkyl, or a

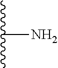

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). The definition pertains whether the term is applied to a substituent itself or to a substituent of a substituent.

The terms “amino benzoic acid analogue,” and “ABA analogue,” are synonymous, and refer to structural analogues of amino benzoic acid. ABA and ABA analogues are non-proteinogenic amino acids. Structural analogues of ABA include amino-substituted 5- or 6-membered rings (e.g., aryl, heteroaryl, heterocycle, cycloalkyl groups), which can be fused to an additional 5- or 6-membered ring. The mono- or bi-cyclic ring can include one or more N, S or O atoms, in place of one or more carbon ring atoms. Additionally or alternatively, the carboxylic acid moiety can be located at any position on the mono- or bi-cyclic ring, instead of the para position (in relation to the amino group). The carboxylic acid group (R₃ group) may contain a linker between the carboxy group and the ring comprising one or more carbon atoms which linker may be saturated or unsaturated, and may be further substituted with a C₁-C₃ alkyl group, amino or hydroxyl group. For example, an ABA analogue may have any of the following acid groups, at any position on the ring:

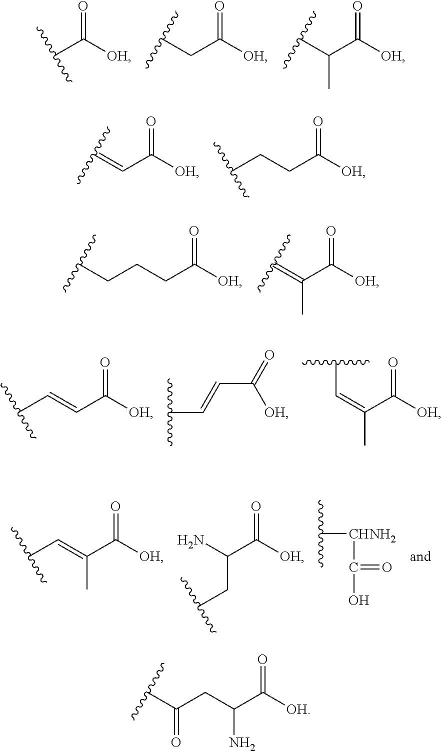

In embodiments, the terms “amino benzoic acid analogue,” and “ABA analogue,” refer to residues having the general structure:

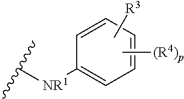

in which R¹, R³, R⁴ and p are as defined above and, in particular, in the aspect and embodiments relating to the compounds of Formula II.

Alternatively or additionally, an ABA analogue may have an additional substituent on the 5- or 6-membered ring (besides the acid and amino groups). For example, the ring of the ABA analogue may be further substituted with a halogen (for example, F, Cl, Br), C₁-C₆ alkyl (for example, C₁, C₂, C₃ or C₄ alkyl), C₁-C₆ alkyl ester (for example, C₁, C₂, C₃ or C₄ alkyl ester), C₁-C₆ substituted alkyl (for example, C₁, C₂, C₃ or C₄ substituted alkyl), substituted C₁-C₆ alkyl ester (for example, C₁, C₂, C₃ or C₄ substituted alkyl

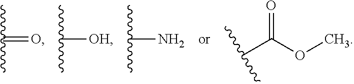

ester), Alternatively or additionally, the amino group in the ABA or ABA analogue can be substituted with an alkyl or substituted alkyl group (for example, a C₁, C₂, C₃ or C₄ alkyl or substituted alkyl). Further, in contrast to ABA, an ABA analogue may have an optionally substituted C₁-C₃ n-alkyl group between the amino group (i.e., ABA's N-terminus) and the 5- or 6-membered ring. In an embodiment, the phenyl ring of the ABA analogue is directly bonded to the amino group of the ABA analogue.

In a preferred embodiment, the ABA or ABA analogue is bound to an opioid through the ABA analogue's amino group, to form a carbamate bond. In one embodiment, the ABA analogue includes a heteroaryl ring, for example a thiazole or pyridine ring. In other embodiments, the ABA analogue does not include a heteroaryl ring.

The terms “para amino benzoic acid analogue,” and “PABA analogue,” are synonymous, and refer to structural analogues of para amino benzoic acid. PABA and PABA analogues are non-proteinogenic amino acids. Structural analogues of PABA include amino substituted 5- or 6-membered rings (e.g., aryl, heteroaryl, heterocycle, cycloalkyl groups), which can be fused to an additional 5- or 6-membered ring. The mono- or bi-cyclic ring can include one or more N, S or O atoms, in place of one or more carbon ring atoms. Additionally or alternatively, the carboxylic acid moiety can be located at any position on the mono- or bi-cyclic ring, instead of the para position (in relation to the amino group). The carboxylic acid group (R³ group) may contain a linker between the carboxy group and the ring comprising one or more carbon atoms which linker may be saturated or unsaturated, and may be further substituted with a C1-C3 alkyl group, amino or hydroxyl group. For example, a PABA analogue may have any of the following acid groups, at any position on the ring:

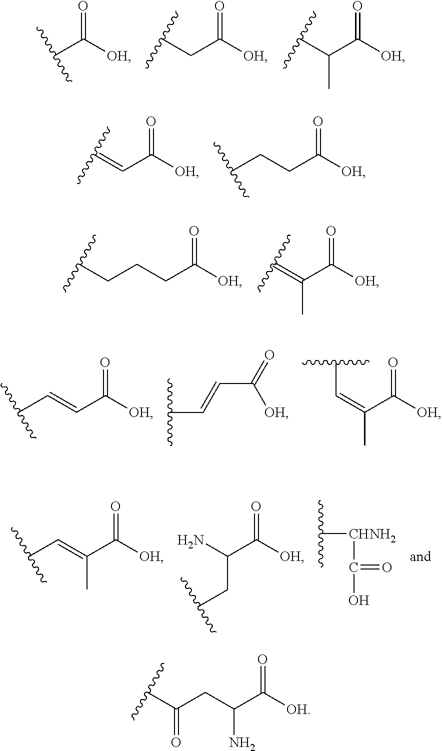

In embodiments, the terms “para amino benzoic acid analogue,” and “PABA analogue,” refer to residues having the general structure:

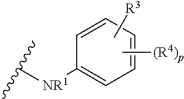

in which R1, R3, R⁴ and p are as defined above and, in particular, in the aspect and embodiments relating to the compounds of Formula II.

Alternatively or additionally, a PABA analogue may have an additional substituent on the 5- or 6-membered ring (besides the acid and amino groups). For example, the ring of the PABA analogue may be further substituted with a halogen (for example, F, Cl, Br), C₁-C₆ alkyl (for example, C₁, C₂, C₃ or C₄ alkyl), C₁-C₆ alkyl ester (for example, C₁, C₂, C₃ or C₄ alkyl ester), C₁-C₆ substituted alkyl (for example, C₁, C₂, C₃ or C₄ substituted alkyl), substituted C₁-C₆ alkyl ester (for example, C₁, C₂, C₃ or C₄ substituted alkyl ester),

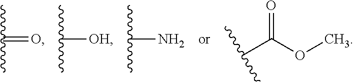

Alternatively or additionally, the amino group in the PABA or PABA analogue can be substituted with an alkyl or substituted alkyl group (for example, a C₁, C₂, C₃ or C₄ alkyl or substituted alkyl). Further, in contrast to PABA, a PABA analogue may have an optionally substituted C₁-C₃ n-alkyl group between the amino group (i.e., PABA's N-terminus) and the 5- or 6-membered ring. In an embodiment, the phenyl ring of the PABA analogue is directly bonded to the amino group of the PABA analogue.

In a preferred embodiment, the PABA or PABA analogue is bound to an opioid through the PABA analogue's amino group, to form a carbamate bond. In one embodiment, the PABA analogue includes a heteroaryl ring, for example a thiazole or pyridine ring. In other embodiments, the PABA analogue does not include a heteroaryl ring.

The term “cycloalkyl” group as used herein refers to a non-aromatic monocyclic hydrocarbon ring of from 3 to 8 carbon atoms. Exemplary are saturated monocyclic hydrocarbon rings having 1, 2, 3, 4, 5, 6, 7 or 8, carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as those recited in the paragraph defining the substitutents of a “substituted alkyl”. The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur. For example, a heterocyclic group may be:

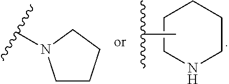

The term “substituted heterocycle” as used herein denotes a heterocycle group further bearing one or more substituents as set forth herein, such as those recited in the paragraph defining the substitutents of a “substituted alkyl”. The definition pertains whether the term is applied to a substituent itself or to a substituent of a substituent. For example, a substituted heterocyclic group may be:

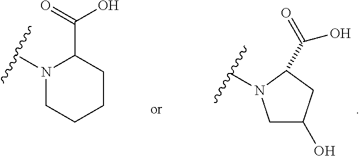

The term “aryl,” as used herein, refers to cyclic, aromatic hydrocarbon groups which have 1 to 3 aromatic rings, for example phenyl or naphthyl. The aryl group may have fused thereto a second or third ring which is a heterocyclo, cycloalkyl, or heteroaryl ring, provided in that case the point of attachment will be to the aryl portion of the ring system. Thus, exemplary aryl groups include

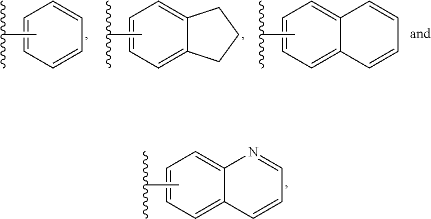

In embodiments, “aryl” refers to a ring structure consisting exclusively of hydrocarbyl groups.

The term “heteroaryl,” as used herein, refers to an aryl group in which at least one of the carbon atoms in the aromatic ring has been replaced by a heteroatom selected from oxygen, nitrogen and sulphur. The nitrogen and/or sulfur heteroatoms may optionally be oxidized and the nitrogen heteroatoms may optionally be quaternized. The heteroaryl group may be a 5 to 6 membered monocyclic, 7 to 11 membered bicyclic, or 10 to 16 membered tricyclic ring system. Thus, exemplary heteroaryl groups include

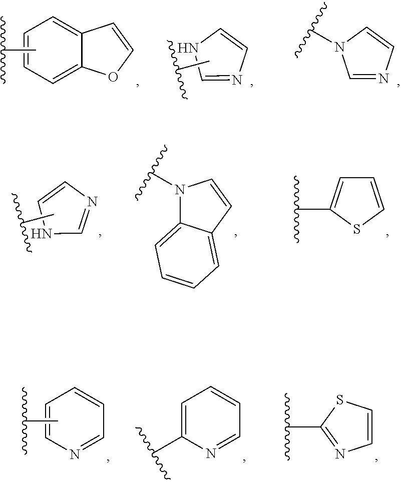

“Substituted aryl” and “substituted heteroaryl” groups refer to either an aryl or heteroaryl group, respectively, substituted by one or more substitutents at any point of attachment to the aryl or heteroaryl ring (and/or any further ring fused thereto). Exemplary substituents include hydroxy, carboxyl, alkoxy (for example, C₁-C₁₀ alkoxy, e.g. methoxy, ethoxy), aryl, phenyl, heterocycle, halogen (for example F, Cl, Br), haloalkyl (for example, C₁-C₁₀ haloalkyl, e.g. trifluoromethyl or pentafluoroethyl), cyano, cyanomethyl, nitro, amino (e.g. a

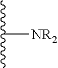

group, wherein each R is independently selected from the group consisting of: H and C₁-C₁₀ alkyl, or a

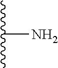

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carboxylic acid (e.g.,

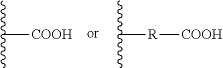

where R is a C₁-C₁₀ alkylene group such as —CH₂—), carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). For example, substituted aryl” and “substituted heteroaryl” groups include:

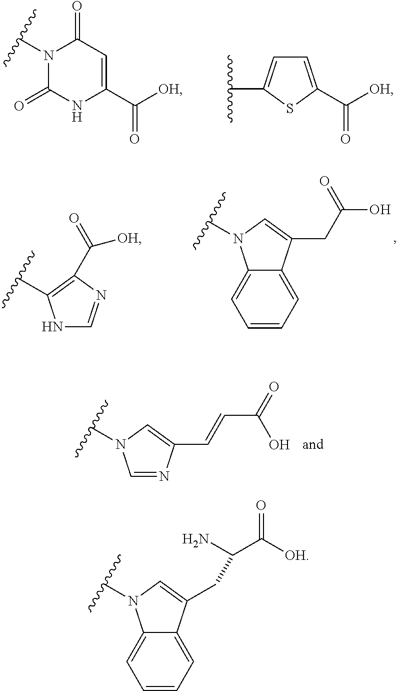

The terms “keto” and “oxo” are synonymous, and refer to the group ═O.

The terms “carbamate group,” “carbamate” and “carbamate linkage” are synonymous, and refer to the group

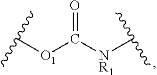

wherein the —O₁— is present in the unbound form of the opioid analgesic (e.g. the phenolic hydroxy group), and the —NR₁ moiety is an amino group present in the ABA or ABA analogue (e.g. PABA or PABA analogue). Prodrug moieties described herein may be referred to based on the ABA or ABA analogue (e.g. PABA or PABA analogue) and the carbamate linkage. The ABA or ABA analogue (e.g. PABA or PABA analogue) reference should be assumed to be bonded via an amino group present in ABA or ABA analogue (e.g. PABA or the PABA analogue) to the carbonyl linker and the opioid analgesic, unless otherwise specified.

The term “thiocarbamate group,” and “thiocarbamate” refer to the group

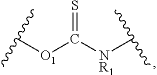

Prodrug moieties described herein may be referred to based on the ABA or ABA analogue (e.g. PABA or PABA analogue) and the thiocarbamate linkage.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

The term “subject” includes humans and other mammals, such as domestic animals (e.g., dogs and cats).

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be analgesia and/or an amelioration of one or more gastrointestinal side effect symptoms that are present when the respective opioid in the prodrug is administered in its active form (i.e., when the opioid is administered alone). It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to the opioid portion of a prodrug of the present invention, as described herein.

“Opioid” refers to the opioid per se, as well as any active metabolites of the respective opioid.

The term “salts” can include acid addition salts or addition salts of free bases. Suitable pharmaceutically acceptable salts (for example, of the carboxyl terminus of the PABA or PABA analogue) include, but are not limited to, metal salts for example sodium potassium and cesium salts; alkaline earth metal salts for example calcium and magnesium salts; organic amine salts for example triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts for example the hydrobromide; and organic acid salts for example trifluoroacetate salts.

The term “bioavailability,” as used herein, generally means the rate and extent to which the active ingredient is absorbed from a drug product and becomes systemically available, and hence available at the site of action. See Code of Federal Regulations, Title 21, Part 320.1 (2003 ed.). For oral dosage forms, bioavailability relates to the processes by which the active ingredient is released from the oral dosage form and moves to the site of action. Bioavailability data for a particular formulation provides an estimate of the fraction of the administered dose that is absorbed into the systemic circulation. Thus, the term “oral bioavailability” refers to the fraction of a dose of a respective opioid given orally that is absorbed into the systemic circulation after a single administration to a subject. A preferred method for determining the oral bioavailability is by dividing the AUC of the opioid given orally by the AUC of the same opioid dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

The term “increase in oral bioavailability” refers to the increase in the bioavailability of a respective opioid when orally administered as a prodrug of the present invention (either a prodrug compound or composition), as compared to the bioavailability when the opioid is orally administered alone. The increase in oral bioavailability can be from 50% to 10,000%, 100% to 10,000%, preferably from 200% to 10,000%, more preferably from 500% to 10,000%, and most preferably from 1000% to 10,000%.

The term “low oral bioavailability,” refers to an oral bioavailability wherein the fraction of a dose of the parent drug given orally that is absorbed into the plasma unchanged after a single administration to a subject is 25% or less, preferably 15% or less, and most preferably 10% or less. Without wishing to be bound by any particular theory, it is believed that the low oral bioavailability of the opioids described herein is the result of the conjugation of a phenolic oxygen to glucuronic acid during first pass metabolism. However, other mechanisms may be responsible for the decrease in oral bioavailability and are contemplated by the present invention.

### Compounds of the Invention

The opioid analgesic of the present invention is conjugated to ABA or an ABA analogue through a carbamate or thiocarbamate linkage, and typically a carbamate linkage, via a nitrogen atom of the ABA or ABA analogue. The ABA or ABA analogue can be conjugated to any free oxygen on the opioid analgesic. In an embodiment, however, the ABA or ABA analogue is conjugated to a phenolic hydroxy residue.

In one embodiment, an ABA or ABA analogue can be bound to one of two (or both) possible locations in the opioid molecule. For example, morphine and dihydromorphine have hydroxyl groups at carbon 3 and carbon 6. An ABA or ABA analogue can be bound at either, or both of these positions. Carbamate or thiocarbamate linkages can be formed at either site, and upon cleavage of the prodrug moiety, the opioid will revert back to its original form. This general process is shown below in scheme A, for three types of morphine prodrugs (i.e., with a ABA or ABA analogue linked at either or both the third and sixth carbons). For scheme A, R₁, R₂, R₃, Cy and n are defined above, as provided for Formula I.

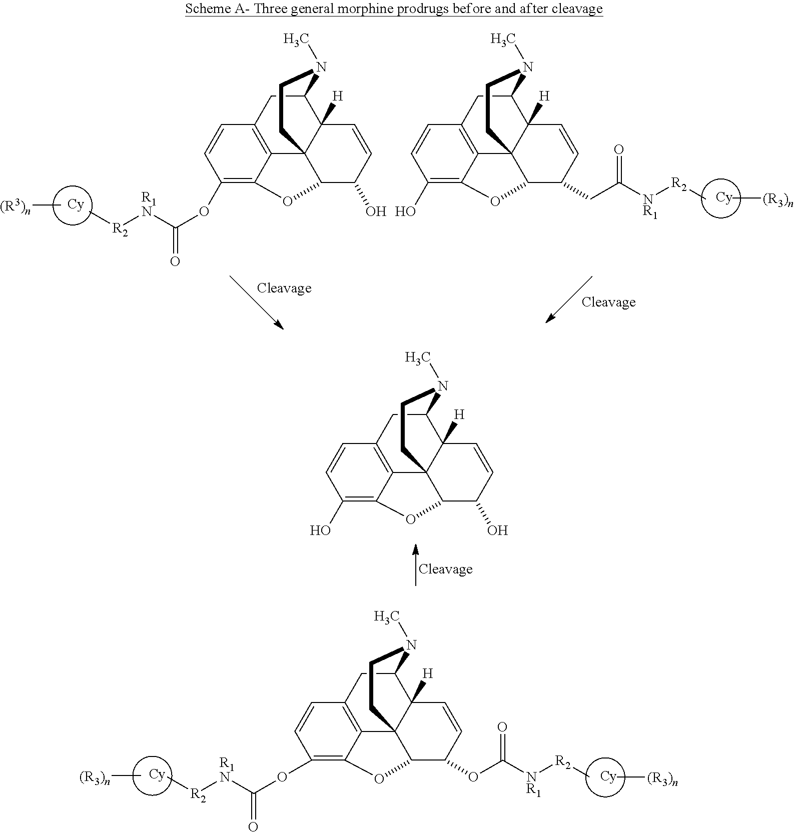

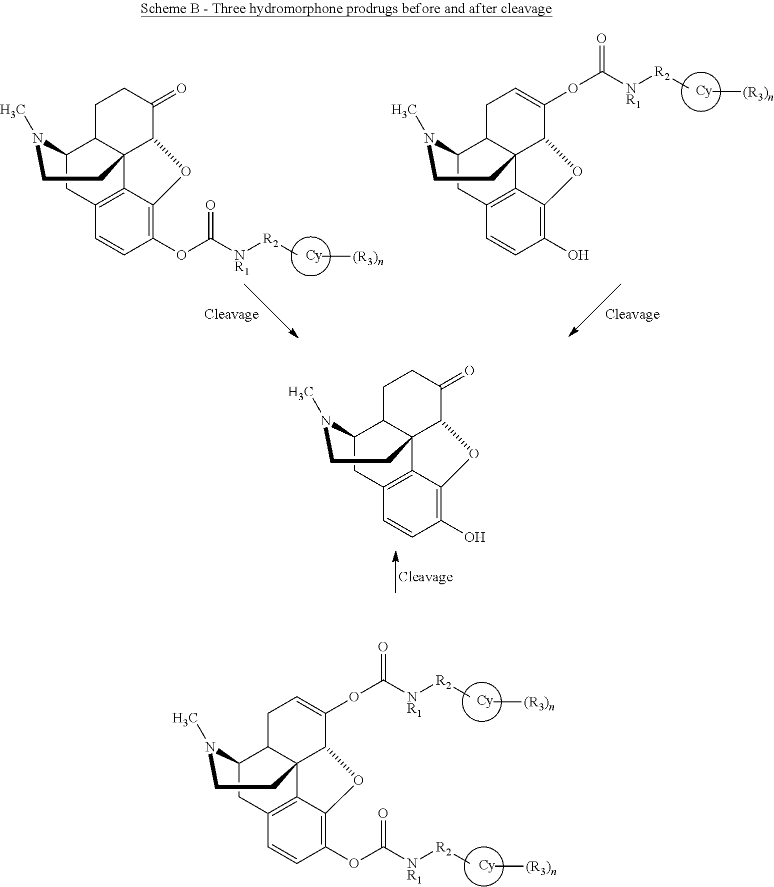

In an embodiment, however, the ABA or ABA analogue is covalently bound to a phenolic hydroxyl group. In this embodiment, there is no possibility in the case of morphine and hydromorphine that the ABA or ABA analogue is conjugated to two possible locations in the opioid molecule (since there is only one phenolic hydroxyl group).

In a preferred embodiment, the prodrug moiety is para amino benzoic acid (PABA). Other ABA analogues within the scope of the invention include, but are not limited to, 2-amino benzoic acid, anthranilic acid, 3-amino benzoic acid, 4-aminomethyl benzoic acid, 4-amino salicylic acid (PAS), 4-amino cyclohexanoic acid, 4-amino-phenyl acetic acid, 4-amino-hippuric acid, 4-amino-2-chlorobenzoic acid, 6-aminonicotinic acid, methyl-6-aminonicotinate, 4-amino methyl salicylate, 2-amino thiazole-4-acetic acid and 2-amino-4-(2-aminophenyl)-4-oxobutanoic acid (L-kynurenine).

### Formula I

It will be recalled that in one aspect, the present invention provides an opioid prodrug of Formula I:

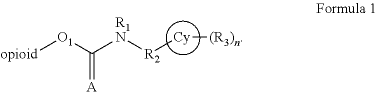

In an embodiment, O₁ is a hydroxylic oxygen atom present in the unbound opioid molecule.

In one Formula I embodiment, R₂ is absent or methylene, R₃ is

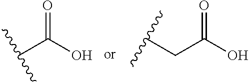

and R₁ is hydrogen. In one Formula I embodiment, R₂ is absent or methylene, at least one R₃ is

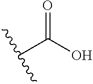

and R₁ is hydrogen. In one Formula I embodiment, R₂ is absent, at least one R₃ is

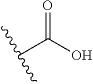

and R₁ is hydrogen. In a further embodiment, R₃ (which is —COON) is located at the para position of a 6 membered ring. In a further embodiment, the ring is a benzene ring. In yet a further embodiment, the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol and a phenolically hydroxylated, e.g. a 2-, 3- or 4-phenolically hydroxylated phenazepine analgesic, e.g., a phenolically hydroxylated, e.g. a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

One embodiment is directed to a prodrug of Formula I where R₁ is hydrogen, R₂ is absent, n is 1 and one occurrence of R₃ is

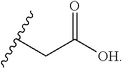

In a further embodiment, the opioid is selected from the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol ora phenolically hydroxylated, e.g. 2-, 3- or 4-phenolically hydroxylated phenazepine analgesic, e.g., a phenolically hydroxylated, e.g. a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification. Another embodiment of the present invention is directed to a compound defined as follows: R₁ is hydrogen, R₂ is absent, n is 1 and R₃ is

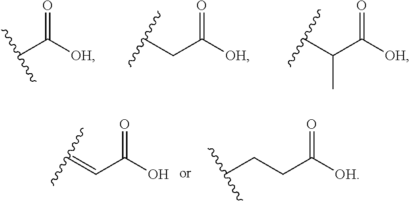

In a further embodiment, Cy is a 5 membered aromatic ring, and the R₃ group is located at position 3 or 4 of the ring, and R₃ is selected from

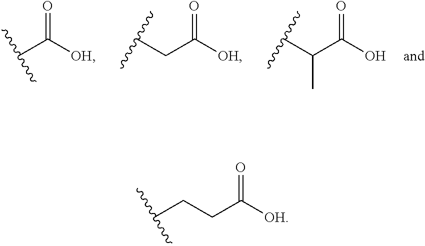

Another embodiment of the present invention is directed to a prodrug of Formula I where R₁ is hydrogen, R₂ is absent, n is 2 and R₃ are

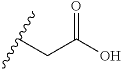

and methyl.

Yet another embodiment is directed to a prodrug of Formula I where R₁ is hydrogen, R₂ is absent, n is 1 and R₃ is

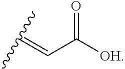

In a further embodiment, the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a phenolically hydroxylated, e.g. a 2-, 3- or 4-phenolically hydroxylated phenazepine analgesic, e.g., a phenolically hydroxylated, e.g. a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

The invention is also directed to a prodrug of Formula I where R₁ is hydrogen, R₂ is absent, n is 1 and R₃ is

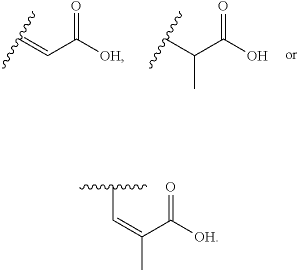

Yet another embodiment is directed to a compound of Formula I where R₁ is hydrogen, R₂ is absent, n is 1 and R₃ is

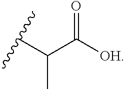

In a further preferred embodiment, Cy is a benzene ring, and R₃ is located at position 3, 4 or 5 of the ring. In a further embodiment, the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a phenolically hydroxylated, e.g. a 2-, 3- or 4-phenolically hydroxylated phenazepine analgesic, e.g., a phenolically hydroxylated, e.g. a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

In one Formula I embodiment, n is 1, R₃ is

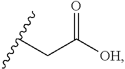

and R₂ is methylene.

In a particular Formula I embodiment, R₁ is hydrogen, R₂ is methylene, n is 1 and R₃ is

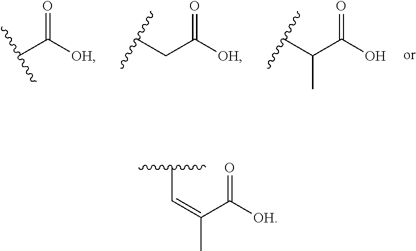

In another Formula I embodiment, R₁ is hydrogen, R₂ is methylene, n is 1 and R₃ is

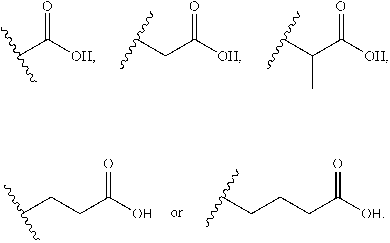

In a preferred Formula I embodiment, R₁ is hydrogen, R₂ is methylene, n is 1 and R₃ is

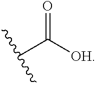

Another embodiment is directed to a compound of Formula I where R₁ is hydrogen, R₂ is methylene, n is 1 and R₃ is

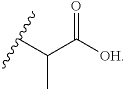

In a further embodiment, Cy is a benzene ring, and R₃ is located at position 3, 4 or 5 of the ring.

Yet another Formula I embodiment is directed to a compound defined as follows: R₁ is hydrogen, R₂ is methylene, n is 1 and R₃ is

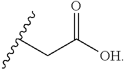

In a further embodiment, Cy is a 5 membered aromatic ring, and

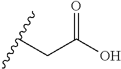

is located at position 3, 4 or 5 of the ring.

Another Formula I embodiment is directed to a compound defined as follows: R₁ is hydrogen, R₂ is methylene, n is 1 and R₃ is

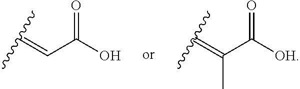

In a further embodiment, Cy is a cyclopentane ring, and R₃ is located at position 3, 4 or 5 of the ring.

Another prodrug is drawn to a compound of Formula I, where R₁ is hydrogen, R₂ is methylene, n is 1 and R₃ is

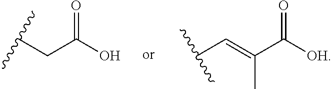

In a further embodiment, Cy is a benzene ring, and R₃ is located at position 3, 4 or 5 of the ring. In yet a further embodiment, the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol ora phenolically hydroxylated, e.g. a 2-, 3- or 4-phenolically hydroxylated phenazepine analgesic, e.g., a phenolically hydroxylated, e.g. a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

In another Formula I embodiment, R₁ is hydrogen, R₂ is methylene, n is 2 and one R₃ is selected from

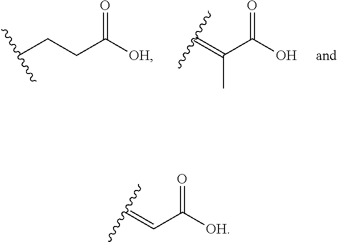

In a further embodiment, Cy is non-aromatic, and the at least one R₃ is located at position 3, 4 or 5 of the ring. In even a further embodiment, a second R₃ is selected from halogen and

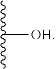

The invention is also directed to compounds of Formula I where R₁ is hydrogen, n is 2, R₂ is absent or methylene, and one occurrence of R₃ is selected from

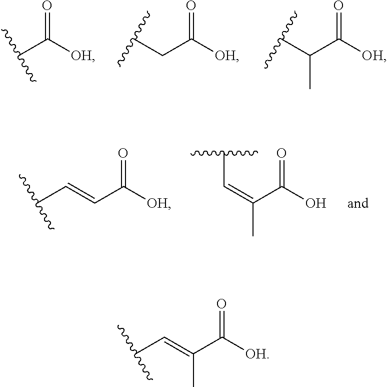

The second R₃ is halogen or hydroxyl. In a further embodiment, Cy is a benzene ring, and at least one R₃ is located at position 4 of the ring.

In another Formula I embodiment, R₁ is hydrogen, R₂ is methylene or absent, n is 2 and each R₃ is independently selected from

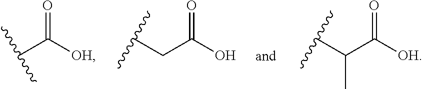

In a further embodiment, Cy is a benzene ring, and R₃ is located at position 3, 4 or 5 of the ring.

In another Formula I embodiment, R₁ is hydrogen, R₂ is methylene or absent, n is 2 and the carboxylic acid R₃ is

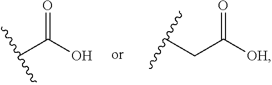

while the second R₃ is selected from halogen, C₁-C₆ alkyl and

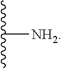

In a further embodiment, Cy is a benzene ring, and the carboxylic acid group is

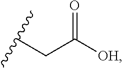

and is located at position 3, 4 or 5 of the ring.

Yet another Formula I embodiment is directed to a compound defined as follows: R₁ is hydrogen, R₂ is methylene, n is 1 and R₃ is

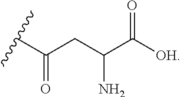

In a further embodiment, Cy is a 5 membered aromatic ring, and R₃ is located at position 3, 4 or 5 of the ring, and the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a 2-, 3- or 4-phenolically hydroxylated phenazepine analgesic, e.g., a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

Another Formula I embodiment includes a prodrug where R₁ is hydrogen, R₂ is absent or methylene, n is 1 and R₃ is either

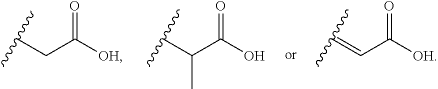

In a further embodiment, the opioid is an opioid antagonist.

Another Formula I embodiment is directed to a prodrug where R₁ is hydrogen, R₂ is absent or methylene, n is 1 and R₃ is

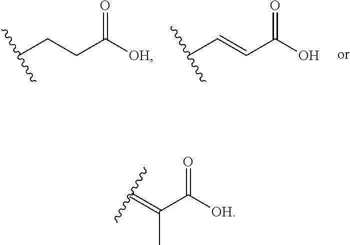

Yet another embodiment of the present invention includes an opioid antagonist prodrug where R₁ is hydrogen, R₂ is absent or methylene, n is 1 and R₃ is

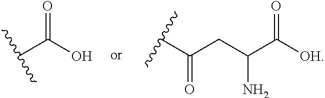

In another Formula I embodiment, R₁ is hydrogen, R₂ is absent or methylene, n is 1 and R₃ is

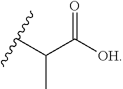

In a further embodiment, the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a 2-, 3- or 4-phenolically hydroxylated phenazepine analgesic, e.g., a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

Even another Formula I embodiment is directed to a compound defined as follows: R₁ is hydrogen, R₂ is absent or methylene, n is 2 and at least one R₃ is halogen or

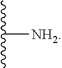

Accordingly, in this embodiment, there are two R₃ groups, and the first R₃ is selected from the carboxylic acids given above. In a further embodiment, Cy is a 5 or 6 membered aromatic ring, and the carboxylic acid is located at position 3 or 4 of the ring, and the opioid is selected fromhydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a 2-, 3- or 4-phenolically hydroxylated phenazepine analgesic, e.g., a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

Other Formula I embodiments are given in Table 2, below.

TABLE 2
Non limiting Formula I embodiments

| Embodiment | R₁ | R₂ | Cy | n | R₃ |
|---|---|---|---|---|---|
| 1 | hydrogen | absent | benzene | 1 | 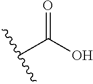 |
| 2 | hydrogen | absent | benzene | 1 | 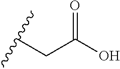 |
| 3 | hydrogen | absent | benzene | 1 | 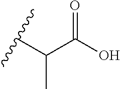 |
| 4 | hydrogen | absent | cyclohexane | 1 | 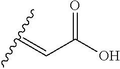 |
| 5 | hydrogen | absent | benzene | 1 | 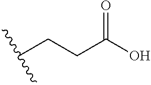 |
| 6 | hydrogen | absent | benzene | 1 | 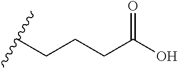 |
| 7 | hydrogen | absent | cyclohexane | 1 | 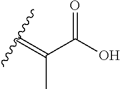 |
| 8 | hydrogen | absent | benzene | 1 | 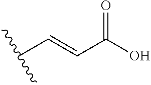 |
| 9 | hydrogen | absent | benzene | 1 | 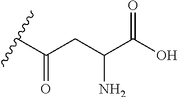 |
| 10 | hydrogen | absent | benzene | 1 | 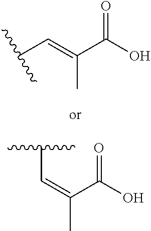 |
| 11 | hydrogen | methylene | benzene | 1 | 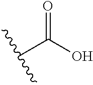 |
| 12 | hydrogen | methylene | benzene | 1 | 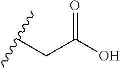 |
| 13 | hydrogen | methylene | benzene | 1 | 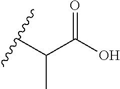 |
| 14 | hydrogen | methylene | cyclohexane | 1 | 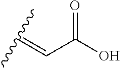 |
| 15 | hydrogen | methylene | benzene | 1 | 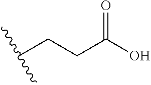 |
| 16 | hydrogen | methylene | benzene | 1 | 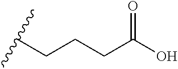 |
| 17 | hydrogen | methylene | cyclohexane | 1 | 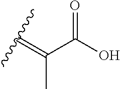 |
| 18 | hydrogen | methylene | benzene | 1 | 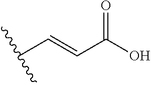 |
| 19 | hydrogen | methylene | benzene | 1 | 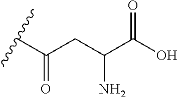 |
| 20 | hydrogen | methylene | benzene | 1 | 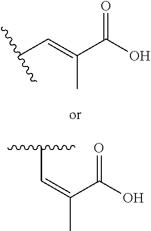 |
| 21 | hydrogen | absent | 5-membered aromatic | 1 | 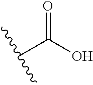 |
| 22 | hydrogen | absent | 5-membered aromatic | 1 | 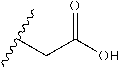 |
| 23 | hydrogen | absent | 5-membered aromatic | 1 | 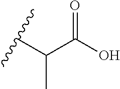 |
| 24 | hydrogen | absent | cyclopentane | 1 | 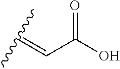 |
| 25 | hydrogen | absent | 5-membered aromatic | 1 | 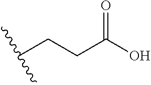 |
| 26 | hydrogen | absent | 5-membered aromatic | 1 | 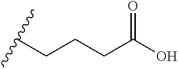 |
| 27 | hydrogen | absent | cyclopentane | 1 | 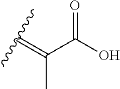 |
| 28 | hydrogen | absent | 5-membered aromatic | 1 | 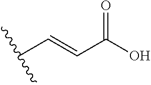 |
| 29 | hydrogen | absent | 5-membered aromatic | 1 | 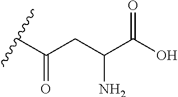 |
| 30 | hydrogen | absent | 5-membered aromatic | 1 | 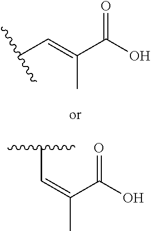 |
| 31 | hydrogen | methylene | 5-membered aromatic | 1 | 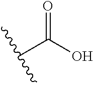 |
| 32 | hydrogen | methylene | 5-membered aromatic | 1 | 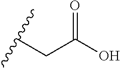 |
| 33 | hydrogen | methylene | 5-membered aromatic | 1 | 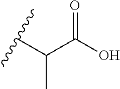 |
| 34 | hydrogen | methylene | cyclopentane | 1 | 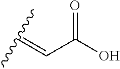 |
| 35 | hydrogen | methylene | 5-membered aromatic | 1 | 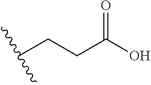 |
| 36 | hydrogen | methylene | 5-membered aromatic | 1 | 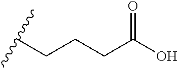 |
| 37 | hydrogen | methylene | cyclopentane | 1 | 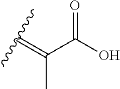 |
| 38 | hydrogen | methylene | 5-membered aromatic | 1 | 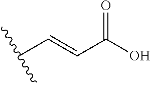 |
| 39 | hydrogen | methylene | 5-membered aromatic | 1 | 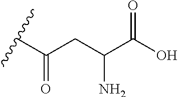 |
| 40 | hydrogen | methylene | 5-membered aromatic | 1 | 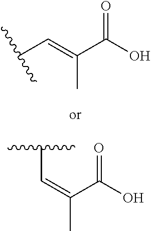 |

In the embodiments described in Table 2, “A” can be either ═O or ═S. In an embodiment, “A” is ═O.

### Formula I(A)

It will be recalled that in one aspect, the present invention provides an opioid prodrug of Formula I(A):

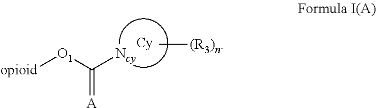

One embodiment is directed to prodrugs of Formula I(A) where A is ═O, n is 1 and Cy is selected from

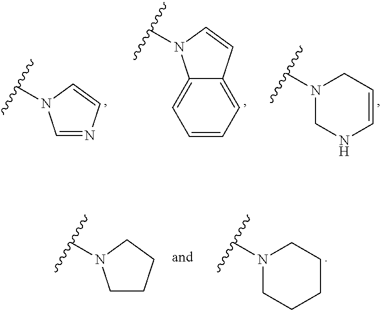

The invention is also directed to compounds of Formula I(A) where n is 1 and R₃ is selected from

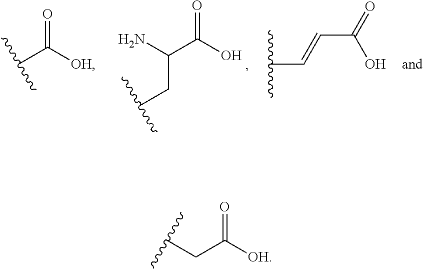

In a further embodiment, Cy is selected from

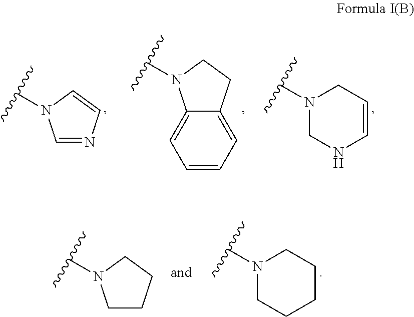

It will be recalled that in one aspect, the present invention provides an opioid prodrug of Formula I(B):

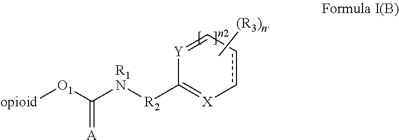

One embodiment is a compound of Formula I(B) where R₁ is hydrogen, R₂ is absent or methylene, n2 is 1, n is 1 or 2 and at least one occurrence of R₃ is

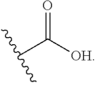

Another Formula I(B) embodiment is directed to an opioid prodrug of Formula I where n2 is 0. In a further embodiment, Y is —S— and X is —N—, and the ring containing X and Y is aromatic, i.e., the ring is a thiazole ring. In a further embodiment, at least one R₃ is selected from

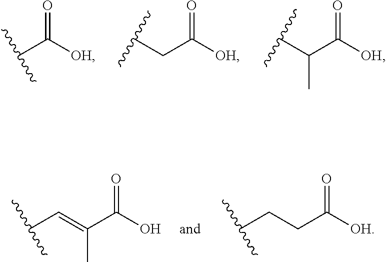

In yet another Formula I(B) embodiment, R₂ is absent, R₁ is H, X and Y are both —C—, n and n2 are both 1 and R₃ is selected from

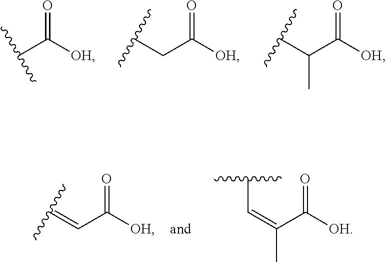

In a further embodiment, the ring is aromatic, the R₃ substituent is located at position 2, 3 or 5 of the ring, and R₃ is selected from

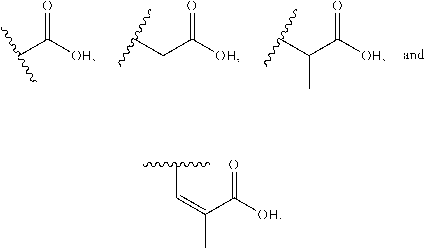

In yet another Formula I(B) embodiment, R₂ is absent, R₁ is H, Y and X are both —C—, n and n2 are both 1 and R₃ is selected from

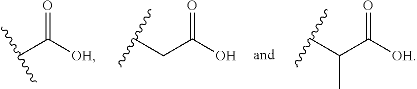

In a further embodiment, the ring is aromatic, and the R₃ substituent is located at position 3, 4 or 5 of the ring.

In yet another Formula I(B) embodiment, R₁ is hydrogen, R₂ is methylene, Y and X are both —C—, n2 is 1 and at least one occurrence of R₃ is selected from

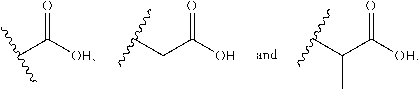

In a further embodiment, the ring is aromatic (benzene ring), and the at least one R₃ substituent is located at position 3, 4 or 5 of the ring. In a further embodiment, there is only one R₃ substituent on the ring (i.e., n is 1), and it is located at position 3 or 4.

The invention is also directed to a compound of Formula I(B) where R₁ is hydrogen, R₂ is methylene, X and Y are both —C—, n and n2 are both 1 and R₃ is selected from

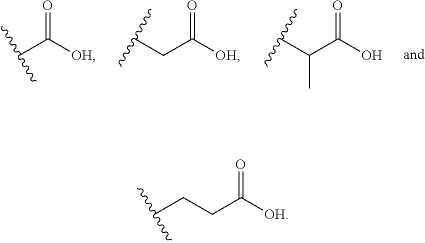

In a further embodiment, the ring is aromatic, and the R₃ substituent is located at position 2, 3 or 5 of the ring.

Another Formula I(B) embodiment R₂ is methylene, R₁ is H, Y and X are both —C—, n and n2 are both 1 and R₃ is selected from

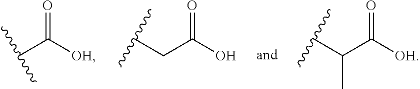

In a further embodiment, the ring is aromatic, and the R₃ substituent is located at position 3, 4 or 5 of the ring.

In even another Formula I(B) embodiment, R₁ is hydrogen, R₂ is absent, Y is —C—, X is —N—, n2 is 1 and at least one occurrence of R₃ is selected from

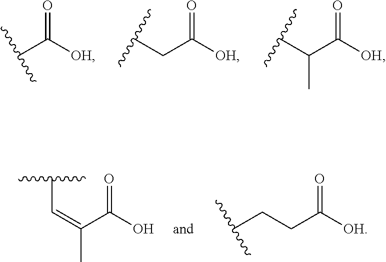

In a further embodiment, the ring is aromatic, n is 1 and the R₃ substituent is located at position 3, 4 or 5 of the ring.

In even another Formula I(B) embodiment, R₁ is H, R₂ is absent, Y is —C—, X is —N—, n2 is 1 and at least one occurrence of R₃ is selected from

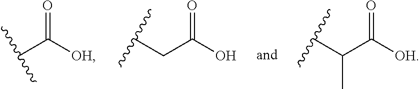

In a further embodiment, the ring is aromatic, and the at least one R₃ substituent is located at position 3, 4 or 5 of the ring.

Another embodiment is directed to a compound of Formula I(B) where R₁ is hydrogen, R₂ is absent, Y is —N—, X is —N—, n2 is 1 and at least one occurrence of R₃ is selected from

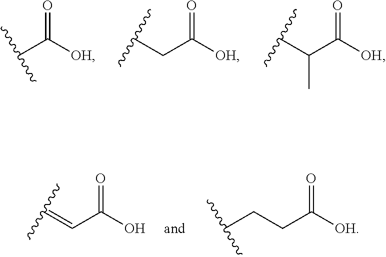

In a further embodiment, the ring is a 5 membered ring, and the at least one R₃ substituent is located at position 3 or 4 of the ring. In even a further Formula I(B) embodiment, the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a 2-, 3- or 4-phenolically hydroxylated phenazepine analgesic, e.g., a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

In yet another Formula I(B) embodiment, R₁ is hydrogen, R₂ is methylene, Y and X are both —C—, n2 is 1 and at least one occurrence of R₃ is selected from

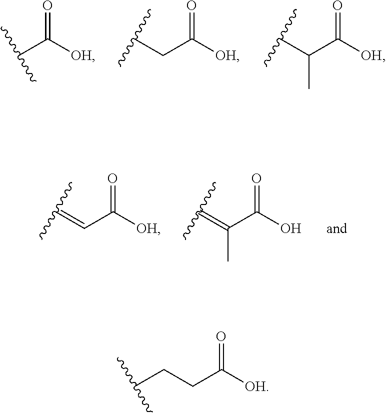

In a further embodiment, the ring is aromatic (benzene ring), and the at least one R₃ substituent is located at position 2, 3 or 5 of the ring. In a further embodiment, there is only one R₃ substituent on the ring (i.e., n is 1), and it is located at position 3. In even a further Formula I(B) embodiment, the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a 2-, 3- or 4-phenolically hydroxylated phenazepine analgesic, e.g., a 2-, 3- or 4-phenolically hydroxylated ethoheptazine, proheptazine, metethoheptazine or metheptazine, or any other analgesicand R₃ is

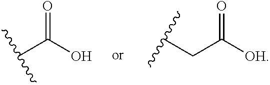

Alternatively the opioid may be a narcotic antagonist such as alvimopan, de-glycinated alvimopan, naloxone, nalorphine or naltrexone. The opioid may be any other phenolic opioid disclosed in this specification.

Another Formula I(B) embodiment includes an opioid prodrug where R₁ is hydrogen, R₂ is methylene, Y is —C— and X is —N—, n2 is 1 (6 membered ring), n is 1 and R₃ is selected from

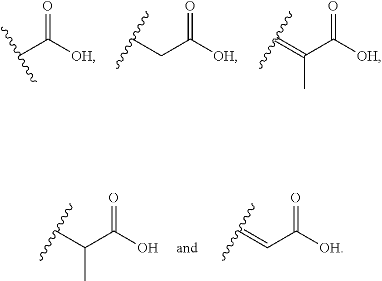

In a further embodiment, the ring is a cyclohexane ring, and R₃ is located at position 2, 3 or 5 of the ring.

In even another Formula I(B) embodiment, R₁ is H, R₂ is methylene, Y is —C— and X is —N—, n2 is 1 (6 membered ring), and at least one R₃ is selected from

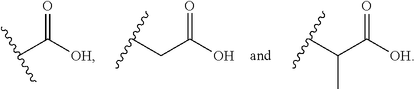

In a further embodiment, the ring is a benzene ring, n is 1 and R₃ is located at position 3, 4 or 5 of the ring.

The present invention is also directed to prodrugs of Formula I(B) where R₁ is hydrogen, R₂ is methylene or absent, Y and X are both —C—, n2 is 1, n is 2 and one occurrence of R₃ is

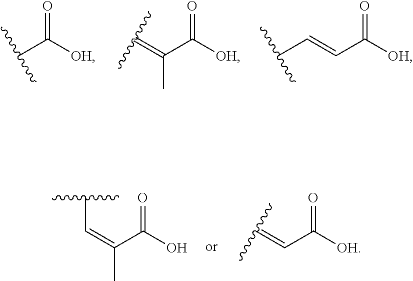

The second occurrence of R₃ is selected from

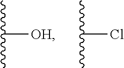

and C₁-C₆ alkyl group. In a further embodiment, the ring is aromatic, and carboxylic acid substituent is located at position 2, 3 or 5 of the ring, while the second R₃ substituent is located at position 4. In a further Formula I(A) embodiment, the carboxylic acid group is

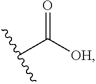

and the opioid is selected from hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol or a 2-, 3- or 4-phenolically hydroxylated phenazepine analgesic, e.g., a 2-, 3- or 4-phenolically hydroxylated ethoheptazine, proheptazine, metethoheptazine or metheptazine, or any other analgesic. Alternatively the opioid may be a narcotic antagonist such as alvimopan, de-glycinated alvimopan, naloxone, nalorphine or naltrexone. The opioid may be any other phenolic opioid disclosed in this specification.

The present invention is also directed to prodrugs of Formula I(B) where R₁ is hydrogen, R₂ is

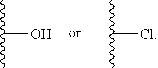

methylene or absent, Y and X are both —C—, n2 is 1, n is 2 and one R₃ is Therefore, in this embodiment, the other occurrence of R₃ is a carboxylic acid, and is selected from

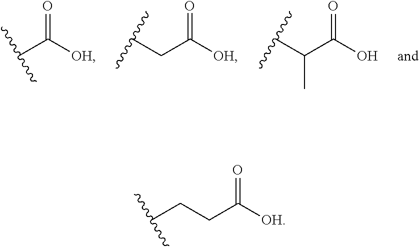

In a further embodiment, the ring is aromatic, and one R₃ substituent is located at position 2, 3 or 5 of the ring, while the second R₃ substituent is located at position 4. In a further Formula I(B) embodiment, at least one R₃ is

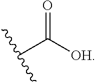

In one Formula I(B) embodiment, R₁ is hydrogen while R₂ is absent or methylene, n is 1 and R₃ is

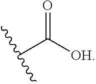

In a further embodiment,

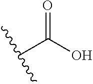

is located at the 4 position of a 6 membered ring. In a further embodiment, the ring is a benzene ring.

Additionally, the invention is also directed to prodrugs of Formula I(B) where R₁ is hydrogen, R₂ is methylene or absent, X is —N—, Y is —C—, n2 is 1, n is 2, and one occurrence of R₃ is a carboxylic acid group selected from

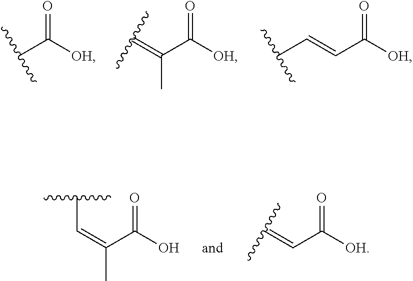

The second occurrence of R₃ is selected from

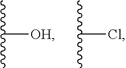

methyl and ethyl. In a further embodiment, the ring is aromatic, and the non-carboxylic acid group R₃ is located at position 2, 3 or 5 of the ring, while the carboxylic acid group R₃ is selected from

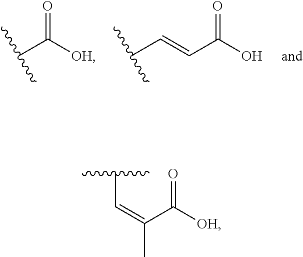

and is located at position 4. In a further formula I(B) embodiment, the carboxylic acid substituent is

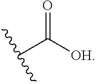

In yet a further embodiment, the opioid is an opioid antagonist.

Additionally, the invention is also directed to prodrugs of Formula I(B) where R₁ is hydrogen, R₂ is methylene or absent, X is —N—, Y is —C—, n is 2, n2 is 1, one R₃ is

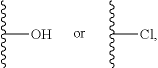

and the carboxylic acid R₃ is selected from

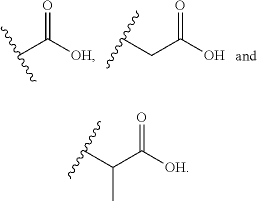

In a further embodiment, the ring is aromatic, and the

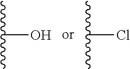

substituent is located at position 2, 3 or 5 of the ring, while the second R₃ substituent is located at position 4. In a further Formula I(B) embodiment, the second R₃ substituent is

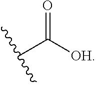

One Formula I(B) embodiment is directed to a compound defined as follows: R₁ is hydrogen, R₂ is absent, n2 is 1, Y and X are both —C—, n is 1 and R₃ is

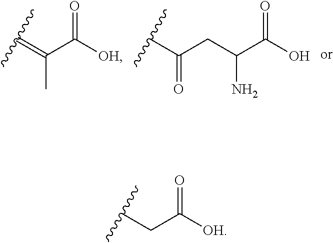

In another Formula I(B) embodiment, R₂ is absent or methylene, n is 1, n2 is 1, Y is —C—, while X is —N— and R₃ is

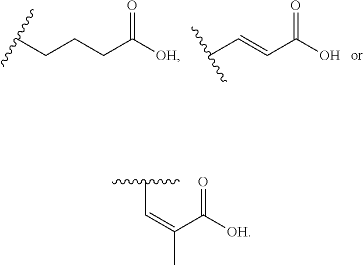

In a further embodiment, the ring is aromatic and R₃ is located at position 4 of the ring.

In one Formula I(B) embodiment, X is —C—, Y is —N—, R₁ is hydrogen while R₂ is absent or methylene, n is 2 and one occurrence of R₃ is

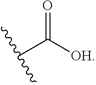

In a further embodiment, R₃ is located at the 4 position of a 6 membered ring. In a further embodiment, the ring is a benzene ring.

Another embodiment is directed to an opioid prodrug of Formula I(B) where n2 is 0. In a further embodiment, Y is —S— and X is —N—, and the ring is aromatic, i.e., the ring is a thiazole ring.

Yet another embodiment is directed to an opioid prodrug of Formula I(B) where n2 is 0, Y is —N— and X is —N—, and the ring is aromatic.

It will be recalled that the present invention also provides an opioid prodrug having a structure according to Formula (II):

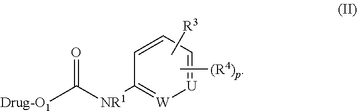

In an embodiment, the opioid drug having a phenolic hydroxyl group is an opioid drug selected from the group consisting of: hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine and a phenolically hydroxylated, e.g. a 2-, 3- or 4-phenolically hydroxylated phenazepine analgesic, e.g., a phenolically hydroxylated, e.g. a 2-, 3- or 4-phenolically hydroxylated of ethoheptazine, proheptazine, metethoheptazine or metheptazine, or any other analgesic. Alternatively the opioid may be a narcotic antagonist for example alvimopan, de-glycinated alvimopan, naloxone, N-methyl naloxone, nalorphine, naltrexone or N-methyl naltrexone. The opioid may be any other phenolic opioid disclosed in this specification.

In an embodiment, R¹ is selected from the group consisting of: H and C₁₋₄ alkyl (e.g. methyl, ethyl or propyl). In a preferred embodiment, R¹ is H.

In an embodiment, R³ is —(CR′R″)ᵣCOOH. In a preferred embodiment, r is 0, and therefore R³ is —COOH.

In an alternative embodiment, R³ is —(CR′R″)ᵣCOOH and r is 1 or 2. In an embodiment, R′ and R″ are each H.

In an embodiment, R³ is

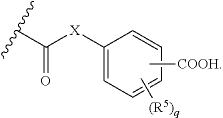

In an embodiment, X is —O—. In an embodiment, X is —NR⁶—. In an embodiment, R⁶ is selected from the group consisting of: H and C₁₋₄ alkyl (e.g. methyl, ethyl or propyl). In a preferred embodiment, R⁶ is H. In an embodiment, R⁵ has the same definition of R⁴.

In an embodiment, R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a more preferred embodiment, R⁴ is selected from the group comprising: F, methyl, ethyl, methoxy and ethoxy.

In an embodiment, R⁵ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R⁵ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a more preferred embodiment, R⁵ is selected from the group comprising: F, methyl, ethyl, methoxy and ethoxy.

In an embodiment, W is —CR′═, preferably —CH═. In an alternative embodiment, W is —N═.

In an embodiment, U is —CR′═, preferably —CH═.

In an embodiment, p is 0. In an embodiment, p is 1.

In an embodiment, q is 0. In an embodiment, q is 1.

In an embodiment, U is —CH═ and W is —CH═. In an alternate embodiment, U is —CH═ and W is —N═.

In an embodiment, R¹ is selected from the group consisting of: H and C₁₋₄ alkyl (e.g. methyl, ethyl or propyl) and R³ is —(CR′R″)ᵣCOOH. In a preferred embodiment, R¹ is H and R³ is —(CR′R″)ᵣCOOH. In a further preferred embodiment, R¹ is H, R³ is —(CR′R″)ᵣCOOH and r is 1. In each of these embodiments, preferably R′ and R″ are each H. In a further preferred embodiment, R¹ is H, R³ is —(CR′R″)ᵣCOOH and r is 0.

In an embodiment, R¹ is selected from the group consisting of: H and C₁₋₄ alkyl (e.g. methyl, ethyl or propyl), R³ is —(CR′R″)ᵣCOOH and p is 0. In a preferred embodiment, R¹ is H, R³ is —(CR′R″)ᵣCOOH, and p is 0. In a further preferred embodiment, R¹ is H, R³ is —(CR′R″)ᵣCOOH, r is 1 and p is 0. In each of these embodiments, preferably R′ and R″ are each H. In a further preferred embodiment, R¹ is H, R³ is —(CR′R″)ᵣCOOH, r is 0 and p is 0.

In an embodiment, R¹ is selected from the group consisting of: H and C₁₋₄ alkyl (e.g. methyl, ethyl or propyl), R³ is —(CR′R″)ᵣCOOH and p is 1. In a preferred embodiment, R¹ is H, R³ is —(CR′R″)ᵣCOOH, and p is 1. In a further preferred embodiment, R¹ is H, R³ is —(CR′R″)ᵣCOOH, r is 1 and p is 1. In each of these embodiments, preferably R′ and R″ are each H. In a further preferred embodiment, R¹ is H, R³ is —(CR′R″)ᵣCOOH, r is 0 and p is 1.

In an embodiment, R¹ is selected from the group consisting of: H and C₁₋₄ alkyl (e.g. methyl, ethyl or propyl), R³ is —(CR′R″)ᵣCOOH, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R¹ is H, R³ is —(CR′R″)ᵣCOOH, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, R¹ is H, R³ is —(CR′R″)ᵣCOOH, p is 1 and R⁴ is selected from the group comprising: F, methyl, ethyl, methoxy and ethoxy.

In an embodiment, R¹ is selected from the group consisting of: H and C₁₋₄ alkyl (e.g. methyl, ethyl or propyl), R³ is —(CR′R″)ᵣCOOH, r is 0, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R¹ is H, R³ is —(CR′R″)ᵣCOOH, r is 0, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, R¹ is H, R³ is —(CR′R″)ᵣCOOH, r is 0, p is 1 and R⁴ is selected from the group comprising: F, methyl, ethyl, methoxy and ethoxy.

In an embodiment, R¹ is selected from the group consisting of: H and C₁₋₄ alkyl (e.g. methyl, ethyl or propyl), R³ is —(CR′R″)ᵣCOOH, r is 1, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R¹ is H, R³ is —(CR′R″)ᵣCOOH, r is 1, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, R¹ is H, R³ is —(CR′R″)ᵣCOOH, r is 1, p is 1 and R⁴ is selected from the group comprising: F, methyl, ethyl, methoxy and ethoxy. In each of these embodiments, preferably R′ and R″ are each H.

In an embodiment, U is —CH═, W is —CH═, R¹ is selected from the group consisting of: H and C₁₋₄ alkyl (e.g. methyl, ethyl or propyl) and R³ is —(CR′R″)ᵣCOOH. In a preferred embodiment, U is —CH═, W is —CH═, R¹ is H and R³ is —(CR′R″)ᵣCOOH. In a further preferred embodiment, U is —CH═, W is —CH═, R¹ is H, R³ is —(CR′R″)ᵣCOOH and r is 1. In each of these embodiments, preferably R′ and R″ are each H. In a further preferred embodiment, U is —CH═, W is —CH═, R¹ is H, R³ is —(CR′R″)ᵣCOOH and r is 0.

In an embodiment, U is —CH═, W is —CH═, R¹ is selected from the group consisting of: H and C₁₋₄ alkyl (e.g. methyl, ethyl or propyl), R³ is —(CR′R″)ᵣCOOH and p is 0. In a preferred embodiment, U is —CH═, W is —CH═, R¹ is H, R³ is —(CR′R″)ᵣCOOH, and p is 0. In a further preferred embodiment, U is —CH═, W is —CH═, R¹ is H, R³ is —(CR′R″)ᵣCOOH, r is 1 and p is 0. In each of these embodiments, preferably R′ and R″ are each H. In a further preferred embodiment, U is —CH═, W is —CH═, R¹ is H, R³ is —(CR′R″)ᵣCOOH, r is 0 and p is 0.

In an embodiment, U is —CH═, W is —CH═, R¹ is selected from the group consisting of: H and C₁₋₄ alkyl (e.g. methyl, ethyl or propyl), R³ is —(CR′R″)ᵣCOOH and p is 1. In a preferred embodiment, U is —CH═, W is —CH═, R¹ is H, R³ is —(CR′R″)ᵣCOOH, and p is 1. In a further preferred embodiment, U is —CH═, W is —CH═, R¹ is H, R³ is —(CR′R″)ᵣCOOH, r is 1 and p is 1. In each of these embodiments, preferably R′ and R″ are each H. In a further preferred embodiment, U is —CH═, W is —CH═, R¹ is H, R³ is —(CR′R″)ᵣCOOH, r is 0 and p is 1.

In an embodiment, U is —CH═, W is —CH═, R¹ is selected from the group consisting of: H and C₁₋₄ alkyl (e.g. methyl, ethyl or propyl), R³ is —(CR′R″)ᵣCOOH, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —CH═, R¹ is H, R³ is —(CR′R″)ᵣCOOH, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —CH═, R¹ is H, R³ is —(CR′R″)ᵣCOOH, p is 1 and R⁴ is selected from the group comprising: F, methyl, ethyl, methoxy and ethoxy.

In an embodiment, U is —CH═, W is —CH═, R¹ is selected from the group consisting of: H and C₁₋₄ alkyl (e.g. methyl, ethyl or propyl), R³ is —(CR′R″)ᵣCOOH, r is 0, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —CH═, R¹ is H, R³ is —(CR′R″)ᵣCOOH, r is 0, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —CH═, R¹ is H, R³ is —(CR′R″)ᵣCOOH, r is 0, p is 1 and R⁴ is selected from the group comprising: F, methyl, ethyl, methoxy and ethoxy.

In an embodiment, U is —CH═, W is —CH═, R¹ is selected from the group consisting of: H and C₁₋₄ alkyl (e.g. methyl, ethyl or propyl), R³ is —(CR′R″)ᵣCOOH, r is 1, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —CH═, R¹ is H, R³ is —(CR′R″)ᵣCOOH, r is 1, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —CH═, R¹ is H, R³ is —(CR′R″)ᵣCOOH, r is 1, p is 1 and R⁴ is selected from the group comprising: F, methyl, ethyl, methoxy and ethoxy. In each of these embodiments, preferably R′ and R″ are each H.

In an embodiment, U is —CH═, W is —N═, R¹ is selected from the group consisting of: H and C₁₋₄ alkyl (e.g. methyl, ethyl or propyl) and R³ is —(CR′R″)ᵣCOOH. In a preferred embodiment, U is —CH═, W is —N═, R¹ is H and R³ is —(CR′R″)ᵣCOOH. In a further preferred embodiment, U is —CH═, W is —N═, R¹ is H, R³ is —(CR′R″)ᵣCOOH and r is 1. In each of these embodiments, preferably R′ and R″ are each H. In a further preferred embodiment, U is —CH═, W is —N═, R¹ is H, R³ is —(CR′R″)ᵣCOOH and r is 0.

In an embodiment, U is —CH═, W is —N═, R¹ is selected from the group consisting of: H and C₁₋₄ alkyl (e.g. methyl, ethyl or propyl), R³ is —(CR′R″)ᵣCOOH and p is 0. In a preferred embodiment, U is —CH═, W is —N═, R¹ is H, R³ is —(CR′R″)ᵣCOOH, and p is 0. In a further preferred embodiment, U is —CH═, W is —N═, R¹ is H, R³ is —(CR′R″)ᵣCOOH, r is 1 and p is 0. In each of these embodiments, preferably R′ and R″ are each H. In a further preferred embodiment, U is —CH═, W is —N═, R¹ is H, R³ is —(CR′R″)ᵣCOOH, r is 0 and p is 0.

In an embodiment, U is —CH═, W is —N═, R¹ is selected from the group consisting of: H and C₁₋₄ alkyl (e.g. methyl, ethyl or propyl), R³ is —(CR′R″)ᵣCOOH and p is 1. In a preferred embodiment, U is —CH═, W is —N═, R¹ is H, R³ is —(CR′R″)ᵣCOOH, and p is 1. In a further preferred embodiment, U is —CH═, W is —N═, R¹ is H, R³ is —(CR′R″)ᵣCOOH, r is 1 and p is 1. In each of these embodiments, preferably R′ and R″ are each H. In a further preferred embodiment, U is —CH═, W is —N═, R¹ is H, R³ is —(CR′R″)ᵣCOOH, r is 0 and p is 1.

In an embodiment, U is —CH═, W is —N═, R¹ is selected from the group consisting of: H and C₁₋₄ alkyl (e.g. methyl, ethyl or propyl), R³ is —(CR′R″)ᵣCOOH, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —N═, R¹ is H, R³ is —(CR′R″)ᵣCOOH, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —N═, R¹ is H, R³ is —(CR′R″)ᵣCOOH, p is 1 and R⁴ is selected from the group comprising: F, methyl, ethyl, methoxy and ethoxy.

In an embodiment, U is —CH═, W is —N═, R¹ is selected from the group consisting of: H and C₁₋₄ alkyl (e.g. methyl, ethyl or propyl), R³ is —(CR′R″)ᵣCOOH, r is 0, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —N═, R¹ is H, R³ is —(CR′R″)ᵣCOOH, r is 0, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —N═, R¹ is H, R³ is —(CR′R″)ᵣCOOH, r is 0, p is 1 and R⁴ is selected from the group comprising: F, methyl, ethyl, methoxy and ethoxy.

In an embodiment, U is —CH═, W is —N═, R¹ is selected from the group consisting of: H and C₁₋₄ alkyl (e.g. methyl, ethyl or propyl), R³ is —(CR′R″)ᵣCOOH, r is 1, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —N═, R¹ is H, R³ is —(CR′R″)ᵣCOOH, r is 1, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —N═, R¹ is H, R³ is —(CR′R″)ᵣCOOH, r is 1, p is 1 and R⁴ is selected from the group comprising: F, methyl, ethyl, methoxy and ethoxy. In each of these embodiments, preferably R′ and R″ are each H.

In an embodiment, R¹ is selected from the group consisting of: H and C₁₋₄ alkyl (e.g. methyl, ethyl or propyl) and R³ is

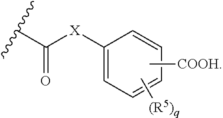

In a preferred embodiment, R¹ is H and R³ is

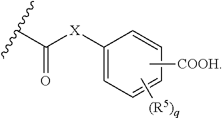

In an embodiment, R¹ is selected from the group consisting of: H and C₁₋₄ alkyl (e.g. methyl, ethyl or propyl), R³ is

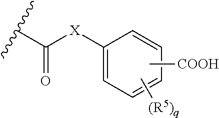

and p is 0. In a preferred embodiment, R¹ is H, R³ is

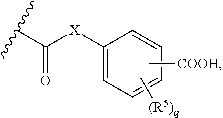

and p is 0.

In an embodiment, R¹ is selected from the group consisting of: H and C₁₋₄ alkyl (e.g. methyl, ethyl or propyl), R³ is

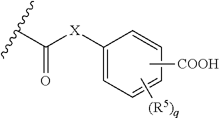

and p is 1. In a preferred embodiment, R¹ is H, R³ is

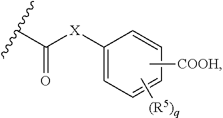

and p is 1.

In an embodiment, R¹ is selected from the group consisting of: H and C₁₋₄ alkyl (e.g. methyl, ethyl or propyl), R³ is

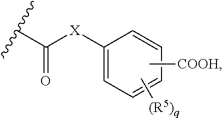

p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R¹ is H, R³ is

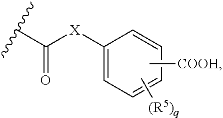

p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, R¹ is H, R³

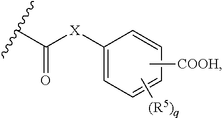

p is 1 and R⁴ is selected from the group comprising: F, methyl, ethyl, methoxy and ethoxy.

In an embodiment, U is —CH═, W is —CH═, R¹ is selected from the group consisting of: H and C₁₋₄ alkyl (e.g. methyl, ethyl or propyl) and R³ is

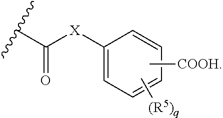

In a preferred embodiment, U is —CH═, W is —CH═, R¹ is H and R³ is

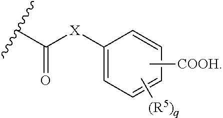

In an embodiment, U is —CH═, W is —CH═, R¹ is selected from the group consisting of: H and C₁₋₄ alkyl (e.g. methyl, ethyl or propyl), R³ is

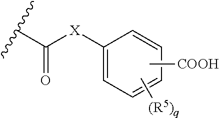

and p is 0. In a preferred embodiment, U is —CH═, W is —CH═, R¹ is H, R³ is

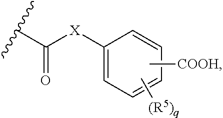

and p is 0.

In an embodiment, U is —CH═, W is —CH═, R¹ is selected from the group consisting of: H and C₁₋₄ alkyl (e.g. methyl, ethyl or propyl), R³ is

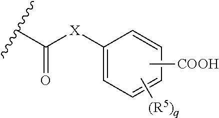

and p is 1. In a preferred embodiment, U is —CH═, W is —CH═, R¹ is H, R³ is

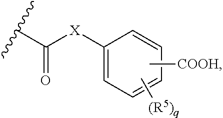

and p is 1.

In an embodiment, U is —CH═, W is —CH═, R¹ is selected from the group consisting of: H and C₁₋₄ alkyl (e.g. methyl, ethyl or propyl),

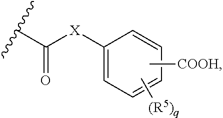

p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —CH═, R¹ is H, R³ is

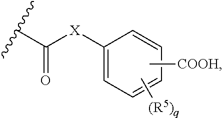

p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —CH═, R¹ is H, R³ is

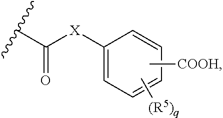

p is 1 and R⁴ is selected from the group comprising: F, methyl, ethyl, methoxy and ethoxy.

In an embodiment, U is —CH═, W is —N═, R¹ is selected from the group consisting of: H and C₁₋₄ alkyl (e.g. methyl, ethyl or propyl) and R³ is

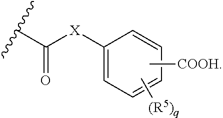

In a preferred embodiment, U is —CH═, W is —N═, R¹ is H and R³ is

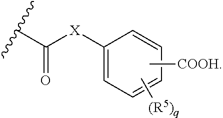

In an embodiment, U is —CH═, W is —N═, R¹ is selected from the group consisting of: H and C₁₋₄ alkyl (e.g. methyl, ethyl or propyl), R³ is

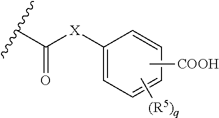

and p is 0. In a preferred embodiment, U is —CH═, W is —N═, R¹ is H, R³ is

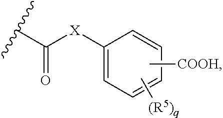

and p is 0.

In an embodiment, U is —CH═, W is —N═, R¹ is selected from the group consisting of: H and C₁₋₄ alkyl (e.g. methyl, ethyl or propyl), R³ is

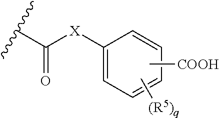

and p is 1. In a preferred embodiment, U is —CH═, W is —N═, R¹ is H, R³ is

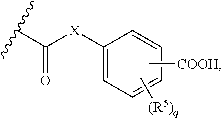

and p is 1.

In an embodiment, U is —CH═, W is —N═, R¹ is selected from the group consisting of: H and C₁₋₄ alkyl (e.g. methyl, ethyl or propyl), R³ is

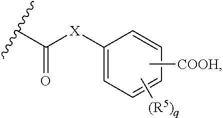

p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —N═, R¹ is H, R³ is

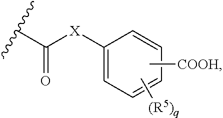

p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —N═, R¹ is H, R³ is

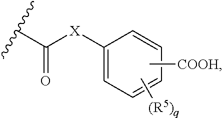

p is 1 and R⁴ is selected from the group comprising: F, methyl, ethyl, methoxy and ethoxy.

In an embodiment, R¹ is selected from the group consisting of: H and C₁₋₄ alkyl (e.g. methyl, ethyl or propyl), R³ is

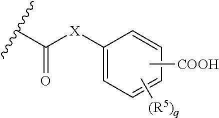

and X is —O—. In a preferred embodiment, R¹ is H, R³ is

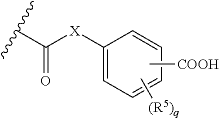

### and X is —O—.

In an embodiment, R¹ is selected from the group consisting of: H and C₁₋₄ alkyl (e.g. methyl, ethyl or propyl), R³ is

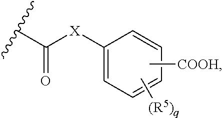

X is —O— and p is 0. In a preferred embodiment, R¹ is H, R³ is

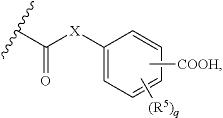

### X is —O— and p is 0.

In an embodiment, R¹ is selected from the group consisting of: H and C₁₋₄ alkyl (e.g. methyl, ethyl or propyl), R³ is

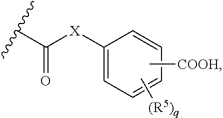

X is —O— and p is 1. In a preferred embodiment, R¹ is H, R³ is

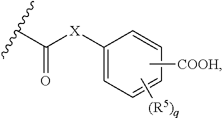

### X is —O— and p is 1.

In an embodiment, R¹ is selected from the group consisting of: H and C₁₋₄ alkyl (e.g. methyl, ethyl or propyl), R³ is

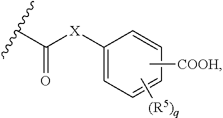

X is —O—, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R¹ is H, R³ is

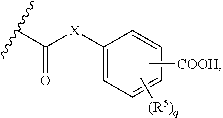

X is —O—, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, R¹ is H, R³

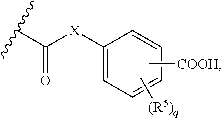

X is —O—, p is 1 and R⁴ is selected from the group comprising: F, methyl, ethyl, methoxy and ethoxy.

In an embodiment, U is —CH═, W is —CH═, R¹ is selected from the group consisting of: H and C₁₋₄ alkyl (e.g. methyl, ethyl or propyl), R³ is

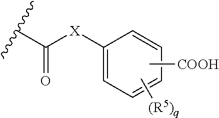

and X is —O—. In a preferred embodiment, U is —CH═, W is —CH═, R¹ is H, R³ is

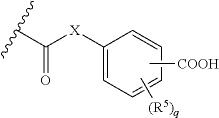

### and X is —O—.

In an embodiment, U is —CH═, W is —CH═, R¹ is selected from the group consisting of: H and C₁₋₄ alkyl (e.g. methyl, ethyl or propyl), R³ is

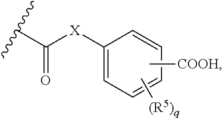

X is —O— and p is 0. In a preferred embodiment, U is —CH═, W is —CH═, R¹ is H, R³ is

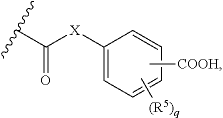

### X is —O— and p is 0.

In an embodiment, U is —CH═, W is —CH═, R¹ is selected from the group consisting of: H and C₁₋₄ alkyl (e.g. methyl, ethyl or propyl), R³ is

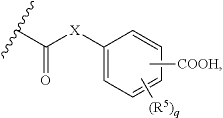

X is —O— and p is 1. In a preferred embodiment, U is —CH═, W is —CH═, R¹ is H, R³ is

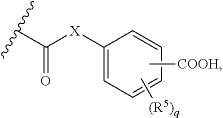

### X is —O— and p is 1.

In an embodiment, U is —CH═, W is —CH═, R¹ is selected from the group consisting of: H and C₁₋₄ alkyl (e.g. methyl, ethyl or propyl), R³ is

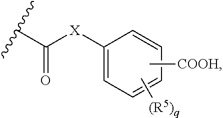

X is —O—, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —CH═, R¹ is H, R³ is

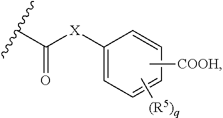

X is —O—, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —CH═, R¹ is H, R³ is

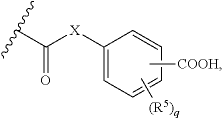

X is —O—, p is 1 and R⁴ is selected from the group comprising: F, methyl, ethyl, methoxy and ethoxy.

In an embodiment, U is —CH═, W is —N═, R¹ is selected from the group consisting of: H and C₁₋₄ alkyl (e.g. methyl, ethyl or propyl), R³ is

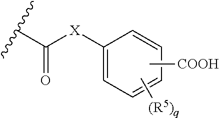

and X is —O—. In a preferred embodiment, U is —CH═, W is —N═, R¹ is H, R³ is

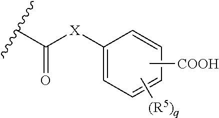

### and X is —O—.

In an embodiment, U is —CH═, W is —N═, R¹ is selected from the group consisting of: H and C₁₋₄ alkyl (e.g. methyl, ethyl or propyl), R³ is

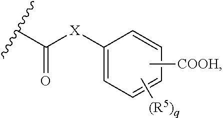

X is —O— and p is 0. In a preferred embodiment, U is —CH═, W is —N═, R¹ is H, R³ is

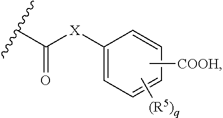

### X is —O— and p is 0.

In an embodiment, U is —CH═, W is —N═, R¹ is selected from the group consisting of: H and C₁₋₄ alkyl (e.g. methyl, ethyl or propyl), R³ is

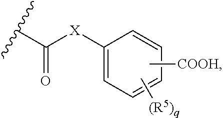

X is —O— and p is 1. In a preferred embodiment, U is —CH═, W is —N═, R¹ is H, R³ is

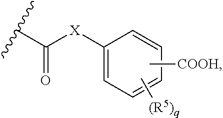

### X is —O— and p is 1.

In an embodiment, U is —CH═, W is —N═, R¹ is selected from the group consisting of: H and C₁₋₄ alkyl (e.g. methyl, ethyl or propyl), R³ is

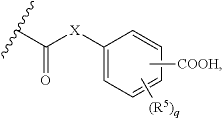

X is —O—, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —N═, R¹ is H, R³ is

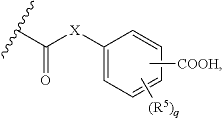

X is —O—, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —N═, R¹ is H, R³ is

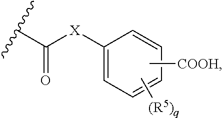

X is —O—, p is 1 and R⁴ is selected from the group comprising: F, methyl, ethyl, methoxy and ethoxy.

In an embodiment, R¹ is selected from the group consisting of: H and C₁₋₄ alkyl (e.g. methyl, ethyl or propyl), R³ is

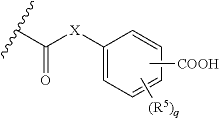

and X is —NH—. In a preferred embodiment, R¹ is H, R³ is

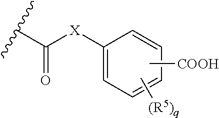

### and X is —NH—.

In an embodiment, R¹ is selected from the group consisting of: H and C₁₋₄ alkyl (e.g. methyl, ethyl or propyl), R³ is

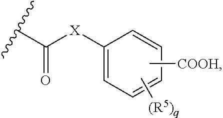

X is —NH— and p is 0. In a preferred embodiment, R¹ is H, R³ is

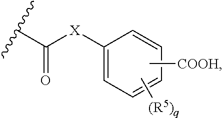

### X is —NH— and p is 0.

In an embodiment, R¹ is selected from the group consisting of: H and C₁₋₄ alkyl (e.g. methyl, ethyl or propyl), R³ is

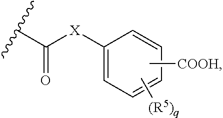

X is —NH— and p is 1. In a preferred embodiment, R¹ is H, R³ is

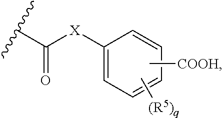

### X is —NH— and p is 1.

In an embodiment, R¹ is selected from the group consisting of: H and C₁₋₄ alkyl (e.g. methyl, ethyl or propyl), R³ is

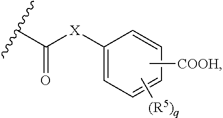

X is —NH—, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R¹ is H, R³ is

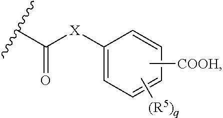

X is —NH—, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, R¹ is H, R³

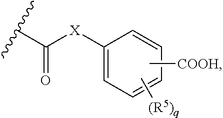

X is —NH—, p is 1 and R⁴ is selected from the group comprising: F, methyl, ethyl, methoxy and ethoxy.

In an embodiment, U is —CH═, W is —CH═, R¹ is selected from the group consisting of: H and C₁₋₄ alkyl (e.g. methyl, ethyl or propyl), R³ is

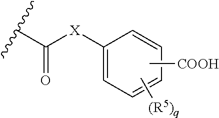

and X is —NH—. In a preferred embodiment, U is —CH═, W is —CH═, R¹ is H, R³ is

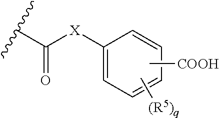

### and X is —NH—.

In an embodiment, U is —CH═, W is —CH═, R¹ is selected from the group consisting of: H and C₁₋₄ alkyl (e.g. methyl, ethyl or propyl), R³ is

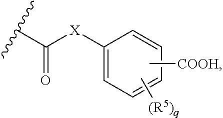

X is —NH— and p is 0. In a preferred embodiment, U is —CH═, W is —CH═, R¹ is H, R³ is

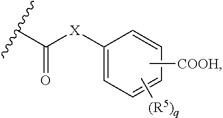

### X is —NH— and p is 0.

In an embodiment, U is —CH═, W is —CH═, R¹ is selected from the group consisting of: H and C₁₋₄ alkyl (e.g. methyl, ethyl or propyl), R³ is

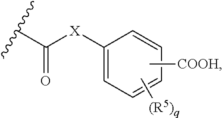

X is —NH— and p is 1. In a preferred embodiment, U is —CH═, W is —CH═, R¹ is H, R³ is

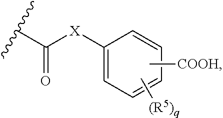

### X is —NH— and p is 1.

In an embodiment, U is —CH═, W is —CH═, R¹ is selected from the group consisting of: H and C₁₋₄ alkyl (e.g. methyl, ethyl or propyl), R³ is

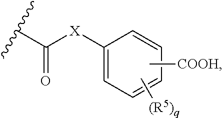

X is —NH—, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —CH═, R¹ is H, R³ is

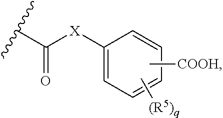

X is —NH—, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —CH═, R¹ is H, R³ is

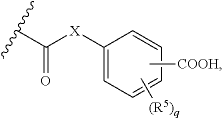

X is —NH—, p is 1 and R⁴ is selected from the group comprising: F, methyl, ethyl, methoxy and ethoxy.

In an embodiment, U is —CH═, W is —N═, R¹ is selected from the group consisting of: H and C₁₋₄ alkyl (e.g. methyl, ethyl or propyl), R³ is

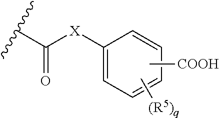

and X is —NH—. In a preferred embodiment, U is —CH═, W is —N═, R¹ is H, R³ is

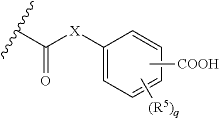

### and X is —NH—.

In an embodiment, U is —CH═, W is —N═, R¹ is selected from the group consisting of: H and C₁₋₄ alkyl (e.g. methyl, ethyl or propyl), R³ is

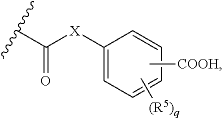

X is —NH— and p is 0. In a preferred embodiment, U is —CH═, W is —N═, R¹ is H, R³ is

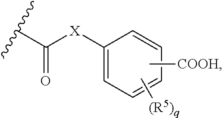

### X is —NH— and p is 0.

In an embodiment, U is —CH═, W is —N═, R¹ is selected from the group consisting of: H and C₁₋₄ alkyl (e.g. methyl, ethyl or propyl), R³ is

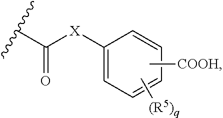

X is —NH— and p is 1. In a preferred embodiment, U is —CH═, W is —N═, R¹ is H, R³ is

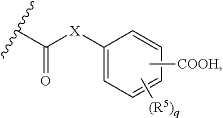

### X is —NH— and p is 1.

In an embodiment, U is —CH═, W is —N═, R¹ is selected from the group consisting of: H and C₁₋₄ alkyl (e.g. methyl, ethyl or propyl), R³ is

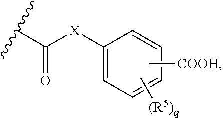

X is —NH—, p is 1 and R⁴ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, U is —CH═, W is —N═, R¹ is H, R³ is

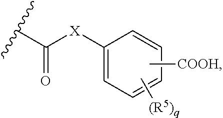

X is —NH—, p is 1 and R⁴ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —N═, R¹ is H, R³ is

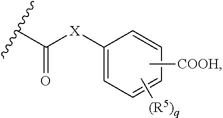

X is —NH—, p is 1 and R⁴ is selected from the group comprising: F, methyl, ethyl, methoxy and ethoxy.

In any of the above embodiments, q may be 0.

In an embodiment, the opioid prodrug of Formula II has the structure:

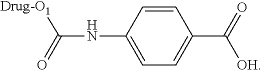

In an embodiment, the opioid prodrug of Formula II has the structure:

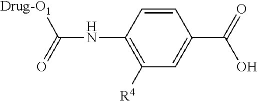

In an embodiment, the opioid prodrug of Formula II has the structure:

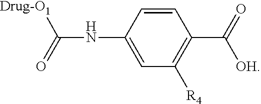

In an embodiment, the opioid prodrug of Formula II has the structure:

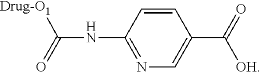

In an embodiment, the opioid prodrug of Formula II has the structure:

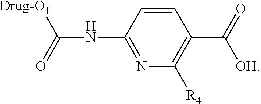

In an embodiment, the opioid prodrug of Formula II has the structure:

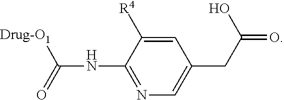

In an embodiment, the opioid prodrug of Formula II has the structure:

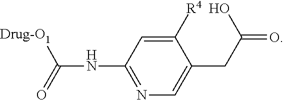

In an embodiment, the opioid prodrug of Formula II has the structure:

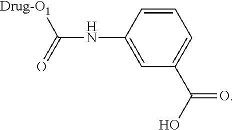

In an embodiment, the opioid prodrug of Formula II has the structure:

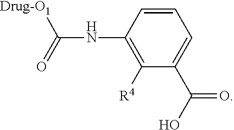

In an embodiment, the opioid prodrug of Formula II has the structure:

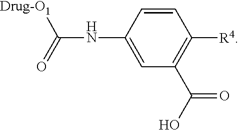

In an embodiment, the opioid prodrug of Formula II has the structure:

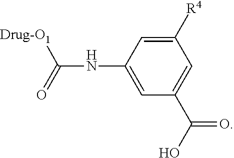

In an embodiment, the opioid prodrug of Formula II has the structure:

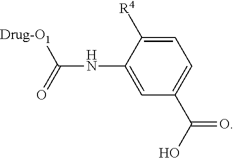

In an embodiment, the opioid prodrug of Formula II has the structure:

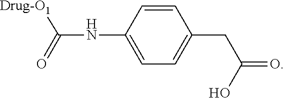

In an embodiment, the opioid prodrug of Formula II has the structure:

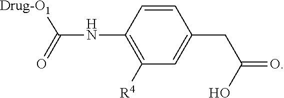

In an embodiment, the opioid prodrug of Formula II has the structure:

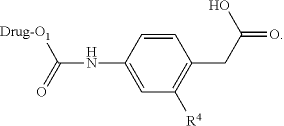

In an embodiment, the opioid prodrug of Formula II has the structure:

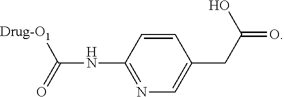

In an embodiment, the opioid prodrug of Formula II has the structure:

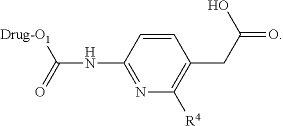

In an embodiment, the opioid prodrug of Formula II has the structure:

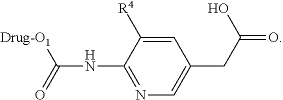

In an embodiment, the opioid prodrug of Formula II has the structure:

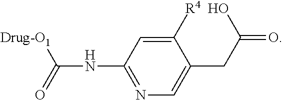

In an embodiment, the opioid prodrug of Formula II has the structure:

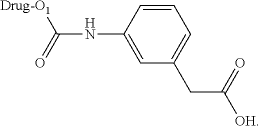

In an embodiment, the opioid prodrug of Formula II has the structure:

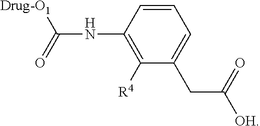

In an embodiment, the opioid prodrug of Formula II has the structure:

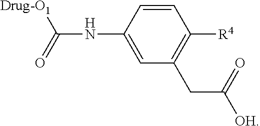

In an embodiment, the opioid prodrug of Formula II has the structure:

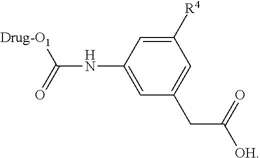

In an embodiment, the opioid prodrug of Formula II has the structure:

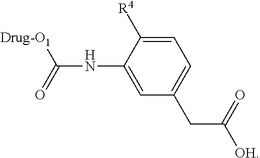

In an embodiment, the opioid prodrug of Formula II has the structure:

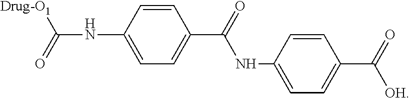

In an embodiment, the opioid prodrug of Formula II has the structure:

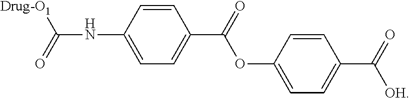

### Meptazinol Prodrugs of the Present Invention

Other single ABA or ABA analogue prodrugs of the present invention include meptazinol-2-amino benzoic acid carbamate, meptazinol-anthranilic acid carbamate, meptazinol-3-amino benzoic acid carbamate, meptazinol-4-aminomethyl benzoic acid carbamate, meptazinol-4-amino salicylic acid carbamate, meptazinol-4-amino cyclohexanoic acid carbamate, meptazinol-4-amino-phenyl acetic acid carbamate, meptazinol-4-amino-hippuric acid acid carbamate, meptazinol-2-amino benzoic acid carbamate, meptazinol-4-amino-2-chlorobenzoic acid carbamate, meptazinol-6-aminonicotinic acid carbamate, meptazinol-methyl-6-aminonicotinate acid carbamate, meptazinol-4-amino methyl salicylic acid carbamate, meptazinol-2-amino thiazole-4-acetic acid acid carbamate, meptazinol-2-amino benzoic acid carbamate and meptazinol-2-amino-4-(2-aminophenyl)-4-oxobutanoic acid. The meptazinol can be substituted with (1) an active metabolite of meptazinol, (2) a different opioid or (3) a similar PABA analogue for the meptazinol portion of the prodrug or the prodrug moiety, respectively.

Prodrugs of Opioid Analgesics from the Phenazepine Family

It will be appreciated by one of ordinary skill in the art that meptazinol has a similar structure to that of opioid analgesics from the phenazepine family (e.g., ethoheptazine). Although the phenazepine analgesics are not hydroxylated on their respective aromatic ring, potentially, active analogues of these analgesics could be ortho-, meta- or para-hydroxylated. Accordingly, the ortho, meta and/or para hydroxylated phenazepine analogues may be prodrugged with an ABA or an ABA analogue, as described throughout the application. The ortho-, meta- or para-hydroxylated analogues of these analgesics may also be considered as being metabolites of the opioid analgesics from the phenazepine family. For example, prodrugs of the following compounds are contemplated by the present invention:

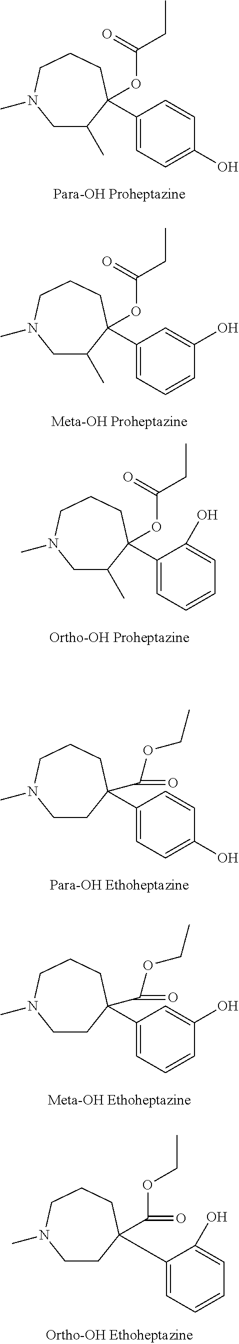

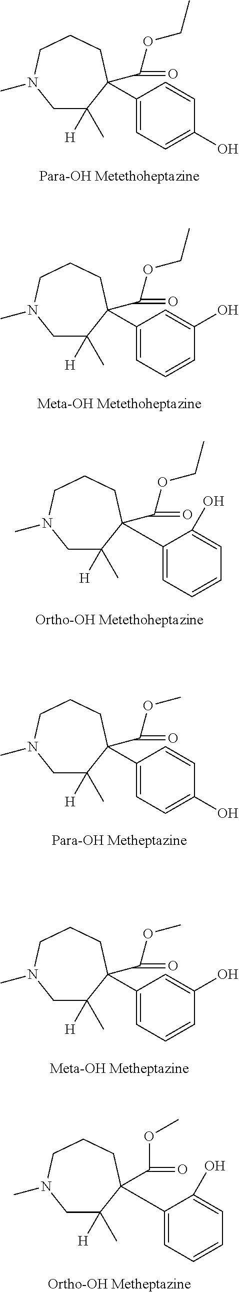

In the phenazepine prodrug embodiments, the prodrug moiety (i.e., ABA or ABA analogue) is conjugated to the hydroxyl group of the respective active analogue. Any of the ABA and ABA analogue prodrug moieties described herein can be conjugated to a phenazepine analogue, in order to arrive at a phenazepine analogue prodrug.

Although the invention has been described above with particular opioids, such as meptazinol (and active metabolites thereof), it is not limited thereto. Any opioid with a hydroxylic or oxo function can be used in the present invention, to arrive at one of the prodrugs described herein. For example, hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, a phenolically hydroxylated, e.g. a 2-, 3- or 4-phenolically hydroxylated phenazepine analgesic, e.g., a 2-, 3- or 4-phenolically hydroxylated ethoheptazine, proheptazine, metethoheptazine or metheptazine, or any other analgesic, or narcotic antagonist such as alvimopan, de-glycinated alvimopan, naloxone, nalorphine or naltrexone, can all be used as the active agent portion of a prodrug of the present invention.

### ADVANTAGES OF THE INVENTION

Without wishing to be bound to any particular theory (including that described in this paragraph), it is believed that the opioid prodrug of the present invention selectively exploits one or more of the inherent nutrient transporter(s) within the digestive tract to effect absorption of the drug. Para amino benzoic acid (PABA) structurally mimics the dipeptide phenylalanine-alanine having a five carbon bond separation between the amine and carboxyl termini. However, unlike that dipeptide, PABA is non-hydrolyzable and may thus be a stable substrate for the di and tripeptide transporter, Pept1. This transporter is utilized in the absorption of the prodrug valacyclovir (Landowski (2003), J. Pharmacol Expt Ther 306, 778-786). Alternatively, PABA, as an aryl carboxylic acid, could be a substrate for the ceftibuten/fluorocein transporter which is known to be involved in the absorption of various aryl carboxylic acids including the oral hypoglycemic agent nateglinide (Itagaki et al. (2005), Biochim & Biophys Acta 90, 190-194). Alternatively, ABA prodrug conjugates may be absorbed by the monocarboxylate (MCT) family of transporters involved in the absorption of gabapentin enacarbil (Revill P et al (2006), Drugs. Future, 31 771-777).

Without wishing to be bound to any particular theory (including that described in this paragraph), it is further believed that, as well as conferring the characteristics necessary for substrate recognition by one or more gut transporters, the proximity of the aryl ring in PABA to the carbamate linkage results in the latter's chemical lability by PABA's electronegative (electron withdrawing) properties. This facilitates the chemical cleavage and release of the active drug at blood pH of about 7.4. Despite such lability at this pH, at pHs prevailing in the gut—from about 1 to about 6.8—the prodrugs of the present invention appear comparatively stable, allowing the prodrug to be absorbed per se. Once in the blood, chemical activation is possible, and release of the active drug can begin. Such chemical activation of the prodrug, as opposed enzymatic release, avoids the almost inevitable species differences in enzyme expression. Thus, exploitation of chemical activation may serve to reduce the uncertainties of extrapolation of animal data to man. Furthermore, interpatient variability—as the result of health status, age or genetic predisposition—associated with metabolic prodrug activation and variable therapeutic benefit, is avoided.

Thus and again without wishing to be bound by any particular theory, the prodrugs of the present invention may overcome the limitations of administration of opioids and previously designed opioid prodrugs, by the exploitation of chemical cleavage, whereby the prodrugs of the present invention are cleaved at pHs greater than the pH of the stomach and gut.

Reduction of the adverse GI side-effects associated with opioid administration may be an added advantage of using a prodrug of the present invention. Derivatization of an opioid in the manner described is likely to reduce or abolish the opioid-like, and potentially other, contributory pharmacological effects as the result of a profound change in physicochemical characteristics of the opioid, after conjugation to PABA or a PABA analogue. Oral administration of a temporarily inactivated opioid would preclude access of active drug species—during the absorption process—to the μ-opioid and other receptors within the gut wall. The importance of interactions with gut receptors has recently been established in respect of both opioid and cholinergic effects. The role of the latter in eliciting the profound emesis associated with the acetyl choline esterase inhibitor galantamine has been demonstrated by Kays et al. 2007, Internet J. of Pharmaceut. 335 138-146. Of the opioids of the present invention, meptazinol has acetylcholine esterase inhibitory (AChEI) activity comparable to that of galantamine and may therefore elicit its emetic side effects through this same mechanism. While Chou Z et al showed the simple phenyl carbamate of meptazinol to have a 1500-fold increased acetyl choline esterase inhibitory activity, the corresponding PABA carbamate was found to be 10-fold less potent. This is consistent with established SAR for AChIE molecules in which the introduction of a carboxyl residue into the molecule is well known to dramatically reduce acetyl choline esterase inhibitory activity (Soriano E et al (2010) Bioorg Med Chem. Lett. 20, 2950-3).

The importance of the intestinal μ-opioid receptors on gut motility has been shown by the beneficial effects of the locally confined narcotic antagonists alvimopan, and naloxone. (Linn and Steinbrook (2007). Tech in Reg. Anaes. and Pain Management 11, 27-32). Oral co-administration of these agents with opioids such as oxycodone has been shown to successfully overcome their usually constipating effects without affecting systemically mediated analgesia. The use of a transiently inactivated prodrug could more effectively fulfill this role by avoiding any possible direct interaction of active drug with gut receptors. Similarly, the cholinergic effects on the gut of some opioids such as meptazinol would be avoided.

Advantageously, a principal advantage of the invention herein described is shielding against first pass metabolism commonly seen with phenolic drugs, and the consquences for poor and erratic systemic drug availability. Oral administration of a prodrug of the present invention may afford temporary protection against such extensive first pass metabolism and the consequential low bioavailability, and resultant variability, in attained plasma drug levels. Such temporary shielding of the metabolically vulnerable phenolic function by a prodrug moiety should ensure reduced first pass metabolism of the drug and improve the oral bioavailability of the respective opioid. Additionally, the administration of such a prodrug could also lead to maintenance of drug plasma levels as the result of continuing generation of drug from a plasma reservoir of prodrug.

The improvements in bioavailability which may be offered by the prodrugs of the present invention are likely to lead to greater predictability of analgesic response both within and between subjects (potential for less variability of analgesic response and drug plasma levels for both (1) individual subjects and (2) a subject population) and hence improve subject compliance.

Another added potential advantage of the use of such prodrugs is a reduced likelihood of intravenous or intranasal abuse. An initially inactive opioid prodrug may reduce the propensity for intravenous abuse because of the prodrug's slower attainment rate of peak active drug levels, compared to administration of free opioid. This should give a reduced “euphoric rush” to potential abusers. intranasal abuse may also be reduced by the greater likelihood of poor absorption of a hydrophilic prodrug via the nasal mucosa. This would be the consequence of the profound difference in physicochemical properties between the parent opioid and a highly water soluble ABA and ABA analogue prodrug described herein. ABA and ABA analogue prodrugs are not likely to be absorbed by simple diffusion due to their high water solubility and also adverse LogP values. Instead, they should rely upon active transporters, such as Pept1, which, while present in the gut, are essentially absent in the nasal mucosa.

It is believed that a further advantage of the invention may be a consequence of the resultant radical change in the physico-chemical characteristics of the drug with the prodrugs of the present invention being acidic or zwitterionic. The consequential reduction in lipophilicity should limit unwanted initial widespread non-specific tissue distribution. Drugs which are particularly lipophilic may be subject to extensive “first pass tissue distribution” whereby they may be taken up initially widely into numerous non target tissues including body fat and released only very slowly. The slow release of sub-therapeutic drug levels may contribute little to the activity of the drug. Furthermore widespread non target distribution may lead to unwanted adverse effects in some of these tissues. In the case of the acidic or zwitterionic prodrug, extensive initial drug “loss” due to first pass tissue distribution is avoided or reduced as the prodrug would be largely confined to the vascular compartment.

A particular advantage to the use of para amino benzoic acid (PABA) as the prodrugging moiety attached to the drugs' phenolic function is that subsequent to cleavage the products are just the active drug, PABA and carbon dioxide. PABA is a so called GRAS substance—generally regarded as safe appearing in the FDA's GRAS Registrary. It is widely used a health food/dietary supplement and taken in typical doses of up to 300 mg/day. The potassium salt of PABA, known as POTABA, is available on prescription and is indicated for Peyronie's Disease and scleroderma. The dose used for these disorders is 12 grams daily taken in four to six divided doses with meals. Thus the clinical safety of PABA would appear to be assured.

### Uses and Methods of the Invention

One embodiment is a method of treating a disorder in a subject in need thereof with an opioid. The method comprises orally administering a therapeutically effective amount (e.g., an analgesic effective amount) of an opioid prodrug of the present invention to the subject (an opioid bonded to ABA or an ABA analogue via a carbamate or thiocarbamate bond, for example, a prodrug of any of the Formulae I, I(A), I(B), or II). The disorder may be one treatable with an opioid. For example, the disorder may be pain, such as neuropathic pain or nociceptive pain.

Specific types of pain which can be treated with the opioid prodrugs include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment. Any of the prodrugs presented herein can be used in a method of treating pain.

In the methods of treating pain, the prodrugs may be administered in conjunction with other therapies and/or in combination with other active agents (e.g., other analgesics). For example, the prodrugs may be administered to a subject in combination with other active agents used in the management of pain. An active agent to be administered in combination with the prodrugs encompassed by the present invention may include, for example, a drug selected from the group consisting of non-steroidal anti-inflammatory drugs (e.g., ibuprofen), anti-emetic agents (e.g., ondansetron, domerperidone, hyoscine and metoclopramide), or unabsorbed or poorly bioavailable opioid antagonists (e.g., naloxone) to reduce the risk of drug abuse. In such combination therapies, the prodrugs encompassed by the present invention may be administered prior to, concurrent with, or subsequent to the other therapy and/or active agent. The prodrug and other active agent(s) may also be incorporated into a single dosage form.

In one embodiment, the present invention is directed to a method for increasing the oral bioavailability of an opioid analgesic which has a significantly lower bioavailability when administered in its unbound form. The method comprises administering, to a subject in need thereof, an opioid prodrug or a pharmaceutically acceptable salt thereof to a subject in need thereof, wherein the opioid prodrug is comprised of an opioid analgesic (or active metabolite thereof) covalently bonded via a carbamate or thiocarbamate linkage, to ABA, or an ABA analogue, and upon oral administration, the oral bioavailability of the opioid is at least 120% that of the opioid, when administered alone. The amount of the opioid is preferably a therapeutically effective amount (e.g., an analgesic effective amount). In this embodiment, the opioid prodrug can be any opioid prodrug of Formulae I, I(A), I(B) or II, or a pharmaceutically acceptable salt thereof.

In another embodiment, the present invention is directed to a method for minimizing the gastrointestinal side effects normally associated with administration of an opioid analgesic, wherein the opioid has a derivatizable phenolic function. The method comprises orally administering an opioid prodrug, or a pharmaceutically acceptable salt thereof, to a subject in need thereof, wherein the opioid prodrug is comprised of an opioid analgesic (or active metabolite thereof) covalently bonded via a carbamate or thiocarbamate linkage, to ABA, or an ABA analogue, and after oral administration, the subject partially avoids or completely avoids the gastrointestinal side effects usually seen after oral administration of the unbound opioid analgesic. The amount of the opioid (or active metabolite) is preferably a therapeutically effective amount (e.g., an analgesic effective amount). The term “unbound opioid analgesic” refers to an opioid analgesic which is not a prodrug. This method is particularly useful for reducing gastrointestinal side effect(s) resulting from or aggravated by administration of the unbound opioid analgesic for pain relief. In this embodiment, the opioid prodrug can be any opioid prodrug of the cited Formulae I, I(A), I(B) or II, or pharmaceutically acceptable salt thereof.

### Salts, Solvates, & Derivatives of the Compounds of the Invention

The compounds, compositions and methods of the present invention further encompass the use of salts, solvates, of the opioid prodrugs described herein. In one embodiment, the invention disclosed herein is meant to encompass all pharmaceutically acceptable salts of opioid prodrugs (including those of the carboxyl function of ABA, or its analogues or the terminal amino acid, as well as those of the weakly basic nitrogen within the opioid.

Typically, a pharmaceutically acceptable salt of a prodrug of an opioid of the present invention is prepared by reaction of the prodrug with a desired acid or base, as appropriate. The salt may precipitate from solution and be collected by filtration or may be recovered by evaporation of the solvent. For example, an aqueous solution of an acid such as hydrochloric acid may be added to an aqueous suspension of the opioid prodrug and the resulting mixture evaporated to dryness (lyophilized) to obtain the acid addition salt as a solid. Alternatively, the prodrug may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain (1999). Polymorphism in Pharmaceutical solids. Marcel Decker, New York). The compounds useful in the practice of the present invention can have one or more chiral centers and, depending on the nature of individual substituents, they can also have geometrical isomers.

### Pharmaceutical Compositions of the Invention

While it is possible that, for use in the methods of the invention, the prodrug of the present invention may be administered as the isolated substance, the active ingredient may be presented in a pharmaceutical composition, e.g., wherein the agent is in admixture with a pharmaceutically acceptable carrier selected with regard to the intended route of administration and standard pharmaceutical practice. In one embodiment of the present invention, a composition comprising an opioid prodrug of the present invention (e.g., a prodrug of any of the Formulae provided. The composition comprises at least one opioid prodrug selected from the Formula provided, and at least one pharmaceutically acceptable excipient or carrier.

The formulations of the invention may be immediate-release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, e.g., in Remington, The Science and Practice of Pharmacy, 20^{th} Edition, 2000, pp. 858-914.

However, since absorption of opioid prodrugs may proceed via active transporters located in specific regions of GI tract, unconventional controlled dosage forms may be desirable. For example, the Pept1 transporter is believed to be largely confined to the upper GI tract, and should it be a contributor to prodrug absorption, may limit the effectiveness for continued absorption along the whole length of the GI tract.

For those opioid prodrugs which do not result in sustained plasma drugs levels due to continuous generation of active agent from a plasma reservoir of prodrug—but which may offer other advantages—gastroretentive or mucoretentive formulations analogueous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption take place and effectively prolonging plasma drug levels. Other drug delivery systems affording delayed progression along the GI tract, such as mucoadhesive formulations, may also be of value.

The formulations of the present invention can be administered, for example, from one to six times daily, depending on the dosage form and dosage.

In one embodiment, the present invention provides a pharmaceutical composition comprising at least one active pharmaceutical ingredient (i.e., an opioid prodrug), or a pharmaceutically acceptable derivative (e.g., a salt or solvate) thereof, and a pharmaceutically acceptable carrier or excipient. In particular, the invention provides a pharmaceutical composition comprising a therapeutically effective amount of at least one opioid prodrug of the present invention (an opioid bonded to ABA or an ABA analogue via a carbamate bond, for example, a prodrug of any of the Formulae I, I(A) or II), or a pharmaceutically acceptable derivative thereof, and a pharmaceutically acceptable carrier or excipient.

The prodrug employed in the present invention may be used in combination with other therapies and/or active agents. Accordingly, the present invention provides, in another embodiment, a pharmaceutical composition comprising at least one compound useful in the practice of the present invention, or a pharmaceutically acceptable salt or solvate thereof, a second active agent, and, optionally a pharmaceutically acceptable carrier or excipient.

When combined in the same formulation, it will be appreciated that the two compounds must be stable and compatible with each other and the other components of the formulation. When formulated separately, they may be provided in any convenient formulation, conveniently in such manner as are known for such compounds in the art.

The prodrugs presented herein may be formulated for administration in any convenient way for use in human or veterinary medicine. The invention therefore includes pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, see, e.g., International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia, cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesiumaluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyan

The pharmaceutical compositions of the invention may contain from 0.01 to 99% weight per volume of the prodrugs encompassed by the present invention.

### Dosages

The doses referred to throughout the specification refer to the amount of the opioid free base equivalents in the particular compound, unless otherwise specified.

Appropriate patients to be treated according to the methods of the invention include any human or animal in need of treatment. Methods for the diagnosis and clinical evaluation of pain, including the severity of the pain experienced by an animal or human are well known in the art. Thus, it is within the skill of the ordinary practitioner in the art (e.g., a medical doctor or veterinarian) to determine if a patient is in need of treatment for pain. The patient is preferably a mammal, more preferably a human, but can be any subject or animal, including a laboratory animal in the context of a clinical trial, screening, or activity experiment employing an animal model. Thus, as can be readily appreciated by one of ordinary skill in the art, the methods and compositions of the present invention are particularly suited to administration to any animal or subject, particularly a mammal, and including, but not limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending on the severity of pain to be treated, a suitable therapeutically effective and safe dosage, as may readily be determined within the skill of the art, can be administered to subjects. For oral administration to humans, the daily dosage level of the prodrug may be in single or divided doses. The duration of treatment may be determined by one of ordinary skill in the art, and should reflect the nature of the pain (e.g., a chronic versus an acute condition) and/or the rate and degree of therapeutic response to the treatment. Typically, a physician will determine the actual dosage which will be most suitable for an individual subject.

The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For highly potent agents such as buprenorphine, the daily dose requirement may, for example, range from 0.5 to 50 mg, e.g. from 1 to 25 mg, and optionally from 1 mg to 10 mg (all with reference to the opioid free base content). For less potent agents such as meptazinol, the daily dose requirement may, for example, range from 1 mg to 1600 mg, e.g. from 1 mg to 800 mg and optionally from 1 mg to 400 mg.

In the methods of treating pain, the prodrugs encompassed by the present invention may be administered in conjunction with other therapies and/or in combination with other active agents. For example, the prodrugs encompassed by the present invention may be administered to a patient in combination with other active agents used in the management of pain. An active agent to be administered in combination with the prodrugs encompassed by the present invention may include, for example, a drug selected from the group consisting of non-steroidal anti-inflammatory drugs (e.g., acetaminophen and ibuprofen), anti-emetic agents (e.g., ondanstron, domerperidone, hyoscine and metoclopramide), unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical composition. For example, a prodrug encompassed by the present invention and another active agent may be administered in a substantially simultaneous manner, such as in a single capsule or tablet having a fixed ratio of these agents, or in multiple separate dosage forms for each agent.

When the prodrugs of the present invention are used in combination with another agent active in the methods for treating pain, the dose of each compound may differ from that when the compound is used alone. Appropriate doses will be readily appreciated by those of ordinary skill in the art.

### EXAMPLES

The present invention is further illustrated by reference to the following Examples. However, it should be noted that these Examples, like the embodiments described above, are illustrative and are not to be construed as restricting the enabled scope of the invention in any way.

### Example 1

### Synthesis of Meptazinol PABA Carbamate

Variations (A) and (B) utilize a t-butyl protected 4-aminobenzoate in the formation of the 4-aminobenzoate isocyanate intermediate. In contrast, variation (C) utilizes a benzyl protected 4-aminobenzoate in the formation of the 4-aminobenzoate isocyanate intermediate. Variation (C) is the preferred synthesis since the benzyl ester protection can be removed under neutral catalytic hydrogenation conditions to yield the free base. The free base can then be converted into any desired salt form.

### Variation (A):

This was undertaken using the scheme shown below:

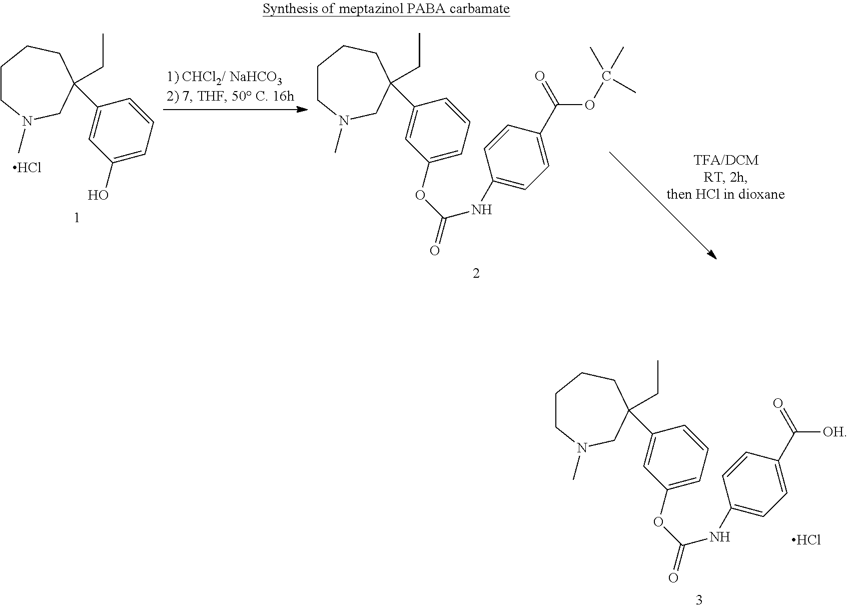

### Stage 1—Preparation of Isocyanate (7)

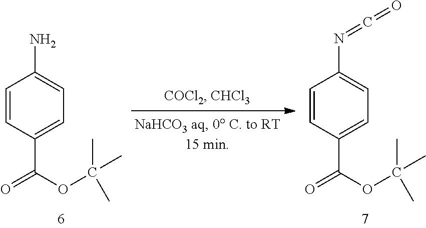

The aniline 6 (966 mg, 5 mmol) was dissolved in dichloromethane (40 ml) and an aqueous solution of NaHCO₃ saturated (40 ml) was added. The reaction mixture (RM) was cooled down to 5° C. with an ice bath and a solution of phosgene (5 ml, 10 mmol, 2M in toluene) was then added dropwise via a syringe. The RM was stirred at room temperature for 15 minutes and then worked up by extracting the aqueous phase with dichloromethane (2×50 ml). The organic layer was dried over sodium sulfate and evaporated to dryness. 1.03 g (94%) of white solid (7) was obtained.

### Stage 2—Preparation of Protected Ester (2)

Meptazinol HCl (966 mg, 3.61 mmol) was converted to the free base in the presence of chloroform (50 ml) and an aqueous solution of NaHCO3 saturated (50 ml). The organic phase was dried over sodium sulphate and evaporated to dryness, leaving a yellow oil. The yellow oil was dissolved in 50 ml of dry THF and isocyanate 7 (876 mg, 4 mmol) was added. The RM was heated at 50° C. for 16 h during which time it was monitored by TLC. The RM was cooled down to room temperature and the THF was removed under vacuum. The residual oil was then purified by column chromatography (100 g of SiO₂, elution with CHCl₃/MeOH; v:v 99/1, 98/2, 96/4 and 94/6). The ester 2 was isolated in moderate yield (998 mg, 61%).

### Stage 3—Preparation of Meptazinol PABA Carbamate

Ester 2 (450 mg, 0.995 mmol) was dissolved in dichloromethane (20 ml) and TFA was added (5 ml). The RM was stirred at room temperature for 2 h and then evaporated to dryness. The yellow residual oil was taken up in HCl (10 ml, 4N in dioxane) and evaporated to dryness and this process was repeated three more times until a foamy solid was obtained. The solid was dissolved in water and freeze dried, giving 3 as a yellow solid (400 mg, 93%).

¹H NMR (300 MHz, D₂O): δ 0.45 (m, 3H) 1.35-2 (m, 7H), 2.45 (m, 1H), 2.8 (s, 3H), 3.15 (m, 2H), 3.5 (d, 1H), 3.75 (d, 1H), 7-7.3 (m, 3H), 7.45 (m, 3H), 7.9 (d, 2H)

MS data: ES⁺397.40 (M+H)

### Variation (B):

Meptazinol p-amino-benzoylcarbamate is made by deprotecting under acidic conditions as shown in the Scheme below yielding a meptazinol p-amino-benzoylcarbamate HCl salt which is converted to a free base.

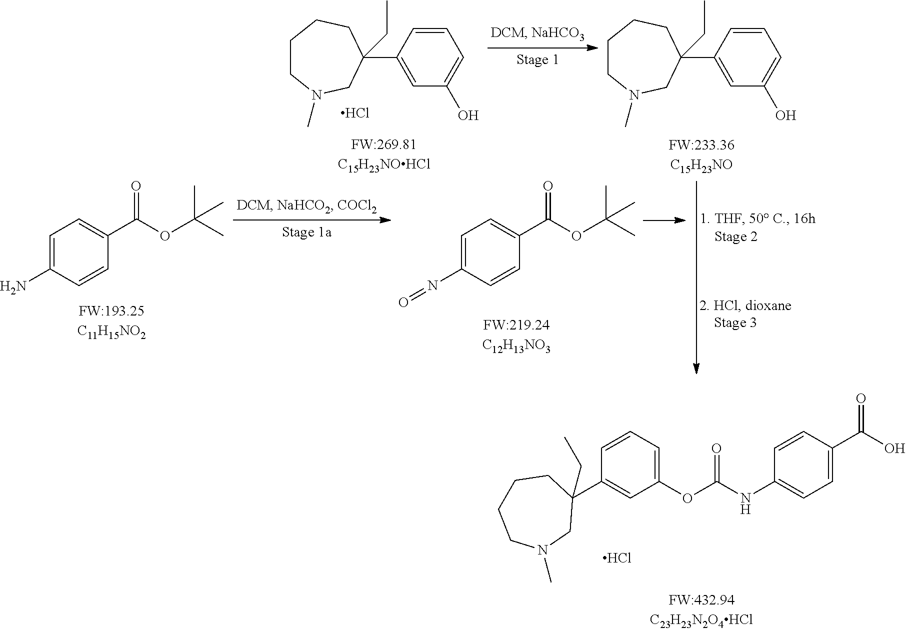

### Stage 1—Salt Release of Meptazinol Freebase

Meptazinol.HCl (20.0 g, 1 eq) was suspended in DCM (25 vol) and water (3 vol) at 15 to 25° C. Sodium hydrogen carbonate (6.5 g, 1.05 eq.) was charged and the mixture was warmed to 30 to 35° C. and stirring continued until gas evolution had ceased (40 min) and then maintained at 30 to 35° C. for a further 40 minutes. The phases were separated, the organic phase dried over Na₂SC₄, maintaining 30 to 35° C., filtered, vessel/cake wash with DCM (2 vol) and the filtrates were concentrated to dryness, which generated meptazinol freebase as a white solid 17.0 g, 98.3% th yield.

Stage 1a: Synthesis of 4-isocyanato-benzoic Acid Tert-butyl Ester

The synthesis of 4-isocyanato-benzoic acid tert-butyl ester was carried out using the scheme:

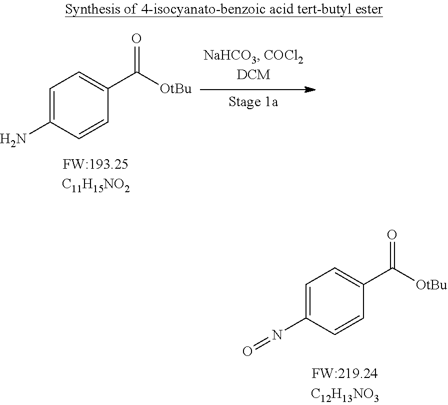

Tert-butyl 4-aminobenzoate (2.0 g, leq.) was dissolved in DCM (10 vol) and a saturated aqueous solution of NaHCO3 (10 vol) was added. The biphasic solution was cooled to 0 to 5° C. and a solution of phosgene (10.4 mL, 2 eq, 2M solution in toluene) was added. The reaction mixture was warmed to ambient and stirred for a further 15 minutes when IPC by ¹H NMR showed full conversion of the starting material to product. The phases were separated and the aqueous extracted with DCM (2×12.5 vol). The organic layer was dried over Na₂SC₄ (3 wt), filtered, vessel/cake rinse with DCM (2×20 mL) and concentrated to dryness under a vacuum which generated the isocyanate as yellow oil that solidified upon standing (2.3 g, yield 100% th).

The approach using phosgene was repeated with a 10.0 g input of tert-butyl 4-aminobenzoate to give a further 11.5 g of the isocyanate (100% th yield after correcting for solvent content. The aqueous method using phosgene generates the desired isocyanate in good yield and purity.

### Stage 2: Synthesis of Meptazinol Benzyl Carbamate Tert-butyl Ester (MBC-TBE)

The synthesis of meptazinol benzyl carbamate tert-butyl ester was carried out using the scheme below:

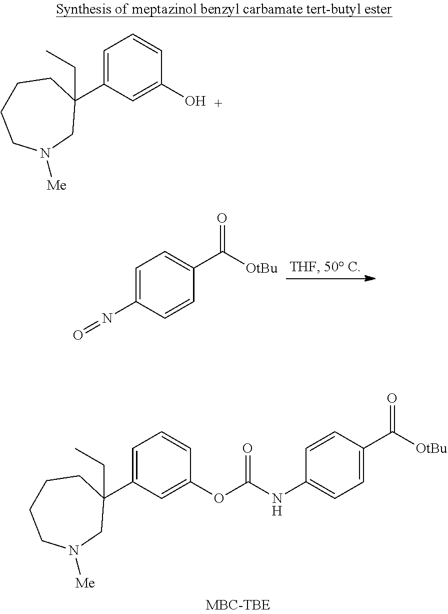

The coupling of meptazinol (free base) with 4-isocyanato-benzoic acid fed-butyl ester to generate MBC-TBE was performed in THF. Meptazinol free base (8.6 g, 1.0 eq.), isocyanate (9.0 g, 1.1 eq.) and THF (15 vol) were charged to a vessel and heated to 50° C., and the reaction monitored by LC-MS, as shown in Table 3 below.

TABLE 3
In Process Check by LC-MS

| % area by LC-MS (UV trace) | | | |
|---|---|---|---|
| RRT 0.67 | RRT 1.0 | RRT 1.26 | |
| Meptazinol | MBC-TBE | Unknown | Comment |
| 7.2 | 80.5 | 12.3 | 1.5 h at 50° C. |
| 7.6 | 78.2 | 14.1 | 30 h at 50° C. |

Additional charge of isocyanate made 0.72 g, 8% of original charge

| | | | |
|---|---|---|---|
| 8.6 | 75.2 | 16.2 | 30.5 h at 50° C. |
| 6.0 | 77.8 | 16.1 | 31.5 h at 50° C. |
| 6.0 | 79.0 | 15.0 | 32 h at 50° C. |

After a 32 hour stir period the reaction was deemed complete. The pale orange reaction mixture was concentrated to dryness and purified by flash column chromatography (600.0 g silica, using gradient elution—DCM:MeOH, 99:1 increasing to 90:10).

Meptazinol benzoyl carbamate benzyl ester (4.3 g, 25.7% th) was isolated containing about 4% w/w meptazinol by LC-MS; 13.7 g of material was also isolated as a mixture of meptazinol benzoyl carbamate tert-butyl ester, isocyanate starting material, meptazinol and an unknown impurity at RRT 1.26.

The structure of the impurity at RRT 1.26 has the same retention time and mass ion pattern (M+ of 413.2 and 825.2) as an impurity that was observed during the preparation of the isocyanate. A small amount of this impurity would have been carried through from the preparation of the isocyanate; however, this impurity is also being generated during the course of the coupling reaction. The coupling reaction generated material that could be used to investigate the subsequent ester cleavage and zwitterion preparation.

Stage 3—Preparation of Meptazinol p-amino-benzoylcarbamate HCl Salt:

The synthesis of meptazinol p-amino-benzoylcarbamate HCl was carried out using the scheme below:

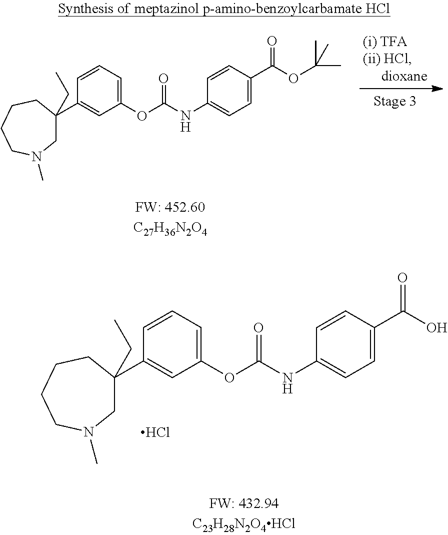

The method for the conversion of meptazinol benzyl carbamate tert-butyl ester to meptazinol p-amino-benzoylcarbamate HCl was to treat meptazinol benzyl carbamate tert-butyl ester with trifluoroacetic acid (TFA) to affect the ester cleavage, followed by treatment with hydrochloric acid (HCl) in dioxane to convert the trifluoroacetic acid salt to the HCl salt. Generation of the desired HCl salt directly was investigated.

Meptazinol benzyl carbamate tert-butyl ester (1.0 g, leq.) and formic acid (14 vol) were charged to the vessel. Water (0.06 mL, 1.5 eq.) was charged, followed by acetyl chloride (0.20 mL, 1.3 eq.). The reaction was stirred at 18 to 23° C. and monitored by LC-MS.

An in process check by LC-MS after 1.5 h at 18 to 23° C. showed the reaction to be almost complete (<5% area MBC-TBE), further reaction time gave no change in reaction profile. The reaction mixture was concentrated to dryness and azeotroped with toluene (3×10 vol) and water (2×10 vol) to remove residual formic acid which gave meptazinol p-amino-benzoylcarbamate HCl as a foam 0.7 g, 78.4% th).

The ester hydrolysis using these conditions was repeated at a 3.0 g input of MBC-TBE, complete conversion was observed (LC-MS) after an 18 hour reaction period, and meptazinol p-amino-benzoylcarbamate HCl isolated, 69.8% th, purity by HPLC 96.7% area).

Stage—Salt Release of Meptazinol p-amino-benzoylcarbamate HCl:

The release of meptazinol p-amino-benzoylcarbamate freebase from meptazinol p-amino-benzoylcarbamate HCl was carried out using the scheme below:

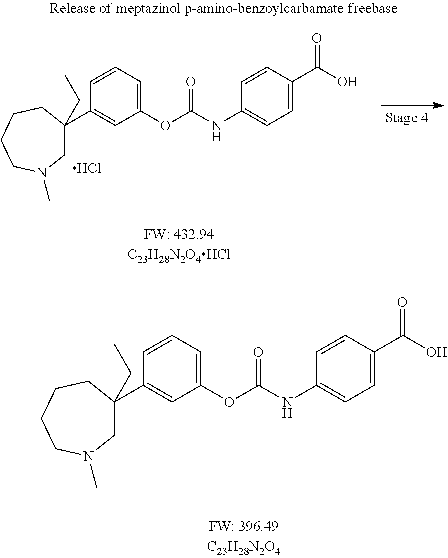

Since the production of both meptazinol p-amino-benzoylcarbamate free base and meptazinol p-amino-benzoylcarbamate HCl were required, if this original route were to be used, a method of generation of the freebase from the hydrochloride salt would be required.

An investigation was made into using aqueous conditions to generate the free base of meptazinol p-amino-benzoylcarbamate by adjusting the pH to 7 using a saturated NaHCO₃, solution. Meptazinol p-amino-benzoylcarbamate HCl was taken up in water (9 vol) and saturated aq. NaHCO₃, solution was added to adjust the pH to 7. After the addition of 1 mL, pH=4.8 a sticky solid had formed in one clump in the vessel, which was broken up with a spatula. After the addition of a total of 5 mL of NaHCO₃ solution the pH had reached 8.1 and a hazy solution was formed. The pH was adjusted back to 7 with 0.5M HCl, however, the pH drifted upwards and was re-adjusted with 0.5M HCl. Once pH 7 was reached, n-butanol (10 vol) was charged to give a hazy solution. Addition of 20 vol of n-heptane gave a phase split. The biphasic solution was separated and the aqueous was extracted with n-butanol (2×20 vol). The organic phase was concentrated to dryness to give 0.23 g of an off-white solid (50% th). ′H NMR of the isolated solid confirmed that the free base had been isolated. (N-Me observed in ′H at 62.3 ppm, this is indicative of the freebase versus about 62.9 ppm for the salt). LC-MS of the organic phase shows M⁺ 397 which is the desired product and M⁺ 234 which is meptazinol.

LC-MS of the aqueous phase suggests that there has been some cleavage of the carbamate to give meptazinol (M⁺ 234) and the aromatic amino carboxylic acid (M⁺ 138). The main peak in the aqueous phase has a M⁺ of 301.

### Variation (C):

Meptazinol p-amino-benzoylcarbamate is made following the Scheme below.

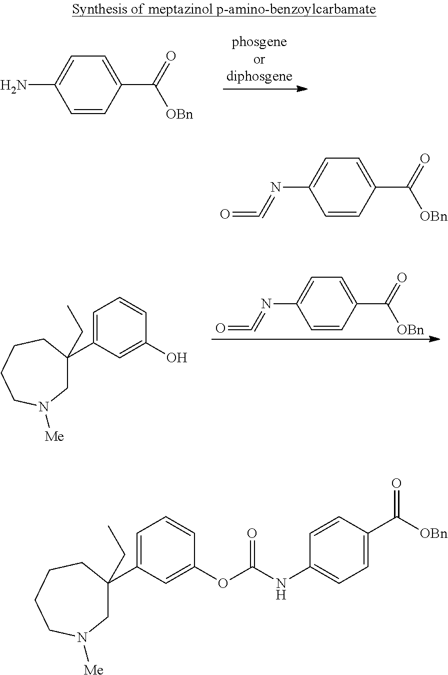

A screen of conditions for the preparation of the isocyanate of the amino benzoate starting material was required; two possible alternatives for the preparation were aqueous conditions using phosgene or anhydrous conditions using diphosgene and an organic base. An initial reaction was performed using phosgene under Schotten-Baumen type conditions. Benzyl-4-aminobenzoate (5.0 g, 1 eq.); DCM (10 vol) and sat. NaHCO3 (10 vol) were charged to a vessel and cooled to 0 to 5° C. Phosgene solution (20% in toluene, 2.0 eq., 23.2 mL) was charged maintaining a temperature of 0 to 5° C. The reaction was warmed to ambient and stirred for 15 mins when IPC by ¹H NMR indicated the reaction to be complete. The phases were separated, and the aqueous phase back-extracted with DCM (2×10 vol). Following drying and concentration to dryness of the combined organic extracts, the product was isolated as an orange mobile oil: 4.3 g uncorr., 4.2 g corr., 75.3% th yield corr. (corrected for solvents—97.1% w/w product, 2.6% w/w toluene and 0.3% w/w DCM).

Using Schotten-Baumen type conditions the isocyanate derivative of benzyl-4-aminobenzoate was formed cleanly in good yield. Benzyl-4-aminobenzoate (2.0 g, 1 eq.) and DCM (5 vol) were charged to a vessel (1), cooled to a to 5° C. and to this was charged pyridine (3.06 eq., 2.18 mL (note: a slurry was produced at this stage). In a separate vessel (2) was charged diphosgene (0.65 eq, 0.69 mL and DCM (8 vol) and cooled to a to 5° C. The contents of vessel (1) were then transferred to vessel (2), maintaining 0 to 5° C. (note: v. exothermic, additional 5 vol of DCM charged to vessel (1) to thin the slurry slightly to allow it to pass through a cannula). The reaction mixture was warmed to 18 to 23° C. and stirred for 45 min when IPC by ¹H NMR indicated the reaction was complete. Stirring for a further 35 min showed no change in profile. The reaction was quenched into 1 M HCl (5 vol) and the phases separated. The combined organics were washed with 1M HCl (10 vol) and brine (2×10 vol), dried over MgSO and concentrated to dryness to give a pink/orange sticky solid, 1.60 g uncorr. ¹H NMR showed the output of the reaction to be predominantly product but also contained some starting material and an unknown impurity (84.8% w/w product (product versus starting material)), therefore corrected weight of 1.36 g, 61% th yield. The preparation of the isocyanate derivative of benzyl-4-aminobenzoate using diphosgene/pyridine generates the desired product in moderate yield, however repeating these conditions using a higher charge of phosgene/pyridine indicated that degradation had occurred during isolation with generation of multiple impurities observed. It has been shown that phosgene may be used under biphasic conditions to generate the required isocyanate. It has also been shown that diphosgene under anhydrous conditions would generate the isocyanate with a clean profile but during the isolation degradation was observed. A reaction was trialled to determine whether diphosgene could be used using the aqueous conditions, Benzyl-4-aminobenzoate (2.0 g, 1 eq.), DCM (10 vol) and sat. NaHCO3 (10 vol) were charged to a vessel and cooled to a to 5° C. To this was charged diphosgene (2 eq., 2.1 mL maintaining a to 5° C. At completion of addition, reaction was warmed to 18 to 23° C. and monitored by ′H NMR as shown in Table 4 below.

TABLE 4
In Process Check by ¹H NMR

| ¹H NMR result | Comment |
|---|---|
| 24.9% w/w amino benzoate, 75.1% w/w product | 15 min at ambient |
| 16.0% w/w amino benzoate, 84.0% w/w product | 1.5 h at ambient |

0.6 eq. of diphosgene charged, reaction cooled to 0-5° C.
during charging and then warmed to ambient

| | |
|---|---|
| 14.6% w/w amino benzoate, 85.4% w/w product | 10 min after additional |
| | charge |
| 10.2% w/w amino benzoate, 89.8% w/w product | 1.25 h after additional |
| | charge |
| Amino benzoate not detected | Overnight stir |

Once the starting benzyl-4-aminobenzoate was not detected by ′H NMR, the phases were separated, and the aqueous back-extracted with DCM (2×10 vol). Following drying and concentration to dryness an orange mobile oil/liquid was isolated: 7 g, 77.0% th yield. Using diphosgene in the Schotten-Baumen type conditions the desired isocyanate was formed cleanly in good yield. The reaction using diphogene was slower than the corresponding reaction using phosgene.

### Example 2

### Synthesis of Meptazinol (2-methyl PABA) Carbamate

This is set out in the scheme below.

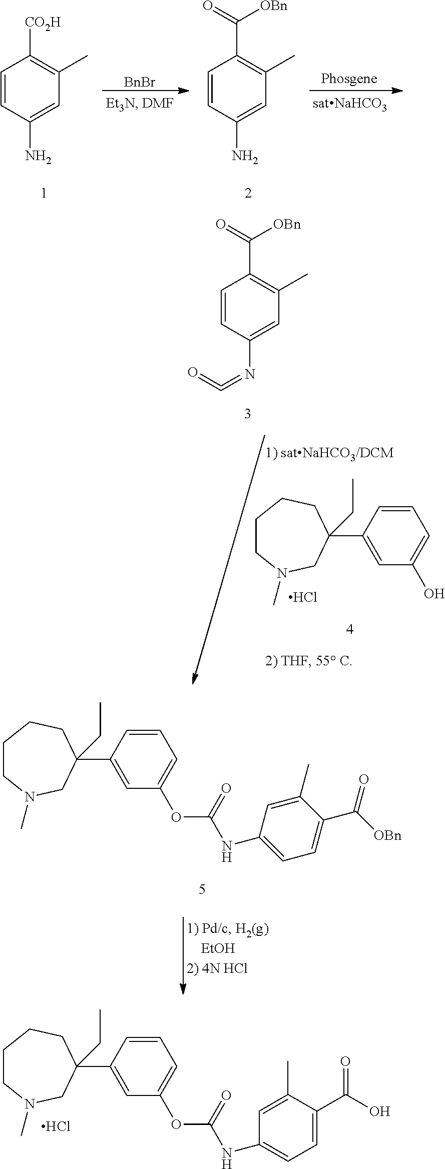

### Detail

### Preparation of 2

The aniline (1, 10 g, 66.16 mmol) was dissolved in DMF (100 mL) and triethyl amine (10.11 mL, 72.77 mmol) at RT. Benzyl bromide (7.86 mL, 66.16 mmol) was then added and the resulting clear brown solution was stirred at RT for 24 h. After pouring into saturated aqueous sodium bicarbonate solution (300 mL) the solution was extracted with EtOAc (2×200 mL). The combined organics were washed with brine (200 mL), dried over MgSO4, filtered, and concentrated in vacuo to yield the crude product which was purified by flash column chromatography (250 g of SiO₂, elution with 10% EtOAc/Petrol) to obtain the clean product in 19% yield. (R_{f}=0.64 in 50% EtOAc/Petrol, visualised by KMnC₄ and UV)

### Preparation of Isocyanate 3

The benzyl ester (2, 1.4 g, 5.81 mmol) was treated a saturated aqueous solution of NaHCO₃ (30 mL) and DCM (50 mL). The reaction mixture (RM) was cooled down to 5° C. using an ice bath and a solution of phosgene (5.8 mL, 11.6 mmol, 2M in toluene) was then added dropwise via a syringe. The RM was stirred at room temperature for 45 minutes and then worked up by extracting the aqueous phase with dichloromethane (2×100 mL). The organic layer was dried over sodium sulphate and evaporated to dryness and dried under high vacuum to give 1.5 g of yellow oil in quantitative yield.

### Preparation of 5

Meptazinol HCl (4, 1.3 g, 4.82 mmol) was converted to the free base in the presence of chloroform (50 mL) and saturated aqueous NaHCO₃ solution (30 mL). The organic phase was dried over sodium sulphate and evaporated to dryness in vacuo, and further dried under high vacuum to give the free base as yellow oil. The yellow oil was dissolved in 100 mL of anhydrous THF and isocyanate 2 (1.5 g, 5.61 mmol) was added. The RM was heated at 58° C. (bath temperature) for 20 h during which time it was monitored by TLC. The RM was cooled down to room temperature and the THF was removed under vacuum. The residual oil was then purified by dry flash column chromatography (100 g of SiO₂, elution with CHCl₃/MeOH; 99/1, 98/2, 96/4). The ester 4 was isolated in good yield (4.2 g, 54%) as yellow oil. (R_{f}=0.38 in 10% MeOH/DCM, visualised by KMnC₄ and UV)

### Preparation of Final Product

Pd/C (10%) (500 mg) was suspended in EtOH (50 mL) under a nitrogen atmosphere and a solution of 5 (1.4 g, 2.79 mmol), was added. The solution was degassed and purged with hydrogen and hydrogenated overnight at 25° C. (oil bath temperature). After 20 h, the mixture was filtered through celite and concentrated to give the crude Int 5 (1 g). This was dissolved in EtOH (10 mL) and 4NHCl in dioxane (1.2 mL, 4.8 mmol) and the resulting yellow solution was stirred at RT for 30 mins followed by concentration in vacuo to give a pale yellow solid. The solid was triturated with ether (50 mL) and filtered under nitrogen to yield 850 mg of pale yellow solid. The solid was dissolved in EtOH and concentrated in vacuo to yield the product meptazinol (2-methyl PABA) carbamate as a pale yellow solid (700 mg, 56% yield).

### NMR Spectrum

(¹H NMR (300 MHz, D₂O): δ 0.5 (m, 3H), 1.2-1.8 (m, 7H), 2.2-2.5 (m and s, 4H), 2.8 (s, 3H), 2.9 (d, 1H), 3.2 (m, 2H), 3.9 (1H, d), 6.9-7.7 (m, 6H), 7.9 (d, 1H).

### Example 3

### Synthesis of Meptazinol Meta-amino Benzoic Acid Carbamate Hydrochloride

The synthesis of meptazinol meta-amino benzoic acid carbamate hydrochloride was achieved in 3 distinct reaction steps (see Scheme below).

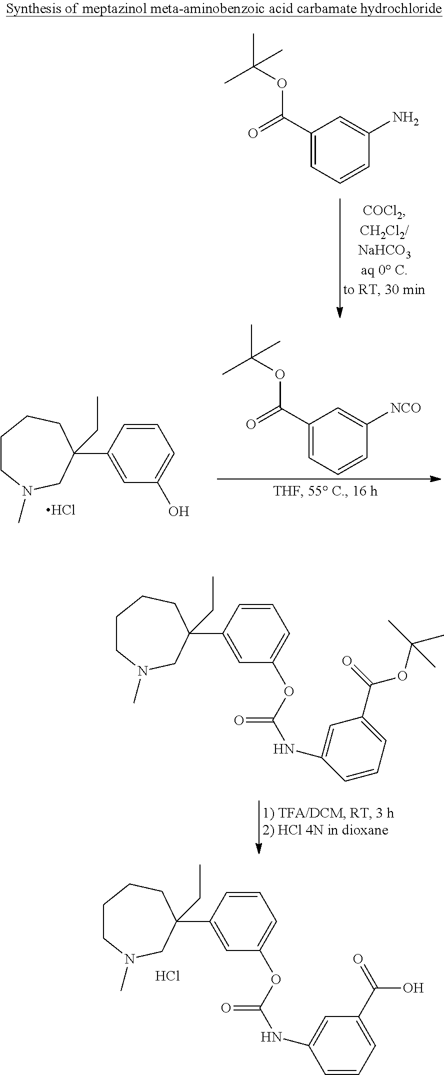

3-Amino benzoic acid tert-butyl ester was treated with an excess of a solution of phosgene in toluene, in dichloromethane in the presence of sodium bicarbonate to yield the corresponding isocyanate. The isolated isocyanate was reacted with meptazinol free base in tetrahydrofuran at 50° C. over night to give the expected coupled product. Cleavage of the tert-butyl ester was accomplished using trifluoroacetic acid to yield after azeotropic treatment with hydrogen chloride in dioxane and freeze drying, meptazinol meta-aminobenzoic acid carbamate hydrochloride as a white solid.

### Detail

### Preparation of Isocyanate

3-Aminobenzoic acid tert-butyl ester (2.58 g, 13.35 mmol) was dissolved in dichloromethane (100 mL) and an aqueous solution of saturated sodium bicarbonate (100 mL) was added. The reaction mixture was cooled to 5° C. using an ice bath and then a solution of phosgene in toluene (2M, 13.35 ml, 26.70 mmol) was added dropwise via a syringe. After stirring at room temperature for 30 minutes the reaction mixture was worked up by extracting the aqueous phase with dichloromethane (2×50 mL). The organic layer was dried over sodium sulfate and evaporated to dryness in vacuo to yield 2.72 g (93%) of the isocyanate which was clean by NMR and used directly in the next step.

### Preparation of Meptazinol Meta-amino Benzoic Acid Carbamate Tert-butyl Ester

Meptazinol hydrochloride (3.24 g, 12 mmol) was converted to the free base in the presence of chloroform (50 mL) and an aqueous solution of saturated sodium bicarbonate (50 mL). The organic phase was dried over sodium sulphate and evaporated to dryness in vacuo, leaving a yellow oil. The oil was dissolved in anhydrous tetrahydrofuran (150 mL) and the isocyanate from the previous step (2.72 g, 12.42 mmol) was added. The reaction mixture was heated at 50° C. for 16 h whilst being monitored by TLC. After leaving to cool to room temperature the tetrahydrofuran was removed in vacuo. The residual oil was purified by column chromatography (100 g of silica, elution with CHCl₃/MeOH; vol:vol 99/1, 98/2, 96/4 and 94/6) to yield meptazinol meta-amino benzoic acid carbamate tert-butyl ester in moderate yield (3.5 g, 64%).

### Preparation of Meptazinol Meta-amino Benzoic Acid Carbamate Hydrochloride

Meptazinol meta-amino benzoic acid carbamate tert-butyl ester (2.9 g, 6.4 mmol) was dissolved in dichloromethane (50 mL) and trifluoroacetic acid (50 mL) was added. The reaction mixture was stirred at room temperature for 3 h followed by evaporating to dryness in vacuo. The yellow residual oil was taken up in tetrahydrofuran (50 mL) and hydrogen chloride (10 mL of 4N hydrogen chloride in dioxane) was added followed by evaporation to dryness in vacuo. The addition of HCl followed by evaporation was repeated twelve further times to yield a ‘foamy’ solid which was dissolved in water and freeze dried, yielding meptazinol meta-aminobenzoic acid carbamate hydrochloride as an off-white solid (1.8 g, 42%).

### NMR Spectrum

¹H NMR (300 MHz, DMSO-d₆): δ 0.5 (t, 3H) 1.5 (m, 1H), 1.6-2 (m, 7H), 2.3 (m, 1H), 2.4 (m, 1H), 2.9 (d, 3H), 3.2 (m, 2H), 3.65 (d, 1H), 4.1 (d, 1H), 7.1-7.3 (m, 2H), 7.35 (s, 1H), 7.45 (m, 2H), 7.6 (d, 1H), 7.7 (t, 1H), 8.15 (s, 1H), 10.45 (br, 1H), 13 (br, 1H).

Purity: >99.4% by HPLC (Isocratic and Gradient methods)

MS data: ES+ 397.33 (M+H)

### Example 4

### Synthesis of Meptazinol Para-amino Phenyl Acetic Acid Carbamate Hydrochloride

The synthesis of meptazinol-para-amino phenyl acetic acid carbamate hydrochloride was achieved in 3 distinct reaction steps (see Scheme below).

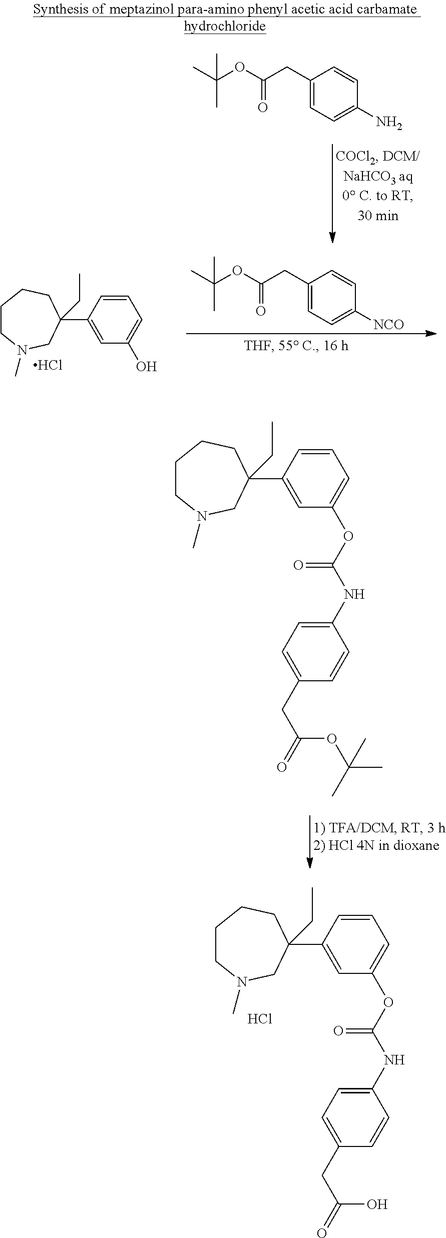

4-Aminophenylacetic acid tert-butyl ester was treated with an excess of a 20% solution of phosgene in toluene, in dichloromethane in the presence of sodium bicarbonate. The resulting isocyanate was isolated before being coupled to meptazinol free base in tetrahydrofuran at 50° C. overnight. Cleavage of the tert-butyl group with trifluoroacetic acid followed by conversion to the corresponding hydrochloride salt and freeze drying gave meptazinol para-amino phenyl acetic acid carbamate hydrochloride as a white solid.

### Detail

### Preparation of the Isocyanate

4-Aminophenyl acetic acid tert-butyl ester (2.27 g, 10.95 mmol) was dissolved in dichloromethane (80 mL) and an aqueous solution of saturated sodium bicarbonate (80 mL) was added. The reaction mixture was cooled down to 5° C. using an ice bath and a solution of phosgene (2M in toluene, 11 mL, 22 mmol) was then added drop wise via a syringe. The reaction mixture was stirred at room temperature for 30 minutes and then worked up by extracting the aqueous phase twice with dichloromethane (50 mL). The organic layer was dried over sodium sulfate and evaporated to dryness in vacuo to yield 2.6 g (>100%, contains traces of toluene) of white solid which was clean enough by NMR for use in the next synthetic step.

Preparation of meptazinol 4-aminophenylacetic Acid Tert-butyl Ester Carbamate

Meptazinol hydrochloride (2.58 g, 9.56 mmol) was converted to the free base in the presence of chloroform (50 mL) and an aqueous solution of saturated sodium bicarbonate (50 mL). The organic phase was dried over sodium sulphate and evaporated to dryness in vacuo, leaving a yellow oil which was then dissolved in anhydrous tetrahydrofuran (150 mL). To this was added the isocyanate (2.6 g, 10.95 mmol) and the resulting mixture was heated at 50° C. for 16 h whilst being monitored by TLC. After allowing to cool to room temperature the tetrahydrofuran was removed in vacuo. The residual oil was then purified by column chromatography (100 g of SiO₂, elution with CHCl₃/MeOH; vol:vol 99/1, 98/2, 96/4 and 94/6). The ester was isolated in moderate yield (1.77 g, 40%).

### Preparation of Meptazinol Para-amino Phenyl Acetic Acid Carbamate Hydrochloride

The ester from the previous step (1.34 g, 0.995 mmol) was dissolved in dichloromethane (50 mL) and trifluoroacetic acid was added (50 mL). The reaction mixture was stirred at room temperature for 3 h and then evaporated to dryness in vacuo. The yellow residual oil was taken up in tetrahydrofuran (50 mL) containing hydrogen chloride (10 mL of 4N HCl in dioxane) and evaporated to dryness in vacuo. This was repeated a further seven times to yield a ‘foamy’ solid. This was dissolved in the minimum of water and freeze dried, giving meptazinol para-amino phenyl acetic acid carbamate hydrochloride as an off-white solid (593 mg, 46%).

### NMR Spectrum

(300 MHz, DMSO-d6): δ 0.55 (t, 3H) 1.4-2 (m, 7H), 2.35 (m, 1H), 2.85 (t, 3H), 3.15 (m, 2H), 3.6 (s, 2H), 3.75 (d, 1H), 4 (d, 1H), 7.1-7.3 (m, 6H), 7.4 (m, 3H), 10.2 (br, 1H), 12.3 (br, 1H).

### Example 5

### Synthesis of meptazinol 6-amino Nicotinic Acid Carbamate Hydrochloride

The synthesis of meptazinol 6-aminonicotinic acid carbamate hydrochloride was achieved in 3 distinct reaction steps (steps 2 and 3 are merged) (see Scheme below).

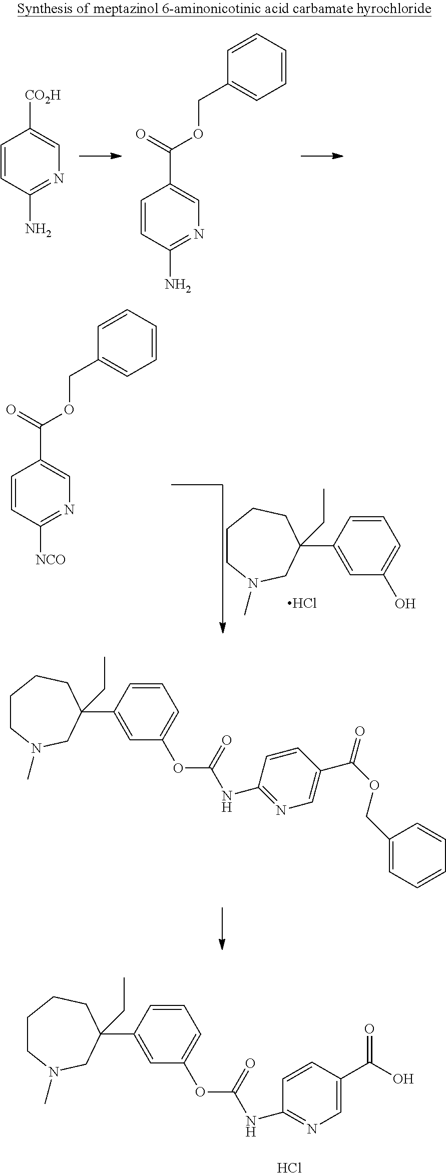

6-amino-pyridine-3-carboxylic acid was protected as the corresponding benzyl ester and treated with an excess of a solution of phosgene in toluene, in dichloromethane in the presence of triethylamine. The resulting isocyanate was not isolated but reacted directly with meptazinol free base in tetrahydrofuran at −78° C. Cleavage of the benzyl ether was accomplished using a standard hydrogenation procedure to yield after treatment with hydrogen chloride in dioxane, meptazinol 6-amino nicotinic acid carbamate hydrochloride as a white solid.

### Detail

Preparation of 6-amino-pyridine-3-carboxylic Acid Benzyl Ester

A suspension of 6-amino-pyridine-3-carboxylic acid (15.89 g, 0.115 mol), benzyl bromide (19.68 g, 0.115 mol) and triethylamine (13.96 g, 0.138 mol) in N,N-dimethylformamide (222 mL) was heated and stirred at 55 to 57° C. for 19 hours. The resulting suspension was allowed to cool to 15 to 20° C. and was then quenched with saturated aqueous sodium carbonate solution (200 mL), water (200 mL) and diethyl ether (200 mL). The phases were separated and the organic phase was washed with saturated brine (3×150 mL). The organic phase was dried over magnesium sulfate (16 g), filtered and the filtrate evaporated to dryness in vacuo at 40° C. to give a pale yellow solid. The solid was triturated in diethyl ether (25 mL) and the resulting solid was collected by filtration and washed with diethyl ether (2×5 mL) and pulled dry in air to yield 6-amino-pyridine-3-carboxylic acid benzyl ester (2.95 g, 11.2% th) as a cream solid.

Preparation of Meptazinol 6-aminonicotinic Acid Carbamate Benzyl Ester

A solution of 6-amino-pyridine-3-carboxylic acid benzyl ester (2.95 g, 0.0129 mol) and triethylamine (3.92 g, 0.0388 mol) in anhydrous tetrahydrofuran (147.5 mL) and dichloromethane (97 mL) was stirred and cooled to −76° C. under nitrogen. A solution of 2M phosgene in toluene (6.5 mL, 0.01292 mol) was charged over 10 s to give an orange/red suspension (Note: exotherm to −67° C.). The resulting suspension was stirred at −70 to −76° C. for 30 min and then meptazinol hydrochloride (3.49 g, 0.0129 mol) was charged in one portion. The resulting suspension was stirred at −70 to −76° C. for 30 min and then the cold bath was removed and the suspension allowed to warm to 15 to 20° C. and was then held at 15 to 20° C. for 1 hour. TLC analysis (eluant: THF 1:1 petrol, R_{f} (SM): 0.5, R_{f} (product) 0.37, R_{f} (meptazinol) 0.17, visualized by UV and in KMnC₄) indicated incomplete but stalled conversion. The reaction mixture was quenched with saturated aqueous sodium carbonate solution (250 mL) and ethyl acetate (250 mL). The phases were separated and the organic phase was washed with saturated brine (250 mL). The organic phase was then extracted into 0.5 M hydrochloric acid (10×100 mL). The aqueous acid phases were combined and the pH was adjusted to pH 7 to 8 with saturated aqueous sodium carbonate solution (250 mL). The product was extracted into diethyl ether 1:1 ethyl acetate (4×500 mL), dried over magnesium sulfate (16 g), filtered and the filtrate evaporated to dryness in vacuo at 40° C. to give a yellow oil. The oil was combined with an earlier crude batch (1.69 g, 0.0074 mol) and purified by column chromatography on silica (160 g), eluting with acetonitrile, taking 100 mL fractions. Fractions 9 to 29, which contained pure material, were combined and evaporated to dryness in vacuo at 40° C. to yield meptazinol 6-aminonicotinic acid carbamate benzyl ester (1.45 g, 14.6% th) as a viscous pale yellow oil.

Preparation of Meptazinol 6-aminonicotinic Acid Carbamate Hydrochloride

A solution of the benzyl ester compound from the previous step (1.45 g, 2.97 mmol) in tetrahydrofuran (29 mL) was cooled to 0 to 5° C. and purged with nitrogen. 10% Pd/C (50% wet) (0.56 g) was charged and the resulting suspension stirred purged with nitrogen/vacuum (3 cycles) then with hydrogen/vacuum (3 cycles). The resulting suspension was allowed to warm to 15 to 20° C. and then stirred under an atmosphere of hydrogen for 3 hours after which time TLC analysis (eluant: 10% MeOH/DCM, R_{f} (SM): 0.4, R_{f} (product) 0.05, visualized by UV and in KMnC₄) indicated complete conversion. The suspension was cooled to 0 to 5° C. and purged with nitrogen and then filtered through GFF (glass fibre filter) under nitrogen and washed with tetrahydrofuran (2×5 mL). The filtrate was evaporated to dryness in vacuo at 40° C. to give a white solid/gum which was triturated in acetone (40 mL) at 10 to 15° C. to give the free acid product (1.15 g, 97% th) as a white powder. The free acid (650 mg, 1.635 mmol) was dissolved in dioxane (50 mL) and tetrahydrofuran (25 mL) at 40° C. Hydrogen chloride (4M in dioxane, 0.86 mL, 3.43 mmol) was charged over 1 min to give a white suspension. The white suspension was allowed to cool to 10 to 15° C. and the white solid was collected by filtration under nitrogen to yield the free acid (785 mg). The solid contained dioxane by NMR analysis which was not removed by overnight drying at 40° C. in vacuo (note: slight decomposition was observed by NMR analysis). Thus, the solid was dissolved in water (6 mL) to give a gel and was freeze dried over 24 hours to yield meptazinol 6-aminonicotinic acid carbamate hydrochloride (625 mg) as a white powder.

NMR Spectrum

¹H NMR (300 MHz, d₆-DMSO): 11.2 ppm (s, 1H), 10.5 (br, d, 0.5H), 8.85 (br, d, 0.5H), 8.85 (s, 1H), 8.3 (dd, 1H), 7.95 (d, 1H), 7.5 (m, 1H), 7.35 (m, 1H), 7.3-7.1 (m, 2H), 4.0 (d, 0.5H), 3.7 (d, 0.5H), 3.6-3.0 (m, 4H), 2.9 (m, 3H), 2.4-2.05 (m, 2H), 2.0-1.6 (m, 3H), 1.6-1.4 (m, 2H), 0.55 (t, 3H).

Mass Spec: ES+: m/z 398.06 (M_{fb}+1); ES−: m/z 396.11 (M_{fb}−1)

### Example 6

### Synthesis of Meptazinol 4-amino 2-fluorobenzoic Acid Carbamate Hydrochloride

The synthesis of meptazinol-para-amino ortho-fluorobenzoic acid carbamate hydrochloride was achieved in 3 distinct reaction steps (see Scheme below).

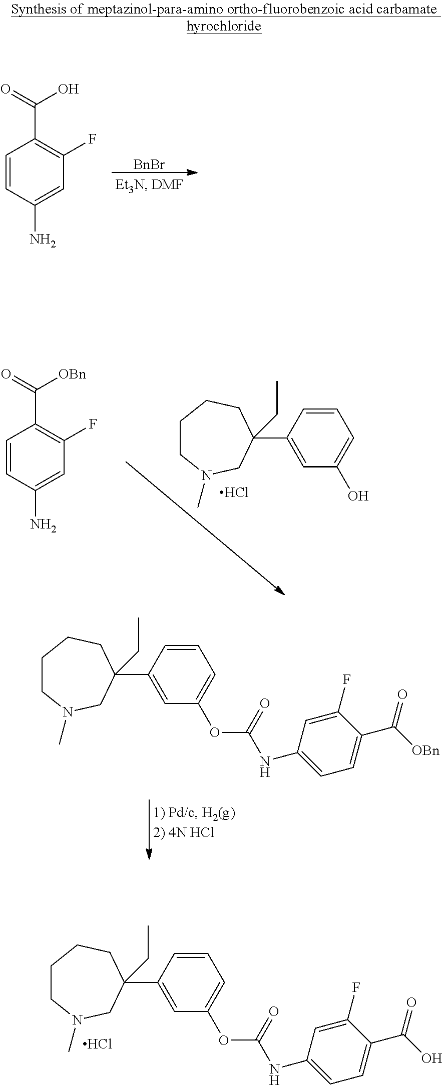

4-amino-2-fluorobenzoic acid was protected as the corresponding benzyl ester and treated with an excess of a solution of phosgene in toluene, in dichloromethane in the presence of triethylamine. The resulting isocyanate was not isolated but reacted directly with meptazinol free base in tetrahydrofuran at −78° C. Cleavage of the benzyl ether was accomplished using a standard hydrogenation procedure to yield after treatment with hydrogen chloride in dioxane, meptazinol 4-amino-2-fluoro benzoic acid carbamate hydrochloride as a white solid.

### Detail

Preparation of Benzyl 4-amino-2-fluorobenzoate

To a stirred solution of 4-amino-2-fluorobenzoic acid (2.00 g, 12.89 mmol) in N,N-dimethylformamide (70 mL) at ambient temperature was added potassium carbonate (1.96 g, 14.18 mmol) followed by the dropwise addition of benzyl bromide (1.53 mL, 12.89 mmol), and the reaction mixture was stirred overnight before being poured into water (400 mL). The mixture was extracted with ethyl acetate, and the combined extracts were washed thoroughly with brine, dried over anhydrous magnesium sulfate and concentrated under reduced pressure. This afforded the title compound as a pale yellow solid (3.16 g, quant.), which was used directly without further purification.

### Preparation of Meptazinol Para-amino Ortho-fluorobenzoic Acid Carbamate Benzyl Ester

To a stirred solution of 4-amino-2-fluorobenzoate (3.16 g, 12.89 mmol) in dichloromethane (150 mL) at −78° C. was added triethylamine (7.18 mL, 51.54 mmol), followed by the dropwise addition of phosgene (6.77 mL, 13.54 mmol, 2M solution in toluene). The resulting solution was stirred at −78° C. for 45 minutes, and then meptazinol hydrochloride (3.48 g, 12.89 mmol) was added portion wise as a solid. Stirring was maintained at −78° C. for 45 minutes, and then the reaction mixture was allowed to warm to ambient temperature, at which point it was poured into brine. The mixture was extracted with chloroform, and the combined extracts were dried over anhydrous magnesium sulfate and concentrated under reduced pressure. Purification by flash column chromatography, eluting with 5% MeOH/CHCl₃ increased to 10% MeOH/CHCl₃, afforded the title compound as a pale yellow solid (2.8 g, 43%).

### Preparation of Meptazinol Para-amino Ortho-fluorobenzoic Acid Carbamate Hydrochloride

To a stirred solution of benzyl ester (1.50 g, mmol) in tetrahydrofuran (50 mL) at ambient temperature was added a catalytic amount of Pd/C, and the solution was purged with N₂ (g). The reaction mixture was hydrogenated under a balloon of H₂ (g) for 18 hours, after which time TLC revealed consumption of the starting material. The solution was purged with N₂ (g), and then filtered through Celite™. The filter cake was well-washed with tetrahydrofuran, and the filtrate was concentrated under reduced pressure. Re-concentration from chloroform afforded an off-white solid (1.48 g), which was triturated with acetone to give, meptazinol 4-amino-2-fluoro benzoic acid carbamate (free base) as a white solid (0.54 g).

Dissolution of the free base in tetrahydrofuran (10 mL) preceded treatment with hydrogen chloride (1.00 mL, 4M solution in 1,4-dioxane), and after stirring for 30 mins at ambient temperature, the solution was concentrated under reduced pressure, slurried with chloroform and re-concentrated. The residue was freeze dried from water, giving, meptazinol 4-amino-2-fluoro benzoic acid carbamate hydrochloride as a white solid (GM342/09, 0.47 g, 35%).

NMR Spectrum

¹H NMR (DMSO-d6) δ: 12.99 (1H, br s); 10.87 (1H, br s); 10.49 (1H, br s); 7.87 (1H, t); 7.53-7.16 (6H, m); 4.02-3.14 (4H, m); 2.85 (3H, br s); 2.44-2.32 (1H, m); 2.27-2.10 (1H, m); 2.00-1.40 (6H, m); 0.53 (3H, t).

### Example 7

### Synthesis of Meptazinol 3-ethyl PABA Carbamate

The synthesis of meptazinol-para-amino meta-ethylbenzoic acid carbamate hydrochloride was achieved in 5 distinct reaction steps (see Scheme below).

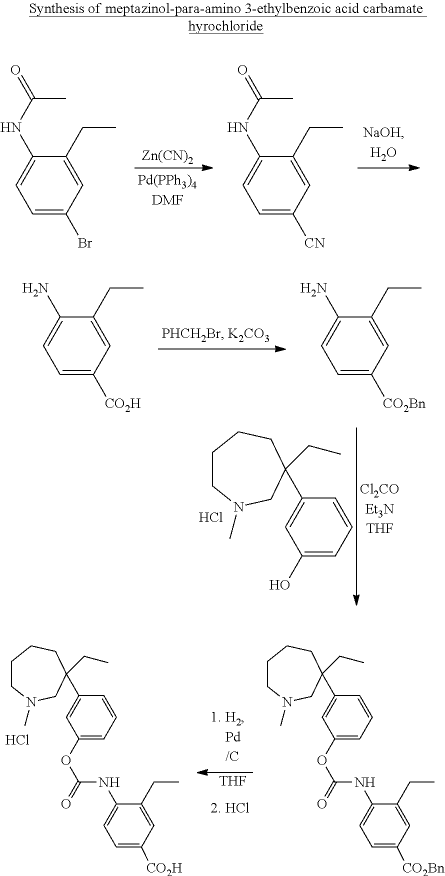

4-Bromo-2-ethylacetanilide was converted to the corresponding 4-cyano derivative using zinc cyanide. Hydrolysis of the amide and cyano group yielded 4-amino-3-ethylbezoic acid which was subsequently protected as the benzyl ester. This was then treated with an excess of a 20% solution of phosgene in toluene, in tetrahydrofuran in the presence of triethylamine to yield the corresponding isocyanate. The resulting isocyanate was coupled to meptazinol free base in tetrahydrofuran. Cleavage of the benzyl ether was accomplished using a standard hydrogenation procedure to yield after treatment with hydrogen chloride in dioxane, meptazinol para-amino meta-ethyl benzoic acid carbamate hydrochloride as a pale yellow solid.

### Detail

Preparation of 4-cyano-2-ethylacetanilide

A solution of 4-bromo-2-ethylacetanilide (25.0 g, 103.3 mmol) in anhydrous N,N-dimethylformamide (200 mL) was treated with zinc cyanide (12.15 g, 103.3 mmol) and tetrakis(triphenylphoshine)palladium (0) (6.0 g, 5.2 mmol). The mixture was then stirred under nitrogen and heated to 80° C. After 2 hours TLC analysis (petroleum ether/ethyl acetate (1:1), UV) of a sample (after a mini work-up) indicated that the reaction had proceeded to completion (starting material R_{f} 0.35, product R_{f} 0.31). The reaction mixture was cooled to room temperature then poured into ethyl acetate (1 L) and water (1 L). A solid was removed by filtration and washed with ethyl acetate (200 mL). The filtrate layers were separated and the aqueous phase was extracted with ethyl acetate (2×200 mL). The combined organic phases were washed with saturated aqueous sodium bicarbonate (500 mL) and water (500 mL), dried (magnesium sulfate) and evaporated in vacuo. The residual solid was treated with petroleum ether then filtered and dried in vacuo. The beige solid, 10.23 g, 52.6%, thus obtained was 4-cyano-2-ethylacetanilide.

The ¹H NMR spectrum was consistent with the structure and indicated good purity apart from a minor amount of a contaminant which is likely to be Pd(PPh₃)₄.

Preparation of 4-amino-3-ethylbenzoic Acid

A suspension of 4-cyano-2-ethylacetanilide (10.1 g, 53.66 mmol) in 20% aqueous sodium hydroxide (38 mL, 188 mmol) was stirred at reflux, under nitrogen atmosphere for 22 hours. The dark solution was cooled to room temperature, diluted with water (200 mL) and washed with ethyl acetate (2×50 mL). The aqueous phase was acidified with concentrated hydrochloric acid to pH 5 and extracted with ethyl acetate (2×100 mL). After further acidifying to pH 4 the aqueous phase was further extracted with ethyl acetate (2×50 mL) and the combined extracts was washed with brine (50 mL), dried (magnesium sulfate) and evaporated in vacuo. The residual solid was triturated with petroleum ether, removed by filtration, washed with petroleum ether and dried in vacuo. The beige solid (7.49 g, 84.5%) was 4-amino-3-ethylbenzoic acid of good purity.

Preparation of Benzyl 4-amino-3-ethylbenzoate

A stirred solution of 4-amino-3-ethylbenzoic acid (7.40 g, 44.80 mmol) in anhydrous N,N-dimethylformamide (110 mL) was treated with potassium carbonate (12.38 g, 89.60 mmol) and benzyl bromide (9.20 g, 53.76 mmol) and the mixture heated to 80° C. under nitrogen for 18 hours. A small sample was removed, treated with 10% aqueous citric acid and extracted with ethyl acetate. TLC analysis (CH₂Cl₂/MeOH, 9:1, UV) of the organic phase showed that complete conversion had occurred to a product at higher R_{f} (starting material R_{f} 0.30, product R_{f} 0.95). The reaction mixture was cooled, poured into water (1100 mL) and extracted with diethyl ether (3×300 mL). The combined extract was washed with water (4×250 mL), dried (magnesium sulfate) and evaporated in vacuo to leave an amber oil (12.15 g). The ¹H NMR spectrum was consistent with the structure of benzyl 4-amino-3-ethylbenzoate but showed some impurity peaks. 6.0 g of this impure material was used in an initial examination of the next step. The remaining impure material (6.1 g) was purified by column chromatography on silica gel, eluting with petroleum ether/EtOAc (83:17). Benzyl 4-amino-3-ethylbenzoate was obtained as a pale yellow viscous oil (4.83 g) of good purity by NMR analysis. This mass of pure material correlates to a yield of 83.7% of the theoretical.

### Preparation of Benzyl Meptazinol Para-amino Meta-ethylbenzoate Carbamate

Benzyl 4-amino-3-ethylbenzoate (4.80 g, 18.80 mmol) in tetrahydrofuran (145 mL) was treated with triethylamine (5.71 g, 56.40 mmol) and stirred at −74° C. while phosgene (2M in toluene, 9.4 mL, 18.80 mmol) was added over 20 seconds. The temperature of the reaction mixture rose to −60° C. during this addition. After 45 minutes maintaining at −70 to −75° C. meptazinol hydrochloride (5.07 g, 18.80 mmol) was charged in a single portion and stirring continued at this temperature for 45 minutes. The mixture was warmed to room temperature for 1 hour and treated with saturated aqueous sodium carbonate solution (400 mL) and extracted with ethyl acetate (400 mL and 100 mL). The combined extract was washed with brine (2×100 mL), dried (magnesium sulfate) and evaporated in vacuo. The residual oil was purified by column chromatography on silica gel, eluting with dichloromethane/methanol (97:3). Benzyl meptazinol para-amino meta-ethylbenzoate carbamate of good purity was obtained as a pale yellow oil (2.10 g 21.7%).

### Preparation of Meptazinol Para-amino Meta-ethylbenzoic Acid Carbamate Hydrochloride

A solution of material from the previous step (2.05 g, 3.98 mmol) in tetrahydrofuran (42 mL) was stirred and cooled to 0-5° C. whilst purging with nitrogen. 10% Pd/C (Aldrich 50% wet) (0.80 g) was added and the resulting suspension was purged with nitrogen/vacuum (3 times) then with hydrogen/vacuum (3 times) before allowing to warm to 10-15° C. under a hydrogen atmosphere for 18 hours. TLC analysis (CH₂Cl₂/MeOH, 70:30, UV) indicated complete conversion of starting material (R_{f} 0.67) to a more polar product (R_{f} 0.2-0.25). The reaction mixture was evacuated and purged with nitrogen, then filtered through Celite, washing with tetrahydrofuran (30 ml). Evaporation of the filtrate in vacuo provided the crude product as a foam (1.78 g) which upon trituration with acetone became a white solid which was removed by filtration. TLC (CH₂Cl₂/MeOH, 70:30) of this solid (1.40 g) indicated the presence of some meptazinol (R_{f} 0.55) as a contaminant. Further trituration and washing of this solid with two portions (20 mL and 10 mL) of hot acetone removed the meptazinol, providing meptazinol 3-ethyl PABA carbamate free base as a white solid (0.88 g). This material was of good purity by TLC and ¹H NMR analysis.

The hydrochloride salt was prepared by dissolving the free base (0.88 g) in tetrahydrofuran (30 mL) and treating with 2N HCl in diethyl ether (1.5 mLl, 3.0 mmol). Evaporation in vacuo provided a foam which was crushed to a white powder and dried in vacuo. This material was dissolved in water (3.5 mL) with warming to 30° C. and freeze-dried to obtain meptazinol 3-ethyl PABA carbamate HCl as a white solid, 0.74 g, 40.4%.

NMR Spectrum

¹H NMR (300 MHz, d₆-DMSO): δ 12.88 (br s, 1H), 10.28 (br s, 0.5H), 9.68 (s, 1H), 8.72 (br s, 0.5H), 7.84 (br d, 1H), 7.80 (dd, 1H), 7.65 (d, 1H), 7.50-7.40 (m, 1H), 7.36-7.12 (m, 3H), 4.00 (d, 0.5H), 3.64 (d, 0.5H), 3.56-3.36 (m, 2H), 3.15 (m, 1H), 2.85 (br d, 3H), 2.77 (q, 2H), 2.48-2.36 (m, 1H), 2.30-2.05 (m, 1H), 2.00-1.60 (m, 4H), 1.58-1.40 (m, 2H), 1.20 (t, 3H), 0.52 (br t, 3H).

### Example 8

### Synthesis of Meptazinol 3-methoxy PABA Carbamate

The synthesis of meptazinol-para-amino meta-methoxybenzoic acid carbamate hydrochloride was achieved in 4 distinct reaction steps (see Scheme below).

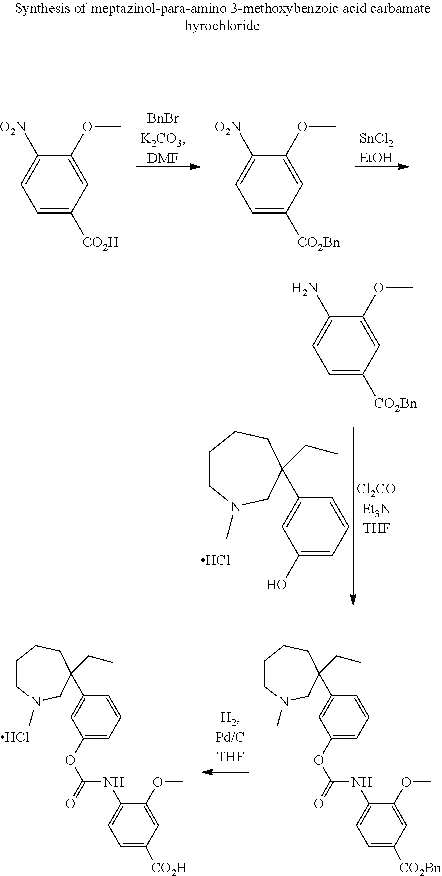

3-Methoxy-2-nitrobenzoic acid was converted to the corresponding benzyl ester and the nitro group was then reduced. This was then treated with an excess of a 20% solution of phosgene in toluene, in dichloromethane in the presence of triethylamine to yield the corresponding isocyanate. The resulting isocyanate was coupled to meptazinol free base in tetrahydrofuran. Cleavage of the benzyl ether was accomplished using a standard hydrogenation procedure to yield after treatment with hydrogen chloride in dioxane, meptazinol 4-amino-3-methoxy benzoic acid carbamate hydrochloride as a yellow solid.

### Detail

Preparation of Benzyl 3-methoxy-4-nitrobenzoate

Benzyl bromide (13.0 g, 76.09 mmol) was added to a stirred mixture of 3-methoxy-4-nitrobenzoic acid (12.5 g, 63.41 mmol) and potassium carbonate (17.5 g, 126.82 mmol) in anhydrous N,N-dimethylformamide (150 mL) under a nitrogen atmosphere. The mixture was then heated to 80° C. for 21 hours. TLC (dichloromethane/methanol, 90:10 visualised by UV) showed all the starting material (R_{f}=0.13) had been consumed producing a single product (R_{f}=0.95). The reaction mixture was cooled to room temperature and poured into water (1500 mL) then extracted with ethyl acetate (500 mL and 3×250 mL). The combined extract was washed with water (4×250 mL), dried (magnesium sulfate) and evaporated in vacuo. The resulting yellow solid was triturated with petroleum ether and collected by filtration, washing with additional petroleum ether. This provided the product as a light beige solid (17.10 g, 93.9%).

Preparation of Benzyl 3-methoxy-4-aminobenzoate

Tin (II) chloride dihydrate (46.7 g, 207 mmol) was added to a stirred solution of benzyl 3-methoxy-4-nitrobenzoate (17.0 g, 59.18 mmol) in ethanol (340 mL) and heated to 80° C., under nitrogen atmosphere for 75 minutes. TLC (petroleum ether/ethyl acetate, 4:1, visualised by UV) indicated that the starting compound had been fully consumed to provide a major product at lower R. The reaction mixture was cooled and evaporated in vacuo. The residual oil was taken up in ethyl acetate (500 mL) and treated with 2N sodium hydroxide (750 mL). A solid was removed by filtration through Celite and washed with ethyl acetate (500 mL). The filtrate layers were separated and the aqueous phase extracted with ethyl acetate (2×200 mL). The combined extract was washed with water (2×200 mL), dried (magnesium sulfate) and evaporated in vacuo. The resulting yellow oil solidified to a waxy solid, 13.94 g, on standing. ¹H NMR was consistent with the required aniline product but showed an impurity present which is thought to be the ethyl ester formed by trans-esterification by the ethanol. Purification was achieved by column chromatography on silica gel eluting with petroleum ether/EtOAc (4:1). The crude product was applied to the column as a solution in dichloromethane and the ethyl ester impurity came off in the fractions prior to the major product. Benzyl 3-methoxy-4-aminobenzoate was thus obtained as a waxy light amber solid, 9.98 g, 65.6%).

Preparation of Benzyl 4-[3-(3-Ethyl-1-methyl-perhydro-azepin-3-yl)-phenoxycarbamoyl]-3-methoxybenzoate

A solution of benzyl 3-methoxy-4-aminobenzoate (5.0 g, 19.43 mmol) in anhydrous tetrahydrofuran (150 mL) was stirred at −75° C., under nitrogen, while 2N phosgene/toluene solution (9.7 mL, 19.4 mmol) was added over 10 seconds. Triethylamine (5.9 g, 58.29 mmol) was then added and the mixture was stirred for 45 minutes keeping temperature in range −70 to −76° C. Meptazinol hydrochloride was then added in a single portion and the suspension was stirred for a further 30 minutes before removing the cooling bath and allowing warming to room temperature (10-12° C.) over 30 minutes. Saturated aqueous sodium carbonate solution (375 mL) was then added and the mixture was extracted with ethyl acetate (375 mL and 100 mL portions). The combined extract was washed with brine, dried (magnesium sulfate) and evaporated in vacuo to leave an amber oil. TLC analysis (dichloromethane/methanol, 9:1, UV) indicated some of each starting material present together with a major product at R_{f} 0.40 and a lesser product at R_{f} 0.44. On standing solid partially crystallised from the oil and treatment with dichloromethane precipitated additional solid which was removed by filtration. TLC analysis indicated this solid was the minor product and NMR suggested it is the urea by-product. The filtrate was evaporated in vacuo and the residual oil was subjected to column chromatography on silica gel, eluting with CH₂Cl₂/MeOH (95:5), collecting 200 mL fractions. Fractions 3-6 contained the highest R_{f} TLC component. Evaporation of these fractions provided an amber oil (2.08 g). ¹H NMR confirmed this was recovered benzyl 3-methoxy-4-aminobenzoate. Fractions 9-13 contained the main product contaminated with the by-product. These fractions were evaporated to obtain a solid/oil mixture, 1.40 g. Fractions 14-24 contained the major product as a single spot by TLC analysis—evaporation of these fractions provided a pale yellow oil (1.47 g, 14.6%). ′H NMR was consistent with the structure of the required product and indicated good purity.

### Preparation of Meptazinol Para-amino Meta-methoxybenzoic Acid Carbamate Hydrochloride

A solution of the product from the previous step (1.40 g, 2.71 mmol) in tetrahydrofuran (28 mL) was stirred and cooled to 0-5° C. whilst purging with nitrogen. 10% Pd/C (Aldrich 50% wet) (0.55 g) was added and the resulting suspension was purged with nitrogen/vacuum (3 times) then with hydrogen/vacuum (3 times) before allowing to warm to 10-15° C. under a hydrogen atmosphere for 18 hours. TLC analysis (CH₂Cl₂/MeOH, 85:15, UV) indicated complete conversion of starting material (R_{f} 0.55) to a more polar product (R_{f} 0.3-0.4). The reaction mixture was evacuated and purged with nitrogen, then filtered through Celite, washing with tetrahydrofuran (30 mL). Evaporation of the filtrate in vacuo provided the crude product as a white foam (1.10 g). TLC analysis showed minor impurities present. Recrystallization from acetone (10 mL), heating to boiling then cooling to 0° C., produced a white crystalline solid which was removed by filtration. After drying in vacuo the yield of meptazinol 3-methoxy PABA carbamate free base was (0.86 g, 74.6%). ¹H NMR was consistent with the structure of meptazinol 3-methoxy PABA carbamate free base and indicated good purity. A portion of meptazinol 3-methoxy PABA carbamate free base (0.55 g, 1.29 mmol) was dissolved in tetrahydrofuran (15 mL) and treated with 2N hydrogen chloride in diethyl ether (1.0 mL, 2.0 mmol) whereupon some material precipitated as a gum. Evaporation in vacuo provided a white foam which was crushed to a powder, but contained some tetrahydrofuran by NMR analysis which was not removed by overnight drying in vacuo. In order to remove the solvent the material was dissolved in water (2 mL) and freeze dried for 24 hours to obtain meptazinol 4-amino-3-methoxy benzoic acid carbamate hydrochloride as a white solid (572 mg).

NMR Spectrum

¹H NMR (300 MHz, d₆-DMSO): δ 12.90 (br s, 1H), 10.45 (br s, 0.5H), 9.40 (s, 1H), 8.78 (br s, 0.5H), 7.88 (d, 1H), 7.62-7.54 (dd, 1H), 7.54 (s, 1H), 7.50-7.40 (m, 1H), 7.38-7.10 (m, 3H), 4.06-3.90 (m, 1H), 3.91 (s, 3H), 3.69-3.35 (m, 2H), 3.14 (br s, 1H), 2.85 (s, 3H), 2.50-2.35 (m, 1H), 2.30-2.10 (m, 1H), 2.06-1.60 (m, 4H), 1.60-1.40 (m, 2H), 0.52 (br t, 3H). Mass Spectrum: ES+: m/z 427.02 (M+H, 100%)

### Example 9

### Synthesis of Meptazinol (PABA-PABA) Carbamate Trifluoroacetate

Meptazinol (PABA-PABA) carbamate trifluoroacetate was synthesised from meptazinol PABA carbamate using a two-step procedure shown in the scheme below:—

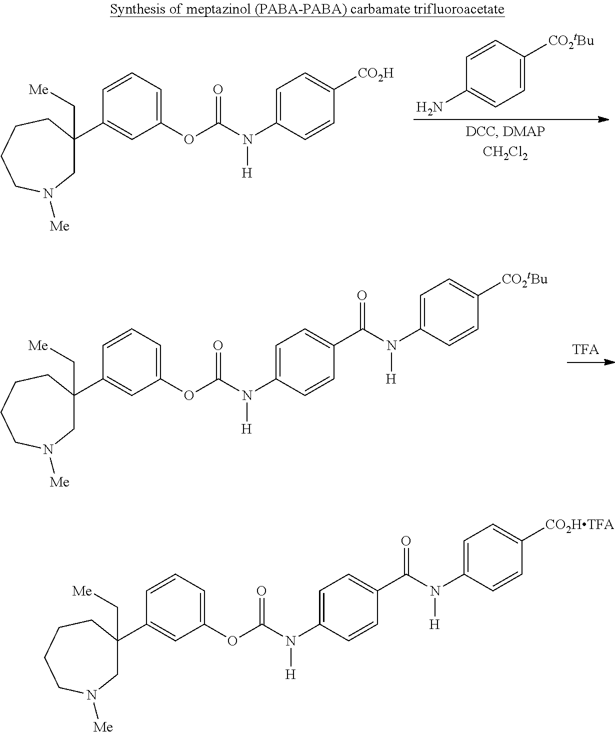

tert-Butyl 4-aminobenzoate was coupled to meptazinol PABA carbamate via a N,N′-dicyclohexylcarbodi-imide (DCC) mediated reaction to give meptazinol (PABA-PABA tert-butyl ester) carbamate. Due to the instability of the product to normal phase column chromatography, the tert-butyl ester was immediately cleaved using trifluoroacetic acid and the product was purified using reversed-phase chromatography to afford the desired meptazinol (PABA-PABA) carbamate trifluoroacetate.

### Detail

To a stirred solution of meptazinol PABA carbamate (0.50 g, 1.26 mmol), tert-butyl 4-aminobenzoate (0.27 g, 1.39 mmol) and 4-dimethylaminopyridine (4 mg, 0.03 mmol) in a mixture of THF and DMF (12 mL, 1:1 v/v) was added N,N′-dicyclohexylcarbodiimide (0.36 g, 1.77 mmol) in one portion and stirring was continued overnight. The resulting suspension was filtered through Celite and the filtrate was concentrated. The residue was dissolved in dichloromethane (50 mL) and washed with water (5×50 mL), brine (50 mL), dried (MgSO₄) and concentrated to afford impure meptazinol (PABA-PABA tert-butyl ester) carbamate (1.00 g), as a yellow oil.

A solution of crude meptazinol (PABA-PABA tert-butyl ester) carbamate (1.00 g) in trifluoroacetic acid (20 mL) was stirred at room temperature for 45 min. The mixture was evaporated to dryness and residual trifluoroacetic acid was removed azeotropically with chloroform (5×30 mL). The residue was purified using a Biotage Isolera automated chromatography system under reversed-phase conditions (C₁₈ column, gradient of 0→100% acetonitrile in 0.1% aqueous TFA) with detection at 297 nm to afford, after freeze-drying, meptazinol (PABA-PABA) carbamate trifluoroacetate (0.13 g, 16% over two steps), as a white solid.

NMR Spectrum

12.76 (bs, 1H, CO₂H), 10.60 (s, 1H, NH), 10.43 (s, 1H, NH), 8.51 (bs, 1H, NH⁺), 7.98 (d, J=8.8 Hz, 2H, 2×PABA ArH), 7.92 (m, 4H, 4×PABA ArH), 7.67 (d, J=9.0 Hz, 2H, 2×PABA ArH), 7.65 (d, J=8.8 Hz, 1H, ArH), 7.48 (m, 1H, ArH), 7.34 (m, 1H, 1 ArH), 7.21 (m, 1H, ArH), 4.00 (d, J=14.5 Hz, 1.5H, 0.75×NCH₂), 3.65 (d, J=13.8 Hz, 0.5H, 0.25×NCH₂), 3.20 (m, 2H, NCH₂), 2.90 (m, 3H, NCH₃), 2.24 (m, 1H, 0.5×CH₂), 1.99-1.62 (m, 5H, 2.5×CH₂), 1.49 (m, 2H, CH₂), 0.55 (m, 3H, CH₃).

### Example 10

### Meptazinol (PABA-PHBA) Carbamate Hydrochloride

Meptazinol (PABA-PHBA) carbamate hydrochloride was synthesised from meptazinol-PABA carbamate in three reaction steps as shown in the scheme below:—

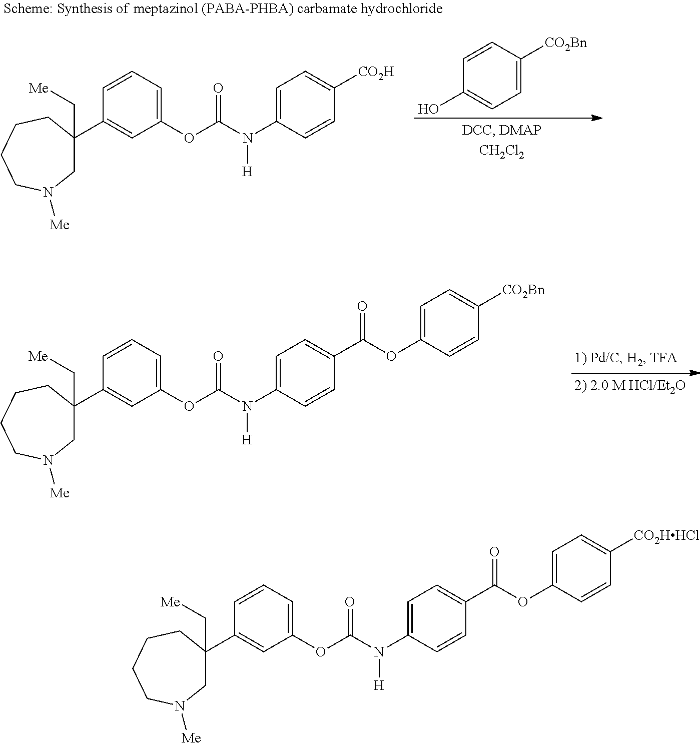

Benzyl 4-hydroxybenzoate was coupled to meptazinol PABA carbamate via a N,N′-dicyclohexylcarbodi-imide (DCC) mediated reaction to give meptazinol (PABA-PHBA benzyl ester) carbamate, after purification by normal phase chromatography. The benzyl ester was removed via catalytic hydrogenolysis to yield meptazinol (PABA-PHBA) carbamate free base which was converted to its hydrochloride salt by treatment with hydrogen chloride in diethyl ether.

### Detail

To a stirred solution of meptazinol PABA carbamate (0.51 g, 1.27 mmol), benzyl 4-hydroxybenzoate (0.32 g, 1.40 mmol) and 4-dimethylaminopyridine (4 mg, 0.03 mmol) in a mixture of THF and DMF (12 mL, 1:1 v/v) was added N,N′-dicyclohexylcarbodi-imide (0.34 g, 1.66 mmol) in one portion and stirring was continued overnight. The resulting suspension was filtered through Celite and the filtrate was concentrated. The residue was dissolved in dichloromethane (50 mL) and washed with water (5×50 mL), brine (50 mL), dried (MgSO₄), concentrated and the residue purified by medium-pressure chromatography on silica eluting with a gradient of 2→4% methanol:ammonia (9:1 v/v) in dichloromethane to afford meptazinol (PABA-PHBA benzyl ester) carbamate (0.27 g, 35%), as an off-white solid. R_{f} 0.35 [10% (MeOH—NH₄OH, 9:1 v/v)—90% dichloromethane]

10% Palladium on carbon (80 mg) was cautiously wetted with ethyl acetate (1 mL) under nitrogen. A solution of meptazinol (PABA-PHBA benzyl ester) carbamate (0.27 g, 0.44 mmol) in anhydrous THF (4 mL) was added and the flask was evacuated. An atmosphere of hydrogen was introduced via a balloon and the mixture was stirred for 5 h at room temperature. The catalyst was removed by filtration of the suspension through a thin layer of Celite and the filtrate was concentrated to afford meptazinol (PABA-PHBA) carbamate (0.24 g), as an off-white solid that was used without further purification.

To a stirred solution of meptazinol (PABA-PHBA) carbamate (0.24 g, 0.46 mmol) in THF (10 mL) was added a solution of 2 M hydrogen chloride in diethyl ether (0.23 mL, 0.46 mmol) and the reaction mixture stirred for 10 min. The suspension was concentrated and the residue was triturated with diethyl ether (2×30 mL) and collected by suction filtration to afford meptazinol (PABA-PHBA) carbamate hydrochloride (0.17 g, 75% over two steps), as a white solid in 90% purity by HPLC and NMR.

A portion of the crude material (0.10 g) was dissolved in water (40 mL), washed with diethyl ether (2×30 mL) and freeze-dried to afford meptazinol (PABA-PHBA) carbamate hydrochloride (65 mg) as a white solid in 98% purity by HPLC.

NMR Spectrum

13.13 (bs, 1H, CO₂H), 10.87 (s, 1H, NH), 8.70 (bs, 1H, NH⁺), 8.20 (d, J=9.2 Hz, 2H, 2×PHBA ArH), 8.10 (d, J=8.7 Hz, 2×PABA ArH), 7.82 (d, J=8.7 Hz, 2×PABA ArH), 7.63-7.38 (m, 4H, 2×PHBA ArH and 2×ArH), 7.29 (m, 2H, 2×ArH), 4.07 (m, 0.5H, 0.25×NCH₂), 3.71 (m, 0.5H, 0.25×NCH₂), 3.56 (m, 1H, 0.5×NCH₂), 3.21 (m, 2H, NCH₂), 2.93 (m, 3H, NCH₃), 2.38-2.30 (m, 1H, 0.5×CH₂), 2.06-1.67 (m, 5H, 2.5×CH₂), 1.64-1.32 (m, 2H, CH₂), 0.61 (t, J=7.1 Hz, 3H, CH₃).

### Example 11

### Synthesis of Buprenorphine PABA Carbamate Hydrochloride

Buprenorphine PABA carbamate hydrochloride was prepared in 3 steps (see Scheme below).

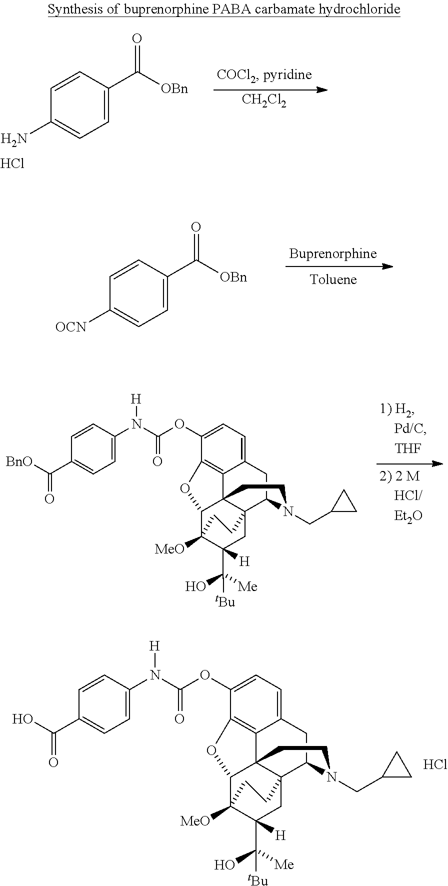

PABA benzyl ester hydrochloride was converted to the corresponding isocyanate by treatment with phosgene in dichloromethane in the presence of pyridine. Following aqueous work-up, the isocyanate was coupled with buprenorphine by heating at reflux in toluene. Purification by column chromatography gave the required buprenorphine PABA carbamate benzyl ester in 90% yield and 99% purity. The benzyl ester was removed via catalytic hydrogenolysis to yield buprenorphine PABA carbamate which was purified by automated chromatography. The free-base was treated with 2M hydrogen chloride in diethyl ether to afford buprenorphine PABA carbamate hydrochloride as a white solid.

### Detail

To a stirred solution of 20% phosgene in toluene (0.28 g, 1.46 mL, 2.78 mmol) in anhydrous dichloromethane (15 mL) at 0° C. under nitrogen was added a solution of benzyl 4-aminobenzoate hydrochloride (0.56 g, 2.14 mmol) and pyridine (0.68 g, 0.69 mL, 8.56 mmol) in anhydrous dichloromethane (10 mL). Stirring was continued for a further 2 h during which the reaction mixture was allowed to warm to room temperature. The resulting mixture was diluted with more dichloromethane (50 mL) and washed with ice-cold 1 M hydrochloric acid (50 mL), followed by saturated brine (50 mL). The organic layer was separated, dried (MgSO₄) and concentrated to give the isocyanate as an oil.

The oil was dissolved in anhydrous toluene (25 mL), buprenorphine (1.00 g, 2.14 mmol) was added and the solution was heated at reflux overnight. After cooling to room temperature, the solvent was evaporated and the residue purified by medium-pressure chromatography on silica eluting with a gradient of 0→2% methanol in dichloromethane to afford buprenorphine PABA benzyl ester carbamate (1.38 g, 90%) as a yellow solid.

R_{f} 0.75 (methanol-dichloromethane, 1:9 v/v).

10% Palladium on carbon (700 mg) was cautiously wetted with ethyl acetate (2 mL) under nitrogen. A solution of the buprenorphine PABA benzyl ester carbamate (1.38 g, 1.92 mmol) in anhydrous THF (20 mL) was added, and the flask was evacuated. An atmosphere of hydrogen was introduced via a balloon, and the mixture was stirred for 5 h at room temperature. The catalyst was removed by filtration of the suspension through a thin layer of Celite and the filtrate concentrated. The residue was purified using a Biotage Isolera automated chromatography system under normal-phase conditions (silica column, gradient of 2.5→20% methanol in dichloromethane) with detection at 265 nm to afford buprenorphine PABA carbamate (885 mg, 73%), as a white solid.

NMR Spectrum

10.68 (br s, 1H, carbamate NH), 9.01 (br s, 1H, NH+), 7.90 (d, J=8.8 Hz, 2H, 2×PABA ArH), 7.57 (d, J=8.8 Hz, 2H, 2×ArH), 7.11 (d, J=8.2 Hz, 1H, ArH), 6.79 (d, J=8.2 Hz, 1H, ArH), 5.43 (br s, 1H, OH), 4.73 (s, 1H, CHO), 3.99-3.97 (m, 1H, CHN), 3.62-3.55 (m, 2H, CH₂N), 3.48-3.41 (m, 1H, CH), 3.37 (s, 3H, CH₃O), 3.30-3.20 (m, 1H, 0.5×CH₂), 3.11-3.05 (m, 1H, 0.5×CH₂), 2.95-2.79 (m, 3H, CH₂+0.5×CH₂), 2.20-2.14 (m, 2H, CH₂), 1.98-1.88 (m, 1H, 0.5×CH₂), 1.54-1.47 (m, 1H, 0.5×CH₂), 1.34-1.36 (m, 3H, CH₂+0.5×CH₂), 1.28 (s, 3H, CH₃), 1.00 (s, 9H, tert-butyl), 0.74-0.58 (m, 3H, 3× cyclopropyl CH), 0.54-0.39 (m, 2H, 2× cyclopropyl CH).

### Example 12

### Synthesis of Buprenorphine-(2-Methoxy-PABA) Carbamate Hydrochloride

Buprenorphine-(2-methoxy-PABA) carbamate hydrochloride was prepared in 8 steps from 2-hydroxy-4-nitrobenzoic acid (see Scheme below).

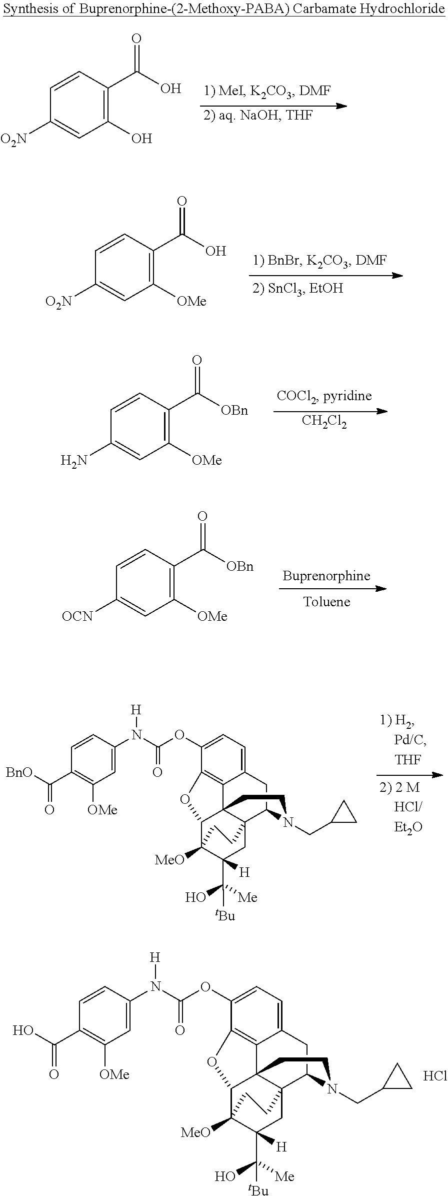

2-Hydroxy-4-nitrobenzoic acid was treated with iodomethane in DMF in the presence of potassium carbonate to give methyl 2-methoxy-4-nitrobenzoate. After purification, the methyl ester was cleaved using aqueous sodium hydroxide in tetrahydrofuran heated at reflux. The benzyl ester was prepared using benzyl bromide in DMF in the presence of potassium carbonate. Reduction of the nitro group was achieved using tin(II) chloride in ethanol to give benzyl 4-amino-2-methoxy benzoate in an overall yield of 71%.

Benzyl 4-amino-2-methoxy-benzoate was converted to the corresponding isocyanate by treatment with phosgene in dichloromethane in the presence of pyridine. Following aqueous work-up, the isocyanate was coupled with buprenorphine by heating at reflux in toluene. Purification by column chromatography gave the required buprenorphine-(2-methoxy-PABA) benzyl ester carbamate in 72% yield and 96% purity. The benzyl ester was removed via catalytic hydrogenolysis to yield buprenorphine-(2-methoxy-PABA) carbamate which was purified by automated chromatography. The free-base was treated with 2 M hydrogen chloride in diethyl ether to afford buprenorphine-(2-methoxy-PABA) carbamate hydrochloride as a white solid.

NMR Spectrum

12.33 (bs, 1H, CO₂H), 10.63 (s, 1H, carbamate NH), 9.48 (bs, 1H, NH⁺), 7.67 (d, J=8.5 Hz, 1H, ArH), 7.33 (s, 1H, ArH), 7.11-7.05 (m, 2H, 2×ArH), 6.79 (d, J=8.2 Hz, 1H, ArH), 5.44 (br, 1H, OH), 4.72 (s, 1H, CHO), 3.98 (d, J=6.4 Hz, 1H, CHN), 3.75 (s, 3H, OCH₃), 3.47-3.31 (m, 5H, CH₂+OCH₃), 3.20-3.04 (m, 3H, 1.5×CH₂), 2.95-2.75 (m, 2H, CH₂), 2.31-2.11 (m, 2H, CH₂), 1.99-1.67 (m, 3H, 1.5×CH₂), 1.53-1.33 (m, 2H, CH₂), 1.28 (s, 3H, CH₃), 1.14-1.06 (m, 1H, CH), 1.00 (s, 9H, tert-butyl), 0.74-0.53 (m, 4H, 4× cyclopropyl CH), 0.45-0.38 (m, 1H, cyclopropyl CH).

### Example 13

### Synthesis of Buprenorphine-(2-Methyl-PABA) Carbamate Hydrochloride

Buprenorphine-(2-methyl-PABA) carbamate hydrochloride was prepared from 2-methyl-4-nitro benzoic acid in 7 steps (Scheme below).

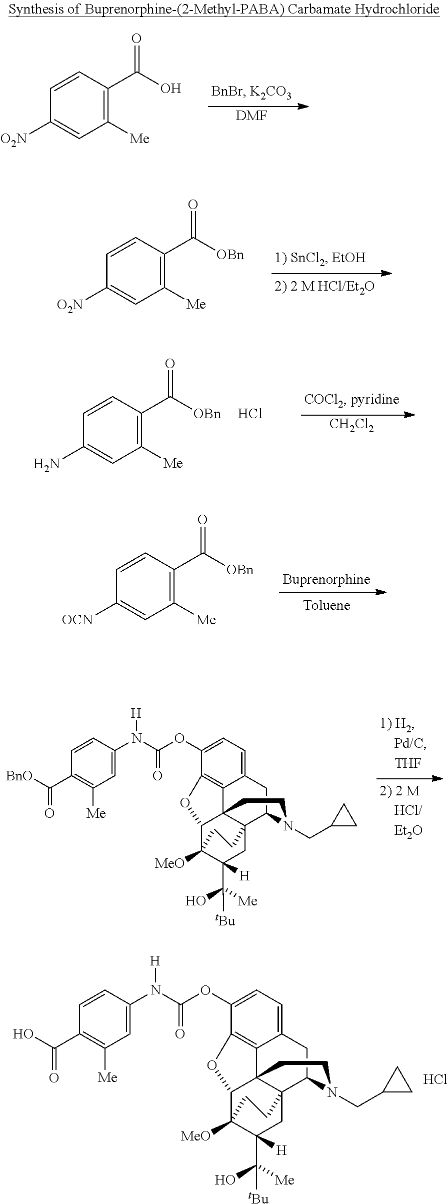

2-Methyl-4-nitrobenzoic acid was treated with benzyl bromide in the presence of potassium carbonate in DMF to give the corresponding benzyl ester. The nitro group was reduced using tin(II) chloride in ethanol, and the resulting aniline was converted to its hydrochloride salt for stability. This was treated with a 20% solution of phosgene in toluene in the presence of pyridine in dichloromethane to give the isocyanate, which was reacted with buprenorphine free-base in refluxing toluene. After purification, the buprenorphine-(2-methyl-PABA benzyl ester) carbamate was subjected to catalytic hydrogenolysis in tetrahydrofuran to give buprenorphine-(2-methyl-PABA) carbamate as a white solid, which was converted to the hydrochloride salt using hydrogen chloride in diethyl ether.

NMR Spectrum

12.60 (br s, 1H, CO2H), 10.56 (s, 1H, carbamate NH), 9.42 (br s, 1H, NH+), 7.83 (d, J=8.4 Hz, 1H, ArH), 7.41-7.37 (m, 2H, 2×ArH), 7.09 (d, J=8.2 Hz, 1H, ArH), 6.79 (d, J=8.2 Hz, 1H, ArH), 5.44 (br s, 1H, OH), 4.72 (s, 1H, CHO), 3.99-3.97 (m, 1H, CHN), 3.47-3.40 (m, 1H, 0.5×CH2), 3.37 (s, 3H, OCH3), 3.36-3.25 (m, 2H, CH2), 3.21-3.14 (m, 1H, 0.5×CH2), 3.10-2.97 (m, 2H, CH2), 2.95-2.74 (m, 2H, CH2), 2.27-2.13 (m, 2H, CH2), 1.96-1.60 (m, 3H, CH2+0.5×CH2), 1.53-1.45 (m, 0.5×CH2), 1.41-1.32 (m, 1H, CH), 1.35 (s, 3H, CH₃), 1.28 (s, 3H, CH3), 1.00 (s, 9H, tert-butyl), 0.76-0.52 (m, 4H, 4× cyclopropyl CH), 0.48-0.38 (m, 1H, cyclopropyl CH).

### Example 14a

### Synthesis of Buprenorphine-(6-Aminonicotinate) Carbamate Dihydrochloride

Buprenorphine-(6-aminonicotinate) carbamate dihydrochloride was prepared in 5 steps (see Scheme below).

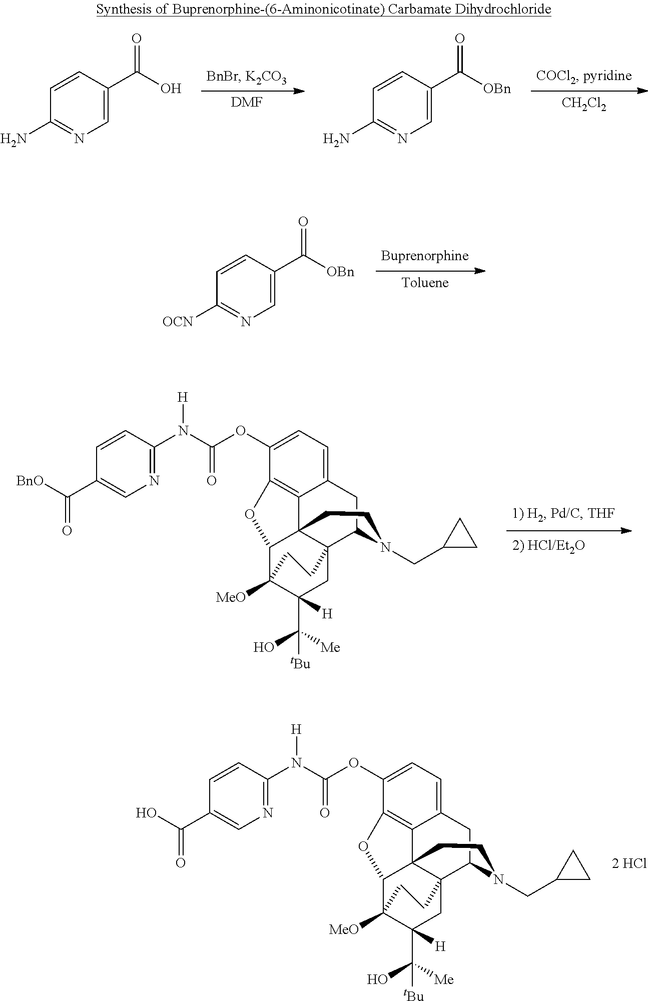

Benzyl 6-aminonicotinate was prepared by the reaction of 6-aminonicotinic acid with benzyl bromide in DMF in the presence of potassium carbonate. Benzyl 6-aminonicotinate was converted to the corresponding isocyanate by treatment with phosgene in dichloromethane in the presence of pyridine. Following aqueous work-up, the isocyanate was coupled with buprenorphine by heating at reflux in toluene followed by purification by column chromatography to give buprenorphine-(6-aminonicotinate benzyl ester) carbamate. The benzyl ester was removed via catalytic hydrogenolysis to yield buprenorphine-(6-aminonicotinate) carbamate which was purified by automated chromatography. This was treated with 2 M hydrogen chloride in diethyl ether to afford buprenorphine-(6-aminonicotinate) carbamate dihydrochloride as a white solid.

NMR Spectrum

11.28 (br s, 1H, carbamate NH), 9.92 (br s, 1H, NH⁺), 8.82 (d, J=2.2 Hz, 1H, ArH), 8.26 (dd, J=2.2, 8.7 Hz, 1H, ArH), 7.86 (d, J=8.7 Hz, 1H, ArH), 7.09 (d, J=8.2 Hz, 1H, ArH), 6.79 (d, J=8.2 Hz, 1H, ArH), 5.76 (s, 1H, OH), 4.71 (s, 1H, CHO), 3.98-3.96 (m, 1H, CHN), 3.47-3.41 (m, 1H, 0.5×CH₂), 3.35 (s, 3H, OCH₃), 3.23-3.03 (m, 3H, CH₂+0.5×CH₂), 2.95-2.73 (m, 2H, CH₂), 2.31-2.25 (m, 1H, 0.5×CH₂), 2.22-2.12 (m, 3H, CH₂+0.5×CH₂), 1.94-1.66 (m, 3H, CH₂+0.5×CH₂), 1.56-1.41 (m, 1H, 0.5×CH₂), 1.27 (s, 3H, CH₃), 1.00 (s, 9H, tert-butyl), 0.89-0.83 (m, 1H, CH), 0.72-0.55 (m, 4H, 4× cyclopropyl CH), 0.44-0.37 (m, 1H, cyclopropyl CH)

### Example 14b

### Synthesis of Buprenorphine (PABA-PABA) Carbamate hydrochloride

Buprenorphine (PABA-PABA) carbamate hydrochloride was prepared using a 6 step synthetic procedure (see scheme below).

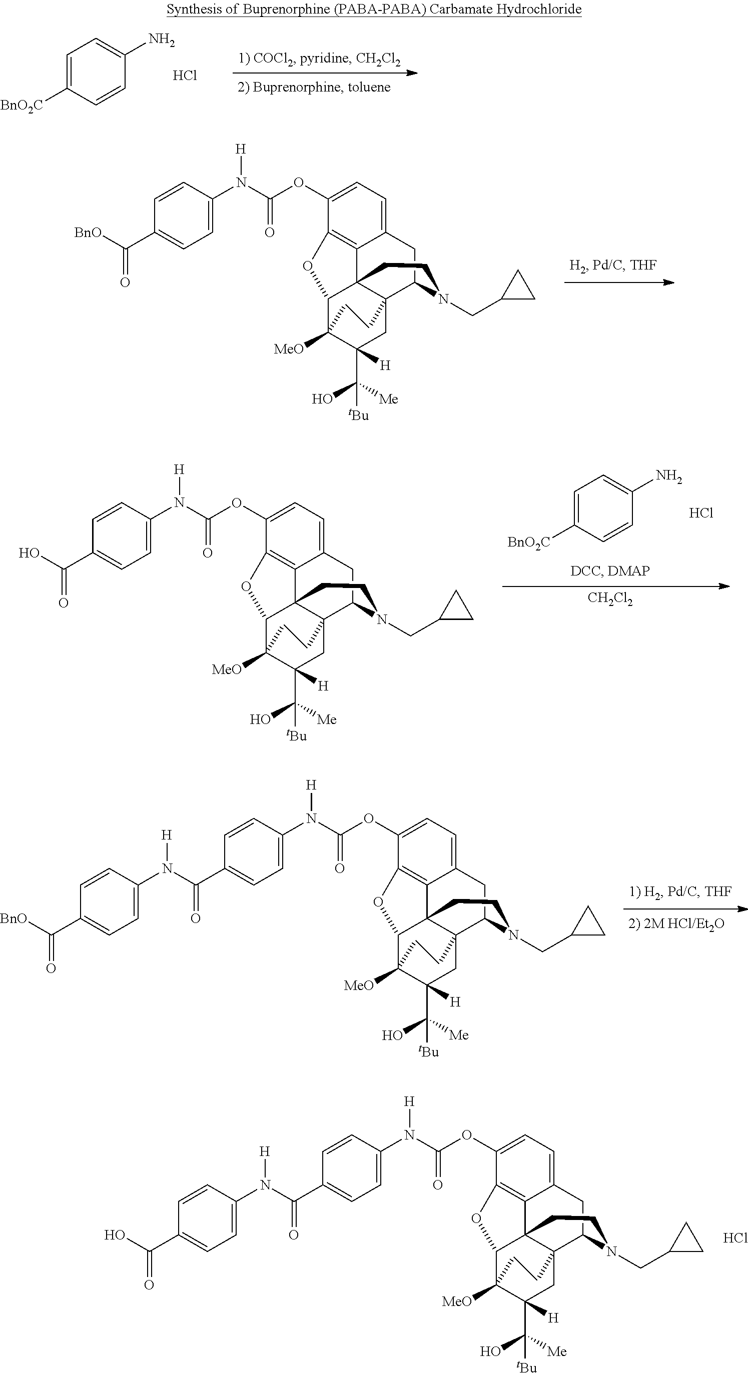

Benzyl 4-aminobenzoate hydrochloride was converted to the corresponding isocyanate by treatment with phosgene in dichloromethane in the presence of pyridine. Following aqueous work-up, the isocyanate was coupled with buprenorphine by heating at reflux in toluene. After purification the benzyl ester was removed via catalytic hydrogenolysis and purified by automated chromatography to yield buprenorphine PABA carbamate free base. This was coupled to benzyl 4-aminobenzoate hydrochloride via an N,N′-dicyclohexylcarbodiimide (DCC) mediated reaction to give buprenorphine (PABA-PABA benzyl ester) carbamate, after purification. The benzyl ester was removed via catalytic hydrogenolysis and purified by automated chromatography to yield buprenorphine (PABA-PABA) carbamate. The free base was treated with hydrogen chloride in diethyl ether to afford buprenorphine (PABA-PABA) carbamate hydrochloride as a white solid.

### Detail

To a stirred solution of buprenorphine PABA carbamate free base (0.46 g, 0.74 mmol), benzyl 4-aminobenzoate hydrochloride (0.21 g, 0.81 mmol) and 4-dimethylaminopyridine (2 mg, 0.02 mmol) in a mixture of anhydrous THF and anhydrous DMF (10 mL, 1:1 v/v) was added N,N′-dicyclohexylcarbodiimide (0.20 g, 0.96 mmol) in one portion and stirring was continued overnight. The resulting suspension was filtered through Celite and the filtrate was concentrated. The residue was purified by medium-pressure chromatography on silica eluting with a gradient of 0→2% methanol in dichloromethane to afford buprenorphine (PABA-PABA benzyl ester) carbamate (0.27 g, 40%), as an off-white solid.

R_{f} 0.30 [10% MeOH—90% dichloromethane]

10% Palladium on carbon (0.13 g) was cautiously wetted with ethyl acetate (1 mL) under nitrogen. A solution of buprenorphine (PABA-PABA benzyl ester) carbamate (0.27 g, 0.32 mmol) in anhydrous THF (5 mL) was added, and the flask was evacuated. An atmosphere of hydrogen was introduced via a balloon, and the mixture was stirred for 5 h at room temperature. The catalyst was removed by filtration of the suspension through a thin layer of Celite and the filtrate was concentrated. The residue was purified using a Biotage Isolera automated chromatography system under normal phase conditions (silica column, gradient of 0→25% methanol in dichloromethane) with detection at 300 nm to afford buprenorphine (PABA-PABA) carbamate (0.20 g, 84%), as a white solid.

To a stirred solution of buprenorphine (PABA-PABA) carbamate (0.20 g, 0.27 mmol) in diethyl ether (30 mL) was added a solution of 2 M hydrogen chloride in diethyl ether (0.14 mL, 0.28 mmol). The resulting suspension was stirred for 10 min and then concentrated The residue was triturated with diethyl ether (2×30 mL), collected by suction filtration and dried in vacuo at room temperature for 16 h to afford the desired buprenorphine (PABA-PABA) carbamate hydrochloride (0.10 g, 48%), as a white solid.

NMR Spectrum

13.71 (br s, 1H, CO₂H), 10.67 (s, 1H, carbamate NH), 10.45 (s, 1H, carbamate NH), 9.30 (br s, 1H, NH⁺), 7.97 (d, J=8.8 Hz, 2H, 2×PHBA ArH), 7.95-7.92 (m, 4H, 2×PHBA ArH and 2×PABA ArH), 7.60 (d, J=8.8 Hz, 2H, 2×PABA ArH), 7.11 (d, J=8.1 Hz, 1H, ArH), 6.80 (d, J=8.3 Hz, 1H, ArH), 5.44 (br s, 1H, OH), 4.73 (s, 1H, CHO), 3.98 (br d, J=6.5 Hz, 1H, CHN), 3.40 (overlap with H₂O, m, CH₂N and CH₃O), 3.28 (m, 1.5H, 0.75×CH₂), 3.09 (m, 1H, 0.5×CH₂), 2.90 (m, 3.5H, 1.75×CH₂), 2.22 (m, 2H, CH₂), 1.96 (m, 1H, 0.5×CH₂), 1.77 (m, 2 H, CH₂), 1.51 (m, 1H, 0.5×CH₂), 1.39 (m, 1H, CH), 1.28 (s, 3H, CH₃), 1.00 (s, 9H, tert-butyl), 0.64 (m, 4H, 4× cyclopropyl CH), 0.43 (m, 1H, cyclopropyl CH).

### Example 14c

### Synthesis of Buprenorphine (PABA-PHBA) Carbamate Hydrochloride

Buprenorphine (PABA-PHBA) carbamate hydrochloride was synthesised from buprenorphine in 3 distinct reaction steps (see scheme below).

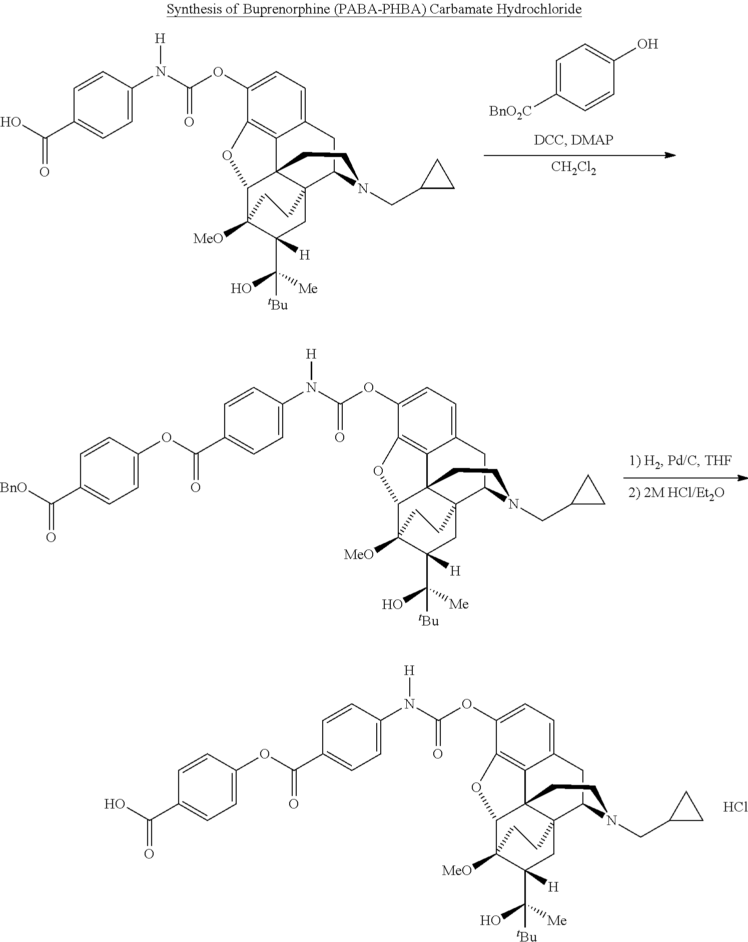

Buprenorphine PABA carbamate free base was coupled to benzyl 4-hydroxybenzoate via an N,N′-dicyclohexylcarbodiimide (DCC) mediated reaction to give buprenorphine (PABA-PHBA benzyl ester) carbamate, after purification. The benzyl ester was removed via catalytic hydrogenolysis and purified by automated chromatography to yield buprenorphine (PABA-PHBA) carbamate. The free base was treated with hydrogen chloride in diethyl ether to afford buprenorphine (PABA-PHBA) carbamate hydrochloride as a white solid.

### Detail

To a stirred solution of buprenorphine PABA carbamate free base (0.50 g, 0.79 mmol), benzyl 4-hydroxybenzoate (0.20 g, 0.87 mmol) and 4-dimethylaminopyridine (2 mg, 0.02 mmol) in anhydrous THF (10 mL) was added N,N′-dicyclohexylcarbodi-imide (0.21 g, 1.03 mmol) in one portion and stirring was continued overnight. The resulting suspension was filtered through Celite and the filtrate was concentrated. The residue was purified by medium-pressure chromatography on silica eluting with a gradient of 0→2% methanol in dichloromethane to afford buprenorphine (PABA-PHBA benzyl ester) carbamate (0.56 g) which was further purified using a Biotage Isolera automated chromatography system under normal phase conditions (silica column, gradient of 7→60% ethyl acetate in petrol) with detection at 257 nm to afford buprenorphine (PABA-PHBA benzyl ester) carbamate (0.31 g, 47%), as an off-white solid.

R_{f} 0.60 [5% MeOH—95% dichloromethane]

10% Palladium on carbon (0.14 g) was cautiously wetted with ethyl acetate (1 mL) under nitrogen. A solution of buprenorphine (PABA-PHBA benzyl ester) carbamate (0.28 g, 0.33 mmol) in anhydrous THF (5 mL) was added, and the flask was evacuated. An atmosphere of hydrogen was introduced via a balloon, and the mixture was stirred for 5 h at room temperature. The catalyst was removed by filtration of the suspension through a thin layer of Celite and the filtrate was concentrated. The residue was purified using a Biotage Isolera automated chromatography system under normal phase conditions (silica column, gradient of 0→25% methanol in dichloromethane) with detection at 272 nm to afford buprenorphine (PABA-PHBA) carbamate (0.22 g, 88%), as a white solid.

To a stirred solution of buprenorphine (PABA-PHBA) carbamate (0.21 g, 0.28 mmol) in diethyl ether (30 mL) was added a solution of 2 M hydrogen chloride in diethyl ether (0.15 mL, 0.30 mmol). The resulting suspension was stirred for 10 min and then concentrated, The residue was triturated with diethyl ether (2×30 mL), collected by suction filtration and dried in vacuo at room temperature for 16 h to afford the desired buprenorphine (PABA-PHBA) carbamate hydrochloride (0.20 g, 95%), as a white solid.

NMR Spectrum

13.08 (br s, 1H, CO₂H), 10.86 (s, 1H, carbamate NH), 9.32-9.00 (m, 1H, NH⁺), 8.11 (d, J=8.8 Hz, 2 H, 2×PHBA ArH), 8.03 (d, J=8.6 Hz, 2H, 2×PHBA ArH), 7.68 (d, J=8.7 Hz, 2H, 2×PABA ArH), 7.41 (d, J=8.7 Hz, 2H, 2×PABA ArH), 7.12 (d, J=8.1 Hz, 1H, ArH), 6.80 (d, J=8.2 Hz, 1H, ArH), 5.44 (br s, 1H, OH), 4.74 (s, 1 H, CHO), 3.98 (br d, J=6.2 Hz, 1H, CHN), 3.40 (overlap with H₂O, m, CH₂N and CH₃O), 3.21 (m, 1.5H, 0.75×CH₂), 3.09 (m, 1H, 0.5×CH₂), 2.89 (m, 3.5H, 1.75×CH₂), 2.20 (m, 2H, CH₂), 1.96 (m, 1H, 0.5×CH₂), 1.78 (m, 2 H, CH₂), 1.51 (m, 1H, 0.5×CH₂), 1.37 (m, 1H, CH), 1.28 (s, 3H, CH₃), 1.00 (s, 9H, tert-butyl), 0.78-0.37 (m, 5H, 5× cyclopropyl CH).

### Example 15

### Synthesis of Racemic Tapentadol PABA Carbamate

This was prepared as shown in the scheme below:—

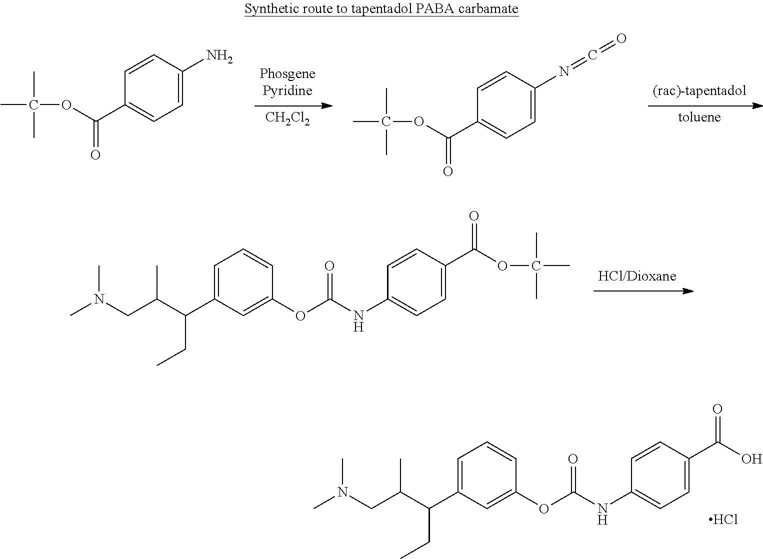

### Detail

tert-Butyl 4-amino benzoate (0.39 g, 2.00 mmol) and pyridine (0.63 g, 0.64 mL, 8.00 mmol) in anhydrous dichloromethane (10 mL) was cooled in an ice-bath under nitrogen. Phosgene (20% solution in toluene, 0.66 mL, 1.33 mmol) was then added cautiously to the stirred mixture. Stirring was continued for a further period of 2 hours while the reaction was warmed to room temperature. The resulting mixture was diluted with more dichloromethane (30 mL) and washed with ice-cold 1M hydrochloric acid (50 mL), followed by brine (50 mL). Next, the mixture was dried (MgSO₄) and concentrated to give the isocyanate (0.78 g), as an oil.

The isocyanate (0.78 g, 2.00 mmol) was dissolved in anhydrous toluene (40 mL). (rac)-tapentadol free base (360 mg, 1.63 mmol) was added and the solution was heated at reflux for 4 hours and then at room temperature overnight. After this time, the solvent was evaporated and the residue was purified using a Biotage Isolera automated chromatography system under reverse phase conditions (gradient acetonitrile:water containing 0.1% TFA) to afford (rac)-tapentadol-PABA carbamate tert-butyl ester (341 mg, 47%), as a brown oil.

The (rac)-tapentadol-PABA carbamate tert-butyl ester (341 mg, 0.77 mmol) was dissolved in a solution of 4M hydrogen chloride in dioxane (1.9 mL, 7.74 mmol) and the resulting solution was stirred at room temperature overnight. The solution was then concentrated and triturated with diethyl ether to afford the (rac)-tapentadol-PABA carbamate hydrochloride (233 mg, 71%), as a brown glassy solid.

NMR Spectrum

12.75 (s, 1H, CO₂H), 10.59 (d, J=7.5 Hz, 1H, carbamate NH), 9.34 (br, 1H, NH⁺), 7.92 (d, J=8.7 Hz, 2H, 2×PABA ArH), 7.62 (d, J=8.7 Hz, 2H, 2×PABA ArH), 7.40 (m, 1H, ArH), 7.12 (m, 3H, 3×ArH), 2.73 (m, 8H, 2×NMe and NCH₂), 2.17 (m, 1H, CH), 1.76 (m, 3H, CH+CH₂), 1.00 (d, J=6.6 Hz, 2H, % Me), 0.80 (d, J=6.6 Hz, 1H, ⅓ Me), 0.68 (m, 3H, Me).

### Example 16

### Synthesis of (R,R)-tapentadol Hydrochloride

(R,R)-Tapentadol free base was prepared starting from the commercially available ketone, 3-(3-methoxyphenyl)propan-2-one. In the first step, bis(dimethylamino)methane was reacted with 3-(3-methoxyphenyl)propan-2-one in a Mannich reaction to give (rac)-3-(dimethylamino)-1-(3-methoxyphenyl)-2-methylpropan-1-one, after purification by chromatography. This racemic material was resolved into its (S)-enantiomer by co-crystallisation with (−)-O,O′-dibenzoyl-(L)-tartaric acid. Liberation of the free base was achieved by treatment with dimethylamine in tert-butyl methyl ether (see Scheme below).

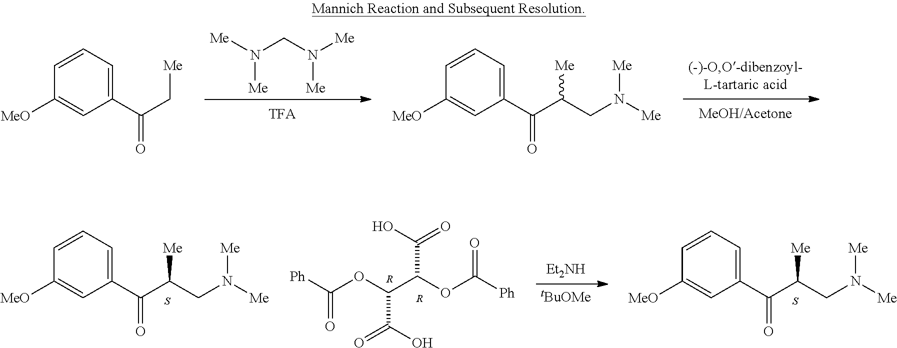

(S)-3-(Dimethylamino)-1-(3-methoxyphenyl)-2-methylpropan-1-one was converted to (S)-1-(dimethylamino)-3-(3-methoxyphenyl)-2-methylpentan-3-ol by a Grignard reaction using ethyl magnesium bromide. This was achieved in good yield after purification by chromatography. Dehydration of (S)-1-(dimethylamino)-3-(3-methoxyphenyl)-2-methylpentan-3-ol with concentrated hydrochloric acid resulted in the formation of (R)-1-(dimethylamino)-3-(methoxyphenyl)-2-methylpent-3-ene (see Scheme below).

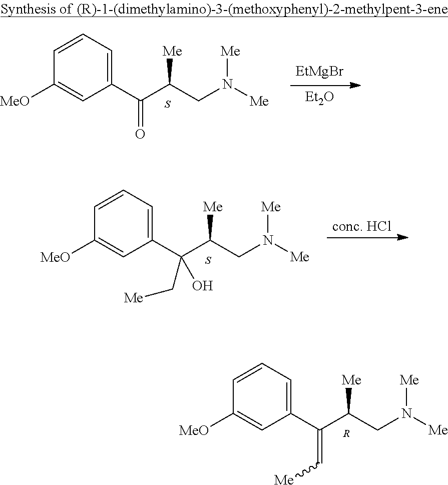

Reduction of the alkene with hydrogen in the presence of catalytic palladium on carbon afforded [(2R,3R)-3-(3-methoxy-phenyl)-2-methyl-pentyl]-dimethyl-amine. Treatment of this material with methanesulfonic acid and methionine resulted in the formation of (R,R)-tapentadol. HPLC analysis showed that the tapentadol contained 90% of the active (R,R) isomer. The free-base was treated with 2 M hydrogen chloride in diethyl ether to form (R,R)-tapentadol hydrochloride (see Scheme below).

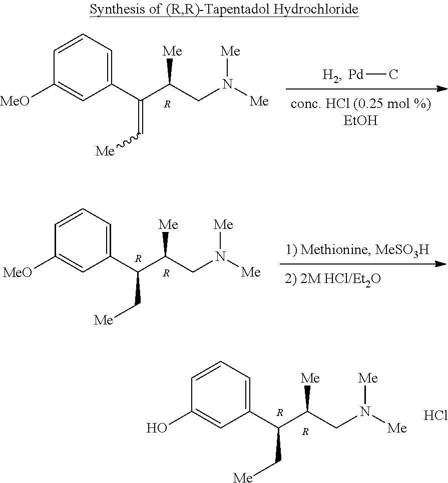

### Detail

To (S)-3-(dimethylamino)-1-(3-methoxyphenyl)-2-methylpropan-1-one (3.60 g, 16.3 mmol) in anhydrous diethyl ether (50 mL) at 10-15° C. was added 2 Methyl magnesium chloride in THF (9.77 mL, 19.5 mmol) and the resulting solution was stirred at this temperature for 2 h. The mixture was cooled to 5° C. and 10% aqueous ammonium chloride (50 mL) was added followed by diethyl ether (20 mL). The layers were separated and the aqueous layer was then washed with diethyl ether (2×50 mL). The ethereal extracts were combined, dried (MgSO₄) and concentrated to give a yellow liquid. This crude material was purified using a Biotage Isolera automated chromatography system under normal phase conditions (silica column, gradient of 2.5→20% methanol in dichloromethane) with detection at 272 nm to afford (S)-1-(dimethylamino)-3-(3-methoxyphenyl)-2-methylpentan-3-ol (2.90 g, 71%), as a waxy white solid.

To (S)-1-(dimethylamino)-3-(3-methoxyphenyl)-2-methylpentan-3-ol (2.90 g, 11.6 mmol) was added concentrated hydrochloric acid (35 mL) and the solution was stirred at 55° C. for 5 h. After cooling to room temperature, the solution was adjusted to pH 12 with 20% sodium hydroxide and the mixture was extracted into ethyl acetate (3×70 mL). The combined organics were dried (MgSO₄) and concentrated. The residue was purified using a Biotage Isolera automated chromatography system under normal phase conditions (silica column, gradient of 1→10% methanol in dichloromethane) with detection at 274 nm to afford (R)-1-(dimethylamino)-3-(methoxyphenyl)-2-methylpent-3-ene (1.98 g, 73%), as a yellow liquid.

10% Palladium on carbon (150 mg) was cautiously wetted with ethanol (10 mL) under nitrogen, followed by concentrated hydrochloric acid (0.18 mL, 2.12 mmol). A solution of (R)-1-(dimethylamino)-3-(methoxyphenyl)-2-methylpent-3-ene (1.98 g, 8.50 mmol) in ethanol (10 mL) was added and the flask was evacuated. An atmosphere of hydrogen was introduced via a balloon and the mixture was stirred overnight at room temperature. The catalyst was removed by filtration of the suspension through a thin layer of Celite and the filtrate was concentrated to yield a residual oil. This crude product was purified by medium-pressure chromatography on silica eluting with a gradient of 5→10% methanol in dichloromethane to afford (R,R)-3-(3-methoxyphenyl)-N,N,2-trimethylpentan-1-amine (1.43 g, 72%) as a white semi-solid.

R_{f} 0.28 [10% methanol—90% dichloromethane].

To (R,R)-3-(3-methoxyphenyl)-N,N,2-trimethylpentan-1-amine (1.43 g, 6.09 mmol) was added methanesulfonic acid (15 mL) followed by D,L-methionine (1.09 g, 7.30 mmol) and the solution was stirred at 80° C. for 3 days. The resulting mixture was cooled to room temperature and adjusted to pH 10-12 with 20% aqueous sodium hydroxide. The solution was extracted with ethyl acetate (4×100 mL), the organic extracts were combined, stirred with activated charcoal for 30 min and then filtered through Celite. The filtrate was dried (MgSO₄) and concentrated to give a yellow oil. This crude product was purified using a Biotage Isolera automated chromatography system under normal phase conditions (silica column, gradient of 3.5→30% methanol in dichloromethane) with detection at 272 nm to afford (R,R)-tapentadol (598 mg, 44%) as a yellow oil.

Ratio of (R,R):(S,S)=90:10.

(R,R)-Tapentadol (189 mg, 0.86 mmol) was stirred in a solution of 2 M hydrogen chloride in diethyl ether (4.3 mL, 8.55 mmol) for 4 h. The solvent was removed in vacuo to yield (R,R)-tapentadol hydrochloride (225 mg, 100%), as a yellow semi-solid.

Ratio of (R,R):(S,S)=90:10.

### NMR Spectrum

9.85 (br s, 1H, NH⁺), 9.37 (s, 1H, OH), 7.10 (t, J=7.6 Hz, 1H, ArH), 6.64-6.58 (m, 3H, 3×ArH), 2.84-2.75 (m, 1H, CH), 2.69-2.61 (m, 6H, 2×NCH₃), 2.33-2.27 (m, 1H, 0.5×NCH₂), 2.13-1.99 (m, 1H, 0.5×NCH₂), 1.78-1.65 (m, 1H, 0.5×CH₂), 1.60-1.43 (m, 1H, 0.5×CH₂), 1.02 (d, J=6.3 Hz, 3H, CH₃), 0.89-0.80 (m, 1H, CH), 0.66 (t, J=7.0 Hz, 3H, CH₃)

### Example 17

### Synthesis of (R,R)-Tapentadol-PABA Carbamate Trifluoroacetate

The synthesis of (R,R)-tapentadol-PABA carbamate trifluoroacetate was achieved in two distinct reaction steps starting from (R,R)-tapentadol free base.

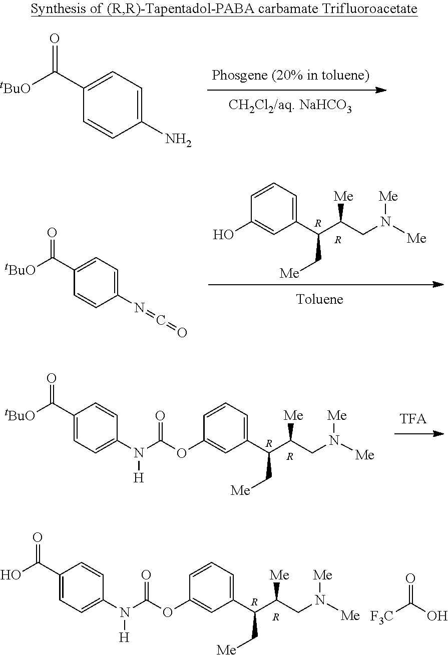

Tert-butyl 4-aminobenzoate was first treated with a 20% solution of phosgene in toluene in a mixture of dichloromethane and saturated aqueous sodium bicarbonate. The resulting isocyanate was treated with (R,R)-tapentadol in toluene to yield (R,R)-tapentadol-(PABA tert-butyl ester) carbamate in good yield after purification by chromatography (see Scheme above). Subsequent deprotection of the tert-butyl ester was achieved using trifluoroacetic acid, to afford (R,R)-tapentadol-PABA carbamate trifluoroacetate as a yellow, gummy semi-solid.

### Detail

A stirred solution of tert-butyl 4-aminobenzoate (398 mg, 2.06 mmol) in a mixture of dichloromethane (4 mL) and saturated aqueous sodium bicarbonate (4 mL) was cooled in an ice-bath. Stirring was stopped and 20% phosgene in toluene (2.04 g, 2.17 mL, 4.13 mmol) was added rapidly to the organic layer. Stirring was resumed and continued for a further 2 h during which time the reaction mixture was allowed to warm to room temperature. The aqueous layer was separated and washed with dichloromethane (2×10 mL). The combined organic layers were dried (MgSO₄) and concentrated to give the isocyanate as a white solid.

The isocyanate was dissolved in anhydrous toluene (5 mL) and added to (R,R)-tapentadol (304 mg, 1.38 mmol) and the resulting solution was heated at reflux overnight. After cooling to room temperature, the mixture was concentrated and the residue was purified using a Biotage Isolera automated chromatography system under normal phase conditions (silica column, gradient of 2.5→20% methanol in dichloromethane) with detection at 273 nm to afford (R,R)-tapentadol-(PABA tert-butyl ester) carbamate (471 mg, 78%), as a white solid.

(R,R)-Tapentadol-(PABA tert-butyl ester) carbamate (471 mg, 1.07 mmol) in trifluoroacetic acid (10 mL) was stirred at room temperature for 1 h. The mixture was evaporated to dryness and residual trifluoroacetic acid was removed azeotropically with chloroform (9×20 mL) to afford (R,R)-tapentadol-PABA carbamate trifluoroacetate (381 mg, 72%), as yellow, gummy semi-solid.

NMR Spectrum

10.60 (br s, 1H, CO₂H), 9.11 (br s, 1H, NH⁺), 7.91 (d, J=8.8 Hz, 2H, 2×PABA ArH), 7.62 (d, J=8.8 Hz, 2×PABA ArH), 7.42-7.37 (m, 1H, ArH), 7.13-7.10 (m, 3H, 3×ArH), 2.92-2.81 (m, 2 H, CH₂N), 2.76 (d, J=4.7 Hz, 3H, NCH₃), 2.69 (d, J=4.7 Hz, 3H, NCH₃), 2.47-2.45 (m, obscured, 1H, CH), 2.22-2.08 (m, 1H, CH), 1.79-1.58 (m, 2H, CH₂), 0.98 (d, J=6.6 Hz, 3H, CH₃), 0.70 (t, J=7.1 Hz, 3H, CH₃)

### Example 18

### Synthesis of Nalbuphine PABA Carbamate

This was effected using the synthetic scheme shown below:—

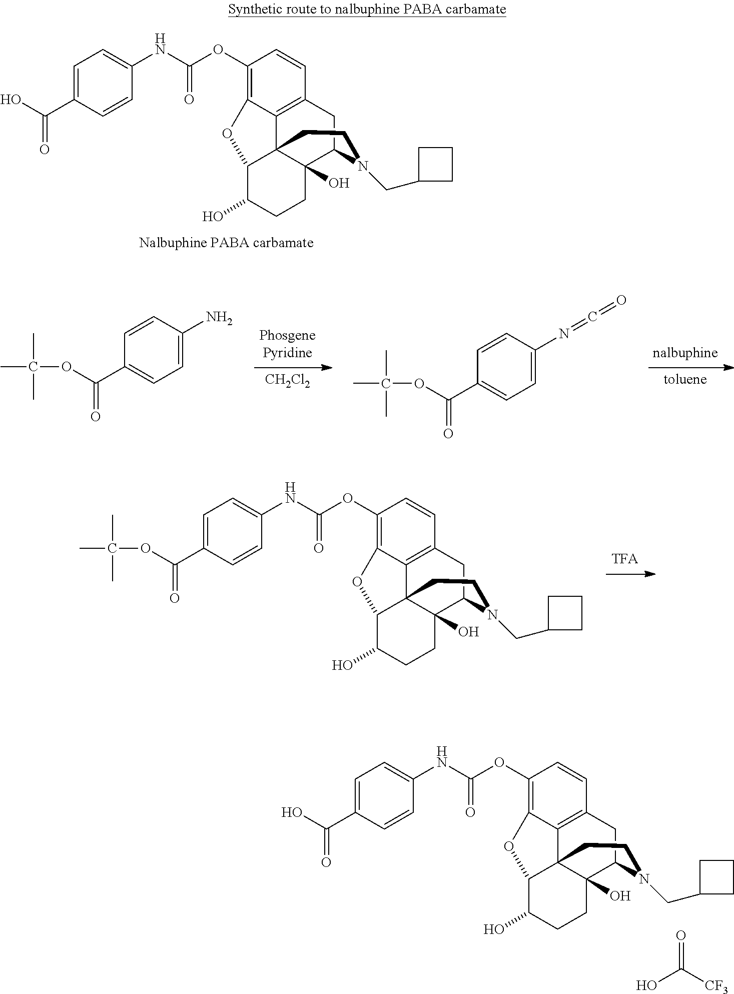

### Detail

tert-Butyl 4-amino benzoate (0.74 g, 3.83 mmol) and pyridine (1.21 g, 1.25 mL, 15.3 mmol) in anhydrous dichloromethane (30 mL) was cooled in an ice-bath under nitrogen. Phosgene (20% solution in toluene, 2.83 mL, 5.38 mmol) was then added cautiously to the stirred mixture. Stirring was continued for a further period of 2 hours while the reaction was warmed to room temperature. The resulting mixture was diluted with more dichloromethane (30 mL) and washed with ice-cold 1M hydrochloric acid (40 mL), followed by brine (40 mL). The mixture was then dried (MgSO₄) and concentrated, to give the isocyanate (0.84 g), as an oil.

The isocyanate (0.84 g, 3.83 mmol) was dissolved in anhydrous toluene (20 mL) and nalbuphine free base (0.65 g, 1.83 mmol) was added. The resulting solution was heated at reflux overnight. The solvent was evaporated and the residue was purified using a Biotage Isolera automated chromatography system under reverse phase conditions (gradient acetonitrile:water containing 0.1% TFA) to give nalbuphine-PABA carbamate tert-butyl ester (286 mg, 11%), as a white solid.

Nalbuphine-PABA carbamate tert-butyl ester (286 mg, 0.50 mmol) was stirred in trifluoroacetic acid (5.7 mL) for 30 minutes. The resulting solution was concentrated and the residual trifluoroacetic acid was removed azeotropically with chloroform (6×25 mL) to give nalbuphine-PABA carbamate trifluoroacetate (249 mg, 79%), as a pale orange, semi-solid.

### NMR Spectrum

10.70 (s, 1H, NH⁺), 8.76 (br s, 1H, NH), 7.91 (d, J=8.8 Hz, 2H, 2×PABA ArH), 7.60 (d, J=8.8 Hz, 2H, 2×PABA ArH), 7.05 (d, J=8.3 Hz, 1H, nalbuphine ArH, 6.75 (d, J=8.3 Hz, 1H, nalbuphine ArH), 6.08 (brs, 1H, OH), 4.69 (d, J=4.6 Hz, 1H, CHO), 4.11 (brs, 1H, OH), 3.50-3.45 (m, 1H, CHOH), 3.40-3.36 (m, 2H, CH₂), 3.17-3.00 (m, 3H, CH₂+CHN), 2.73-2.62 (m, 2H, CH₂), 2.45-2.38 (m, 2H, CH₂ {partially obscured by residual DMSO}), 2.13-2.02 (m, 2H, CH₂), 1.95-1.78 (m, 4H, 2×CH₂), 1.65-1.40 (m, 4H, 2×CH₂), 1.23-1.10 (m, 1H, CH).

### Example 19

### Synthesis of Butorphanol-PABA Carbamate Trifluoroacetate

The synthesis of butorphanol-PABA carbamate trifluoroacetate was achieved in 3 reaction steps (see Scheme below).

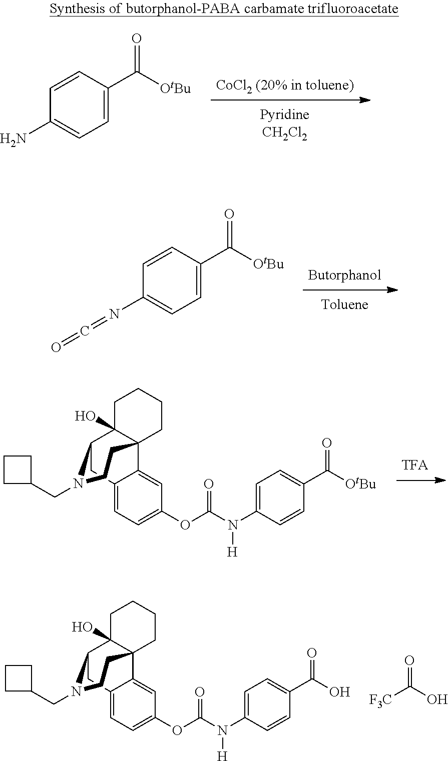

tert-Butyl 4-aminobenzoate was treated with an excess of a 20% solution of phosgene in toluene, in dichloromethane in the presence of triethylamine. The resulting isocyanate was coupled to butorphanol free base in refluxing toluene overnight. Cleavage of the tert-butyl group with trifluoroacetic acid, purification by reversed-phase automated chromatography and precipitation gave butorphanol-PABA carbamate trifluoroacetate as a white solid.

### Detail

A stirred solution of tert-butyl 4-aminobenzoate (649 mg, 3.36 mmol) and pyridine (966 mg, 0.98 mL, 12.2 mmol) in anhydrous dichloromethane (50 mL) was cooled in an ice-bath under nitrogen and 20% phosgene in toluene (2.11 g, 2.25 mL, 4.27 mmol) was added dropwise. Stirring was continued for a further 2 h during which time the reaction mixture was allowed to warm to room temperature. The resulting mixture was diluted with more dichloromethane (20 mL) and washed with ice-cold 2M hydrochloric acid (50 mL), followed by saturated brine (50 mL). The organic layer was separated, dried (MgSO₄) and concentrated to give an oil.

The oil was dissolved in anhydrous toluene (35 mL), butorphanol (1.00 g, 3.05 mmol) was added and the solution was heated at reflux overnight. After cooling to room temperature, the solvent was evaporated and the residue purified using a Biotage Isolera automated chromatography system under normal phase conditions (silica column, dichloromethane-methanol; 92:8 v/v) with detection at 262 nm to give butorphanol-(PABA tert-butyl ester) carbamate (1.07 g, 60%), as an off-white solid.

Butorphanol-(PABA tert-butyl ester) carbamate (1.07 g, 1.96 mmol) in trifluoroacetic acid (5 mL) was stirred at room temperature for 45 min. The mixture was evaporated to dryness and residual trifluoroacetic acid was removed azeotropically with chloroform (5×20 mL) followed by diethyl ether (2×20 mL). The residue was purified using a Biotage Isolera automated chromatography system under reversed-phase conditions (C₁₈ column, gradient of 10→100% MeCN in 0.1% aqueous trifluoroacetic acid) with detection at 262 nm to give after freeze-drying a white solid (750 mg). The residue was dissolved in ethanol (3 mL) and diluted with diethyl ether (200 mL). The precipitate was collected by suction filtration and dried in vacuo at 50° C. overnight to give butorphanol-PABA carbamate trifluoroacetate (515 mg, 43%), as a white solid.

NMR Spectrum

12.78 (s, 1H, COOH), 10.59 (s, 1H, carbamate NH), 8.81 (s, 1H, NH⁺), 7.91 (d, J=8.7 Hz, 2 H, 2×PABA ArH), 7.61 (d, J=8.7 Hz, 2H, 2×PABA ArH), 7.27 (d, J=8.4 Hz, 1H, ArH), 7.19 (d, J=2.1 Hz, 1H, ArH), 7.13 (dd, J=2.1, 8.4 Hz, 1H, ArH), 5.68 (s, 1H, OH), 3.43-3.36 (m, 3H, CHN and Cyclobutylmethyl CH₂), 3.24-3.15 (m, 2H, CH₂N), 3.02-2.92 (m, 2H, benzylic CH₂), 2.71-2.65 (m, 1 H, Cyclobutyl CH), 2.45-2.42 (m, 1H, ½×CH₂), 2.23 (m, 1H, ½×CH₂), 2.09-2.03 (m, 3H, ½×CH₂ and CH₂), 1.88-1.81 (m, 5H, 2×CH₂ and ½ CH₂), 1.36 (m, 2H, CH₂), 1.33 (m, 2H, CH₂), 1.15 (m, 2H, CH₂).

### Example 20

### Synthesis of Levorphanol-PABA Carbamate Trifluoroacetate

The synthesis of levorphanol-PABA carbamate trifluoroacetate was achieved in 3 distinct reaction steps (see Scheme below).

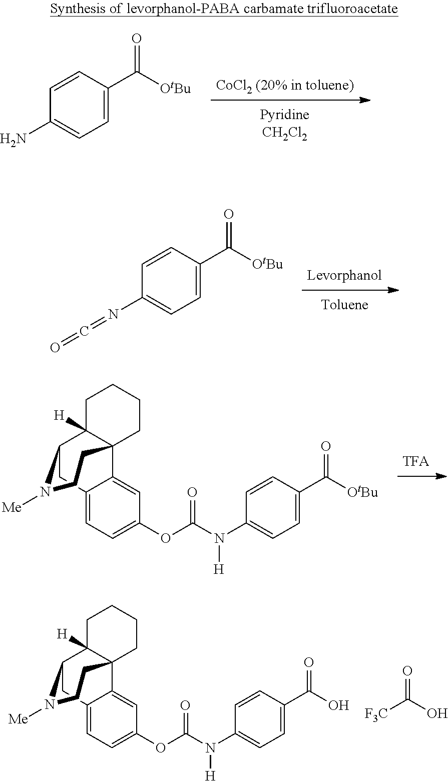

tert-Butyl 4-aminobenzoate was treated with an excess of a 20% solution of phosgene in toluene, in dichloromethane in the presence of triethylamine. The resulting isocyanate was coupled to levorphanol free base in toluene overnight. Cleavage of the tert-butyl group with trifluoroacetic acid, purification by reversed-phase automated chromatography and subsequent precipitation gave levorphanol-PABA carbamate trifluoroacetate as a white solid.

### Detail

A solution of 20 wt % phosgene in toluene (21.3 g, 22.7 mL, 43.1 mmol) was mixed with anhydrous dichloromethane (222 mL) under nitrogen. To the stirred solution was added dropwise a solution of tert-butyl 4-aminobenzoate (833 mg, 4.31 mmol) and triethylamine (873 mg, 1.20 mL, 8.63 mmol) in anhydrous dichloromethane (22 mL), and stirring was continued for 1 h. The solution was concentrated to remove the dichloromethane and excess phosgene (CAUTION, use rotary evaporator inside fume-hood), leaving a solution of the isocyanate in toluene as a slurry with precipitated triethylamine hydrochloride. This was filtered into a 100 mL flask and washed in with further anhydrous toluene (15 mL). Levorphanol free base (1.00 g, 3.89 mmol) was added and the mixture was heated at reflux for 4 h followed by stirring at room temperature overnight. The solvent was evaporated and the residue was purified using a Biotage Isolera automated chromatography system under normal phase conditions (silica column, dichloromethane-methanol; 85:15 v/v) with detection at 261 nm to give levorphanol-(PABA tert-butyl ester) carbamate (1.05 g, 54%), as a white solid.

Levorphanol-(PABA tert-butyl ester) carbamate (1.05 g, 2.20 mmol) in trifluoroacetic acid (5 mL) was stirred at room temperature for 45 min. The mixture was evaporated to dryness and residual trifluoroacetic acid was removed azeotropically with chloroform (4×10 mL) followed by diethyl ether (2×10 mL). The residue was purified using a Biotage Isolera automated chromatography system under reversed-phase conditions (C₁₈ column, gradient of 10→100% MeCN in 0.1% aqueous trifluoroacetic acid) with detection at 265 nm to give after freeze-drying a white solid (950 mg). A portion of this material (300 mg) was dissolved in ethanol (5 mL) and diluted with diethyl ether (200 mL). The precipitate was collected by suction filtration and dried in vacuo at 50° C. overnight to give levorphanol-PABA carbamate trifluoroacetate (202 mg, 46%), as a white solid.

NMR Spectrum

10.58 (s, 1H, carbamate NH), 9.90 (s, 1H, NH⁺), 7.91 (d, J=8.7 Hz, 2H, 2×PABA ArH), 7.61 (d, J=8.7 Hz, 2H, 2×PABA ArH), 7.27 (d, J=8.4 Hz, 1H, ArH), 7.19 (d, J=2.4 Hz, 1H, ArH), 7.12 (dd, J=2.4, 8.4 Hz, 1H, ArH), 3.64 (m, 1H, CHN), 3.34 (m, 1H, ½×CH₂N), 3.23-3.09 (m, 3H, benzylic CH₂+½×CH₂N), 2.86 (m, 3H, CH₃N), 2.43 (m, 1H, CH), 1.98 (m, 1H, ½×CH₂), 1.81 (m, 1H, ½×CH₂), 1.64 (m, 1H, ½×CH₂), 1.48 (m, 3H, ½×CH₂+CH₂), 1.31 (m, 2H, CH₂), 1.15 (m, 1H, ½×CH₂), 0.95 (m, 1H, ½×CH₂).

### Example 21

### Synthesis of Dextrorphan-PABA Carbamate

This was conducted as shown in the Scheme below:—4-(((((4bS,8aS,9S)-11-Methyl-6,7,8,8a,9,10-hexahydro-5H-9,4-b-(epiminoethano)phenanthren-3-yl)oxy)carbonyl)amino)benzoic acid hydrochloride

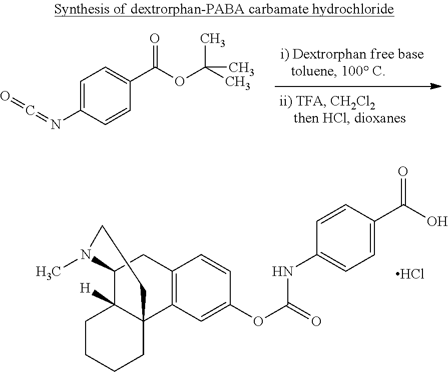

### Detail

Dextrorphan free base (1.44 g, 5.60 mmol) was added to a stirred solution of tert-butyl 4-isocyanatobenzoate (1.35 g, 6.15 mmol) in toluene (30 mL) and heated to 100° C. After 1 hour the reaction mixture was cooled to room temperature and the solvent removed in vacuo. The residue was dissolved in dichloromethane (20 mL), trifluoroacetic acid (10 mL) added and the resulting mixture stirred at room temperature. After 2 hours the reaction mixture was evaporated, the residue was then treated with 4M hydrogen chloride in dioxane (5 mL) and the solvent evaporated; this process of evaporation and treatment with 4M hydrogen chloride in dioxane was repeated 3 times to give a white powder. This solid was triturated successively from diethyl ether (100 mL) and 1:3 acetonitrile:ethyl acetate (150 mL). The isolated solid was dissolved in water (100 mL) and the resultant fine suspension filtered through a plug of celite to give a clear solution. The celite plug was washed with additional water (100 mL) and the combined aqueous filtrates were freeze-dried to afford dextrorphan-PABA-carbamate hydrochloride (4-(((((4bS,8aS,9S)-11-methyl-6,7,8,8a,9,10-hexahydro-5H-9,4-b-(epiminoethano)phenanthren-3-yl)oxy)carbonyl)amino)benzoic acid hydrochloride) (620 mg, 1.474 mmol, 26.4% yield) as a white fluffy solid.

LC-MS: single peak m/z=421.4 [M+H];

NMR Spectrum

¹H NMR (DMSO-d₆, 400 MHz): δ 12.62 (broad, s, 1H, HCl), 10.56 (s, 1H, carbamate NH), 10.46 (broad, s, 1H, CO₂H), 7.92-7.90 (m, 2H, 2ArH-PABA), 7.62-7.60 (m, 2H, 2ArH-PABA), 7.26 (d, J=8.4 Hz, 1H, ArH-dextrorphan), 7.19 (d, J=2.4 Hz, 1H, ArH-dextrorphan), 7.10 (dd, J=8.4, 2.4 Hz, 1H, ArH-dextrorphan), 3.61-3.59 (m, 1H, CH), 3.27-3.23 (m, 1H, CH₂), 3.09-3.06 (m, 2H, CH₂), 2.79 (s, 3H, CH₃), 2.48-2.43 (m, 2H, CH₂), 2.11-2.09 (m, 1H, CH₂), 1.90-1.88 (m, 1H, CH₂), 1.64-1.62 (m, 1H, CH₂), 1.55-1.27 (m, 5H, CH₂), 1.15-1.13 (m, 1H, CH₂), 0.99-0.95 (m, 1H, CH₂).

### Example 22a

### Synthesis of Naloxone-PABA Carbamate Trifluoroacetate

The synthesis of naloxone-PABA carbamate trifluoroacetate was achieved in 3 distinct reaction steps (Scheme below).

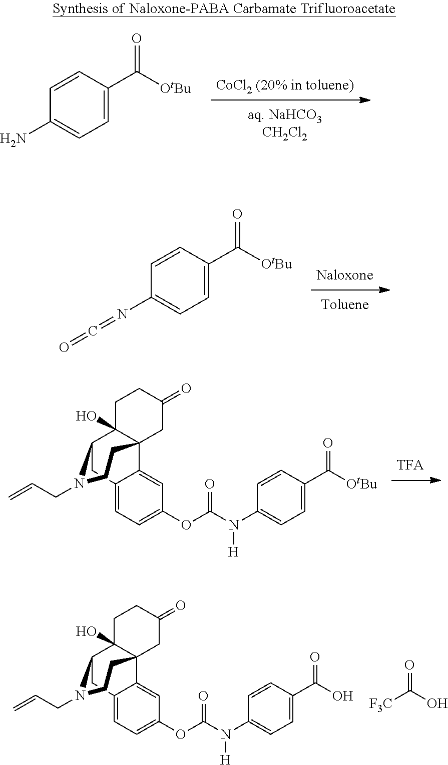

tert-Butyl 4-aminobenzoate was treated with a 20% solution of phosgene in toluene in a two-phase mixture of dichloromethane and saturated aqueous sodium bicarbonate to give the corresponding isocyanate. This was coupled to naloxone in toluene at reflux overnight. Purification by reversed-phase automated chromatography gave naloxone-(PABA tert-butyl ester) carbamate as an off-white solid. Cleavage of the tert-butyl group with trifluoroacetic acid afforded naloxone-PABA carbamate trifluoroacetate as a white solid.

### Detail

A two-phase mixture of tert-butyl 4-aminobenzoate (0.87 g, 4.59 mmol) in dichloromethane (15 mL) and saturated aqueous sodium bicarbonate (15 mL) was cooled to 0° C. A solution of 20% phosgene in toluene (0.91 g, 4.83 mL, 9.17 mmol) was added quickly to the organic layer and the reaction mixture was then stirred for 90 min. The layers were separated and the aqueous layer was extracted with dichloromethane (3×50 mL). The organic layers were combined, dried (MgSO₄) and concentrated to give the isocyanate as an oil.

The oil was dissolved in anhydrous toluene (30 mL), naloxone (1.00 g, 3.06 mmol) was added and the suspension was heated at reflux overnight. After cooling to room temperature, the solvent was evaporated and the residue was purified using a Biotage Isolera automated chromatography system under reversed-phase conditions (C₁₈ column, gradient of 0→100% MeCN in 0.02% hydrochloric acid) with detection at 254 nm. The required fractions were combined, concentrated and saturated aqueous sodium bicarbonate was added till pH=8-9 (50 mL). The aqueous solution was extracted with ethyl acetate (3×100 mL), the organics were combined, dried (MgSO₄) and concentrated to give naloxone-(PABA tert-butyl ester) carbamate (0.96 g, 59%), as an off-white solid.

R_{f} 0.44 (dichloromethane-methanol, 95:5 v/v).

Naloxone-(PABA tert-butyl ester) carbamate (0.69 g, 1.27 mmol) in trifluoroacetic acid (10 mL) was stirred at room temperature for 1 h. The mixture was evaporated to dryness and residual trifluoroacetic acid was removed azeotropically with chloroform (3×20 mL) followed by diethyl ether (2×20 mL). The residue was dissolved in ethanol (2 mL), diluted with diethyl ether (100 mL) and the solid was collected by suction filtration and dried in vacuo at 50° C. for 4 h to afford naloxone-PABA carbamate trifluoroacetate (0.58 g, 75%) as a white solid.

NMR Spectrum

12.78 (s, 1H, CO₂H), 10.75 (s, 1H, NH), 9.43 (broad s, 1H, NH⁺), 7.92 (d, J=8.7 Hz, 2H, 2×PABA ArH), 7.61 (d, J=8.7 Hz, 2H, 2×PABA ArH), 7.12 (d, J=8.4 Hz, 1H, ArH), 6.86 (d, J=8.1 Hz, 1H, ArH), 6.64 (s, 1H, OH), 5.91 (m, 1H, allyl CH), 5.67-5.54 (m, 2H, allyl CH₂), 5.13 (s, 1H, CHO), 3.99 (m, 1H, ½× allyl CH₂N), 3.83 (m, 1H, ½× allyl CH₂N), 3.64 (m, 1H, CHN), 3.54 (d, 1H, J=20.1 Hz, ½×CH₂N), 3.38 (obscured m, 1H, ½×CH₂N), 3.22 (m, 1H, ½× benzylic CH₂), 3.11 (m, 1H, ½× benzylic CH₂), 2.91 (m, 1H, ½×CH₂), 2.61 (m, 1H, ½×CH₂), 2.18 (m, 1H, ½×CH₂), 1.97 (m, 1H, ½×CH₂), 1.54 (m, 2H, CHO.

### Example 22b

### Synthesis of Alvimopan-PABA Carbamate Trifluoroacetate

Alvimopan-PABA carbamate may be synthesised in the manner shown in the scheme below:—

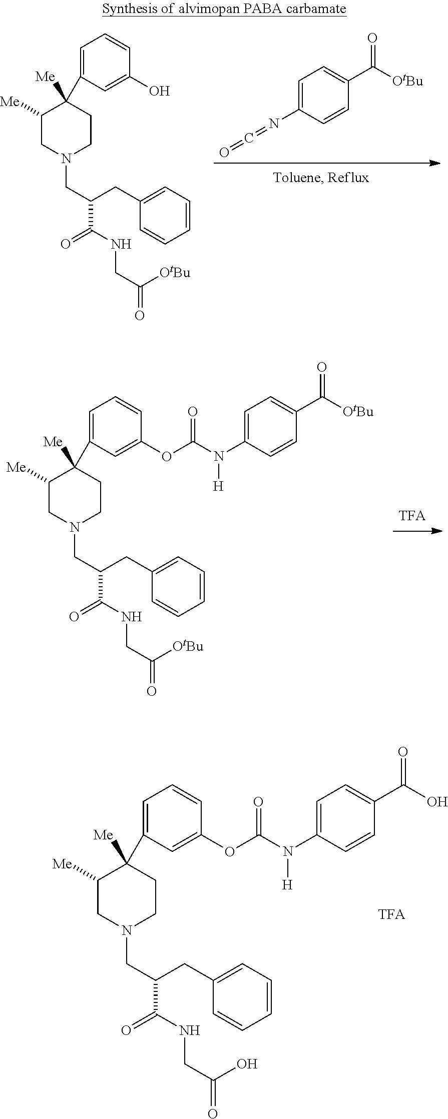

### Example 22c

### Synthesis of Deglycinated Alvimopan-PABA Carbamate Trifluoroacetate

Deglycinated alvimopan-PABA carbamate may be synthesised in the manner shown in the scheme below:—

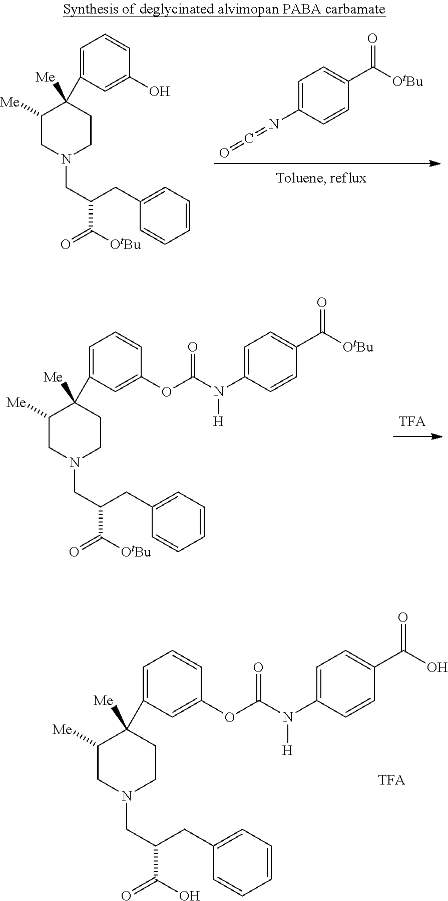

### Example 23

### Investigation of Influence of pH on the Cleavage of Various Amino Acid Carbamate Prodrugs of Meptazinol

In order to demonstrate the role of chemical cleavage and drug release for prodrugs of this type, a comparative examination was undertaken of the effect of pH on release of meptazinol from its PABA carbamate prodrug with other amino acid carbamate prodrugs of meptazinol. An additional consideration was the stability of the PABA prodrug of the present invention under the conditions prevailing in the GI tract. If a prodrug is prematurely hydrolyzed, the gut opioid and other receptors, e.g., cholinergic receptors would be exposed to the parent active drug which may result in locally mediated adverse GI side-effects. Additionally, premature hydrolysis of the prodrug would negate the opportunity for protection against first pass metabolism in the liver and/or subsequent continuing generation of the opioid from the prodrug in the systemic circulation.

It was anticipated that the prodrug would be chemically activated at the higher pH in the blood (compared to those in the GI tract), and therefore, it was important to assess the rate and extent of active drug release at pH 7.4.

### Methodology

To investigate the stability of the PABA prodrug of the present invention under conditions mimicking the gut, meptazinol PABA carbamate alongside some other meptazinol carbamate prodrugs was incubated at 37° C. in simulated gastric and simulated intestinal juice (USP defined composition) for 2 hours. Additionally the potential activation at blood pH of 7.4 was investigated over a similar period. The concentrations of the prodrug & drug were assayed by HPLC.

### Results

As can be seen in Table 5, below, meptazinol PABA carbamate along with the other carbamate prodrugs tested were stable under the conditions existing in the GI tract. However, only the PABA conjugate was hydrolyzed to any significant degree at pH 7.4, the pH prevailing in blood. Thus, while all these compounds would be expected to be absorbed intact, and therefore, have no direct effect on the opioid receptors in the gut only the PABA conjugate would be expected to be released by chemical hydrolysis once in the blood.

TABLE 5
Meptazinol Prodrugs - Chemical and Biological Stability Data

| | Biological/chemical stability - | Chemical Stability - |
|---|---|---|
| | SIF - pH 6.8, 37 C. | pH 7.4, 37° C. |
| Compound | % prodrug remaining at 2 h | % prodrug remaining at 2 h |
| Meptazinol (S) valine carbamate | 99% | ND |
| | | 66% pH 10.0 |
| Meptazinol methionine carbamate | 100% | ND |
| | | 60% pH 10.0 |
| Meptazinol isoleucine carbamate | 99% | ND |
| | | 73% pH 10.0 |
| Meptazinol mono-propyl carbamate | 109% | 94% |
| Meptazinol (S)-phenylalanine carbamate | 63% | 96% |
| Meptazinol (R)-valine carbamate | 74% | 96% |
| Meptazinol glycine Carbamate | 111% | 99% |
| Meptazinol (S)-alanine carbamate | 110% | 97% |
| Meptazinol Phenylglycine carbamate | 98% | 93% |
| Meptazinol tryptophan carbamate | 105% | 98% |
| Meptazinol S-glutamic acid carbamate | 98% | 96% |
| Meptazinol proline carbamate | 125% | 99% |
| Meptazinol di-n-propyl carbamate | 116% | 99% |
| Meptazinol sarcosine carbamate | 104% | 99% |
| Meptazinol-(S)-keto-proline carbamate | 108% | 98% |
| Meptazinol-para-aminobenzoic acid carbamate | 126% | 14% |

### Example 24

### Investigation of Influence of pH on the Cleavage of Meptazinol Para Amino Benzoic Acid Carbamate

The pH lability profile of meptazinol PABA carbamate will not only determine the amount of this transiently metabolically “protected” prodrug available for absorption but also the completeness of its subsequent conversion to meptazinol once in the blood stream. While it is desirable to have good stability at the pH's prevailing in the GI tract subsequent hydrolysis at pH 7.4 in the blood is required to regenerate the active drug A further more detailed investigation of the pH lability profile of meptazinol PABA carbamate was therefore undertaken.

### Methodology

To investigate the pH lability profile of meptazinol PABA carbamate, solutions of the prodrug were incubated at varying pHs (pH 6.6 to 7.6, increments of 0.2 pH units). Samples were drawn at various times during this period, and the concentrations of the prodrug and drug were assayed by HPLC

### Results

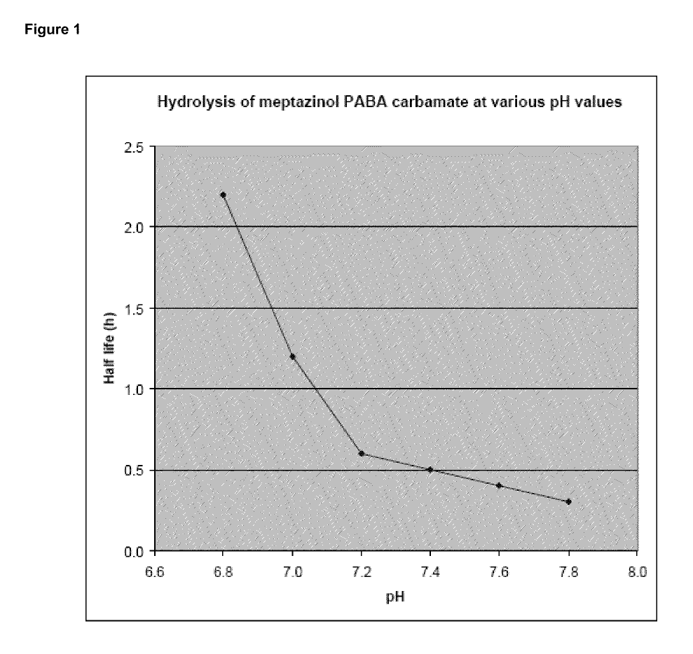

The results from this study (Table 6 and FIG. 1) show that at the pH prevailing in the small intestine, pH 6.8, the majority (˜73%) of the prodrug remains intact at 1 h after the start of the incubation. However, at higher pHs, the prodrug becomes progressively less stable, degrading to meptazinol. At pH 7.4, at 1 h, 64% of the prodrug had been converted to meptazinol increasing to 80% by 2 h. This pH lability profile lends itself to ensuring the prodrug is largely absorbed intact but at plasma pH is subsequently hydrolyzed to the active drug.

TABLE 6
pH lability profile of meptazinol
para-amino benzole acid carbamate

Time (h)

| pH | 0.00* | 0.5* | 1.0* | 2.0* |
|---|---|---|---|---|
| 6.8 | 98/2% | 86/14% | 73/24% | 41/52% |
| 7.0 | 98/2% | 76/21% | 57/36% | 32/56% |
| 7.2 | 97/3% | 58/39% | 32/61% | 10/79% |
| 7.4 | 97/3% | 55/41% | 27/64% | 7/80% |
| 7.6 | 97/3% | 40/51% | 15/71% | 3/81% |
| 7.8 | 96/4% | 33/57% | 11/75% | 1/83% |

*Approximate prodrug remaining/active drug formed

### Example 25

### Comparative Bioavailability of Meptazinol After Oral Administration of Various Meptazinol Carbamate Prodrugs to Dogs and Monkeys

### Methodology

Test substances (i.e., (1) meptazinol or (2) meptazinol PABA carbamate and some other carbamate prodrugs were administered by oral gavage to groups of monkeys and dogs. Animals also received meptazinol intravenously at 1 mg/kg to enable the absolute oral bioavailability to be determined.

Blood samples were taken at various times after administration and submitted to analysis for the parent drug and prodrug using a validated LC-MS-MS assay. Pharmacokinetic parameters derived from the plasma analytical data were determined using Win Nonlin. The results are given in Table 7.

### Results

As can be seen in Table 6, the meptazinol PABA prodrug provides a significantly higher oral bioavailability, compared to the proteinogenic amino acid carbamate prodrugs, a property which appears to be related to their respective chemical stabilities. Meptazinol PABA carbamate showed the greatest propensity for chemical hydrolysis at pH 7.4 and was associated with the highest bioavailability.

TABLE 7
Comparative bioavailability of meptazinol
from various carbamate prodrugs

| | Dog Oral | Monkey Oral | Chemical |
|---|---|---|---|
| | Bioavailability | Bioavailability | stability |
| Compound Name | (%) | (%) | at pH 7.4 |
| Meptazinol HCl (oral) | 6.0 ± 2.1 | 1.8 ± 0.2 | N/A |
| Meptazinol-valine | 25.6 ± 9.9 | 8.8 ± 5.0 | ~100% |
| carbamate | | | |
| Meptazinol-alanine | 5.8 ± 1.7 | <1 | 97% |
| carbamate | | | |
| Meptazinol-(S)-glycine | 3.8 ± 1.2 | <1 | 99% |
| carbamate | | | |
| Meptazinol-leucine | 3.7 | <1 | 94% |
| carbamate | | | |
| Meptazinol - para-amino | 66.7 ± 11.1 | 42 ± 12 | 14% |
| benzoic acid carbamate | | | |

### Example 26

### Comparative Bioavailability of Meptazinol After Oral Administration of Various PABA Analogue Prodrugs of Meptazinol to Dogs and Monkeys

### Methodology

Test substances (i.e., (1) meptazinol or (2) various meptazinol PABA-like carbamate prodrugs were administered by oral gavage to groups of monkeys and dogs.

Blood samples were taken at various times after administration and submitted to analysis for the parent drug and prodrug using a validated LC-MS-MS assay. Pharmacokinetic parameters derived from the plasma analytical data were determined using Win Nonlin.

### Results

As can be seen in Table 8, there was a wide range in achieved oral bioavailabilities not readily predictable from the structure of these analogues.

TABLE 8
Comparative pharmacokinetics of meptazinol from various meptazinol prodrugs (PABA
analogues) following their oral administration to monkey and dog (n = 2). Dose levels:
monkey 2 mg/kg po and dog 1 mg/kg po.

| | Monkey | | | Dog | | |
|---|---|---|---|---|---|---|
| | Cmax | AUC* | | Cmax | AUC* | |
| | (mean | (mean | Mean | (mean | (mean | Mean |
| Compound name | ng/mL) | ngh/mL) | relative F % | ng/mL) | ngh/mL) | relative F % |
| Meptazinol | 1.3 ± 0.3 | 3.1 ± 0.7 | — | 4.0 ± 5.5 | 5.4 ± 5.9 | — |
| Meptazinol PABA | 116 | 207 | 6677 | 69 | 144 | 2667 |
| carbamate | | | | | | |
| Meptazinol (2-methyl | 100 | 198 | 6387 | 50 | 104 | 1926 |
| PABA) carbamate | | | | | | |
| Meptazinol (3-ethyl | 115 | 191 | 6161 | 75 | 102 | 1889 |
| PABA) carbamate | | | | | | |
| Meptazinol (3-methoxy | 104 | 164 | 5290 | 85 | 152 | 2815 |
| PABA) carbamate | | | | | | |
| Meptazinol (6-amino | 41 | 74 | 2387 | 25 | 43 | 796 |
| nicotinate) carbamate | | | | | | |
| Meptazinol MABA | 38 | 63 | 2032 | 14 | 31 | 574 |
| carbamate | | | | | | |
| Meptazinol (p-amino | 17 | 35 | 1129 | 14 | 34 | 629 |
| phenyl acetic acid) | | | | | | |
| carbamate | | | | | | |
| Meptazinol (4-amino 2- | 13 | 26 | 826 | 8.3 | 18 | 333 |
| fluorobenzoic acid) | | | | | | |
| carbamate | | | | | | |
| Meptazinol (p-amino | 8.8 | 19 | 606 | 1.6 | 3.5 | 65 |
| methyl benzoic acid) | | | | | | |
| carbamate | | | | | | |
| Meptazinol (2-hydroxy | 1.8 | 4 | 122 | 3.3 | 6.9 | 128 |
| PABA) carbamate | | | | | | |
| Meptazinol (N-Methyl- | 0.9 | 2 | 60 | 0.5 | 0.5 | 9 |
| PABA) carbamate | | | | | | |

*AUC0-t

### Example 27

### Comparative Bioavailability of Meptazinol After Oral Administration of Meptazinol or Meptazinol PABA Carbamate Prodrug to Dogs

### Methodology

Test substances (i.e., meptazinol or meptazinol para amino benzoic acid carbamate) were administered by oral gavage to groups of five or six dogs at a dose of 1 mg/kg (meptazinol base equivalents) in a parallel group study design. One group of dogs also received meptazinol intravenously at 1 mg/kg.

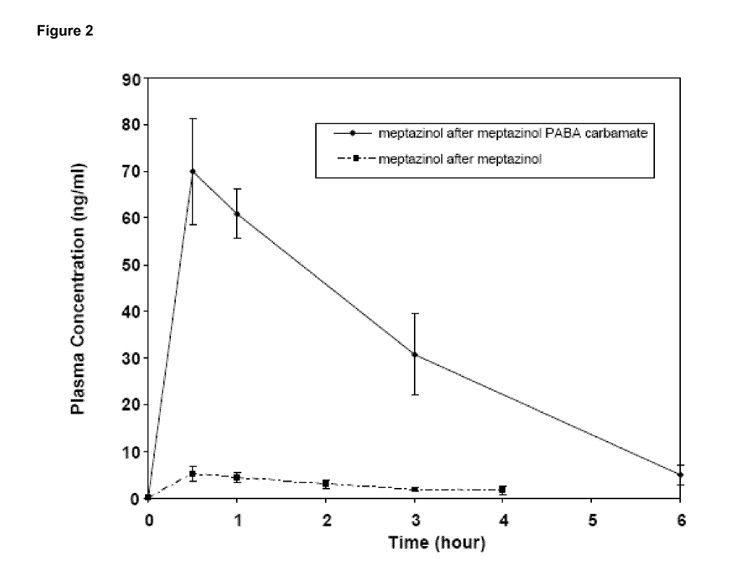

Blood samples (4 per animal) were taken at various times after administration and submitted to analysis for the parent drug and prodrug using a validated LC-MS-MS assay. Pharmacokinetic parameters derived from the plasma analytical data were determined using Win Nonlin. The results are given in Tables 8 to 11 and shown graphically in FIG. 2.

### Results

The pharmacokinetic data on meptazinol in the dog confirm the inherently low oral bioavailability of meptazinol when given in its underivatized form (FIG. 2). The mean absolute oral bioavailability of meptazinol in the dog was 6.0±2.1% (Table 9). By contrast, oral administration of the PABA carbamate prodrug to dogs resulted in over an 11 fold increase in oral bioavailability, to 66.7±11.1 (Table 11, FIG. 2). Prodrug levels were substantial, suggesting both efficient absorption and effective protection of the drug during its first pass through the liver.

TABLE 9
Pharmacokinetic parameters for meptazinol following oral
administration of meptazinol (1 mg meptazinol free base
equivalents/kg) to dogs

| Pharmacokinetic | Dog | | Dog | | Dog | | |
|---|---|---|---|---|---|---|---|
| parameter | 1 | Dog 2 | 3 | Dog 4 | 5 | Mean | SD |
| Cₘₐₓ (ng/mL) | 4.22 | 6.82 | 6.79 | 5.04 | 3.52 | 5.28 | 1.49 |
| Tₘₐₓ (h) | 0.5 | 0.5 | 0.5 | 1 | 1 | 0.5^{a} | |
| AUC (ng · h/mL) | 15.1 | 15.8^{b} | 23.3 | 19.6^{b} | 8.52 | 15.6 | 7.4 |
| t½ (h) | 2.5 | 2.0^{b} | 2.5 | 2.3 | 1.2 | 1.8 | |
| T_{>50%Cmax} (h) | 2.5 | 1.1 | 1.9 | 2.4 | 1.8 | 1.9 | 0.6 |
| Fabs (%) | 6.1 | 5.8 | 8.0 | 6.4 | 3.8 | 6.0 | 2.1 |

^{a}Median value
^{b}Calculated using tlast/Kₑₗ iv to extrapolate to ∞

TABLE 10
Pharmacokinetic parameters for meptazinol following iv administration of
meptazinol (1 mg meptazinol free base equivalents/kg) to dogs

| Pharmacokinetic | | | | | | | |
|---|---|---|---|---|---|---|---|
| parameter | Dog 1 | Dog 2 | Dog 3 | Dog 4 | Dog 5 | Mean | SD |
| Cₒ (ng/mL) | 340 | 308 | 350 | 402 | 477 | 375 | 66 |
| AUC (ng · h/mL) | 247 | 274 | 291 | 305 | 227 | 269^{b} | 32 |
| t½ (h) | 2.5 | 1.7 | 2.3 | 1.9 | 0.6^{a} | 2.0 | |
| CL (L/h/kg) | 4.04 | 3.65 | 3.43 | 3.27 | 4.40 | 3.76 | 0.460 |
| Vss (L) | 4.14 | 3.82 | 3.80 | 3.34 | 2.64 | 3.55 | 0.58 |

^{a}Discounted value
^{b}Calculated using last/Kₑₗ iv to extrapolate to ∞

TABLE 11
Pharmacokinetic parameters for meptazinol following oral administration of
meptazinol PABA carbamate (1 mg meptazinol free base equivalents/kg) to dogs

| Pharmacokinetic | | | | | | | |
|---|---|---|---|---|---|---|---|
| parameter | Dog 6 | Dog 7 | Dog 8 | Dog 9 | Dog 10 | Dog 11 | Mean ± SD |
| Cₘₐₓ (ng/mL) | 69 | 82 | 84 | 57 | 67 | 71 | 71 ± 10 |
| Tₘₐₓ (h) | 0.5 | 0.5 | 0.5 | 0.5 | 1.0 | 0.5 | 0.5^{a} |
| AUC (ng · h/mL) | 186 | 199 | 208 | 130 | 196 | 158 | 179^{b} ± 30 |
| t½ (h) | 1.5 | 1.5 | 1.6 | 1.0 | 3.4 | 1.4 | 1.7 ± 0.8 |
| Fabs (%) | 69 | 74 | 77 | 48 | 73 | 59 | 66.7 ± 11.1 |
| T_{>50%Cmax} (h) | 2.5 | 2.0 | 2.1 | 1.9 | 2.7 | 2.0 | 2.2 ± 0.3 |

^{a}Median value
^{b}Calculated using tlast/Kₑₗ iv to extrapolate to ∞

TABLE 12
Pharmacokinetic parameters for meptazinol PABA carbamate following oral
administration of meptazinol PABA carbamate (1 mg meptazinol free base
equivalents/kg) to dogs

| Pharmacokinetic | | | | | | | |
|---|---|---|---|---|---|---|---|
| parameter | Dog 6 | Dog 7 | Dog 8 | Dog 9 | Dog 10 | Dog 11 | Mean ± SD |
| Cₘₐₓ (ng/mL) | 570 | 606 | 531 | 540 | 922 | 839 | 668 ± 168.7 |
| Tₘₐₓ (h) | 0.5 | 0.5 | 0.5 | 0.5 | 0.5 | 0.5 | 0.5 |
| AUC (ng · h/mL) | 946 | 917 | 992 | 868 | 1568 | 1203 | 1082 ± 265 |
| t½ (h) | 1.15 | 1.01 | 1.07 | 1.0 | 0.79 | 0.72 | 0.97 ± 1.7 |

### Example 28

### Comparative Bioavailability of Meptazinol After Oral Administration of Meptazinol PABA Carbamate Prodrug to Dogs

### Methodology

In an additional study to the study described in Example 27, to better define the pharmacokinetics of meptazinol in the dog after giving the PABA carbamate prodrug, a larger number of blood samples (8 blood samples per animal vs. 4 in Example 26) were collected and analyzed from a further group of dogs. Test substance (meptazinol para amino benzoic acid carbamate) was administered by oral gavage to this further group of five dogs at a dose of 1 mg/kg (meptazinol base equivalents).

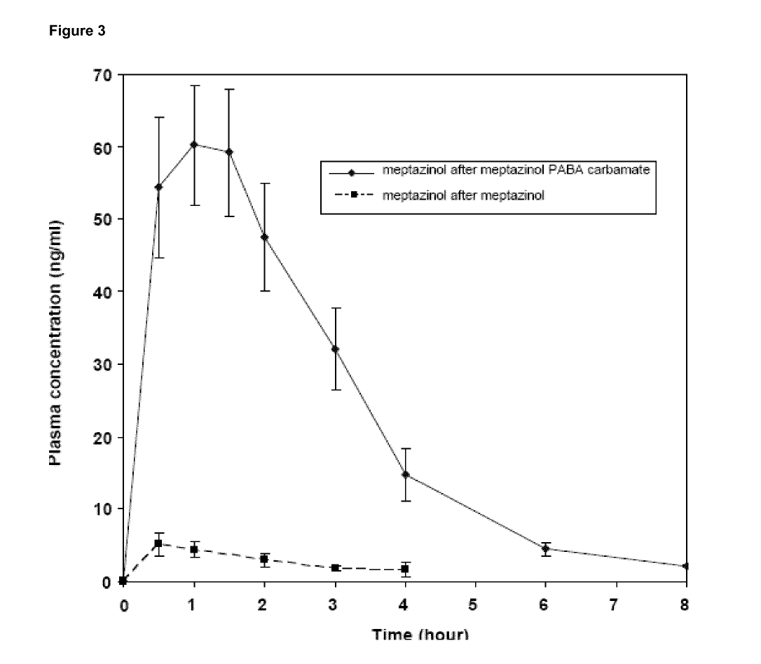

Blood samples were taken at various times after administration and submitted to analysis for the parent drug and prodrug using a validated LC-MS-MS assay. Pharmacokinetic parameters derived from the plasma analytical data were determined using Win Nonlin. The results are given in Tables 12 and 13, and shown graphically in FIG. 3.

For reference and comparison purposes, meptazinol plasma concentration data from the earlier dog study (Example 27, i.e., meptazinol plasma concentration after administration of meptazinol itself to dogs), was shown graphically in FIG. 3.

### Results

The pharmacokinetic data on meptazinol after oral administration of the PABA carbamate prodrug to dogs demonstrated good bioavailability (˜70%) of the drug with minimal variability in the Cmax and AUC (Table 13), the observed relative standard deviation being only ˜13%. Again prodrug levels were substantial (Table 14), suggesting both efficient absorption and effective protection of the drug during its first pass through the liver.

TABLE 13
Pharmacokinetic parameters for meptazinol following
oral administration of meptazinol PABA carbamate to
dogs (1 mg meptazinol free base equivalents/kg)

| Pharmacokinetic | Dog No. | | | | | | |
|---|---|---|---|---|---|---|---|
| parameter | 12 | 13 | 14 | 15 | 16 | Mean | SD |
| Cₘₐₓ (ng/mL) | 68 | 69 | 64 | 50 | 61 | 62 | 8 |
| Tₘₐₓ (h) | 1.5 | 1.0 | 0.5 | 1.5 | 1.0 | 0.5 to 1.5 | — |
| AUC (ng · h/mL) | 215 | 207 | 191 | 159 | 164 | 187 | 25 |
| t½ (h) | 1.2 | 1.2 | 1.3 | 1.4 | 1.1 | 1.2 | 0.1 |
| Bioavailability | 80 | 77 | 71 | 59 | 61 | 70 | 9 |
| (%)* | | | | | | | |

*Calculated using data from a different group of dogs dosed intravenously with meptazinol

TABLE 14
Pharmacokinetic parameters for meptazinol PABA
carbamate following oral administration to dogs
(1 mg meptazinol free base equivalents/kg)

| Pharmacokinetic | Dog No. | | | | | | |
|---|---|---|---|---|---|---|---|
| parameter | 12 | 13 | 14 | 15 | 16 | Mean | SD |
| Cₘₐₓ (ng/mL) | 695 | 629 | 641 | 548 | 492 | 601 | 80 |
| Tₘₐₓ (h) | 0.5 | 0.5 | 0.5 | 0.5 | 0.5 | 0.5 | — |
| AUC (ng · h/mL) | 1041 | 429 | 832 | 662 | 693 | 731 | 226 |
| t½ (h) | 1.0 | 1.6 | 1.1 | 1.6 | 1.3 | 1.3 | 0.3 |

### Example 29

### Comparative Bioavailability of Meptazinol after Oral Administration of Meptazinol or Meptazinol PABA Carbamate Prodrug to Monkeys

### Methodology

Test substances (meptazinol at 10 mg meptazinol free base equivalents/kg or meptazinol PABA carbamate at 2 mg meptazinol free base equivalents/kg) were administered by oral gavage to groups of five or six monkeys in a parallel group study design. A further group of monkeys received meptazinol intravenously at 1 mg/kg.

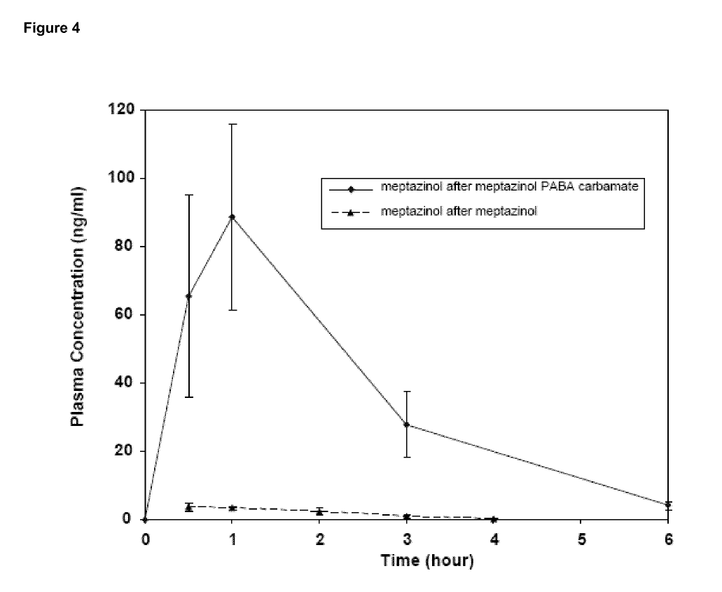

Blood samples were taken at various times after administration, and submitted to analysis for the parent drug and prodrug using a validated LC-MS-MS assay. Pharmacokinetic parameters derived from the plasma analytical data were determined using Win Nonlin. The results are given in Tables 15 to 18 and shown graphically in FIG. 4.

### Results

TABLE 15
Pharmacokinetic parameters for meptazinol following
oral administration of meptazinol (10 mg meptazinol
free base equivalents/kg) to monkeys

| Pharma- | | | | | | | |
|---|---|---|---|---|---|---|---|
| cokinetic | Monkey | Monkey | Monkey | Monkey | Monkey | | |
| parameter | 1 | 2 | 3 | 4 | 5 | Mean | SD |
| Cₘₐₓ | 26.1 | 18.3 | 19.8 | 22.5 | 19 | 21.1 | 3.2 |
| (ng/mL) | | | | | | | |
| Tₘₐₓ (h) | 0.5 | 2.0 | 0.5 | 1.0 | 0.5 | 0.5^{a} | |
| AUC | 39.4 | 47.7 | 37.7 | 38.2 | 47.8 | 42.2 | 5.2 |
| (ng · h/mL) | | | | | | | |
| t½ (h) | 1.0 | 0.8 | 0.9 | 0.7 | 0.7 | 0.8 | 0.1 |
| T_{>50%Cmax} | 0.9 | 2.4 | 1.7 | 1.4 | 2.3 | 1.7 | 0.6 |
| (h) | | | | | | | |
| Fabs (%) | 1.6 | 2.0 | 1.6 | 1.6 | 2.0 | 1.8 | 0.2 |

^{a}Median value
^{b}Calculated using tlast/Kₑₗ iv to extrapolate to ∞

TABLE 16
Pharmacokinetic parameters for meptazinol following iv administration
of meptazinol (1 mg meptazinol free base equivalents/kg) to monkeys

| Pharmacokinetic | Monkey | Monkey | Monkey | Monkey | Monkey | | |
|---|---|---|---|---|---|---|---|
| parameter | 1 | 2 | 3 | 4 | 5 | Mean | SD |
| C₀ (ng/mL) | 211 | 334 | 226 | 369 | 226 | 273 | 73 |
| AUC (ng · h/mL) | 227 | 252 | 228 | 215 | 237 | 232 | 13.7 |
| t½ (h) | 0.81 | 1.0 | 0.83 | 0.68 | 0.87 | 0.84 | 0.13 |
| CL (L/h/kg) | 4.42 | 3.97 | 4.39 | 4.64 | 4.22 | 4.33 | 0.251 |
| Vss (L) | 4.83 | 4.83 | 4.88 | 4.11 | 4.96 | 4.72 | 0.345 |

TABLE 17
Pharmacokinetic parameters for meptazinol following oral administration
of meptazinol PABA carbamate (2 mg meptazinol free base
equivalents/kg) to monkeys

| Pharmacokinetic | Monkey | Monkey | Monkey | Monkey | Monkey | Monkey | |
|---|---|---|---|---|---|---|---|
| parameter | 6 | 7 | 8 | 9 | 10 | 11 | Mean ± SD |
| Cₘₐₓ (ng/mL) | 65 | 77 | 85 | 95 | 90 | 142 | 92 ± 27 |
| Tₘₐₓ (h) | 1.0 | 1.0 | 1.0 | 0.5 | 0.5 | 1.0 | 1.0^{a} |
| AUC (ng · h/mL) | 143 | 177 | 184 | 200 | 192 | 308 | 201^{b} ± 56 |
| t½ (h) | 1.2 | 1.1 | 1.3 | 1.2 | 1.2 | 1.2 | 1.2 ± .01 |
| T_{>50%Cmax} (h) | 1.7 | 1.9 | 1.8 | 1.6 | 1.8 | 1.8 | 1.8 ± 0.1 |
| F (%) | 30 | 37 | 38 | 42 | 40 | 64 | 42 ± 12 |

^{a}Median value
^{b}Calculated using tlast/Kₑₗ iv to extrapolate to ∞

TABLE 18
Pharmacokinetic parameters for meptazinol PABA carbamate, following
oral administration of meptazinol PABA carbamate (2 mg meptazinol
free base equivalents/kg) to monkeys

| Pharmacokinetic | Monkey | Monkey | Monkey | Monkey | Monkey | Monkey | |
|---|---|---|---|---|---|---|---|
| parameter | 6 | 7 | 8 | 9 | 10 | 11 | Mean ± SD |
| Cₘₐₓ (ng/mL) | 441 | 576 | 523 | 846 | 1110 | 1540 | 844 ± 426 |
| Tₘₐₓ (h) | 1 | 1 | 1 | 0.5 | 0.5 | 0.5 | 0.8± |
| AUC (ng · h/mL) | 692 | 900 | 855 | 926 | 1051 | 1760 | 1031 ± 376 |
| t½ (h) | 0.69 | 0.68 | 0.74 | 0.74 | 0.53 | 0.57 | 0.66 ± 0.09 |

These results illustrate that the oral bioavailability of meptazinol when given to monkeys is extremely low—just 1.8% (Table 15, FIG. 4). By contrast, administration of the PABA prodrug resulted in a dramatic increase in bioavailability to a mean of 42% (Table 17).

### Example 30

### Comparative Bioavailability of Meptazinol after Oral Administration of Meptazinol or Meptazinol PABA Carbamate to Rats

### Methodology

Test substances (meptazinol at 1 mg meptazinol free base equivalents/kg or meptazinol para amino benzoic acid carbamate at 1 mg meptazinol free base equivalents/kg) were administered by oral gavage to groups of rats. Animals also received meptazinol intravenously at 1 mg/kg to enable the absolute oral bioavailability to be determined.

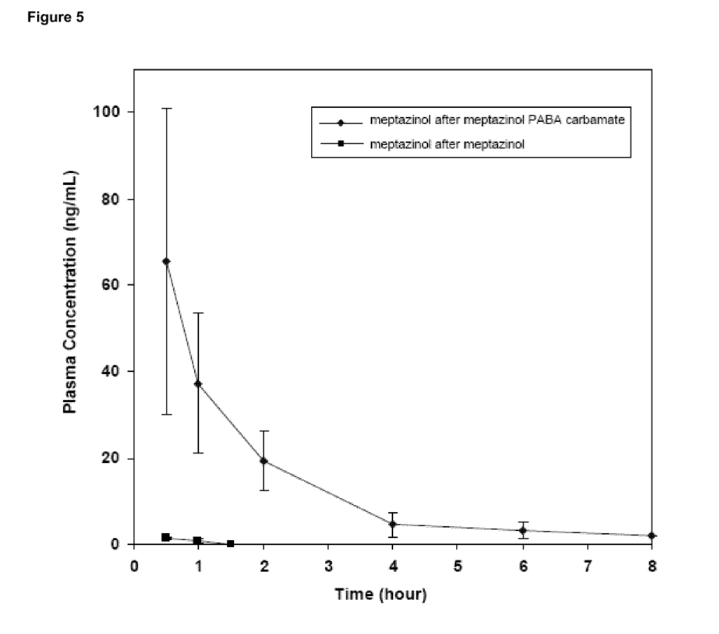

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Tables 19-22 and FIG. 5.

### Results

TABLE 19
Pharmacokinetic parameters for meptazinol following oral
administration of meptazinol (1 mg meptazinol free base
equivalents/kg) to rats

| Pharmacokinetic | Rat number | | | | | | |
|---|---|---|---|---|---|---|---|
| parameter | 1 | 2 | 3 | 4 | 5 | Mean | SD |
| Cₘₐₓ (ng/mL) | 1.93 | 1.12 | 1.49 | 1.6 | 1.44 | 1.6 | 0.2 |
| Tₘₐₓ (h) | 0.5 | 0.5 | 0.5 | 0.5 | 0.5 | 0.5 | — |
| AUC (ng · h/mL)* | 2.9 | ND | 3.1 | 2.8 | 4.9 | 3.4 | 1.0 |
| t½ (h)* | 0.9 | ND | 1.3 | 1.0 | 2.2 | 1.3 | 0.6 |
| Fabs (%) | 1.6 | ND | 1.6 | 1.5 | 2.6 | 1.8 | 0.5 |

*Calculated based on half-life from previous iv study
ND = not determined

TABLE 20
Pharmacokinetic parameters for meptazinol following iv administration
of meptazinol (1 mg meptazinol free base equivalents/kg) to rats

| | Rat number | | | | | | |
|---|---|---|---|---|---|---|---|
| Pharmacokinetic parameter | 6 | 7 | 8 | 9 | 10 | Mean | SD |
| C₀ (ng/mL) | 191 | 246 | 259 | 267 | 244 | 241 | 30 |
| AUC (ng · h/mL) | 161 | 222 | 189 | 187 | 181 | 188 | 22 |
| t½ (h) | 0.8 | 0.6 | 0.7 | 0.7 | 0.7 | 0.7 | 0.1 |
| CL (L/h/kg) | 6.21 | 4.51 | 5.30 | 5.36 | 5.53 | 5.38 | 0.61 |
| Vss (L) | 6.8 | 4.0 | 5.4 | 5.1 | 5.4 | 5.3 | 1.0 |

TABLE 21
Pharmacokinetic parameters for meptazinol following
oral administration of meptazinol PABA carbamate
(1 mg meptazinol free base equivalents/kg) to rats

| Pharmacokinetic | Rat number | | | | | | |
|---|---|---|---|---|---|---|---|
| parameter | 11 | 12 | 13 | 14 | 15 | Mean | SD |
| Cₘₐₓ (ng/mL) | 108.0 | 46.5 | 43.3 | 30.5 | 99.0 | 65.5 | 35.4 |
| Tₘₐₓ (h) | 0.5 | 0.5 | 0.5 | 0.5 | 0.5 | 0.5 | — |
| AUC (ng · h/mL) | 129 | 123 | 81 | 57 | 107 | 99 | 30 |
| t½ (h) | 0.7 | 1.7 | 1.4 | 1.1 | 0.7 | 1.1 | 0.4 |
| Fabs (%) | 69 | 65 | 43 | 30 | 57 | 53 | 16 |

TABLE 22
Pharmacokinetic parameters for meptazinol PABA carbamate
following oral administration of meptazinol PABA carbamate
(1 mg meptazinol free base equivalents/kg) to rats

| Pharmacokinetic | Rat number | | | | | | |
|---|---|---|---|---|---|---|---|
| parameter | 11 | 12 | 13 | 14 | 15 | Mean | SD |
| Cₘₐₓ (ng/mL) | 222 | 120 | 126 | 99 | 355 | 184 | 106 |
| Tₘₐₓ (h) | 0.5 | 0.5 | 0.5 | 0.5 | 0.5 | 0.5 | 0.0 |
| AUC (ng · h/mL) | 242 | 350 | 237 | 146 | 315 | 258 | 79 |
| t½ (h) | 0.5 | 1.5 | 1.7 | 1.0 | 0.5 | 1.0 | 0.5 |

The low oral bioavailability of meptazinol in rats was confirmed after giving the drug itself (Table 19). The mean absolute oral bioavailability of meptazinol in the rat was 1.8±0.5% (Table 19). In contrast, oral administration of the PABA carbamate prodrug resulted in a 30-fold increase in meptazinol oral bioavailability to 53.0±16 (Table 21, FIG. 5). Prodrug levels were substantial suggesting both efficient absorption and effective protection of the drug during its first pass through the liver (Table 22).

### Example 31

### Comparative In Vitro Assessment of Human Acetylcholine Esterase Inhibition by Meptazinol and Meptzinol Para-amino Benzoic Acid Carbamate Prodrug

The local cholinergic effects of meptazinol in the stomach are believed, at least in part, to be responsible for the unwanted emetic activity of the drug. Therefore, the temporary elimination of these cholinergic effects may potentially avoid the emetic activity seen after meptazinol administration. Accordingly, the effects of meptazinol and meptazinol PABA carbamate on human acetyl choline esterase were assessed.

### Methodology

TABLE 23
Assay used to measure human acetylcholine esterase inhibition

| | | Reference | |
|---|---|---|---|
| Assay | Origin | Compound | Bibliography |
| Acetyl- | human recombinant | neostigmine | Ellman et |
| cholinesterase (h) | (HEK-293 cells) | | al. (1961) |

### Experimental Conditions

TABLE 24
Experimental Conditions used for assay

| | Substrate/ | | Reaction | Method of |
|---|---|---|---|---|
| Assay | Tracer | Incubation | Product | Detection |
| Acetyl- | AMTCh | 30 min./ | thio- | Photometry |
| cholinesterase (h) | (50 μM) | 37° C. | conjugate | |

### Analysis and Expression of Results

The results are expressed as a percent of control specific activity ((measured specific activity/control specific activity)×100) obtained in the presence of the test compounds.

The IC₅₀ values (concentration causing a half-maximal inhibition of control specific activity), and Hill coefficients (nh) were determined by non-linear regression analysis of the inhibition curves generated with mean replicate values using hill equation curve fitting (y=d+[(a−d)/(1+(c/c₅₀)^{nh})], where y=specific activity, d=minimum specific activity, a=maximum specific activity, c=compound concentration, C₅₀=IC₅₀, and nh=slope factor).

This analysis was performed using software developed at Cerep (Hill software) and validated by comparison with data generated by the commercial software Sigmaplot® 4.0 for Windows®.

The apparent IC₅₀ value for meptazinol was 1.5 μm while that for the para-amino benzoic acid carbamate was 15 μM. These results suggest that when in contact with the stomach/gut wall, the prodrug would be unlikely to directly elicit a cholinergic response. Since the local cholinergic response associated with meptazinol is believed to be at least in part responsible for the emesis seen after oral administration of meptazinol, administration of the prodrug would be less likely to elicit this response

### Example 32

### Comparative In Vitro Metabolism of Meptazinol PABA Carbamate by Hepatocytes from Rat, Dog, Monkey and Man

In order to determine whether the generation of meptazinol from the prodrug, meptazinol PABA carbamate, would translate from animals to man, a comparative in vitro metabolism study was undertaken across the species

### Methodology

The comparative metabolism of meptazinol PABA carbamate was investigated using cryo-preserved hepatocytes (pooled from a minimum of 3 individuals) collected from Sprague-Dawley rats, Beagle dogs, Cynomolgus monkeys and humans. Incubate samples were removed at various time points over the course of the 2 h experiment and assayed for released meptazinol (its subsequent glucuronide metabolite) by LC-MS/MS. For comparative purposes a similar study was conducted using meptazinol itself.

### Results

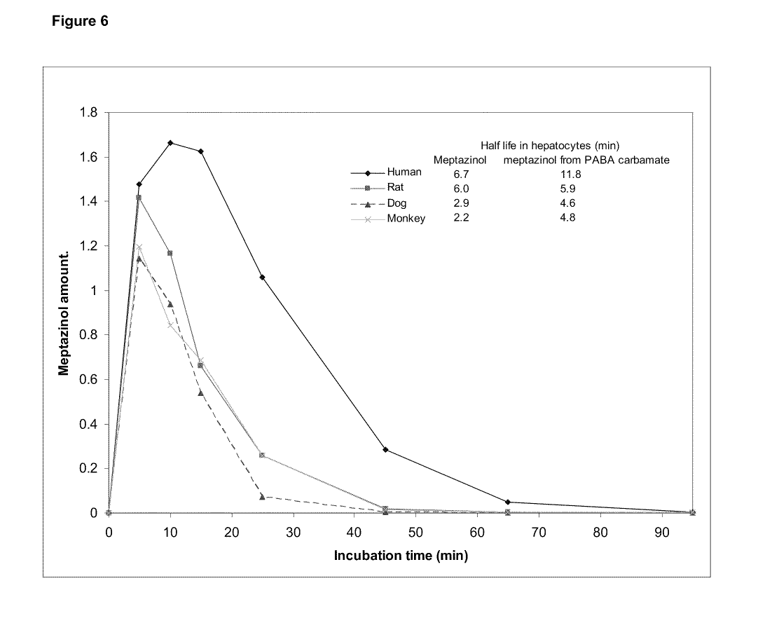

Meptazinol was liberated from meptazinol PABA carbamate in incubations of hepatocytes from all species and subsequently eliminated subsequently by further metabolism, typically as meptazinol glucuronide. Maximal amounts of meptazinol generated were comparable in all species. Subsequent elimination by glucuronidation occurred at a similar in rats, dogs and monkey hepatocyte incubations but was somewhat slower in human hepatocytes where the calculated half life of meptazinol was approx. two-fold longer. The similar amounts of meptazinol generated in human hepatocytes compared to the animal species (see FIG. 6) suggests that the beneficial improvement in oral bioavailability seen in animals should translate to man.

### Example 33

### Comparative Bioavailability of Buprenorphine after Oral Administration of Various PABA Analogue Prodrugs of Buprenorphine to Dogs and Monkeys

### Methodology

Test substances (i.e. (1) buprenorphine or (2) various buprenorphine PABA carbamate prodrugs were administered by oral gavage to groups of monkeys and dogs.

Blood samples were taken at various times after administration and submitted to analysis for the parent drug and prodrug using a validated LC-MS-MS assay. Pharmacokinetic parameters derived from the plasma analytical data were determined using Win Nonlin.

### Results

As can be seen in Table 25, in the dog, only the buprenorphine PABA prodrug and the 2-methoxy PABA carbamate provided significantly higher oral bioavailabilities than the parent drug (4-5-fold). In the monkey, the PABA carbamate provided a significant increase (2.6-fold) in bioavailability. However the most profound improvement appeared to be after the 2-methoxy PABA analogue (14.8-fold). In the dog the respective increases were 4.3 and 5.2 fold.

TABLE 25
Comparative pharmacokinetics of buprenorphine from various buprenorphine prodrugs (PABA
analogues) following their oral administration to monkey and dog (n = 5).
Dose levels: monkey 0.2 mg/kg po and dog 0.1 mg/kg po (except were stated otherwise).

| | Cmax | AUC | | Cmax | AUC | Mean relative |
|---|---|---|---|---|---|---|
| | (mean | (mean | Mean relative | (mean | (mean | bioavailability |
| | ng/mL) | ngh/mL) | bioavailability % | ng/mL) | ngh/mL) | % |
| Compound name | Monkey | | | Dog | | |
| Buprenorphine^{$} | 0.3 | 2.1 | — | 0.6 | 7.9 | — |
| Buprenorphine | 0.9 ± 0.2 | 5.3 ± 1.2 | 260 | 2.3 ± 0.2 | 34 ± 10 | 430 |
| PABA carbamate | | | | | | |
| *Buprenorphine (2- | 7.9 ± 3.7 | 31.2 ± 11.9 | 1485 | 1.5 ± 0.5 | 4.1 ± 0.6 | 519 |
| methoxy-PABA) | | | | | | |
| carbamate | | | | | | |
| **Buprenorphine (2- | 1.2 ± 0.2 | 6.9 ± 2.1 | 329 | 1.1 ± 0.3 | 4.7 ± 1.2 | 118 |
| methyl PABA) | | | | | | |
| carbamate | | | | | | |
| *Buprenorphine (6- | 1.2 ± 0.5 | 4.1 ± 1.2 | 195 | 0.34 ± 0.09 | 0.77 | 19 |
| amino nicotinate) | | | | | | |
| carbamate | | | | | | |
| Buprenorphine (2- | 0.33 | 0.65 | 29 | 0.17 | 0.27 | 3 |
| hydroxy-PABA) | | | | | | |
| carbamate# | | | | | | |
| ***Buprenorphine | 0.11 | 0.32 | 20 | BLQ | NC | 0 |
| (glycine-PABA) | | | | | | |
| carbamate# | | | | | | |
| ***Buprenorphine | BLQ | NC | 0 | BLQ | NC | 0 |
| (N-methyl-PABA) | | | | | | |
| carbamate# | | | | | | |

^{$}AUC values extrapolated from composite profile.
*Dog doses were 0.01 mg/kg
**Dog dose 0.05 mg/kg
***Dog doses were 0.075 mg/kg po
BLQ = below level of quantification.
NC = not calculable.
#n = 2 abbreviated PK dataset.

### Example 34

### Comparative Bioavailability of Buprenorphine after Oral Administration of Buprenorphine or Buprenorphine PABA Carbamate to Monkeys

The following study provides a more comprehensive account of the pharmacokinetics of buprenorphine PABA carbamate in the monkey.

### Methodology

Test substances (buprenorphine or buprenorphine PABA carbamate) were orally administered to five male cynomolgus monkeys_in equimolar doses of 0.2 mg buprenorphine free base equivalents/kg in a crossover study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

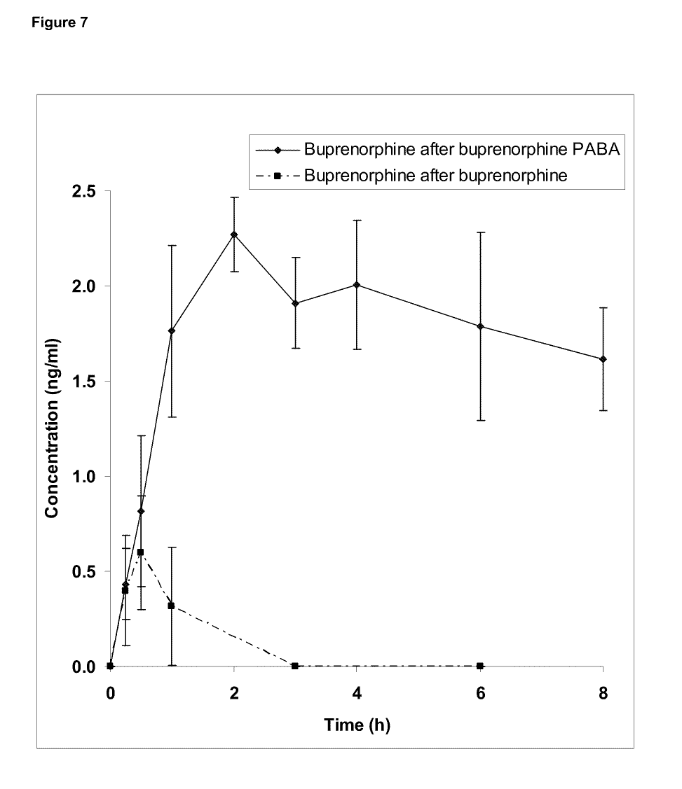

These are presented in Tables 26-28 and FIG. 7 and shows the mean 2.6-fold increase in bioavailability of the drug from this prodrug in comparison to that seen after giving the drug itself. If this translated to man this should result in less variable exposure to the parent drug and a more consistent analgesic response.

TABLE 26
Pharmacokinetic parameters* for buprenorphine following
oral administration of buprenorphine to monkeys
(0.2 mg buprenorphine free base equivalents/kg)

| Pharmacokinetic | | | | | | |
|---|---|---|---|---|---|---|
| parameter | 167 | 169 | 171 | 173 | 175 | Mean |
| Cₘₐₓ (ng/mL) | — | — | — | — | — | 0.3 |
| Tₘₐₓ (h) | — | — | — | — | — | 1.0 |
| AUC (ng · h/mL) | — | — | — | — | — | 2.1 |
| t½ (h) | — | — | — | — | — | 3.4 |

*As only a limited number of plasma drug concentrations were measurable in any single animal a composite PK profile was constructed from which parameters were derived. The t_{1/2} value was taken from data after prodrug

TABLE 27
Pharmacokinetic parameters for buprenorphine following
oral administration of buprenorphine PABA carbamate to monkeys
(0.2 mg buprenorphine free base equivalents/kg)

| Pharma- | | | | | | | |
|---|---|---|---|---|---|---|---|
| cokinet- | | | | | | | |
| ic pa- | | | | | | | |
| rameter | 167 | 169 | 171 | 173 | 175 | Mean | SD |
| Cₘₐₓ | 1.13 | 0.91 | 0.86 | 0.78 | 0.78 | 0.89 | 0.14 |
| (ng/mL) | | | | | | | |
| Tₘₐₓ (h) | 2 | 2 | 3 | 2 | 2 | 2.20 | 0.45 |
| AUC | 5.2 | 7.5 | 4.2 | 4.8 | 5.5 | 5.4 | 1.3 |
| (ng · h/ | | | | | | | |
| mL) | | | | | | | |
| t½ (h) | 3.9 | 3.6 | 2.3 | 3.3 | 3.9 | 3.4 | 0.7 |
| Frel % | 248 | 357 | 200 | 229 | 262 | 260 | 53 |

TABLE 28
Pharmacokinetic parameters for buprenorphine PABA carbamate
following oral administration of buprenorphine PABA carbamate to
monkeys (0.2 mg buprenorphine free base equivalents/kg)

| Pharmacokinetic | | | | | | | |
|---|---|---|---|---|---|---|---|
| parameter | 167 | 169 | 171 | 173 | 175 | Mean | SD |
| Cₘₐₓ (ng/mL) | 32 | 50 | 39 | 33 | 42 | 39 | 7 |
| Tₘₐₓ (h) | 1.0 | 1.0 | 1.0 | 2.0 | 1.0 | 1.2 | 0.4 |
| AUC (ng · h/mL) | 87 | 136 | 104 | 97 | 112 | 107 | 19 |
| t½ (h) | 1.1 | 1.2 | 1.2 | 1.3 | 1.3 | 1.2 | 0.1 |

### Example 35

### Comparative Bioavailability of Buprenorphine after Oral Administration of Buprenorphine or Buprenorphine PABA Carbamate Prodrug to Dogs

The following study provides a more comprehensive account of the pharmacokinetics of buprenorphine PABA carbamate in the dog.

### Methodology

Test substances (buprenorphine or buprenorphine PABA carbamate) were orally administered to five Beagle dogs in equimolar doses of 0.1 mg buprenorphine free base equivalents/kg in a crossover study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

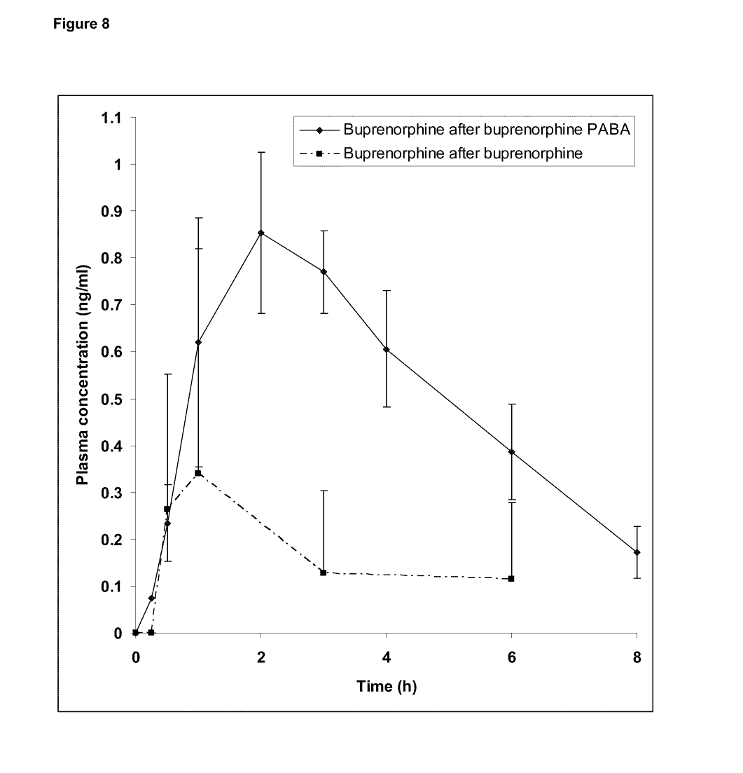

These are presented in Tables 29-31 and FIG. 8 and show an approximate 4.3-fold increase in systemic availability of buprenorphine after giving the prodrug. If this translated to man this should result in less variable exposure to the parent drug and a more consistent analgesic response.

TABLE 29
Pharmacokinetic parameters for buprenorphine following
oral administration of buprenorphine to dogs
(0.1 mg buprenorphine free base equivalents/kg)

| Pharmacokinetic | | | | | | |
|---|---|---|---|---|---|---|
| parameter | 335 | 337 | 339 | 341 | 347 | Mean |
| Cₘₐₓ (ng/mL) | | — | — | — | — | 0.6 |
| Tₘₐₓ (h) | — | — | — | — | — | 0.5 |
| AUC (ng · h/mL) | — | — | — | — | — | 7.9** |
| t½ (h) | — | — | — | — | — | 9* |

As only a limited number of plasma drug concentrations were measurable in any single animal a composite PK profile was constructed from which parameters were derived
*As no half-life could be generated from these data, a literature value of 9 h (Abbo 2008) was assumed
**Calculated using literature half-life of 9 h

TABLE 30
Pharmacokinetic parameters for buprenorphine following
oral administration of buprenorphine PABA carbamate to dogs
(0.1 mg buprenorphine free base equivalents/kg)

| Pharma- | | | | | | | |
|---|---|---|---|---|---|---|---|
| cokinetic | | | | | | | |
| parameter | 335 | 337 | 339 | 341 | 347 | Mean | SD |
| Cₘₐₓ | 2.37 | 2.56 | 2.04 | 2.25 | 2.5 | 2.3 | 0.2 |
| (ng/mL) | | | | | | | |
| Tₘₐₓ (h) | 1 | 2 | 2 | 2 | 4 | 2.2 | 1.1 |
| AUC | 28.4 | 38.6 | 28.4 | 25.0 | 49.5 | 34.0 | 10.0 |
| (ng · h/mL) | | | | | | | |
| t½ (h) | 7.9 | 9.9 | 7.8 | 4.9 | 11.6 | 8.4 | 2.5 |
| T > 50% | 7.5 | 7.2 | 7.3 | 7.0 | 7.5 | 7.3 | 0.2 |
| Cmax (h) | | | | | | | |
| Frel % | 360 | 489 | 360 | 317 | 626 | 430 | 127 |

TABLE 31
Pharmacokinetic parameters for buprenorphine PABA carbamate
following oral administration of buprenorphine to dogs
(0.1 mg buprenorphine free base equivalents/kg)

| Pharmacokinetic | | | | | | | |
|---|---|---|---|---|---|---|---|
| parameter | 335 | 337 | 339 | 341 | 347 | Mean | SD |
| Cₘₐₓ (ng/mL) | 94.4 | 82.8 | 91.8 | 67.7 | 106.1 | 88.5 | 14.3 |
| Tₘₐₓ (h) | 2 | 1 | 2 | 1 | 0.5 | 1.3 | 0.7 |
| AUC (ng · h/mL) | 563 | 506 | | 502 | 656 | 557 | 71.8 |
| t½ (h) | 3.0 | 3.6 | NI | 4.4 | 3.8 | 3.7 | 0.6 |

### Example 36

### Comparative Bioavailability of Buprenorphine after Oral Administration of Buprenorphine or Buprenorphine PABA Carbamate to Rats

### Methodology

Test substances (buprenorphine at 5.0 mg buprenorphine free base equivalents/kg or buprenorphine para amino benzoic acid carbamate at 5.0 mg buprenorphine free base equivalents/kg) were administered by oral gavage to groups of rats.

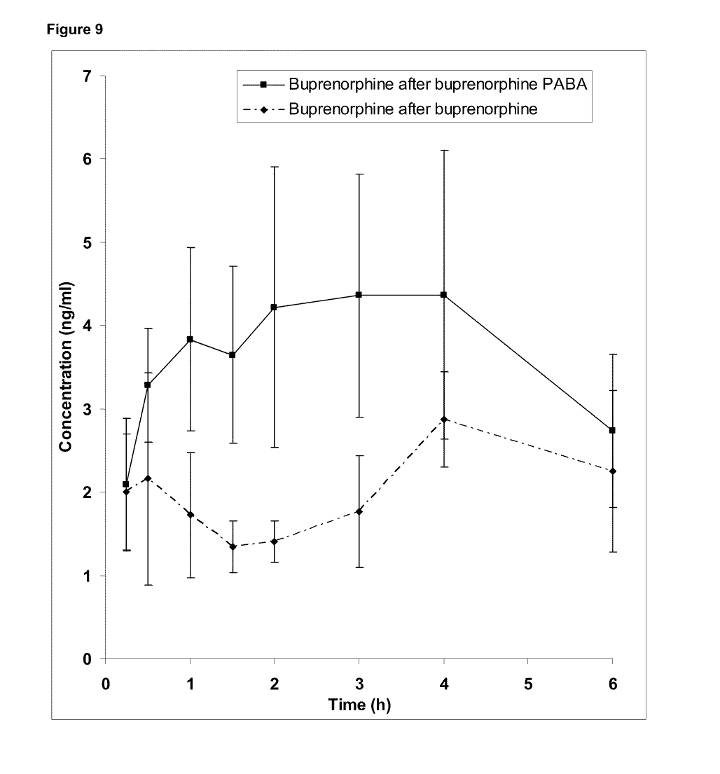

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Tables 32-34 and FIG. 9.

### Results

TABLE 32
Pharmacokinetic parameters for buprenorphine following
oral administration of buprenorphine
(5.0 mg buprenorphine free base equivalents/kg) to rats

| Pharmacokinetic | Rat number | | | | | | |
|---|---|---|---|---|---|---|---|
| parameter | 1 | 2 | 3 | 4 | 5 | Mean | SD |
| Cₘₐₓ (ng/mL) | 26.5 | 21.8 | 24.0 | 30.3 | 26.6 | 25.8 | 3.2 |
| Tₘₐₓ (h) | 0.25 | 4.0 | 3.0 | 3.0 | 0.5 | 2.2 | 1.7 |
| AUCt (ng · h/mL) | 63.7 | 72.0 | 97.7 | 113.1 | 106.0 | 90.5 | 21.6 |
| t½ (h) | 1.4 | 1.1 | ND | 2.4 | 4.7 | 2.4 | 1.6 |
| T > 50% Cmax (h) | 1.3 | 2.9 | 4.2 | 4.1 | 4.2 | 3.3 | 1.3 |

ND = not determined

TABLE 33
Pharmacokinetic parameters for buprenorphine following
oral administration of buprenorphine PABA carbamate
(5.0 mg buprenorphine free base equivalents/kg) to rats

| Pharmacokinetic | Rat number | | | | | | |
|---|---|---|---|---|---|---|---|
| parameter | 6 | 7 | 8 | 9 | 10 | Mean | SD |
| Cₘₐₓ (ng/mL) | 50.4 | 54.4 | 41.4 | 62.2 | 56.3 | 52.9 | 7.7 |
| Tₘₐₓ (h) | 3 | 4 | 1 | 3 | 1.5 | 3a | |
| AUCt (ng · h/mL) | 220 | 219 | 169 | 268 | 195 | 214 | 37 |
| t½ (h) | 2.4 | NC | 2.2 | 2.3 | 1.7 | 2.2 | 0.3 |
| T > 50% Cmax (h) | 4.9 | 4.7 | 5.4 | 4.8 | 3.8 | 4.7 | 0.6 |
| Frel (%) | 276 | 294 | 215 | 331 | 216 | 266 | 51 |

TABLE 34
Pharmacokinetic parameters for buprenorphine PABA carbamate
following oral administration of buprenorphine PABA carbamate
(5.0 mg buprenorphine free base equivalents/kg) to rats

| Pharma- | | | | | | | |
|---|---|---|---|---|---|---|---|
| cokinetic | Rat number | | | | | | |
| parameter | 6 | 7 | 8 | 9 | 10 | Mean | SD |
| Cₘₐₓ (ng/mL) | 323 | 391 | 426 | 318 | 820 | 456 | 209 |
| Tₘₐₓ (h) | 2 | 3 | 0.5 | 0.5 | 0.5 | 1.3 | 1.2 |
| AUCt | 885 | 1249 | 594 | 845 | 1512 | 1017 | 362 |
| (ng · h/mL) | | | | | | | |
| t½ (h) | 1.1 | 1.0 | 1.5 | 1.4 | 0.9 | 1.2 | 0.3 |

The comparative bioavailability from the PABA carbamate prodrug was at least 2.7-fold greater (based on AUCt only) than after the parent drug. The improvement was likely higher than this due to the greater drug persistence after giving the prodrug, with the 50% Cmax increasing from 3.3 to at least 4.7 h. If these pharmacokinetic improvements translated to man there should be an increased consistency in analgesic response with a longer duration of action.

### Example 37

### Comparative In Vitro Metabolism of Buprenorphine PABA Carbamate by Hepatocytes from Rat, Dog, Monkey and Man

In order to determine whether the generation of buprenorphine from the prodrug, buprenorphine PABA carbamate, would translate from animals to man, a comparative in vitro metabolism study was undertaken across the species

### Methodology

The comparative metabolism of buprenorphine PABA carbamate was investigated using cryo-preserved hepatocytes (pooled from a minimum of 3 individuals) collected from Sprague-Dawley rats, Beagle dogs, Cynomolgus monkeys and humans. Incubate samples were removed at various time points over the course of the 2 h experiment and assayed for released meptazinol (its subsequent glucuronide metabolite) by LC-MS/MS. For comparative purposes a similar study was conducted using meptazinol itself.

### Results

Buprenorphine was liberated from buprenorphine PABA carbamate in incubations of hepatocytes from all species and subsequently eliminated subsequently by further metabolism. Maximal amounts of buprenorphine generated were lowest in the rat but comparable in dog monkey and human hepatocytes. Subsequent elimination occurred at a similar rate in dogs and monkey and human hepatocyte incubations but was somewhat slower in rat hepatocytes where the calculated half life of buprenorphine was approx. two-fold longer.

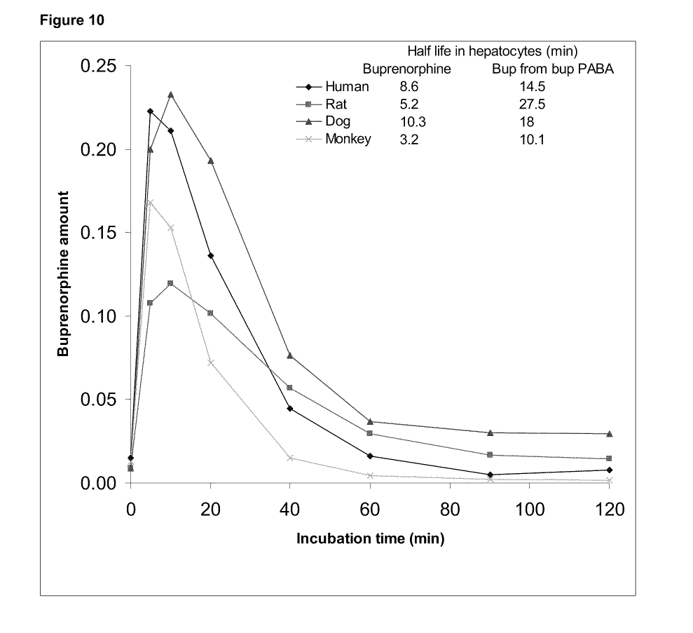

The similar amounts of buprenorphine generated in human hepatocytes compared to the animal species (see FIG. 10) suggests that the beneficial improvement in oral bioavailability seen in animals should translate to man.

### Example 38

### Buprenorphine and Buprenorphine PABA Carbamate in the Rat Tail Flick Model

In order to demonstrate that improvement in oral availability of buprenorphine from this prodrug translated into increased analgesic potency, a comparison was made of buprenorphine vs buprenorphine PABA carbamate in in the classical in vivo animal model of nociceptive pain, the rat tail flick test.

### Methodology

Groups of 8 male Sprague Dawley rats were habituated to the tail flick apparatus and baseline flick latencies were measured prior to drug administration. At time zero, rats received one of three treatments, vehicle, or buprenorphine at 0.5, 1, 1.5, 2.5, 5, 7.5, 10 and 20 mg/kg orally or buprenorphine PABA carbamate 0.1, 0.2, 0.5, 1, 1.5, 2.5, 5 and 10 mg as buprenorphine base equivalents/kg in a dose volume of 5 mL/kg. Latency to tail flick from an infrared heat source was measured 0.5 h later after dosing with buprenorphine and 0.5, 1 and 2 h after dosing with buprenorphine PABA carbamate. Maximum possible effect was calculated as (tail flick latency−mean vehicle tail flick latency)/(15−mean vehicle tail flick latency)×100.

### Results

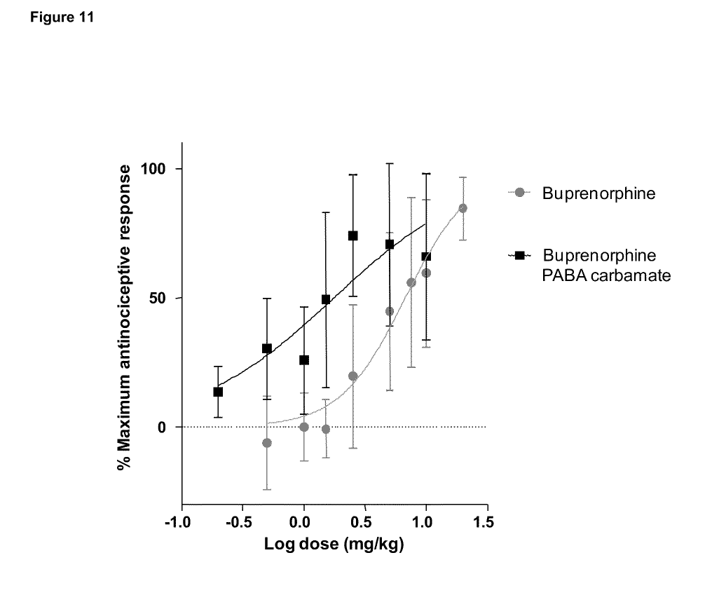

The results presented in FIG. 11 reveal that treatment with buprenorphine dose-dependently increased tail flick latency indicative of an analgesic effect. Treatment with buprenorphine PABA carbamate also dose-dependently increased tail flick latency with maximal effect at 1 h post treatment. The ED50 value was determined as 1.75 mg buprenorphine base/kg for buprenorphine PABA carbamate and 6.75 mg base/kg for buprenorphine showing a statistically significant 3.8-fold improvement in potency. This ratio between the ED50 determinations was similar to the observed increase in bioavailability of buprenorphine in the rat after administration of buprenorphine PABA carbamate.

### Example 39

### Bioavailability of Tapentadol in the Cynomolgus Monkey After Oral Administration of Either Racemic Tapentadol or Racemic Tapentadol PABA Carbamate

Initial examination of the chemical stability of tapentadol PABA carbamate showed that the compound was stable over 2 hours in distilled water (pH 5.7) at room temperature. However, at pH7.4 (37° C.), over a 2 hour period, about 50% of this prodrug was hydrolyzed to tapentadol, thereby demonstrating a potential for chemical activation in the blood. Consequently, an in vivo pharmacokinetic study was performed in monkeys, to ascertain the potential extent of exposure to tapentadol following oral administration of tapentadol PABA carbamate.

### Methodology

To examine the comparative pharmacokinetics of tapentadol after tapentadol or tapentadol PABA carbamate administration, five male cynomolgus monkeys were orally dosed with tapentadol itself (as a mixture of all four isomers SS, RR, RS & SR at 1 mg/kg tapentadol free base equivalents/kg). Another two animals received tapentadol PABA carbamate, (also as a mixture of all four isomers, at 3 mg equivalents/kg tapentadol free base equivalents/kg).

Blood samples were taken at various times after administration and submitted to analysis for the parent drug (measured as two pairs of isomers RS+SR and RR+SS) and pro-drug (again as two pairs of isomers) using a validated LC-MS-MS assay. Pharmacokinetic parameters derived from the plasma analytical data (for the combined four isomers) were determined using Win Nonlin.

### Results

These are presented in Tables 35-37. After oral administration of tapentadol, plasma concentrations of tapentadol (all four isomers) were negligible (i.e., less than the lower limit of quantification (LLOQ) of the bioanalytical method, <0.4 ng/mL for each isomer), suggesting very low oral bioavailability of the drug. Nevertheless, an estimate could be made of the maximal possible exposure (AUC) to the drug when scaled up to a higher 3 mg/kg dose and assuming the Cmax (all four isomers) to be equal to the LLOQ value (3×0.4+0.4=2.4 ng/mL) and a subsequent plasma half life comparable to that seen for the drug (all isomers) after giving the PABA carbamate prodrug 1.5 h. On this basis, the estimate of exposure was 5.4 ng·h/mL (Table 35).

This estimate enabled a comparison of tapentadol exposure after administration of either tapentadol or tapentadol PABA carbamate (administered at 3 mg/kg). Here, the Cmax (all four isomers) was 29 ng/mL (after tapentadol PABA carbamate), 12-fold higher than when tapentadol itself was administered. The AUC after prodrug administration was 104 ng·h/mL, some 20-fold higher than when tapentadol itself was administered (Table 36, FIG. 6). These data demonstrate a very considerable improvement in the oral bioavailability after giving the PABA carbamate prodrug. If translated to man such improvements should lead to less variability in attained plasma drug levels and a more consistent analgesic response.

TABLE 35
Estimated mean pharmacokinetic parameters (n = 5) for racemic
tapentadol following oral administration of tapentadol to monkeys
(scaled up to from 1 to 3 mg tapentadol free base equivalents/kg)

| Pharmacokinetic | |
|---|---|
| parameter | Average |
| Cₘₐₓ * (ng/mL) | <2.4 |
| AUC (ng · h/mL) | <5.4 |
| t½ ** (h) | 1.2 |

* Concn <LLOQ (2.4 ng/mL) - taken as Cₘₐₓ
** Half-life taken from tapentadol plasma levels measured in other animals after oral PABA tapentadol

TABLE 36
Pharmacokinetic parameters for racemic tapentadol following
oral administration of tapentadol PABA carbamate to monkeys
(3 mg tapentadol free base equivalents/kg).

| Pharmacokinetic | | | |
|---|---|---|---|
| parameter | Monkey 1 | Monkey 2 | Average |
| Cₘₐₓ (ng/mL) | 30.6 | 27.1 | 28.9 |
| Tₘₐₓ (h) | 2 | 1 | 1.5 |
| AUC (ng · h/mL) | 115 | 93.0 | 104 |
| t½ (h) | 1.23 | 1.17 | 1.20 |
| T_{>50% Cmax} (h) | 3.61 | 3.29 | 3.45 |
| Fᵣₑₗ (%) | >2170% | >1750% | >1960% |

TABLE 37
Pharmacokinetic parameters for racemic tapentadol PABA carbamate
following oral administration of tapentadol PABA carbamate
to monkeys (3 mg tapentadol free base equivalents/kg).

| Pharmacokinetic | | | |
|---|---|---|---|
| parameter | Monkey 3 | Monkey 4 | Average |
| Cₘₐₓ (ng/mL) | 587 | 603 | 595 |
| Tₘₐₓ (h) | 1 | 1 | 1 |
| AUC (ng · h/mL) | 1713 | 1472 | 1593 |
| t½ (h) | 1.00 | 0.95 | 0.98 |
| T_{>50% Cmax} (h) | 2.79 | 1.78 | 2.29 |

### Example 40

### Comparative Bioavailability of R,R Tapentadol after Oral Administration of R,R Tapentadol or R,R Tapentadol PABA Carbamate Prodrug to Monkeys

As the currently marketed clinical formulation of tapentadol uses the R,R enantiomer it was considered appropriate to investigate whether the prodrug of this single enantiomer behaved pharmacokinetically in a manner comparable to that seen after the racemic mixture prodrug.

### Methodology

Test substances (R,R tapentadol or R,R tapentadol PABA carbamate) were orally administered to four male cynomolgus monkeys _in equimolar doses of 3 mg tapentadol free base equivalents/kg in a crossover study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

These are shown in Tables 38-40 and reveal a mean 23-fold increase in bioavailability of the drug from the prodrug in comparison to that seen after giving the drug itself. If this translated to man this should result in less variable exposure to the parent drug and a more consistent analgesic response.

TABLE 38
Pharmacokinetic parameters for R,R tapentadol following
oral administration of R,R tapentadol to monkeys
(3 mg R,R tapentadol free base equivalents/kg)

| Pharmacokinetic | | | | | | |
|---|---|---|---|---|---|---|
| parameter | 169 | 171 | 173 | 175 | Mean | SD |
| Cₘₐₓ (ng/mL) | 2.9 | 2.6 | 2.1 | 1.1 | 2.2 | 0.8 |
| Tₘₐₓ (h) | 0.5 | 1.0 | 2.0 | 1.0 | 1.1 | 0.6 |
| AUC (ng · h/mL) | 10 | 8.6 | 7.7 | 4.0 | 7.6 | 2.6 |
| t½ (h) | 1.5 | 1.5 | 1.7 | 1.6 | 1.6 | 0.1 |
| T > 50% Cmax (h) | 2.8 | 2.7 | 2.9 | 2.8 | 2.8 | 0.1 |

TABLE 39
Pharmacokinetic parameters for R,R tapentadol following
oral administration of R,R tapentadol PABA carbamate to monkeys
(3 mg R,R tapentadol free base equivalents/kg)

| Pharma- | | | | | | |
|---|---|---|---|---|---|---|
| cokinetic | | | | | | |
| parameter | 169 | 171 | 173 | 175 | Mean | SD |
| Cₘₐₓ (ng/mL) | 53 | 38 | 26 | 56 | 43 | 14 |
| Tₘₐₓ (h) | 2.0 | 2.0 | 2.0 | 1.0 | 1.8 | 0.5 |
| AUC | 208 | 128 | 88 | 190 | 154 | 55 |
| (ng · h/mL) | | | | | | |
| t½ (h) | 5.0 | 6.2 | 1.8 | 1.8 | 3.7 | 2.3 |
| T > 50% | 3.3 | 2.3 | 2.6 | 2.7 | 2.7 | 0.4 |
| Cmax (h) | | | | | | |
| Frel % | 2060 | 1490 | 1150 | 4700 | 2350 | 1610 |

TABLE 40
Pharmacokinetic parameters for R,R tapentadol PABA carbamate
following oral administration of R,R tapentadol PABA carbamate
to monkeys (3 mg R,R tapentadol free base equivalents/kg)

| Pharma- | | | | | | |
|---|---|---|---|---|---|---|
| cokinetic | | | | | | |
| parameter | 169 | 171 | 173 | 175 | Mean | SD |
| Cₘₐₓ (ng/mL) | 1070 | 848 | 608 | 2070 | 1150 | 642 |
| Tₘₐₓ (h) | 0.5 | 1.0 | 1.0 | 1.0 | 0.88 | 0.25 |
| AUC | 2480 | 1940 | 1090 | 3350 | 2220 | 948 |
| (ng · h/mL) | | | | | | |
| t½ (h) | 2.7 | 5.1 | 1.2 | 2.2 | 2.8 | 1.6 |

### Example 41

### Comparative Bioavailability of R,R Tapentadol After Oral Administration of R,R Tapentadol or R,R Tapentadol PABA Carbamate Prodrug to Dogs

### Methodology

Test substances (R,R tapentadol or R,R tapentadol PABA carbamate) were orally administered to four female Beagle dogs in equimolar doses of 1 mg R,R tapentadol free base equivalents/kg in a crossover study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

These are shown in Tables 41-43 and reveal a mean ˜20-fold increase in bioavailability of the drug from the prodrug in comparison to that seen after giving the drug itself. Furthermore the plasma levels of R,R tapentadol after giving the prodrug persisted for ˜6 times longer than those seen after giving the drug itself. The T50% Cmax was increased from 0.7 h to 4.2 h. If this better PK profile translated to man such improvements should lead to less variability in attained plasma drug levels and a more consistent analgesic response and a greater duration of therapeutic effect.

TABLE 41
Pharmacokinetics of RR-tapentadol after oral dosing of 1 mg
RR-tapentadol free base equivalents/kg to female beagle dogs

| Pharmacokinetic | | | | | | |
|---|---|---|---|---|---|---|
| parameter | 626 | 628 | 632 | 634 | Mean | SD |
| Cₘₐₓ (ng/mL) | 1.25 | 8.48 | 0.39 | 2.71 | 3.21 | 3.64 |
| Tₘₐₓ (h) | 0.50 | 0.50 | 0.25 | 0.50 | 0.44 | 0.13 |
| AUC (ng · h/mL) | 1.74 | 8.35 | NC | 2.38 | 4.16 | 3.65 |
| t½ (h) | 0.7 | 0.6 | NC | 0.5 | 0.6 | 0.1 |
| T_{>50%Cmax} (h) | 1.0 | 0.6 | NC | 0.6 | 0.7 | 1.2 |

TABLE 42
Pharmacokinetics of RR-tapentadol after oral dosing of 1 mg
RR-tapentadol equivs/kg of the p-aminobenzoic acid carbamate
prodrug to female beagle dogs

| Pharmacokinetic | | | | | | |
|---|---|---|---|---|---|---|
| parameter | 626 | 628 | 632 | 634 | Mean | SD |
| Cₘₐₓ (ng/mL) | 8.95 | 25.9 | 13.4 | 11.0 | 14.8 | 7.6 |
| Tₘₐₓ (h) | 1.0 | 2.0 | 4.0 | 2.0 | 2.3 | 1.3 |
| AUC (ng · h/mL) | 40 | 119 | 84 | 58 | 72 | 42 |
| t½ (h) | 3.0 | 1.8 | 3.2 | 2.1 | 2.3 | 0.6 |
| T_{>50% Cmax} (h) | 3.7 | 3.8 | 5.0 | 4.3 | 4.2 | 0.6 |
| Fᵣₑₗ (%) | 2270 | 1420 | | 2430 | 2040 | 540 |

TABLE 43
Pharmacokinetics of RR-tapentadol PABA carbamate after oral doing
of 1 mg RR-tapentadol equivs/kg of the p-aminobenzoic acid
carbamate prodrug to female beagle dogs

| Pharmacokinetic | | | | | | |
|---|---|---|---|---|---|---|
| parameter | 626 | 628 | 632 | 634 | Mean | SD |
| Cₘₐₓ (ng/mL) | 401 | 832 | 492 | 172 | 474 | 274 |
| Tₘₐₓ (h) | 0.25 | 0.50 | 1.0 | 0.5 | 0.56 | 0.31 |
| AUC (ng · h/mL) | 683 | 2090 | 1820 | 734 | 1330 | 728 |
| t½ (h) | 4.0 | 3.4 | 4.1 | 4.1 | 3.9 | 0.3 |

### Example 42

### Comparative Bioavailability of Nalbuphine after Oral Administration of Nalbuphine or Nalbuphine PABA Carbamate to Rats

### Methodology

Test substances (nalbuphine at 3 mg nalbuphine free base equivalents/kg or nalbuphine para amino benzoic acid carbamate at 3 mg nalbuphine free base equivalents/kg) were administered by oral gavage to groups of male Wistar rats in a parallel group study design.

Blood samples were taken at various times after administration and submitted to analysis for the parent drug and pro-drug using a validated LC-MS-MS assay. Pharmacokinetic parameters derived from the plasma analytical data were determined using Win Nonlin.

### Results

These are shown in Tables 44-46 and reveal a mean 27-fold increase in bioavailability of the drug from the prodrug in comparison to that seen after giving the drug itself. As a consequence the variability around this parameter dropped from ˜58% to 15% which, if this translated to man, should result in a much more consistent clinical response.

TABLE 44
Pharmacokinetics of nalbuphine after oral dosing of
3 mg nalbuphine free base equivalents/kg to male rats

| Pharmacokinetic | | | | | |
|---|---|---|---|---|---|
| parameter | 10 | 11 | 12 | Mean | sd |
| Cₘₐₓ (ng/mL) | 1.4 | 0.7 | 0.8 | 1.0 | 0.4 |
| Tₘₐₓ (h) | 0.5 | 1.0 | 1.0 | 0.8 | 0.3 |
| AUC (ng · h/mL) | 1.8 | 0.4 | 1.3 | 1.2 | 0.7 |
| t½ (h) | 1.2 | — | 1.6 | 1.4 | — |
| T_{>50% Cmax} (h) | 1.5 | 0.7 | 1.8 | 1.3 | 0.6 |

TABLE 45
Pharmacokinetics of nalbuphine after oral dosing
of 3 mg nalbuphine equivs/kg of the p-aminobenzoic
acid carbamate prodrug to male rats

| Pharmacokinetic | | | | | |
|---|---|---|---|---|---|
| parameter | 7 | 8 | 9 | Mean | sd |
| Cₘₐₓ (ng/mL) | 19 | 16 | 16 | 17 | 2.0 |
| Tₘₐₓ(h) | 0.25 | 0.25 | 0.25 | 0.25 | 0.0 |
| AUC (ng · h/mL) | 31 | 31 | 39 | 33 | 5.0 |
| t½ (h) | 0.7 | 1.8 | 1.8 | 1.4 | 0.6 |
| T_{>50% Cmax} (h) | 1.3 | 1.2 | 1.6 | 1.4 | 0.2 |
| Fᵣₑₗ (%) | 2540 | 2490 | 3180 | 2740 | 380 |

TABLE 46
Pharmacokinetics of nalbuphine PABA carbamate after
oral dosing of 3 mg nalbuphine equivs/kg of the p-
aminobenzoic acid carbamate prodrug to male rats

| Pharmacokinetic | | | | | |
|---|---|---|---|---|---|
| parameter | 7 | 8 | 9 | Mean | sd |
| Cₘₐₓ (ng/mL) | 16 | 11 | 9.2 | 12 | 3.3 |
| Tₘₐₓ (h) | 0.25 | 0.25 | 0.25 | 0.25 | 0 |
| AUC (ng · h/mL) | 14 | 9.4 | 11 | 11 | 2.2 |
| t½ (h) | 0.5 | 0.8 | 1.2 | 0.8 | 0.3 |

### Example 43

### Comparative Bioavailability of Butorphanol after Oral Administration of Butorphanol or Butorphanol PABA Carbamate Prodrug to Monkeys

### Methodology

Test substances (butorphanol or butorphanol PABA carbamate were orally administered to two male cynomolgus monkeys at equimolar doses of 1 mg butorphanol free base equivalents/kg in a parallel group study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

These are shown in Tables 47-49 and reveal a dramatic 95-fold increase in bioavailability of the drug from the prodrug in comparison to that seen after giving the drug itself. If this translated to man this should result in less variable exposure to the parent drug and a more consistent analgesic response.

TABLE 47
Pharmacokinetic parameters for butorphanol following
oral administration of butorphanol (1.0 mg butorphanol
free base/kg) to the monkey.

| Pharmacokinetic | | | |
|---|---|---|---|
| parameter | Monkey 1 | Monkey 2 | Average |
| Cmax (ng/mL) | <0.1* | <0.1* | 0.1 |
| Tmax (h) | — | — | 0.5* |
| AUC (ng · h/mL) | — | — | 0.62 |
| t½ (h) | — | — | 6.1** |

*Plasma concentration were below the LLOQ of 0.1 ng/mL
**Value after giving prodrug and used for estimating maximal systemic exposure

TABLE 48
Pharmacokinetic parameters for butorphanol following oral
administration of butorphanol PABA carbamate to the monkey
(1 mg butorphanol free base equivalents/kg).

| Pharmacokinetic | | | |
|---|---|---|---|
| parameter | Monkey 3 | Monkey 4 | Average |
| Cmax (ng/mL) | 11.7 | 3.3 | 7.5 |
| Tmax (h) | 3.0 | 3.0 | 3.0 |
| AUC (ng · h/mL) | 65.3 | 52.8 | 59.1 |
| t½ (h) | 2.5 | 9.7 | 6.1 |
| T > 50% Cmax (h) | 4.5 | 5.1 | 4.8 |
| Frel (%) | | | 9500 |

TABLE 49
Pharmacokinetic parameters for butorphanol PABA carbamate
following oral administration of butorphanol PABA carbamate
to monkey (1 mg butorphanol free base equivalents/kg).

| Pharmacokinetic | | | |
|---|---|---|---|
| parameter | Monkey 3 | Monkey 4 | Average |
| Cmax (ng/mL) | 178 | 121 | 150 |
| Tmax (h) | 1.0 | 3.0 | 2.0 |
| AUCt (ng · h/mL) | 405 | 462 | 434 |
| AUC (ng · h/mL) | 412 | 500 | 456 |
| t½ (h) | 1.0 | 1.2 | 1.1 |

### Example 44

### Comparative Bioavailability of Butorphanol after Oral Administration of Butorphanol or Butorphanol PABA Carbamate Prodrug to Dogs

### Methodology

Test substances, butorphanol or butorphanol_PABA carbamate were orally administered to two male beagle dogs at 0.5 mg butorphanol free base equivalents/kg in a parallel group study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

These are shown in Tables 50-52 & once again reveal a dramatic increase (37-fold) in bioavailability of the drug from the prodrug in comparison to that seen after giving the drug itself._If this translated to man this should result in less variable exposure to the parent drug and a more consistent analgesic response.

TABLE 50
Pharmacokinetic parameters for butorphanol following oral administration
of butorphanol (0.5 mg butorphanol free base/kg) to the dog.

| Pharmacokinetic | | | |
|---|---|---|---|
| parameter | Dog 1 | Dog 2 | Average |
| Cmax (ng/mL) | 0.90 | 0.56 | 0.73 |
| Tmax (h) | 0.50 | 1.00 | 0.75 |
| AUC (ng · h/mL) | 2.30 | 1.80 | 2.05 |
| t½ (h) | 1.6 | 1.7 | 1.6 |
| T > 50% Cmax (h) | 1.6 | 2.4 | 2.0 |

TABLE 51
Pharmacokinetic parameters for butorphanol following
oral administration of butorphanol PABA carbamate to
dogs (0.5 mg butorphanol free base equivalents/kg).

| Pharmacokinetic | | | |
|---|---|---|---|
| parameter | Dog 3 | Dog 4 | Average |
| Cmax (ng/mL) | 14.5 | 15.4 | 15.0 |
| Tmax (h) | 3 | 3 | 3 |
| AUC (ng · h/mL) | 88 | 136 | 112 |
| t½ (h) | 3.1 | 4.8 | 4.0 |
| T > 50% Cmax (h) | 4.5 | 5.1 | 4.8 |
| Frel (%) | >3285% | >4063% | >3674% |

TABLE 52
Pharmacokinetic parameters for butorphanol PABA carbamate
following oral administration of butorphanol PABA carbamate
to dogs (0.5 mg butorphanol free base equivalents/kg).

| Pharmacokinetic | | | |
|---|---|---|---|
| parameter | Dog 3 | Dog 4 | Average |
| Cmax (ng/mL) | 472.0 | 379.0 | 425.5 |
| Tmax (h) | 1 | 1 | 1.0 |
| AUC (ng · h/mL) | 2154 | 1787 | 1971 |
| t½ (h) | 2.6 | 2.8 | 2.7 |

### Example 45

### Comparative Bioavailability of Levorphanol after Oral Administration of Levorphanol or Levorphanol PABA Carbamate Prodrug to Monkeys

### Methodology

Test substances (levorphanol or levorphanol PABA carbamate) were orally administered to four male cynomolgus monkeys in equimolar doses of 5 mg levorphanol free base equivalents/kg in a crossover study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

These are shown in Tables 53-55 and reveal a mean ˜1.7-fold increase in bioavailability of the drug from the prodrug in comparison to that seen after giving the drug itself. There was also an indication of some modest sustainment in plasma drug concentrations after giving the prodrug.

TABLE 53
Pharmacokinetic parameters of levorphanol after oral
dosing of 5 mg levorphanol free base equivalent/kg
to male cynomolgus monkeys

| Pharmacokinetic | | | | | | |
|---|---|---|---|---|---|---|
| parameter | 12 | 13 | 14 | 15 | Mean | sd |
| Cₘₐₓ (ng/mL) | 6.2 | 6.6 | 8.2 | 7.6 | 7.1 | 0.9 |
| Tₘₐₓ (h) | 2.0 | 1.0 | 2.0 | 1.0 | 1.5 | 0.6 |
| AUC (ng · h/mL) | 22 | 28 | 34 | 27 | 28 | 4.8 |
| t½ (h) | 1.5 | 2.1 | 1.5 | 1.6 | 1.7 | 0.3 |
| T > 50% Cmax | 3.0 | 3.1 | 3.5 | 2.8 | 3.1 | 0.3 |

TABLE 54
Pharmacokinetic parameters of levorphanol after oral dosing
of 5 mg levorphanol free base equivalents/kg of the PABA
carbamate pordrug/kg to male cynomolgus monkeys

| Pharmacokinetic | | | | | | |
|---|---|---|---|---|---|---|
| parameter | 12 | 13 | 14 | 15 | Mean | sd |
| Cₘₐₓ (ng/mL) | 8.4 | 12 | 7.1 | 4.75 | 8.1 | 3.0 |
| Tₘₐₓ (h) | 2.0 | 2.0 | 3.0 | 2.0 | 2.3 | 0.5 |
| AUC (ng · h/mL) | 41 | 74 | 46 | 24 | 46 | 21 |
| t½ (h) | 2.1 | 3.1 | 3.6 | 2.6 | 2.9 | 0.6 |
| T > 50% Cmax | 3.9 | 4.8 | 4.5 | 4.0 | 4.3 | 0.4 |
| Frel (%) | 183% | 266% | 134% | 88% | 168 | 76 |

TABLE 55
Pharamcokinetics of levorphanol PABA carbamate after oral
doing of 1 mg levorphanol equivs/kg of the p-aminobenzoic
acid carbamate prodrug to male cynomolgus monkeys

| Pharmacokinetic | | | | | | |
|---|---|---|---|---|---|---|
| parameter | 12 | 13 | 14 | 15 | Mean | sd |
| Cₘₐₓ (ng/mL) | 138 | 212 | 98 | 80 | 132 | 59 |
| Tₘₐₓ (h) | 2.0 | 1.0 | 0.5 | 0.5 | 1.0 | 0.7 |
| AUC (ng · h/mL) | 622 | 696 | 517 | 376 | 553 | 139 |
| t½ (h) | 11.5 | 9.1 | 8.2 | 11.0 | 10 | 1.6 |

### Example 46

### Comparative Bioavailability of Levorphanol after Oral Administration of Levorphanol or Levorphanol PABA Carbamate Prodrug to Dogs

### Methodology

Test substances (levorphanol or levorphanol PABA carbamate) were orally administered to five female Beagle dogs in equimolar doses of 5 mg levorphanol free base equivalents/kg in a parallel group study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

These are shown in Tables 56-58 and reveal a mean ˜6 fold increase in bioavailability of the drug from the prodrug in comparison to that seen after giving the drug itself although there was significant inter-animal variability in the magnitude of this increase. Similarly there was a substantial prolongation of sustainment of drug in the plasma (5-fold) with the T_{>50%Cmax} values increasing from 1.4 to 6.4 h. If this translated to man, this could enable less frequent dosing and increased patient compliance.

TABLE 56
Pharmacokinetics of levorphanol after oral dosing of 5 mg
levorphanol free base equivalents/kg to female beagle dog

| Pharmacokinetic | | | | | | | |
|---|---|---|---|---|---|---|---|
| parameter | 18 | 19 | 20 | 21 | 22 | Mean | sd |
| Cₘₐₓ (ng/mL) | 4.2 | 3.2 | 5.3 | 4.3 | 1.9 | 3.8 | 1.3 |
| Tₘₐₓ (h) | 1.0 | 0.5 | 0.5 | 0.5 | 0.5 | 0.6 | 0.2 |
| AUC (ng.h/mL) | 5.8 | 19 | 16 | 6.6 | 2.8 | 10 | 7.0 |
| t½ (h) | 0.8 | — | — | 1.5 | — | 1.2 | — |
| T_{>50%Cmax} (h) | 1.5 | — | — | 1.3 | — | 1.4 | — |

All AUC values are AUCₗₐₛₜ. A maximum of 30% extrapolation has been allowed for determination of t½ and T_{>50%Cmax}.

TABLE 57
Pharmacokinetics of levorphanol after oral dosing of 5 mg
levorphanol equivalents/kg of the p-aminobenzoic acid
carbamate prodrug to female beagle dog

| Pharmacokinetic | | | | | | | |
|---|---|---|---|---|---|---|---|
| parameter | 18 | 19 | 20 | 21 | 22 | Mean | sd |
| Cₘₐₓ (ng/mL) | 3.9 | 1.8 | 4.0 | 8.3 | 5.7 | 4.7 | 2.4 |
| Tₘₐₓ (h) | 2.0 | 3.0 | 6.0 | 2.0 | 2.0 | 3.0 | 1.7 |
| AUC (ng · h/mL) | 17 | 9.8 | 61 | 68 | 30 | 37 | 26 |
| t½ (h) | 2.7 | — | — | 3.1 | 3.9 | 3.2 | 0.6 |
| T_{>50%Cmax} (h) | 5.1 | — | — | 8.2 | 6.0 | 6.4 | 1.6 |
| Fᵣₑₗ (%) | 300% | 52% | 385% | 1030% | 1090% | 571% | 463% |

All AUC values are AUCₗₐₛₜ. A maximum of 30% extrapolation has been allowed for determination of t½ and T_{>50%Cmax}.

TABLE 58
Pharmacokinetics of levorphanol PABA carbamate after oral dosing
of 5 mg levorphanol equivalents/kg of the p-aminobenzoic acid
carbamate prodrug to female beagle dog

| Pharmacokinetic | | | | | | | |
|---|---|---|---|---|---|---|---|
| parameter | 18 | 19 | 20 | 21 | 22 | Mean | sd |
| Cₘₐₓ (ng/mL) | 94 | 24 | 96 | 184 | 85 | 97 | 57 |
| Tₘₐₓ (h) | 0.5 | 2.0 | 0.5 | 0.5 | 0.5 | 0.8 | 0.7 |
| AUC (ng · h/mL) | 285 | 136 | 343 | 686 | 362 | 362 | 201 |
| t½ (h) | 2.3 | 2.1 | 2.1 | 1.7 | 1.7 | 2.0 | 0.3 |

All AUC values are AUCₗₐₛₜ. A maximum of 30% extrapolation has been allowed for determination of t½ and T_{>50%Cmax}.

### Example 47

### Comparative Bioavailability of Dextrorphan after Oral Administration of Dextrorphan or Dextrorphan PABA Carbamate Prodrug to Monkeys

### Methodology

Test substances, dextrorphan or dextrorphan PABA carbamate were orally administered to five male cynomolgus monkeys in equimolar doses of 5.0 mg dextrorphan free base equivalents/kg in a crossover study design.

Blood samples were taken at various times after administration, and submitted to analysis for the parent drug and prodrug using a validated LC-MS-MS assay. Pharmacokinetic parameters derived from the plasma analytical data were determined using Win Nonlin.

### Results

These are shown in Tables 59-61 and Figure and reveal a mean ˜3-fold increase in bioavailability of the drug from the prodrug in comparison to that seen after giving the drug itself.

TABLE 59
Pharmacokinetics of dextrorphan after oral dosing
of 5 mg dextrorphan free base equivalents/kg to
male cynomolgus monkeys

| Pharmacokinetic | | | | | | | |
|---|---|---|---|---|---|---|---|
| parameter | 11 | 12 | 13 | 14 | 15 | Mean | sd |
| Cₘₐₓ (ng/mL) | 4.3 | 3.0 | 3.5 | 5.5 | 3.3 | 3.9 | 1.0 |
| Tₘₐₓ (h) | 2.0 | 1.0 | 2.0 | 1.0 | 2.0 | 1.6 | 0.6 |
| AUC (ng · h/mL) | 20 | 14* | 19 | 19 | 13 | 17 | 3.0 |
| t½ (h) | 2.3 | 2.6 | 2.3 | 1.8 | 1.6 | 2.1 | 0.4 |
| T_{>50%Cmax} (h) | 3.7 | 3.5 | 4.5 | 2.5 | 3.3 | 3.5 | 0.7 |

*extrapolation of AUC data 39%

TABLE 60
Pharmacokinetics of dextrorphan after oral dosing of 5 mg
dextrorphan equivs/kg of the p-aminobenzoic acid
carbamate prodrug to male cynomolgus monkeys

| Pharmacokinetic | | | | | | | |
|---|---|---|---|---|---|---|---|
| parameter | 11 | 12 | 13 | 14 | 15 | Mean | sd |
| Cₘₐₓ (ng/mL) | 17 | 5.9 | 9.9 | 14 | 16 | 13 | 4.6 |
| Tₘₐₓ (h) | 1.0 | 2.0 | 2.0 | 1.0 | 2.0 | 1.6 | 0.6 |
| AUC (ng.h/mL) | 48 | 28 | 42 | 52 | 57 | 45 | 11 |
| t½ (h) | 1.3 | 2.1 | 2.1 | 1.7 | 2.0 | 1.9 | 0.4 |
| T_{>50%Cmax} (h) | 2.6 | 3.9 | 3.4 | 2.9 | 2.4 | 3.0 | 0.6 |
| Fᵣₑₗ (%) | 247% | 199% | 221% | 270% | 428% | 273% | 91% |

TABLE 61
Pharmacokinetics of dextrorphan PABA carbamate after oral
dosing of 5 mg dextrorphan equivs/kg of the p-aminobenzoic
acid carbamate prodrug to male cynomolgus monkeys

| Pharmacokinetic | | | | | | | |
|---|---|---|---|---|---|---|---|
| parameter | 11 | 12 | 13 | 14 | 15 | Mean | sd |
| Cₘₐₓ (ng/mL) | 301 | 89 | 111 | 273 | 202 | 195 | 94 |
| Tₘₐₓ (h) | 1.0 | 2.0 | 2.0 | 1.0 | 1.0 | 1.4 | 0.5 |
| AUC (ng · h/mL) | 502 | 432 | 420 | 571 | 531 | 491 | 65 |
| t½ (h) | 1.4 | 7.8 | 6.3 | 2.2 | 2.1 | 4.0 | 2.9 |

### Example 48

### Comparative Bioavailability of Dextrorphan after Oral Administration of Dextrorphan or Dextrorphan PABA Carbamate Prodrug to Dogs

### Methodology

Test substances, dextrorphan or dextrorphan PABA carbamate were orally administered to five female beagle dogs in at 1.0 mg dextrorphan free base equivalents/kg for the former & 5 mg dextrorphan free base equivalents/kg for the latter, in a crossover study design.

Blood samples were taken at various times after administration, and submitted to analysis for the parent drug and prodrug using a validated LC-MS-MS assay. Pharmacokinetic parameters derived from the plasma analytical data were determined using Win Nonlin. Data were normalized to 1 mg/kg

### Results

These are shown in Tables 62-64 and reveal a mean 1.7-fold increase in bioavailability of the drug from the prodrug in comparison to that seen after giving the drug itself. There was also some prolongation of sustainment of plasma drug levels with the T>50% Cmax increased from 2.4 h to 3.9 h

TABLE 62
Pharmacokinetics of dextrorphan after oral dosing of 5 mg
dextrorphan free base equivalents/kg to female dogs

| Pharmacokinetic | | | | | | | |
|---|---|---|---|---|---|---|---|
| parameter | 19 | 20 | 21 | 24 | 27 | Mean | sd |
| Cₘₐₓ (ng/mL) | 3.6 | 4.2 | 2.1 | 3.8 | 2.7 | 3.3 | 0.8 |
| Tₘₐₓ (h) | 0.5 | 0.5 | 0.5 | 0.5 | 1.0 | 0.6 | 0.2 |
| AUC (ng · h/mL) | 18.9 | 13.4 | 8.3 | 10.7 | 12.4 | 12.7 | 3.9 |
| t½ (h) | 4.2 | 1.9 | 3.0 | 3.4 | 3.1 | 3.1 | 0.8 |
| T_{>50%Cmax} (h) | 2.9 | 2.4 | 2.2 | 1.6 | 2.9 | 2.4 | 0.5 |

TABLE 63
Pharmacokinetics of dextrorphan after oral dosing of 1 mg
dextorphan equivs/kg of the p-aminobenzoic acid
carbamate prodrug to male dogs

| Pharmacokinetic | | | | | | | |
|---|---|---|---|---|---|---|---|
| parameter | 19 | 20 | 21 | 24 | 27 | Mean | sd |
| Cₘₐₓ (ng/mL) | 0.5 | 1.3 | 0.8 | 0.5 | 1.4 | 0.9 | 0.4 |
| Tₘₐₓ (h) | 3.0 | 2.0 | 2.0 | 1.0 | 2.0 | 2.0 | 0.7 |
| AUC (ng.h/mL) | 2.5 | 5.3 | 3.4 | 2.7 | 5.7 | 3.9 | 1.5 |
| t½ (h) | 2.6 | 1.4 | 1.8 | 2.0 | 1.4 | 1.8 | 0.5 |
| T_{>50%Cmax} (h) | 4.5 | 3.6 | 3.7 | 4.3 | 3.6 | 3.9 | 0.4 |
| Fᵣₑₗ (%)* | 66 | 198 | 204 | 128 | 229 | 165 | 67 |

*Normalised to 5 mg/kg

TABLE 64
Pharmacokinetics of dextrorphan PABA carbamate after oral dosing
of 5 mg dextorphan equivs/kg of the p-aminobenzoic acid carbamate
prodrug to male dogs (normalised to 1 mg/kg)

| Pharmacokinetic | | | | | | | |
|---|---|---|---|---|---|---|---|
| parameter | 19 | 20 | 21 | 24 | 27 | Mean | sd |
| Cₘₐₓ (ng/mL) | 6.1 | 21 | 14 | 10 | 23 | 15 | 6.9 |
| Tₘₐₓ (h) | 0.5 | 1.0 | 1.0 | 1.0 | 1.0 | 0.9 | 0.2 |
| AUC (ng · h/mL) | 19 | 42 | 29 | 21 | 49 | 32 | 13 |
| t½ (h) | 1.9 | 1.0 | 1.1 | 1.1 | 0.9 | 1.2 | 0.4 |

### Example 49

### Comparative Bioavailability of Naloxone after Oral Administration of Naloxone or Naloxone PABA Carbamate to Rats

### Methodology

Test substances (naloxone at 10 mg naloxone free base equivalents/kg or naloxone para amino benzoic acid carbamate at 10 mg naloxone free base equivalents/kg) were administered by oral gavage to groups of rats in a parallel group design.

Blood samples were taken at various times after administration and submitted to analysis for the parent drug and pro-drug using a validated LC-MS-MS assay. Pharmacokinetic parameters derived from the plasma analytical data were determined using Win Nonlin.

### Results

The results presented in Tables 65-67 and show a mean ˜3-fold increase in bioavailability of the drug from the prodrug in comparison to that seen after giving the drug itself. As a consequence, the variability around this parameter dropped from over 10% to just over 5% which if translated to man should result in a more consistent clinical response.

TABLE 65
Pharmacokinetics of naloxone after oral dosing of 10 mg
naloxone free base equivalents/kg to male rats

| Pharmacokinetic | | | | | | | |
|---|---|---|---|---|---|---|---|
| parameter | 1 | 2 | 3 | 4 | 5 | Mean | sd |
| Cₘₐₓ (ng/mL) | 7.1 | 6.3 | 7.6 | 8.1 | 4.9 | 6.8 | 1.3 |
| Tₘₐₓ (h) | 0.25 | 0.25 | 0.50 | 0.50 | 0.25 | 0.35 | 0.14 |
| AUC (ng · h/mL) | 22 | 20 | 25 | 24 | 22 | 22 | 2.3 |
| t½ (h) | — | — | 3.9 | — | 3.8 | 3.9 | — |
| T_{>50%Cmax} (h) | 1.2 | 1.5 | 1.8 | 1.3 | 4.5 | 2.1 | 1.4 |

TABLE 66
Pharmacokinetics of naloxone after oral dosing of 10 mg
naloxone equivs/kg of the p-aminobenzoic acid
carbamate prodrug to male rats

| Pharmacokinetic | | | | | | | |
|---|---|---|---|---|---|---|---|
| parameter | 6 | 7 | 8 | 9 | 10 | Mean | sd |
| Cₘₐₓ (ng/mL) | 35 | 42 | 25 | 36 | 28 | 33 | 6.6 |
| Tₘₐₓ (h) | 0.25 | 0.25 | 0.25 | 0.50 | 0.50 | 0.35 | 0.14 |
| AUC (ng · h/mL) | 67 | 72 | 65 | 68 | 62 | 67 | 3.8 |
| t½ (h) | 1.5 | 2.3 | 3.3 | 2.3 | 3.2 | 2.5 | 0.7 |
| T_{>50%Cmax} (h) | 1.2 | 1.1 | 1.1 | 1.2 | 1.2 | 1.2 | −.1 |
| Fᵣₑₗ (%) | 304 | 326 | 295 | 309 | 279 | 303 | 17 |

TABLE 67
Pharmacokinetics of naloxone PABA carbamate after oral
dosing of 10 mg naloxone equivs/kg of the p-aminobenzoic
acid carbamate prodrug to male rats

| Pharmacokinetic | | | | | | | |
|---|---|---|---|---|---|---|---|
| parameter | 6 | 7 | 8 | 9 | 10 | Mean | sd |
| Cₘₐₓ (ng/mL) | 14 | 16 | 8.4 | 17 | 11 | 13 | 3.6 |
| Tₘₐₓ (h) | 0.25 | 0.25 | 0.25 | 0.25 | 0.25 | 0.25 | 0 |
| AUC (ng · h/mL) | 7.5 | 7.9 | 5.3 | 9.8 | 5.7 | 7.3 | 1.8 |
| t½ (h) | | | | | | | |

NB All AUC values are AUCₗₐₛₜ

Patents, patent applications, publications, product descriptions, and protocols which are cited throughout this application are incorporated herein by reference in their entireties.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. An opioid prodrug having a structure according to Formula (II): 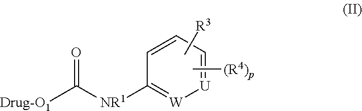
or a pharmaceutically acceptable salt thereof, wherein:
the term “Drug-O₁” is an opioid drug having a phenolic hydroxyl residue and O₁ is said phenolic hydroxyl residue of the opioid;
R³ is selected from the group consisting of: —(CR′R″)ᵣCOOH and 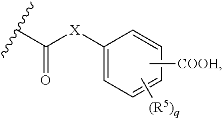
wherein X is —O— or —NR⁶— and wherein R′ and R″ are each independently selected from the group consisting of: H, hydroxy, carboxy, carboxamido, imino, alkanoyl, cyano, cyanomethyl, nitro, amino, halogen, C₁₋₆ alkyl, C₁₋₆ haloalkyl, C₁₋₆ alkoxy, C₁₋₆ haloalkoxy, C₃₋₆ cycloalkyl, aryl, aryl-C₁₋₆ alkyl and C₁₋₆ alkyl aryl;
R¹ and R⁶ are each independently selected from the group consisting of: H, C₁₋₄ alkyl, C₁₋₄ haloalkyl, C₁₋₄ alkoxy and C₁₋₄ haloalkoxy;
R⁴ and R⁵ are each independently selected from the group consisting of: hydroxy, carboxy, carboxamido, imino, alkanoyl, cyano, cyanomethyl, nitro, amino, halogen, C₁₋₆ alkyl, C₁₋₆ haloalkyl, C₁₋₆ alkoxy, C₁₋₆ haloalkoxy, C₃₋₆ cycloalkyl, aryl, aryl-C₁₋₆ alkyl and C₁₋₆ alkyl aryl;
W and U are each independently selected from the group consisting of: —CR′═ and —N═;
p is 0, 1 or 2;
q is 0, 1 or 2; and
r is 0, 1 or 2;
wherein each moiety R′ is independently selected.

2. The prodrug of claim 1 wherein the opioid drug is selected from the group consisting of:
hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine, alvimopan, de-glycinated alvimopan, naloxone, N-methyl naloxone, nalorphine, naltrexone, N-methyl naltrexone and a phenolically hydroxylated phenazepine analgesic.

3. The prodrug of claim 1 wherein R¹ is selected from the group consisting of: H and C₁₋₄ alkyl.

4. The prodrug of claim 1 wherein R³ is —(CR′R″)ᵣCOOH.

5. The prodrug of claim 4 wherein r is 0.

6. The prodrug of claim 4 wherein r is 1 or 2.

7. The prodrug of claim 6 wherein R′ and R″ are each H.

8. The prodrug of claim 1 wherein R⁴ is selected from the group comprising: halogen, C₁₋₆ alkyl, C₁₋₆ haloalkyl, C₁₋₆ alkoxy and C₁₋₆ haloalkoxy.

9. The prodrug of claim 1 wherein R³ is

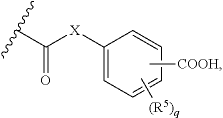

10. The prodrug of claim 9 wherein X is —O—.

11. The prodrug of claim 9 wherein X is —NR⁶— and further wherein R⁶ is selected from the group consisting of: H and C₁₋₄ alkyl.

12. The prodrug of claim 11 wherein R⁶ is H.

13. The prodrug of claim 9 wherein q is 0.

14. The prodrug of claims 9 to 12 wherein R⁵ is selected from the group comprising: halogen, C₁₋₆ alkyl, C₁₋₆ haloalkyl, C₁₋₆ alkoxy and C₁₋₆ haloalkoxy.

15. The prodrug of claim 9 wherein q is 1.

16. The prodrug of claim 1 wherein W is —CR′═.

17. The prodrug of claim 1 wherein W is —N═.

18. The prodrug of claim 1 wherein U is —CR′═.

19. The prodrug of claim 1 wherein p is 0.

20. The prodrug of claim 1 wherein p is 1.

21. The prodrug of claim 1 wherein the opioid prodrug moiety is selected from group comprising:

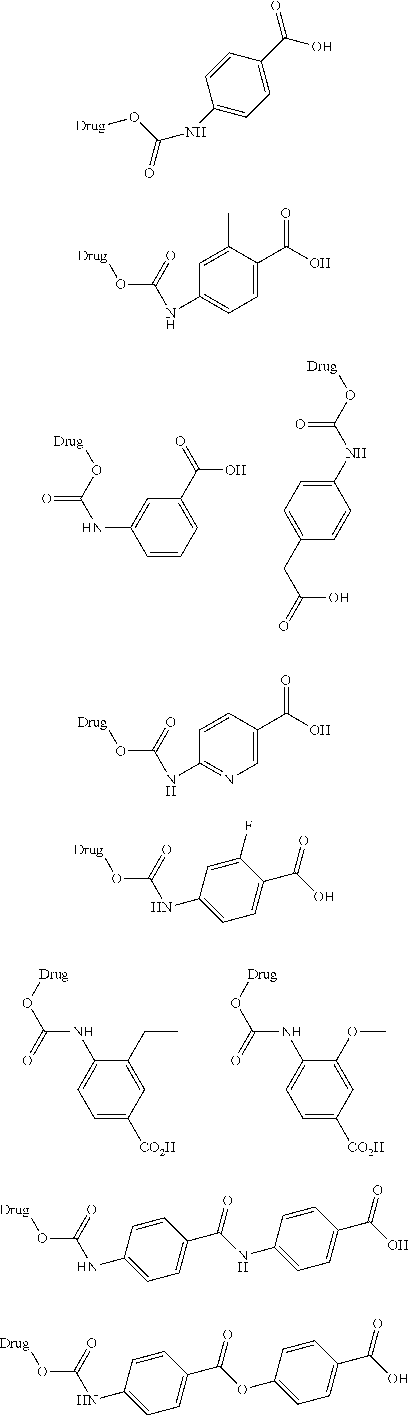

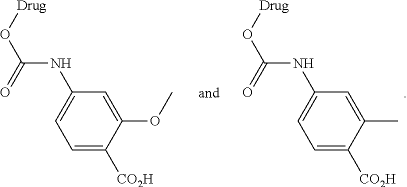

22. A pharmaceutical composition comprising a compound of claim 1 and a pharmaceutically acceptable excipient.

23. A method of treating a disorder treatable by an opioid, the method comprising orally administering to a subject suffering from such a disorder a therapeutically effective amount of an opioid prodrug of claim 1 or a pharmaceutically acceptable salt thereof.

24. The method of claim 23 wherein the disorder is pain.

25. The method of claim 24 wherein the pain is acute pain, chronic pain, post-operative pain, pain due to neuralgia (optionally post herpetic neuralgia or trigeminal neuralgia), pain due to diabetic neuropathy, dental pain, pain associated with arthritis or osteoarthritis, or pain associated with cancer or its treatment.

26. The method of claim 25 wherein the pain is neuropathic pain or nociceptive pain.

27. The prodrug of claim 2 wherein the phenolically hydroxylated phenazepine analgesic is a 2-, 3- or 4-phenolically hydroxylated phenazepine analgesic.

28. The prodrug of claim 27 wherein the 2-, 3- or 4-phenolically hydroxylated phenazepine analgesic is a 2-, 3- or 4-phenolically hydroxylated ethoheptazine, proheptazine, metethoheptazine or metheptazine.

29. The prodrug of claim 16 wherein W—CH═.

30. The prodrug of claim 18 wherein U is —CH═.
